(19) United States
(12) Patent Application Publication
(10) Pub. No.: US 2022/0255010 A1
(43) Pub. Date: Aug. 11, 2022

(54) LIGHT EMITTING DEVICE AND AMINE COMPOUND FOR LIGHT EMITTING DEVICE

(71) Applicant: Samsung Display Co., Ltd., Yongin-si (KR)

(72) Inventors: HANKYU PAK, Suwon-si (KR); EUNJAE JEONG, Hwaseong-si (KR); SANGHYUN HAN, Hwaseong-si (KR); DONGJUN KIM, Suwon-si (KR); MINJI KIM, Hwaseong-si (KR); SOHEE JO, Cheonan-si (KR); YOUNGEUN CHOI, Jeonju-si (KR)

(21) Appl. No.: 17/453,809

(22) Filed: Nov. 5, 2021

(30) Foreign Application Priority Data
Feb. 4, 2021 (KR) 10-2021-0016148

(51) Int. Cl. H01L 51/00; C07D 209/88; C09K 11/06

(52) U.S. Cl. CPC H01L 51/0061; C07D 209/88; H01L 51/5088; H01L 51/006; C09K 11/06

(57) ABSTRACT

A light emitting device includes a first electrode, a second electrode facing the first electrode, and an organic layer between the first electrode and the second electrode, and the organic layer includes an amine compound represented by Formula 1. In Formula 1, the substituents are the same as respectively defined in the Detailed Description. The light emitting device may exhibit improved luminous efficiency and long service life characteristics.

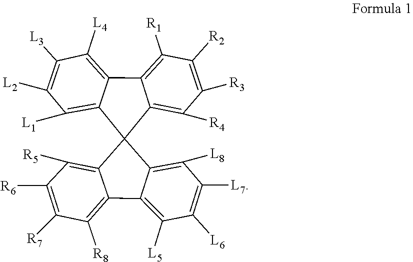

### CROSS-REFERENCE TO RELATED APPLICATION

This application claims priority to and the benefit of Korean Patent Application No. 10-2021-0016148, filed on Feb. 4, 2021, the entire content of which is hereby incorporated by reference.

### BACKGROUND

### 1. Field

The subject matter of the present disclosure herein relates to a light emitting device and an amine compound utilized therein, and, for example, to an amine compound utilized as a hole transport material and a light emitting device including the same.

### 2. Description of the Related Art

Recently, the development of an organic electroluminescence display apparatus as an image display apparatus is being actively conducted. Unlike liquid crystal display apparatuses and/or the like, the organic electroluminescence display apparatus is a so-called self-luminescent display apparatus in which holes and electrons injected from a first electrode and a second electrode recombine in an emission layer, and thus a luminescent material including an organic compound in the emission layer emits light to implement display (e.g., to display an image).

In the application of an organic electroluminescence device to a display apparatus, there is a demand for an organic electroluminescence device having a low driving voltage, high luminous efficiency, and a long service life, and the development on materials, for an organic electroluminescence device, capable of stably attaining such characteristics is being continuously conducted.

In addition, materials of a hole transport layer are being developed in order to realize a highly efficient organic electroluminescence device.

### SUMMARY

An aspect according to embodiments of the present disclosure is directed toward a light emitting device in which luminous efficiency and a device service life are improved.

An aspect according to embodiments of the present disclosure is also directed toward an amine compound capable of improving luminous efficiency and the device service life of a light emitting device.

According to an embodiment of the present disclosure, a light emitting device includes a first electrode, a second electrode facing the first electrode, and an organic layer between the first electrode and the second electrode, wherein the organic layer includes an amine compound represented by Formula 1 below:

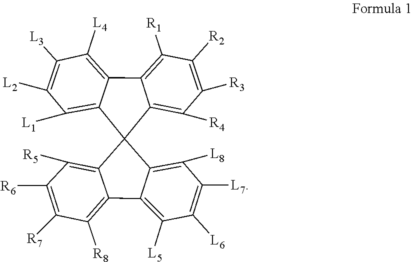

In Formula 1 above, R₁ to R₈ are each independently a hydrogen atom, a deuterium atom, a halogen atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms; L₁ to L₈ are each independently a hydrogen atom, a deuterium atom, a halogen atom, a substituted or unsubstituted amine group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms; at least one among L₁ to L₄ is a group represented by Formula 2 below, and at least one among L₅ to L₈ is a group represented by Formula 3 below:

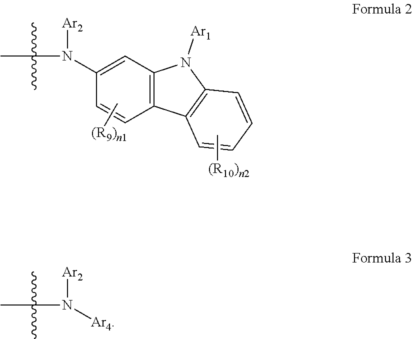

In Formula 2 and Formula 3 above, R₉ and R₁₀ are each independently a hydrogen atom, a deuterium atom, a halogen atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms; Ar₁ to Ar₄ are each independently a substituted or unsubstituted aryl group having 6 to 30 ring-forming carbon atoms, or a substituted or unsubstituted heteroaryl group having 2 to 30 ring-forming carbon atoms; n1 is an integer of 0 to 3, and n2 is an integer of 0 to 4.

In an embodiment, the organic layer may include a hole transport region on the first electrode, an emission layer on the hole transport region, and an electron transport region on the emission layer, and the hole transport region may include the amine compound represented by Formula 1 above.

In an embodiment, the hole transport region may include a hole injection layer on the first electrode, and a hole transport layer on the hole injection layer, and the hole injection layer or the hole transport layer may include the amine compound represented by Formula 1 above.

In an embodiment, the hole transport region may include the hole transport layer on the first electrode and an electron blocking layer on the hole transport layer, and the electron blocking layer may include the amine compound represented by Formula 1 above.

In an embodiment, the amine compound represented by Formula 1 above may be a diamine compound.

In an embodiment, the amine compound represented by Formula 1 above may be represented by any one among Formula 4-1 to Formula 4-4 below:

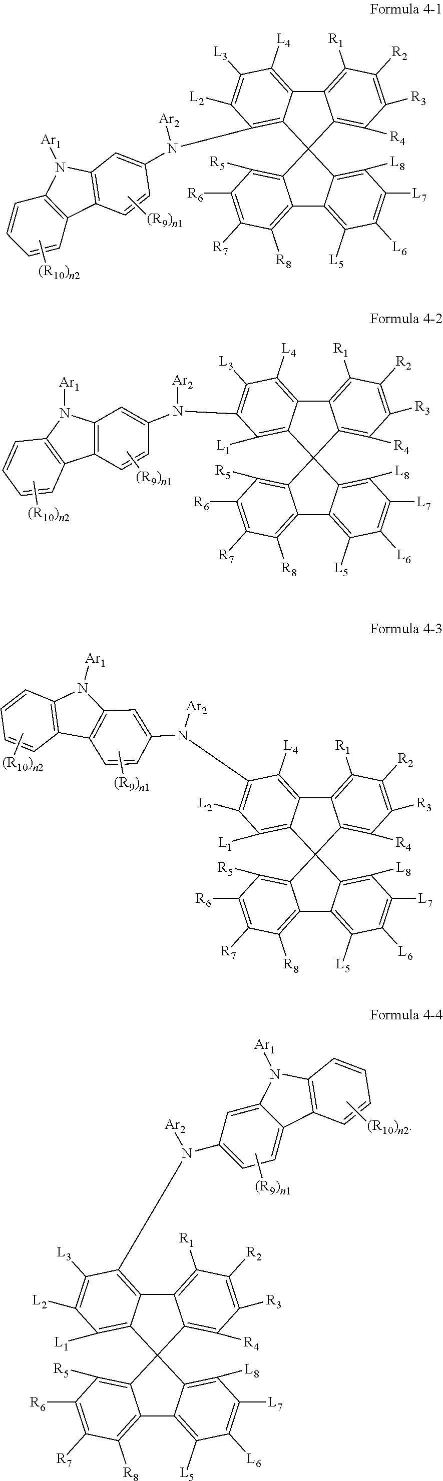

In Formula 4-1 to Formula 4-4 above, R₁ to R₁₀, L₁ to L₈, Ar₁, Ar₂, n1 and n2 are the same as respectively defined in connection with Formula 1 and Formula 2.

In an embodiment, the amine compound represented by Formula 1 above may be represented by any one among Formula 5-1 to Formula 5-4 below:

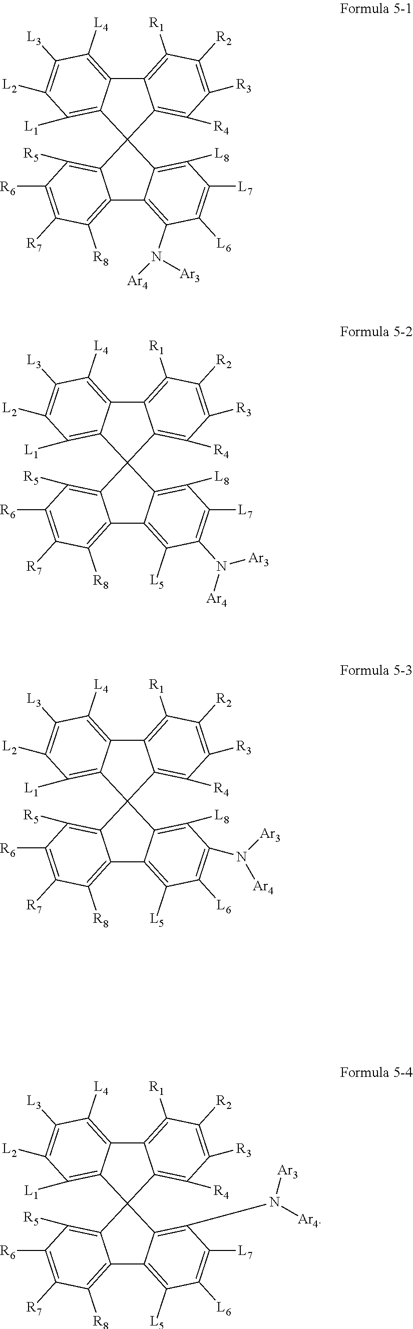

In Formula 5-1 to Formula 5-4 above, R₁ to R₈, L₁ to L₈, Ar₃, and Ar₄ are the same as respectively defined in connection with Formula 1 and Formula 3.

In an embodiment, R₁ to R₁₀ above may be each independently a hydrogen atom or a deuterium atom.

In an embodiment, Ar₁ to Ar₄ above may be each independently a substituted or unsubstituted phenyl group, a substituted or unsubstituted naphthyl group, or a substituted or unsubstituted biphenyl group.

In an embodiment, the group represented by Formula 2 above may be represented by Formula 2-1 below:

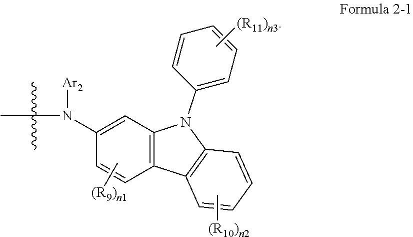

In Formula 2-1 above, Ru is a hydrogen atom, a deuterium atom, a halogen atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms; n3 is an integer of 0 to 5, and R₉, R₁₀, Ar₂, n1, and n2 are the same as respectively defined in connection with Formula 2 above.

In an embodiment, Ar₁ to Ar₄ above may be each independently represented by any one among Formula 6-1 to Formula 6-6 below:

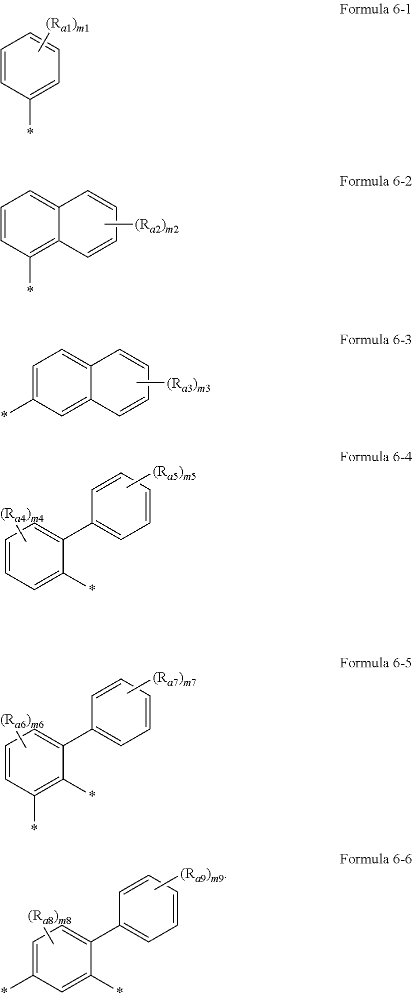

In Formula 6-1 to Formula 6-6 above, Rₐ₁ to Rₐ₉ are each independently a hydrogen atom, a deuterium atom, a halogen atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms; m1, m5, m7, and m9 are each independently an integer of 0 to 5, m2 and m3 are each independently an integer of 0 to 7, and m4, m6, and m8 are each independently an integer of 0 to 4.

In an embodiment, the amine compound represented by Formula 1 above may be any one among the compounds represented by Compound Group 1.

### BRIEF DESCRIPTION OF THE FIGURES

The accompanying drawings are included to provide a further understanding of the subject matter of the present disclosure, and are incorporated in and constitute a part of this specification. The drawings illustrate example embodiments of the present disclosure and, together with the description, serve to explain principles of the present disclosure. In the drawings:

FIG. 1 is a plan view illustrating a display apparatus according to an embodiment of the present disclosure;

FIG. 2 is a cross-sectional view illustrating a display apparatus according to an embodiment of the present disclosure;

FIG. 3 is a cross-sectional view schematically illustrating a light emitting device according to an embodiment of the present disclosure;

FIG. 4 is a cross-sectional view schematically illustrating a light emitting device according to an embodiment of the present disclosure;

FIG. 5 is a cross-sectional view schematically illustrating a light emitting device according to an embodiment of the present disclosure;

FIG. 6 is a cross-sectional view schematically illustrating a light emitting device according to an embodiment of the present disclosure;

FIG. 7 is a cross-sectional view illustrating a display apparatus according to an embodiment of the present disclosure; and

FIG. 8 is a cross-sectional view illustrating a display apparatus according to an embodiment of the present disclosure.

### DETAILED DESCRIPTION

The subject matter of the present disclosure may be modified in many alternate forms, and thus specific embodiments will be illustrated in the drawings and described in more detail. It should be understood, however, that it is not intended to limit the present disclosure to the particular forms disclosed, but rather, is intended to cover all modifications, equivalents, and alternatives falling within the spirit and scope of the present disclosure.

When explaining each of the drawings, like reference numbers are used for referring to like elements. In the accompanying drawings, the dimensions of each structure may be exaggerated for clarity. It will be understood that, although the terms “first,” “second,” etc. may be used herein to describe various elements, these elements should not be limited by these terms. These terms are only used to distinguish one element from another. For example, a first element may be referred to as a second element, and, similarly, the second element may be referred to as the first element, without departing from the scope of the present disclosure. The singular forms are intended to include the plural forms unless the context clearly indicates otherwise.

In the present application, it will be understood that the terms “include,” “have,” etc., specify the presence of a feature, a fixed number, a step, an operation, an element, a component, or a combination thereof disclosed in the specification, but do not exclude the possibility of presence or addition of one or more other features, fixed numbers, steps, operations, elements, components, or combination thereof.

In the present application, when a part such as a layer, a film, a region, and/or a plate is referred to as being “on” or “above” another part, it can be directly on the other part, or an intervening part may also be present. On the contrary, when a part such as a layer, a film, a region, and/or a plate is referred to as being “under” or “below” another part, it can be directly under the other part, or an intervening part may also be present. In addition, it will be understood that when a part is referred to as being “on” another part, it can be on the other part, or under the other part as well.

Hereinafter, embodiments of the present disclosure will be described with reference to the accompanying drawings.

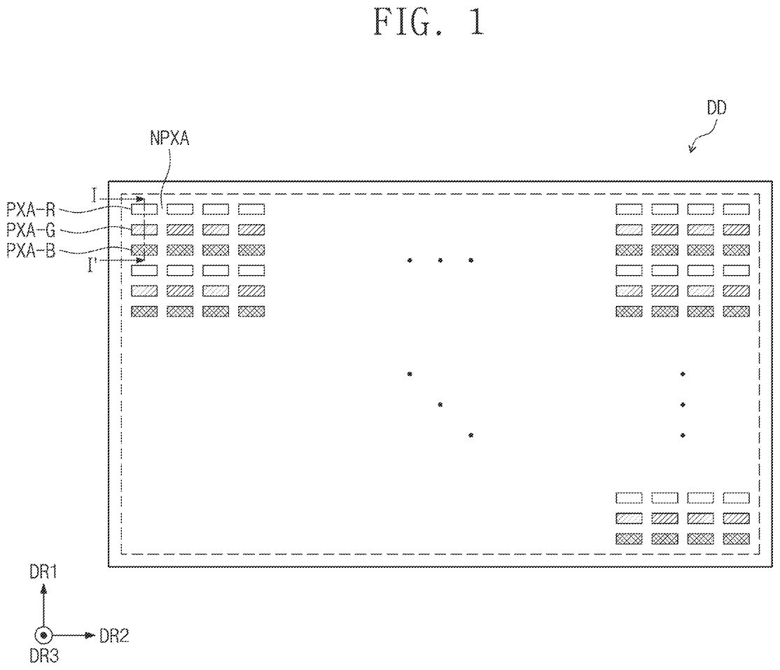

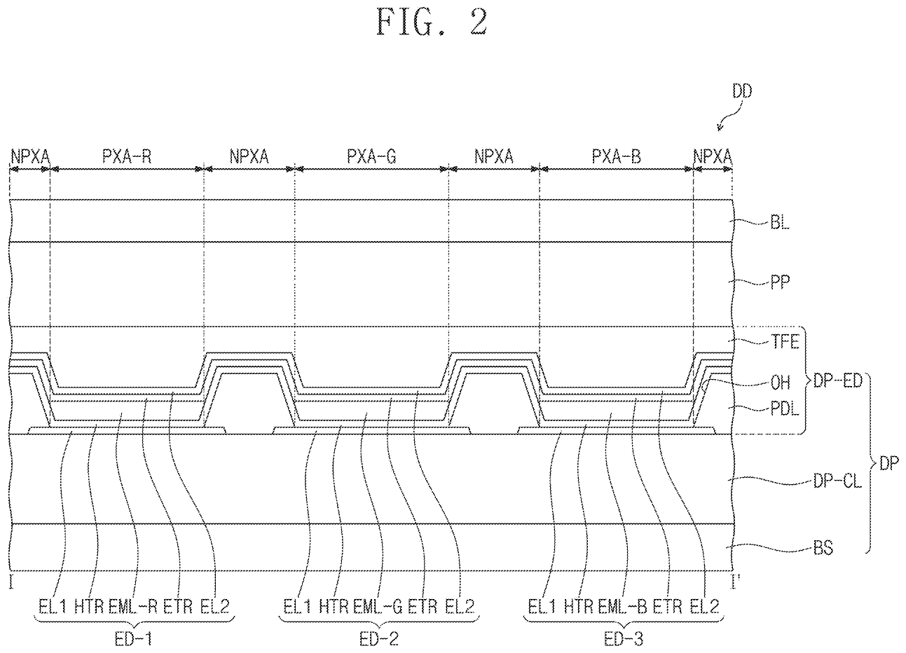

FIG. 1 is a plan view illustrating an embodiment of a display apparatus DD. FIG. 2 is a cross-sectional view of the display apparatus DD of the embodiment. FIG. 2 is a cross-sectional view illustrating a part taken along the line I-I′ of FIG. 1.

The display apparatus DD may include a display panel DP and an optical layer PP on the display panel DP. The display panel DP includes light emitting devices ED-1, ED-2, and ED-3. The display apparatus DD may include a plurality of light emitting devices ED-1, ED-2, and ED-3. The optical layer PP may be on the display panel DP and control reflected light in the display panel DP due to external light. The optical layer PP may include, for example, a polarization layer and/or a color filter layer. In one or more embodiments, unlike the view illustrated in the drawing, the optical layer PP may be omitted from the display apparatus DD.

A base substrate BL may be on the optical layer PP. The base substrate BL may be a member which provides a base surface on which the optical layer PP is located. The base substrate BL may be a glass substrate, a metal substrate, a plastic substrate, etc. However, the embodiment of the present disclosure is not limited thereto, and the base substrate BL may be an inorganic layer, an organic layer, or a composite material layer. In addition, unlike what is illustrated in the drawings, in an embodiment, the base substrate BL may be omitted.

The display apparatus DD according to an embodiment may further include a filling layer. The filling layer may be between a display device layer DP-ED and the base substrate BL. The filling layer may be an organic material layer. The filling layer may include at least one of an acrylic-based resin, a silicone-based resin, or an epoxy-based resin.

The display panel DP may include a base layer BS, a circuit layer DP-CL provided on the base layer BS, and a display device layer DP-ED. The display device layer DP-ED may include a pixel defining film PDL, the light emitting devices ED-1, ED-2, and ED-3 between portions of the pixel defining film PDL, and an encapsulation layer TFE on the light emitting devices ED-1, ED-2, and ED-3.

The base layer BS may be a member which provides a base surface on which the display device layer DP-ED is located. The base layer BS may be a glass substrate, a metal substrate, a plastic substrate, etc. However, the embodiment of the present disclosure is not limited thereto, and the base layer BS may be an inorganic layer, an organic layer, or a composite material layer.

In an embodiment, the circuit layer DP-CL is on the base layer BS, and the circuit layer DP-CL may include a plurality of transistors. Each of the transistors may include a control electrode, an input electrode, and an output electrode. For example, the circuit layer DP-CL may include a switching transistor and a driving transistor in order to drive the light emitting devices ED-1, ED-2, and ED-3 of the display device layer DP-ED.

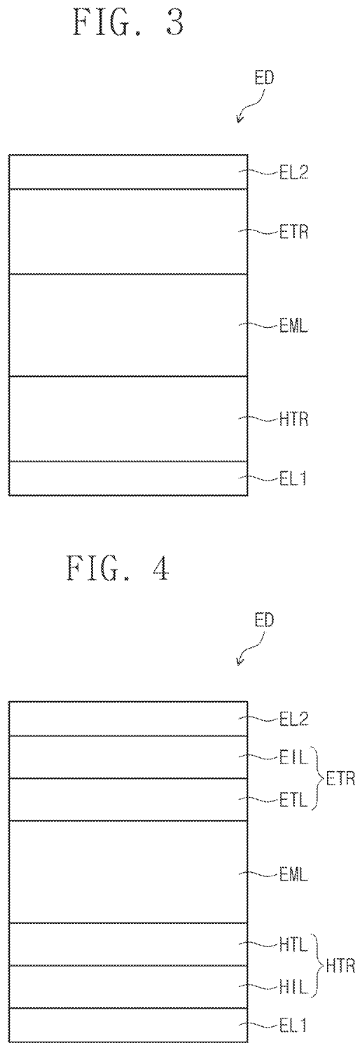

Each of the light emitting devices ED-1, ED-2, and ED-3 may have a structure of a light emitting device ED of an embodiment according to FIGS. 3-6, which will be described in more detail herein below. Each of the light emitting devices ED-1, ED-2 and ED-3 may include a first electrode EL1, a hole transport region HTR, emission layers EML-R, EML-G and EML-B, an electron transport region ETR, and a second electrode EL2.

FIG. 2 illustrates an embodiment in which the emission layers EML-R, EML-G, and EML-B of the light emitting devices ED-1, ED-2, and ED-3 are in the openings OH defined in the pixel defining film PDL, and the hole transport region HTR, the electron transport region ETR, and the second electrode EL2 are provided as a common layer in the entire light emitting devices ED-1, ED-2, and ED-3. However, the embodiment of the present disclosure is not limited thereto, and unlike the feature illustrated in FIG. 2, the hole transport region HTR and the electron transport region ETR in an embodiment may be provided by being patterned inside the opening hole OH defined in the pixel defining film PDL. For example, the hole transport region HTR, the emission layers EML-R, EML-G, and EML-B, and the electron transport region ETR of the light emitting devices ED-1, ED-2, and ED-3 in an embodiment may be provided by being patterned in an inkjet printing method.

The encapsulation layer TFE may cover the light emitting devices ED-1, ED-2 and ED-3. The encapsulation layer TFE may seal the display device layer DP-ED. The encapsulation layer TFE may be a thin film encapsulation layer. The encapsulation layer TFE may be formed by laminating one layer or a plurality of layers. The encapsulation layer TFE includes at least one insulation layer. The encapsulation layer TFE according to an embodiment may include at least one inorganic film (hereinafter, an encapsulation-inorganic film). The encapsulation layer TFE according to an embodiment may also include at least one organic film (hereinafter, an encapsulation-organic film) and at least one encapsulation-inorganic film.

The encapsulation-inorganic film protects the display device layer DP-ED from moisture/oxygen, and the encapsulation-organic film protects the display device layer DP-ED from foreign substances such as dust particles. The encapsulation-inorganic film may include silicon nitride, silicon oxynitride, silicon oxide, titanium oxide, aluminum oxide, and/or the like, but the embodiment of the present disclosure is not particularly limited thereto. The encapsulation-organic film may include an acrylic-based compound, an epoxy-based compound, and/or the like. The encapsulation-organic film may include a photopolymerizable organic material, but the embodiment of the present disclosure is not particularly limited thereto.

The encapsulation layer TFE may be on the second electrode EL2 and may fill the opening hole OH.

Referring to FIGS. 1 and 2, the display apparatus DD may include a non-light emitting region NPXA and light emitting regions PXA-R, PXA-G and PXA-B. The light emitting regions PXA-R, PXA-G and PXA-B each may be a region which emits light generated from the light emitting devices ED-1, ED-2 and ED-3, respectively. The light emitting regions PXA-R, PXA-G, and PXA-B may be spaced apart from each other in a plan view (e.g., on a plane).

Each of the light emitting regions PXA-R, PXA-G, and PXA-B may be a region divided by the pixel defining film PDL. The non-light emitting regions NPXA may be regions between the adjacent light emitting regions PXA-R, PXA-G, and PXA-B, which correspond to portions of the pixel defining film PDL. In one or more embodiments, in the specification, each of the light emitting regions PXA-R, PXA-G, and PXA-B may correspond to a pixel. The pixel defining film PDL may separate the light emitting elements ED-1, ED-2, and ED-3. The emission layers EML-R, EML-G and EML-B of the light emitting devices ED-1, ED-2 and ED-3 may be in openings OH defined by the pixel defining film PDL and separated from each other.

The light emitting regions PXA-R, PXA-G and PXA-B may be divided into a plurality of groups according to the color of light generated from the light emitting devices ED-1, ED-2 and ED-3. In the display apparatus DD of an embodiment shown in FIGS. 1 and 2, three light emitting regions PXA-R, PXA-G, and PXA-B which emit red light, green light, and blue light, respectively, are illustrated as an example. For example, the display apparatus DD of an embodiment may include the red light emitting region PXA-R, the green light emitting region PXA-G, and the blue light emitting region PXA-B which are separated from one another.

In the display apparatus DD according to an embodiment, the plurality of light emitting devices ED-1, ED-2 and ED-3 may emit light having wavelengths different from one another. For example, in an embodiment, the display apparatus DD may include a first light emitting device ED-1 that emits red light, a second light emitting device ED-2 that emits green light, and a third light emitting device ED-3 that emits blue light. That is, the red light emitting region PXA-R, the green light emitting region PXA-G, and the blue light emitting region PXA-B of the display apparatus DD may correspond to the first light emitting device ED-1, the second light emitting device ED-2, and the third light emitting device ED-3, respectively.

However, the embodiment of the present disclosure is not limited thereto, and the first to third light emitting devices ED-1, ED-2, and ED-3 may emit light in the same wavelength range or at least one light emitting device may emit light in a wavelength range different from the others. For example, the first to third light emitting devices ED-1, ED-2, and ED-3 may all emit blue light.

The light emitting regions PXA-R, PXA-G, and PXA-B in the display apparatus DD according to an embodiment may be arranged in a stripe form. Referring to FIG. 1, the plurality of red light emitting regions PXA-R may be arranged along a second direction DR2, the plurality of green light emitting regions PXA-G may be arranged along the second direction DR2, and the plurality of blue light emitting regions PXA-B may be arranged along the second direction DR2. In addition, the red light emitting region PXA-R, the green light emitting region PXA-G, and the blue light emitting region PXA-B may be alternately arranged in this stated order along a first direction DR1.

FIGS. 1 and 2 illustrate that all the light emitting regions PXA-R, PXA-G, and PXA-B have similar areas, but the embodiment of the present disclosure is not limited thereto, and the light emitting regions PXA-R, PXA-G, and PXA-B may have different areas from each other according to a wavelength range of the emitted light. In this case, the areas of the light emitting regions PXA-R, PXA-G, and PXA-B may refer to areas in a plan view (e.g., when viewed in or on a plane defined by the first direction DR1 and the second direction DR2).

In one or more embodiments, the arrangement form of the light emitting regions PXA-R, PXA-G, and PXA-B is not limited to the feature illustrated in FIG. 1, and the order in which the red light emitting region PXA-R, the green light emitting region PXA-G, and the blue light emitting region PXA-B are arranged may be variously combined and provided according to characteristics of a display quality required in the display apparatus DD. For example, the arrangement form of the light emitting regions PXA-R, PXA-G, and PXA-B may be a PENTILE® arrangement form (e.g., an RGBG matrix, RGBG structure, or RGBG matrix structure), or a diamond arrangement form. PENTILE® is a duly registered trademark of Samsung Display Co., Ltd.

In addition, the areas of the light emitting regions PXA-R, PXA-G, and PXA-B may be different from each other. For example, in an embodiment, the area of the green light emitting region PXA-G may be smaller than that of the blue light emitting region PXA-B, but the embodiment of the present disclosure is not limited thereto.

FIGS. 3-6 are cross-sectional views schematically illustrating light emitting devices according to embodiments of the present disclosure. Referring to FIGS. 3-6, in each of light emitting devices ED of embodiments, a first electrode EU and a second electrode EL2 face each other, and a plurality of organic layers may be between the first electrode EL1 and the second electrode EL2. The plurality of organic layers may include a hole transport region HTR, an emission layer EML, and an electron transport region ETR. That is, each of the light emitting devices ED according to embodiments may include the first electrode EL1, the hole transport region HTR, the emission layer EML, the electron transport region ETR, and the second electrode EL2 that are sequentially stacked in the stated order. A capping layer CPL may be further on the second electrode EL2.

Each of the light emitting devices ED of embodiments may include an amine compound of an embodiment described below in at least one organic layer among the plurality of organic layers between the first electrode EL1 and the second electrode EL2. For example, each of the light emitting devices ED of embodiments may include an amine compound of an embodiment described below in the hole transfer region HTR between the first electrode EL1 and the second electrode EL2. However, the embodiment of the present disclosure is not limited thereto, and each of the light emitting devices ED of embodiments may include an amine compound according to an embodiment described below in at least one organic layer from among the plurality of organic layers between the first electrode EU and the second electrode EL2, which includes the emission layer EML and the electron transfer region ETR, and/or may include an amine compound according to an embodiment described below in the capping layer CPL on the second electrode EL2.

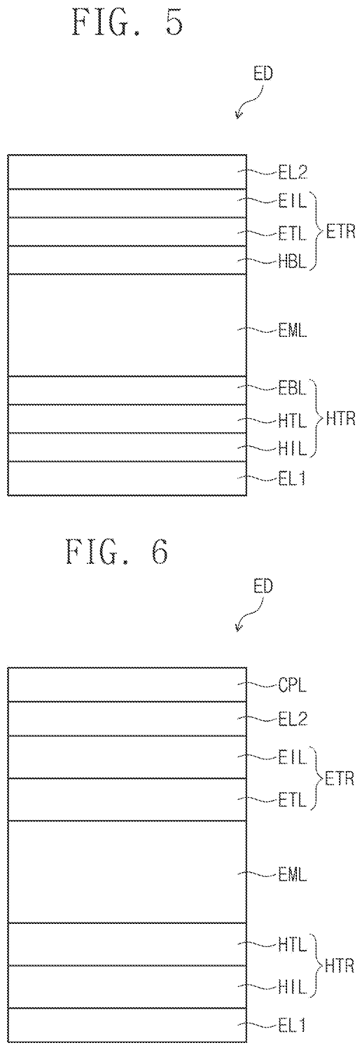

In one or more embodiments, compared to FIG. 3, FIG. 4 illustrates a cross-sectional view of a light emitting device ED of an embodiment, in which a hole transport region HTR includes a hole injection layer HIL and a hole transport layer HTL, and an electron transport region ETR includes an electron injection layer EIL and an electron transport layer ETL. In addition, compared to FIG. 3, FIG. 5 illustrates a cross-sectional view of a light emitting device ED of an embodiment, in which a hole transport region HTR includes a hole injection layer HIL, a hole transport layer HTL, and an electron blocking layer EBL, and an electron transport region ETR includes an electron injection layer EIL, an electron transport layer ETL, and a hole blocking layer HBL. Compared to FIG. 4, FIG. 6 illustrates a cross-sectional view of a light emitting device ED of an embodiment including a capping layer CPL on a second electrode EL2.

Hereinafter, in the description of the light emitting device ED of an embodiment, it is described that the light emitting device ED includes an amine compound according to an embodiment described below in the hole transport region HTR, but the embodiment of the present disclosure is not limited thereto, and an amine compound according to an embodiment described below may be included in the emission layer EML, and/or the electron transport region ETR.

The first electrode EL1 has conductivity (e.g., electrical conductivity). The first electrode EL1 may be formed of a metal material, a metal alloy, or a conductive compound. The first electrode EL1 may be an anode or a cathode. However, the embodiment of the present disclosure is not limited thereto. In addition, the first electrode EL1 may be a pixel electrode. The first electrode EL1 may be a transmissive electrode, a transflective electrode, or a reflective electrode. When the first electrode EL1 is the transmissive electrode, the first electrode EL1 may include a transparent metal oxide such as indium tin oxide (ITO), indium zinc oxide (IZO), zinc oxide (ZnO), and/or indium tin zinc oxide (ITZO). When the first electrode EL1 is the transflective electrode or the reflective electrode, the first electrode EL1 may include Ag, Mg, Cu, Al, Pt, Pd, Au, Ni, Nd, Ir, Cr, Li, Ca, LiF/Ca, LiF/Al, Mo, Ti, W, a compound thereof, or a mixture thereof (e.g., a mixture of Ag and Mg). In one or more embodiments, the first electrode EU may have a multilayer structure including a reflective film or a transflective film formed of the above-described materials, and a transparent conductive film formed of ITO, IZO, ZnO, ITZO, etc. For example, the first electrode EU may have a three-layer structure of ITO/Ag/ITO, but the embodiment of the present disclosure is not limited thereto. In addition, the embodiment of the present disclosure is not limited thereto, and the first electrode EL1 may include the above-described metal materials, combinations of two or more metal materials of the above-described metal materials, oxides of the above-described metal materials, and/or the like. The thickness of the first electrode EU may be from about 700 Å to about 10,000 Å. For example, the thickness of the first electrode EU may be from about 1,000 Å to about 3,000 Å.

The hole transport region HTR is provided on the first electrode EL1. The hole transport region HTR may include at least one of a hole injection layer HIL, a hole transport layer HTL, a buffer layer, an emission-auxiliary layer, or an electron blocking layer EBL. The thickness of the hole transport region HTR may be, for example, from about 50 Å to about 15,000 Å.

The hole transport region HTR may have a single layer formed of a single material, a single layer formed of a plurality of different materials, or a multilayer structure including a plurality of layers formed of a plurality of different materials.

For example, the hole transport region HTR may have a single layer structure of the hole injection layer HIL or the hole transport layer HTL, and may have a single layer structure formed of a hole injection material and a hole transport material. In addition, the hole transport region HTR may have a single layer structure formed of a plurality of different materials, or a structure in which a hole injection layer HIL/hole transport layer HTL, a hole injection layer HIL/hole transport layer HTL/buffer layer, a hole injection layer HIL/buffer layer, a hole transport layer HTL/buffer layer, or a hole injection layer HIL/hole transport layer HTL/electron blocking layer EBL are stacked in the respective stated order from the first electrode EL1, but the embodiment of the present disclosure is not limited thereto.

The hole transport region HTR in the light emitting device ED of an embodiment includes an amine compound according to an embodiment of the present disclosure. The light emitting device ED of an embodiment may include the amine compound according to an embodiment of the present disclosure in at least one of the hole injection layer HIL, the hole transport layer HTL, or the electron blocking layer EBL. The light emitting device ED of an embodiment may include the amine compound according to an embodiment of the present disclosure in a layer, adjacent to the emission layer EML, of the hole transport region HTR.

The amine compound of an embodiment includes a spirobifluorene group as a central core. The amine compound of an embodiment includes two amine groups, and a structure in which the two amine groups are substituted at the spirobifluorene group. The spirobifluorene group may be a substituent including two fluorene groups and having a spiro structure at a spa-carbon, as the center, which is the 9-position carbon of the two fluorene groups. That is, the spirobifluorene group may include a bicyclic structure including two fluorene groups. The amine compound has a structure in which the two amine groups are connected to the two fluorene groups included in the spirobifluorene group, respectively. In addition, one of the two amine groups included in the amine compound includes (e.g., must include) a carbazole group as a substituent. In one or more embodiments, the amine compound may include a structure in which at least one of the two amine groups is connected to the 2-position carbon of the carbazole group.

The amine compound of an embodiment may be a diamine compound. For example, the amine compound may include two amine groups in the compound structure.

In the specification, the term “substituted or unsubstituted” may refer to substituted or unsubstituted with at least one substituent selected from the group consisting of a deuterium atom, a halogen atom, a cyano group, a nitro group, a silyl group, an oxy group, a thio group, a sulfinyl group, a sulfonyl group, a carbonyl group, a boron group, a phosphine oxide group, a phosphine sulfide group, an alkyl group, an alkenyl group, an alkynyl group, an alkoxy group, a hydrocarbon ring group, an aryl group, and a heterocyclic group. In addition, each of the substituents described above may be substituted or unsubstituted. For example, a biphenyl group may be interpreted as an aryl group or a phenyl group substituted with a phenyl group.

In the specification, examples of the halogen atom may include a fluorine atom, a chlorine atom, a bromine atom, or an iodine atom.

In the specification, the alkyl group may be a linear, branched or cyclic alkyl group. The number of carbon atoms in the alkyl group may be 1 to 50, 1 to 30, 1 to 20, 1 to 10, or 1 to 6. Non-limiting examples of the alkyl group may include a methyl group, an ethyl group, an n-propyl group, an isopropyl group, an n-butyl group, a s-butyl group, a t-butyl group, an i-butyl group, a 2-ethylbutyl group, a 3,3-a dimethylbutyl group, an n-pentyl group, an i-pentyl group, a neopentyl group, a t-pentyl group, a cyclopentyl group, a 1-methylpentyl group, a 3-methylpentyl group, a 2-ethylpentyl group, a 4-methyl-2-pentyl group, an n-hexyl group, a 1-methylhexyl group, a 2-ethylhexyl group, a 2-butylhexyl group, a cyclohexyl group, a 4-methylcyclohexyl group, a 4-t-butylcyclohexyl group, an n-heptyl group, a 1-methylheptyl group, a 2,2-dimethylheptyl group, a 2-ethylheptyl group, a 2-butylheptyl group, an n-octyl group, a t-octyl group, a 2-ethyloctyl group, a 2-butyloctyl group, a 2-hexyloctyl group, a 3,7-dimethyloctyl group, a cyclooctyl group, an n-nonyl group, an n-decyl group, an adamantyl group, a 2-ethyldecyl group, a 2-butyldecyl group, a 2-hexyldecyl group, a 2-octyldecyl group, an n-undecyl group, an n-dodecyl group, a 2-ethyldodecyl group, a 2-butyldodecyl group, a 2-hexyldocecyl group, a 2-octyldodecyl group, an n-tridecyl group, an n-tetradecyl group, an n-pentadecyl group, an n-hexadecyl group, a 2-ethylhexadecyl group, a 2-butylhexadecyl group, a 2-hexylhexadecyl group, a 2-octylhexadecyl group, an n-heptadecyl group, an n-octadecyl group, an n-nonadecyl group, an n-eicosyl group, a 2-ethyleicosyl group, a 2-butyleicosyl group, a 2-hexyleicosyl group, a 2-octyleicosyl group, an n-henicosyl group, an n-docosyl group, an n-tricosyl group, an n-tetracosyl group, an n-pentacosyl group, an n-hexacosyl group, an n-heptacosyl group, an n-octacosyl group, an n-nonacosyl group, an n-triacontyl group, etc.

The aryl group herein may refer to any functional group or substituent derived from an aromatic hydrocarbon ring. The aryl group may be a monocyclic aryl group or a polycyclic aryl group. The number of ring-forming carbon atoms in the aryl group may be 6 to 30, 6 to 20, or 6 to 15. Examples of the aryl group may include a phenyl group, a naphthyl group, a fluorenyl group, an anthracenyl group, a phenanthryl group, a biphenyl group, a terphenyl group, a quaterphenyl group, a quinquephenyl group, a sexiphenyl group, a triphenylenyl group, a pyrenyl group, a benzofluoranthenyl group, a chrysenyl group, etc., but the embodiment of the present disclosure is not limited thereto.

In the specification, the fluorenyl group may be substituted, and two substituents may be bonded to each other to form a spiro structure. Examples of a substituted fluorenyl group may be as follows. However, the embodiment of the present disclosure is not limited thereto.

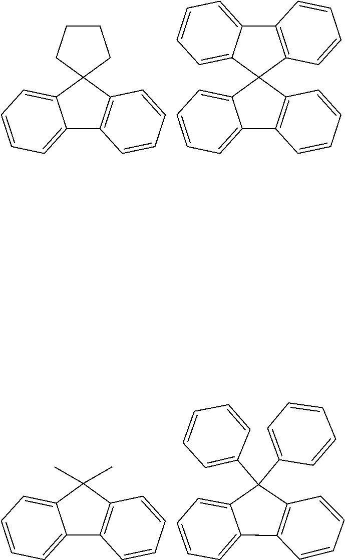

In the specification, the heteroaryl group may include at least one of B, O, N, P, Si, or S as a heteroatom. When the heteroaryl group contains two or more hetero atoms, the two or more hetero atoms may be the same as or different from each other. The heteroaryl group may be a monocyclic heteroaryl group or a polycyclic heteroaryl group. The number of ring-forming carbon atoms in the heteroaryl group may be 2 to 30, 2 to 20, or 2 to 10. Examples of the heteroaryl group may include a thiophene group, a furan group, a pyrrole group, an imidazole group, a triazole group, a pyridine group, a bipyridine group, a pyrimidine group, a triazine group, a triazole group, an acridyl group, a pyridazine group, a pyrazinyl group, a quinoline group, a quinazoline group, a quinoxaline group, a phenoxazine group, a phthalazine group, a pyrido pyrimidine group, a pyrido pyrazine group, a pyrazino pyrazine group, an isoquinoline group, an indole group, a carbazole group, an N-arylcarbazole group, an N-heteroarylcarbazole group, an N-alkylcarbazole group, a benzoxazole group, a benzoimidazole group, a benzothiazole group, a benzocarbazole group, a benzothiophene group, a dibenzothiophene group, a thienothiophene group, a benzofuran group, a phenanthroline group, a thiazole group, an isoxazole group, an oxazole group, an oxadiazole group, a thiadiazole group, a phenothiazine group, a dibenzosilole group, a dibenzofuran group, etc., but the embodiment of the present disclosure is not limited thereto.

In the specification, the number of carbon atoms in an amine group is not specifically limited, but may be 1 to 30. The amine group may include an alkyl amine group and an aryl amine group. Examples of the amine group may include a methylamine group, a dimethylamine group, a phenylamine group, a diphenylamine group, a naphthylamine group, a 9-methyl-anthracenylamine group, a triphenylamine group, etc., but the embodiment of the present disclosure is not limited thereto.

The amine compound of an embodiment may be represented by Formula 1 below:

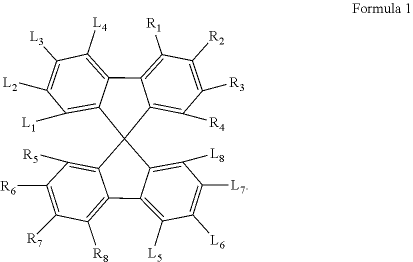

In Formula 1, R₁ to R₈ are each independently a hydrogen atom, a deuterium atom, a halogen atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. For example, R₁ to R₈ may be each independently a hydrogen atom or a deuterium atom.

In Formula 1, L₁ to L₈ are each independently a hydrogen atom, a deuterium atom, a halogen atom, a substituted or unsubstituted amine group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. For example, L₁ to L₈ may be each independently a hydrogen atom, a deuterium atom, or a substituted or unsubstituted amine group.

In Formula 1, at least one among L₁ to L₄ is represented by Formula 2 below, and at least one among L₅ to L₈ is represented by Formula 3 below. In Formula 1, any one among L₁ to L₄ may be represented by Formula 2 below, and the remainder (e.g., the rest) may be a hydrogen atom, a deuterium atom, a halogen atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. In Formula 1, any one among L₅ to L₈ may be represented by Formula 3 below, and the remainder (e.g., the rest) may be a hydrogen atom, a deuterium atom, a halogen atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms.

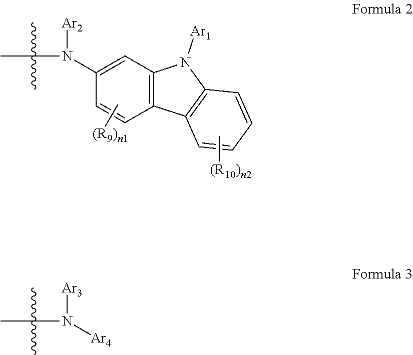

In Formula 2, R₉ and R₁₀ are each independently a hydrogen atom, a deuterium atom, a halogen atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. For example, R₉ and R₁₀ may be each independently a hydrogen atom or a deuterium atom.

In Formula 2 and Formula 3, Ar₁ to Ar₄ are each independently a substituted or unsubstituted aryl group having 6 to 30 ring-forming carbon atoms, or a substituted or unsubstituted heteroaryl group having 2 to 30 ring-forming carbon atoms. For example, Ar₁ to Ar₄ may be each independently a substituted or unsubstituted phenyl group, a substituted or unsubstituted naphthyl group, or a substituted or unsubstituted biphenyl group.

In Formula 2, n1 is an integer of 0 to 3, and n2 is an integer of 0 to 4. In Formula 2, when n1 is 0, the amine compound of an embodiment may not be substituted with R₉. In Formula 2, the case where n1 is 3 and R₉s are all hydrogen atoms may be the same as the case where n1 is 0. In Formula 2, when n1 is an integer of 2 or more, a plurality of R₉s may be the same as or different from each other. In Formula 2, when n2 is 0, the amine compound of an embodiment may not be substituted with R₁₀. In Formula 2, the case where n2 is 4 and Rios are all hydrogen atoms may be the same as the case where n2 is 0. In Formula 2, when n2 is an integer of 2 or more, a plurality of Rios may be the same as or different from each other.

In an embodiment, the amine compound represented by Formula 1 may be represented by any one among Formula 4-1 to Formula 4-4 below:

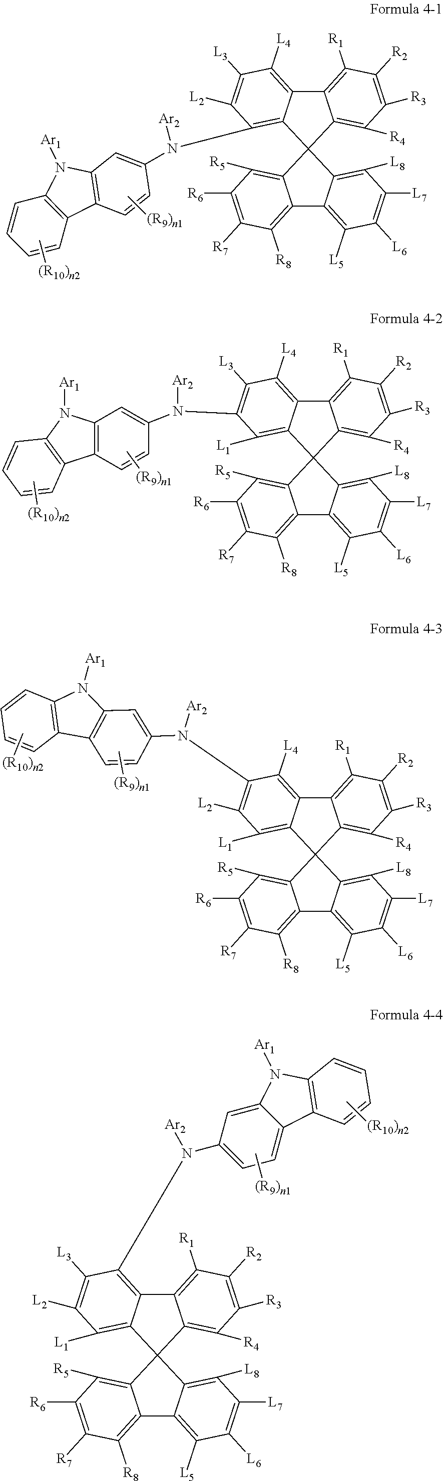

Formula 4-1 to Formula 4-4 represent that in Formula 1, a carbon position in the spirobifluorene moiety to which the central nitrogen of the amine group represented by Formula 2 is connected is specified. Formula 4-1 is the case where in Formula 1, a spirobifluorene moiety is connected, through L₁, to the central nitrogen of the amine group represented by Formula 2. Formula 4-2 is the case where in Formula 1, a spirobifluorene moiety is connected, through L₂, to the central nitrogen of the amine group represented by Formula 2. Formula 4-3 is the case where in Formula 1, a spirobifluorene moiety is connected, through L₃, to the central nitrogen of the amine group represented by Formula 2. Formula 4-4 is the case where in Formula 1, a spirobifluorene moiety is connected, through L₄, to the central nitrogen of the amine group represented by Formula 2.

In one or more embodiments, the same as those described in connection with Formula 1 and Formula 2 above may be applied with regard to R₁ to R₁₀, L₁ to L₈, Ar₁, Ar₂, n1 and n2 in Formula 4-1 to Formula 4-4.

In an embodiment, the amine compound represented by Formula 1 may be represented by any one among Formula 5-1 to Formula 5-4 below:

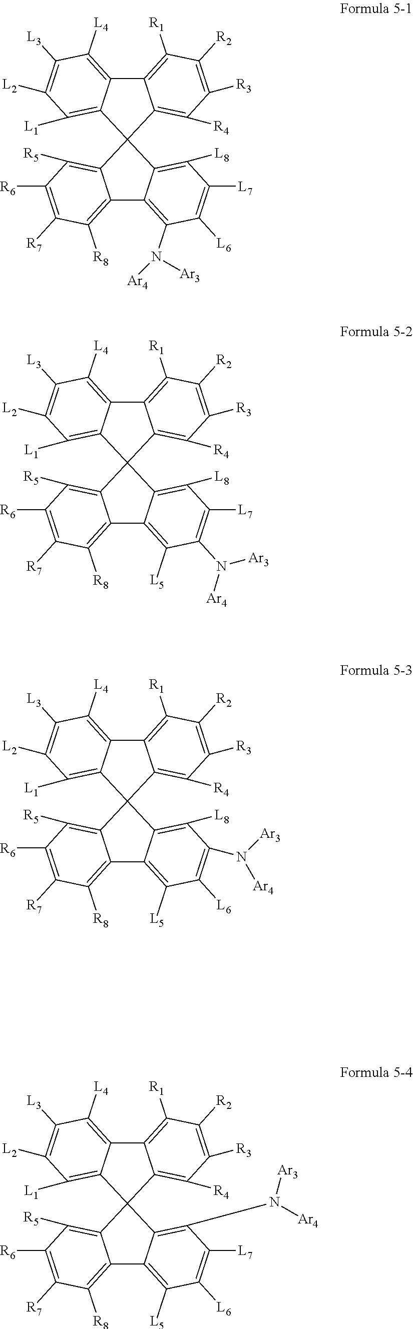

Formula 5-1 to Formula 5-4 represent that in Formula 1, a carbon position in the spirobifluorene moiety to which the central nitrogen of the amine group represented by Formula 3 is connected is specified. Formula 5-1 is the case where in Formula 1, a spirobifluorene moiety is connected, through L₅, to the central nitrogen of the amine group represented by Formula 3. Formula 5-2 is the case where in Formula 1, a spirobifluorene moiety is connected, through L₆, to the central nitrogen of the amine group represented by Formula 3. Formula 5-3 is the case where in Formula 1, a spirobifluorene moiety is connected, through L₇, to the central nitrogen of the amine group represented by Formula 3. Formula 5-4 is the case where in Formula 1, a spirobifluorene moiety is connected, through L₈, to the central nitrogen of the amine group represented by Formula 3.

In one or more embodiments, the same as those described in connection with Formula 1 and Formula 3 above may be applied with regard to R₁ to R₈, L₁ to L₈, Ar₃, and Ar₄ in Formula 5-1 to Formula 5-4.

In an embodiment, the substituent represented by Formula 2 may be represented by Formula 2-1 below:

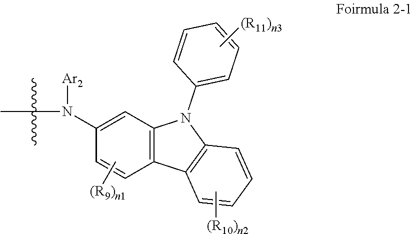

Formula 2-1 represents the case where Ar₁ in Formula 2 is specified as a substituted or unsubstituted phenyl group.

In Formula 2-1, Ru may be a hydrogen atom, a deuterium atom, a halogen atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. For example, Ru may be a hydrogen atom or a deuterium atom.

In Formula 2-1, n3 is an integer of 0 to 5. In Formula 2-1, when n3 is 0, the amine compound of an embodiment may not be substituted with Rut In Formula 2-1, the case where n3 is 4 and Riis are all hydrogen atoms may be the same as the case where n3 is 0. In Formula 2-1, when n3 is an integer of 2 or more, a plurality of Riis may be the same as or different from each other.

In one or more embodiments, the same as those described in connection with Formula 2 above may be applied with regard to R₉, R₁₀, Ar₂, n1 and n2 in Formula 2-1.

In an embodiment, Ar₁ to Ar₄ may be each independently represented by any one among Formula 6-1 to Formula 6-6 below:

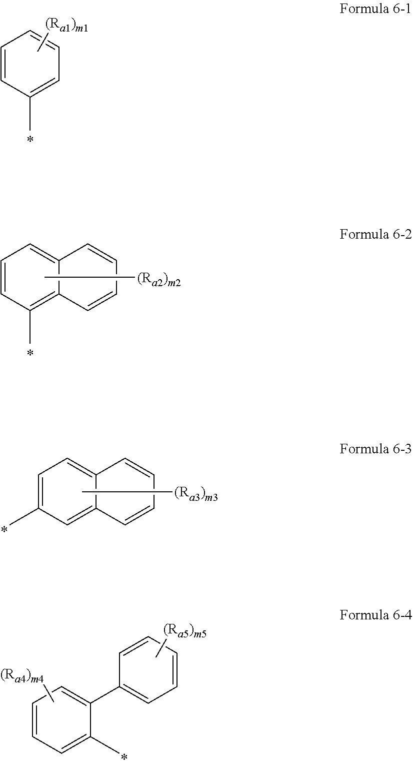

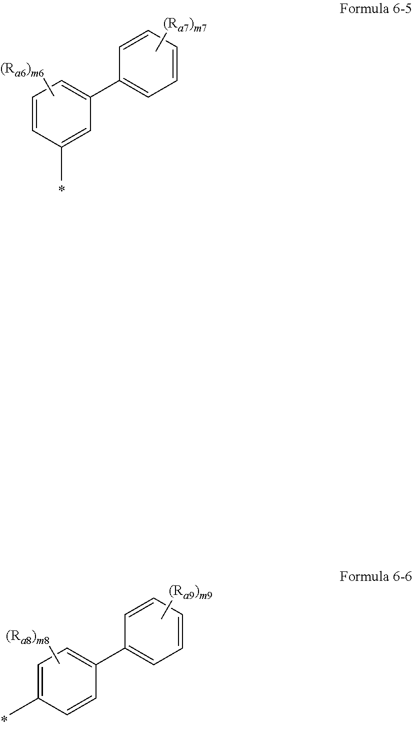

In Formula 6-1 to Formula 6-6, Rₐ₁ to Rₐ₉ may be each independently a hydrogen atom, a deuterium atom, a halogen atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. For example, Rₐ₁ to Rₐ₉ may be each independently a hydrogen atom or a deuterium atom.

In Formula 6-1 to Formula 6-6, m1, m5, m7, and m9 are each independently an integer of 0 to 5, m2 and m3 are each independently an integer of 0 to 7, and m4, m6, and m8 are each independently an integer of 0 to 4.

In Formula 6-1, when m1 is 0, the amine compound of an embodiment may not be substituted with Rₐ₁. In Formula 6-1, the case where m1 is 5 and Rₐ₁s are all hydrogen atoms may be the same as the case where m1 is 0.

In Formula 6-2, when m2 is 0, the amine compound of an embodiment may not be substituted with Rₐ₂. In Formula 6-2, the case where m2 is 7 and Rₐ₂s are all hydrogen atoms may be the same as the case where m2 is 0.

In Formula 6-3, when m3 is 0, the amine compound of an embodiment may not be substituted with Rₐ₁. In Formula 6-3, the case where m3 is 7 and Rₐ₃s are all hydrogen atoms may be the same as the case where m3 is 0.

In Formula 6-4, when m4 is 0, the amine compound of an embodiment may not be substituted with Rₐ₄. In Formula 6-4, the case where m4 is 4 and Rₐ₄s are all hydrogen atoms may be the same as the case where m4 is 0.

In Formula 6-4, when m5 is 0, the amine compound of an embodiment may not be substituted with Rₐ₅. In Formula 6-4, the case where m5 is 5 and Rₐ₅s are all hydrogen atoms may be the same as the case where m5 is 0.

In Formula 6-5, when m6 is 0, the amine compound of an embodiment may not be substituted with Rₐ₆. In Formula 6-5, the case where m6 is 4 and Rₐ₆s are all hydrogen atoms may be the same as the case where m6 is 0.

In Formula 6-5, when m7 is 0, the amine compound of an embodiment may not be substituted with Rₐ₇. In Formula 6-5, the case where m7 is 5 and Rₐ₇s are all hydrogen atoms may be the same as the case where m7 is 0.

In Formula 6-6, when m8 is 0, the amine compound of an embodiment may not be substituted with Rₐ₈. In Formula 6-6, the case where m8 is 4 and Rₐ₈s are all hydrogen atoms may be the same as the case where m8 is 0.

In Formula 6-6, when m9 is 0, the amine compound of an embodiment may not be substituted with Rₐ₉. In Formula 6-6, the case where m9 is 5 and Rₐ₉s are all hydrogen atoms may be the same as the case where m9 is 0.

The amine compound may be any one of the compounds represented by Compound Group 1 below. The light emitting device ED of an embodiment may include at least one amine compound among the compounds represented by Compound Group 1 below in the hole transport region HTR. The light emitting device ED of an embodiment may include at least one amine compound among the compounds represented by Compound Group 1 below in the hole transport layer HTL. In one or more embodiments, the light emitting device ED of an embodiment may include at least one amine compound among the compounds represented by Compound Group 1 below in the electron blocking layer EBL.

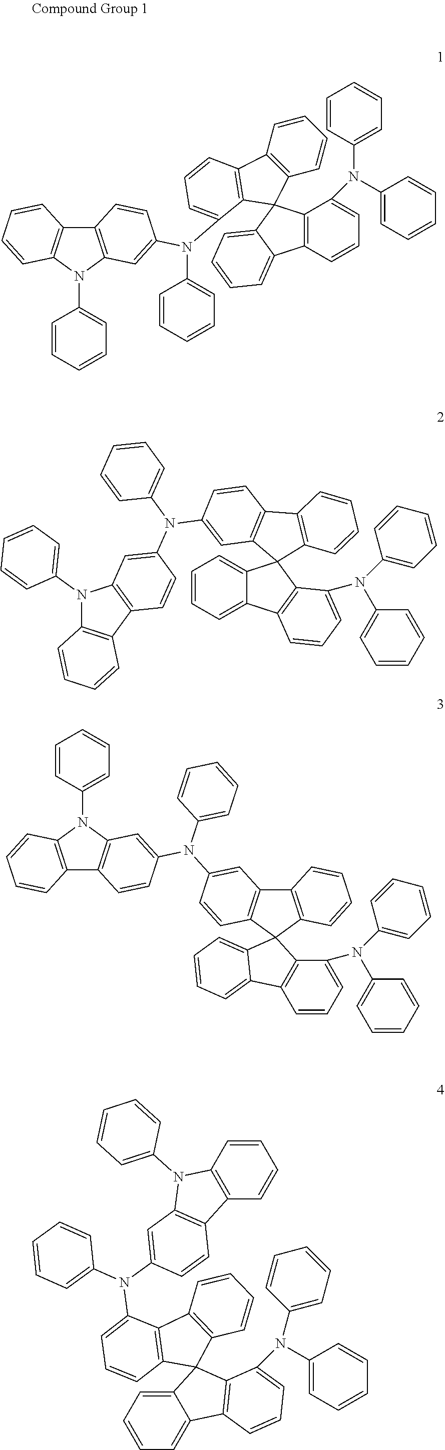

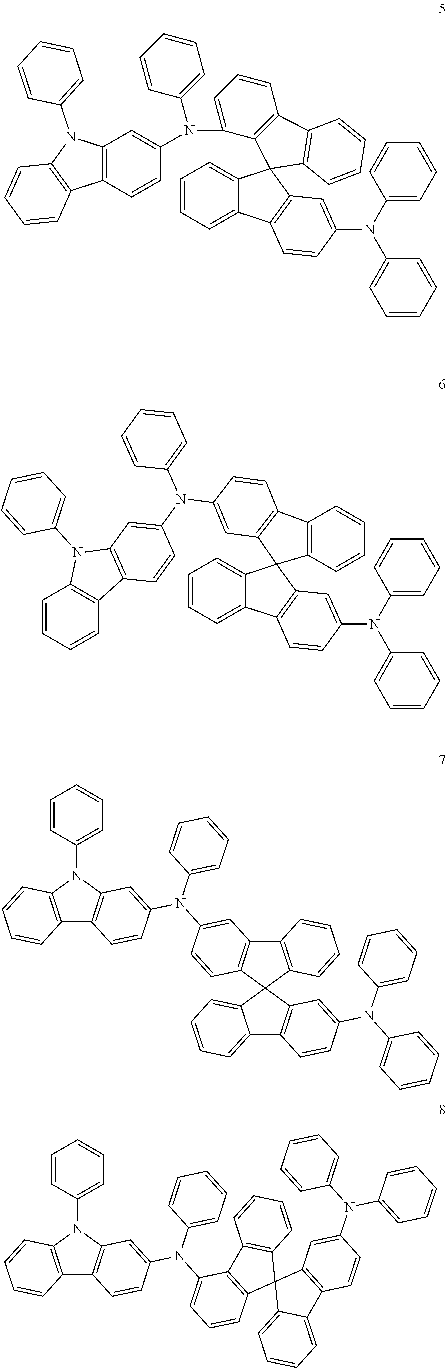 [SMILES: c1ccc(N(c2ccccc2)c2ccc3c(c2)C2(c4ccccc4-3)c3ccccc3-c3cccc(N(c4ccccc4)c4ccc5c6ccccc6n(-c6ccccc6)c5c4)c32)cc1 ; c1ccc(N(c2ccccc2)c2ccc3c(c2)C2(c4ccccc4-3)c3ccccc3-c3ccc(N(c4ccccc4)c4ccc5c6ccccc6n(-c6ccccc6)c5c4)cc32)cc1 ; c1ccc(N(c2ccccc2)c2ccc3c(c2)C2(c4ccccc4-c4cc(N(c5ccccc5)c5ccc6c7ccccc7n(-c7ccccc7)c6c5)ccc42)c2ccccc2-3)cc1 ; c1ccc(N(c2ccccc2)c2ccc3c(c2)C2(c4ccccc4-3)c3ccccc3-c3c(N(c4ccccc4)c4ccc5c6ccccc6n(-c6ccccc6)c5c4)cccc32)cc1]

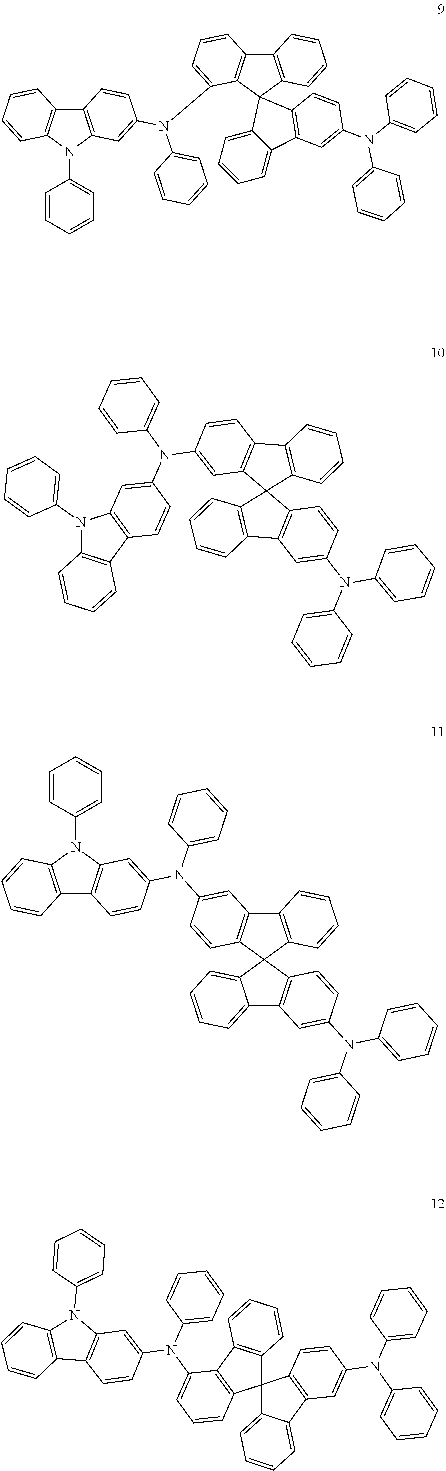 [SMILES: c1ccc(N(c2ccccc2)c2ccc3c(c2)-c2ccccc2C32c3ccccc3-c3cccc(N(c4ccccc4)c4ccc5c6ccccc6n(-c6ccccc6)c5c4)c32)cc1 ; c1ccc(N(c2ccccc2)c2ccc3c(c2)-c2ccccc2C32c3ccccc3-c3ccc(N(c4ccccc4)c4ccc5c6ccccc6n(-c6ccccc6)c5c4)cc32)cc1 ; c1ccc(N(c2ccccc2)c2ccc3c(c2)-c2ccccc2C32c3ccccc3-c3cc(N(c4ccccc4)c4ccc5c6ccccc6n(-c6ccccc6)c5c4)ccc32)cc1 ; c1ccc(N(c2ccccc2)c2ccc3c(c2)-c2ccccc2C32c3ccccc3-c3c(N(c4ccccc4)c4ccc5c6ccccc6n(-c6ccccc6)c5c4)cccc32)cc1]

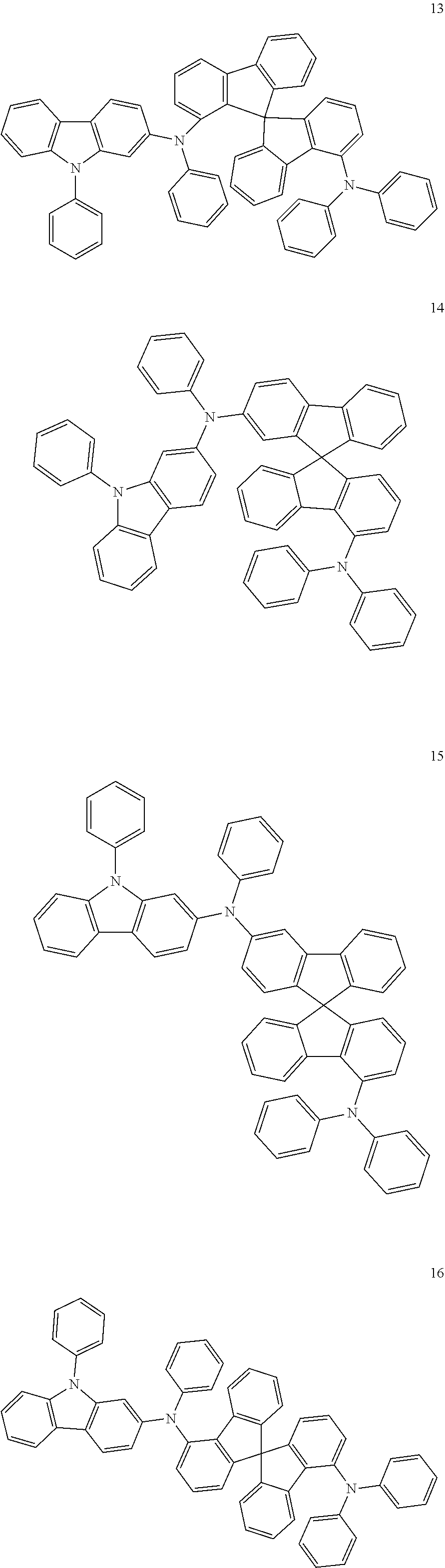 [SMILES: c1ccc(N(c2ccccc2)c2cccc3c2-c2ccccc2C32c3ccccc3-c3cccc(N(c4ccccc4)c4ccc5c6ccccc6n(-c6ccccc6)c5c4)c32)cc1 ; c1ccc(N(c2ccc3c(c2)C2(c4ccccc4-3)c3ccccc3-c3c(N(c4ccccc4)c4ccccc4)cccc32)c2ccc3c4ccccc4n(-c4ccccc4)c3c2)cc1 ; c1ccc(N(c2ccc3c(c2)-c2ccccc2C32c3ccccc3-c3c(N(c4ccccc4)c4ccccc4)cccc32)c2ccc3c4ccccc4n(-c4ccccc4)c3c2)cc1 ; c1ccc(N(c2ccccc2)c2cccc3c2-c2ccccc2C32c3ccccc3-c3c(N(c4ccccc4)c4ccc5c6ccccc6n(-c6ccccc6)c5c4)cccc32)cc1]

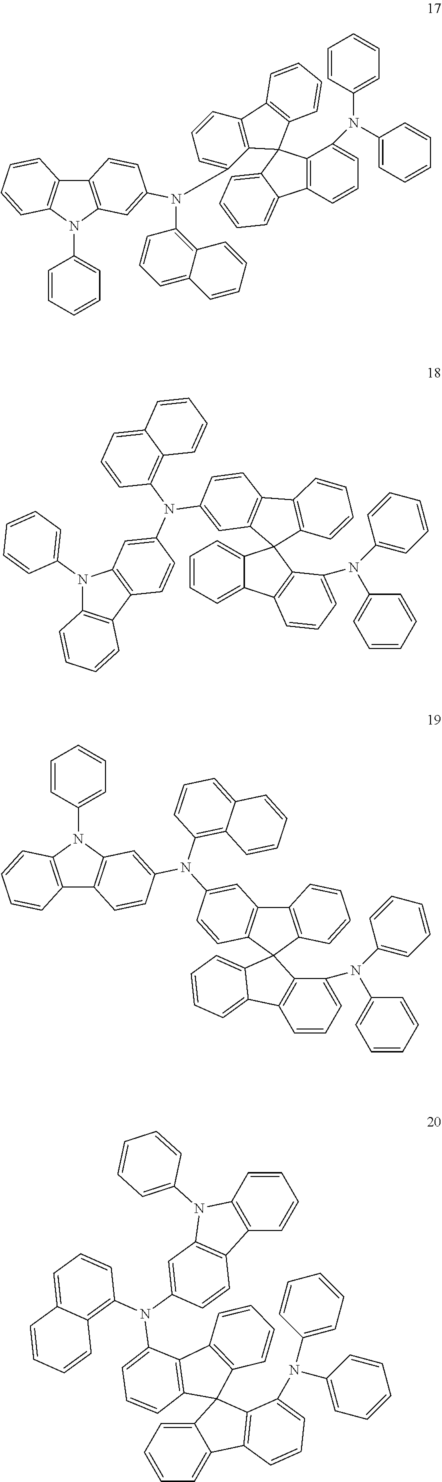 [SMILES: c1ccc(N(c2ccccc2)c2cccc3c2C2(c4ccccc4-3)c3ccccc3-c3cccc(N(c4ccc5c6ccccc6n(-c6ccccc6)c5c4)c4cccc5ccccc45)c32)cc1 ; c1ccc(N(c2ccccc2)c2cccc3c2C2(c4ccccc4-c4ccc(N(c5ccc6c7ccccc7n(-c7ccccc7)c6c5)c5cccc6ccccc56)cc42)c2ccccc2-3)cc1 ; c1ccc(N(c2ccccc2)c2cccc3c2C2(c4ccccc4-c4cc(N(c5ccc6c7ccccc7n(-c7ccccc7)c6c5)c5cccc6ccccc56)ccc42)c2ccccc2-3)cc1 ; c1ccc(N(c2ccccc2)c2cccc3c2C2(c4ccccc4-3)c3ccccc3-c3c(N(c4ccc5c6ccccc6n(-c6ccccc6)c5c4)c4cccc5ccccc45)cccc32)cc1]

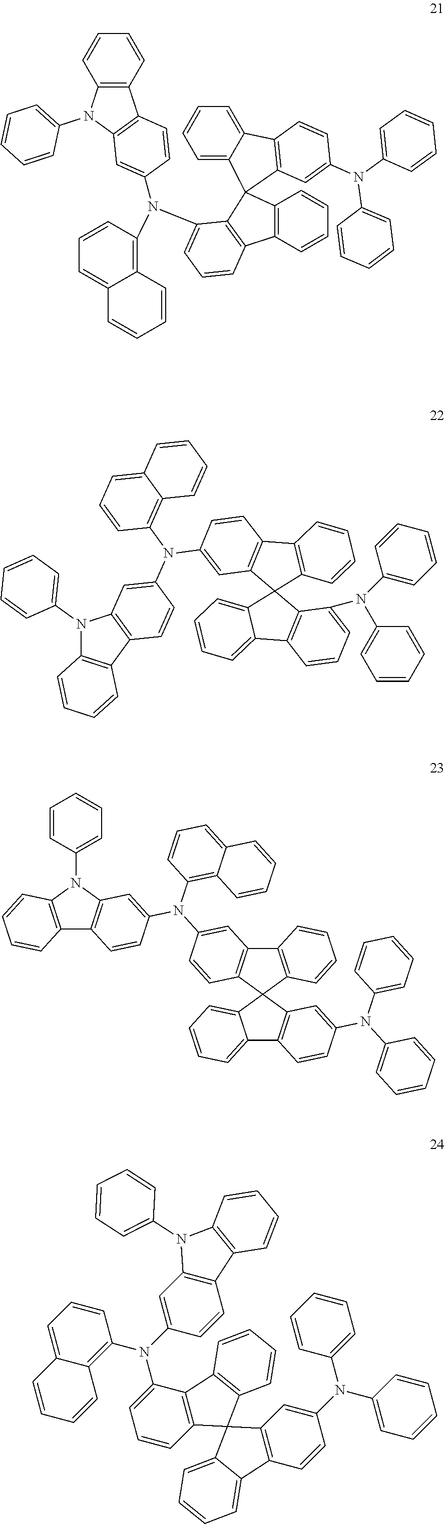 [SMILES: c1ccc(N(c2ccccc2)c2ccc3c(c2)C2(c4ccccc4-3)c3ccccc3-c3cccc(N(c4ccc5c6ccccc6n(-c6ccccc6)c5c4)c4cccc5ccccc45)c32)cc1 ; c1ccc(N(c2ccccc2)c2cccc3c2C2(c4ccccc4-c4ccc(N(c5ccc6c7ccccc7n(-c7ccccc7)c6c5)c5cccc6ccccc56)cc42)c2ccccc2-3)cc1 ; c1ccc(N(c2ccccc2)c2ccc3c(c2)C2(c4ccccc4-c4cc(N(c5ccc6c7ccccc7n(-c7ccccc7)c6c5)c5cccc6ccccc56)ccc42)c2ccccc2-3)cc1 ; c1ccc(N(c2ccccc2)c2ccc3c(c2)C2(c4ccccc4-3)c3ccccc3-c3c(N(c4ccc5c6ccccc6n(-c6ccccc6)c5c4)c4cccc5ccccc45)cccc32)cc1]

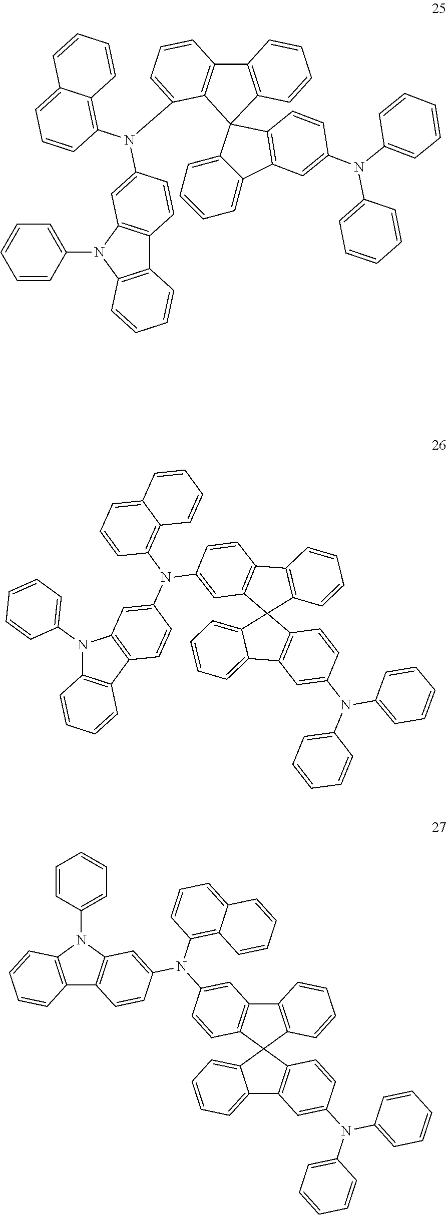 [SMILES: c1ccc(N(c2ccccc2)c2ccc3c(c2)-c2ccccc2C32c3ccccc3-c3cccc(N(c4ccc5c6ccccc6n(-c6ccccc6)c5c4)c4cccc5ccccc45)c32)cc1 ; c1ccc(N(c2ccccc2)c2ccc3c(c2)-c2ccccc2C32c3ccccc3-c3ccc(N(c4ccc5c6ccccc6n(-c6ccccc6)c5c4)c4cccc5ccccc45)cc32)cc1 ; c1ccc(N(c2ccccc2)c2ccc3c(c2)-c2ccccc2C32c3ccccc3-c3cc(N(c4ccc5c6ccccc6n(-c6ccccc6)c5c4)c4cccc5ccccc45)ccc32)cc1]

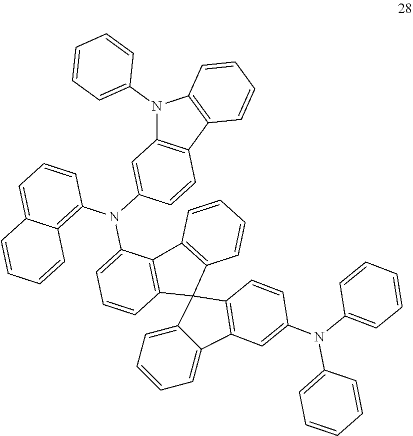 [SMILES: c1ccc(N(c2ccccc2)c2ccc3c(c2)-c2ccccc2C32c3ccccc3-c3c(N(c4ccc5c6ccccc6n(-c6ccccc6)c5c4)c4cccc5ccccc45)cccc32)cc1]

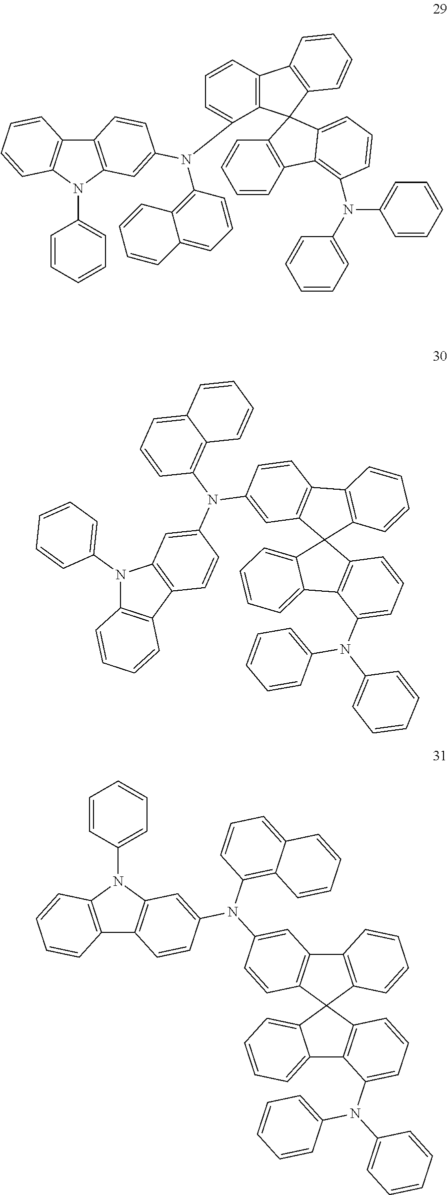 [SMILES: c1ccc(N(c2ccccc2)c2cccc3c2-c2ccccc2C32c3ccccc3-c3cccc(N(c4ccc5c6ccccc6n(-c6ccccc6)c5c4)c4cccc5ccccc45)c32)cc1 ; c1ccc(N(c2ccccc2)c2cccc3c2-c2ccccc2C32c3ccccc3-c3ccc(N(c4ccc5c6ccccc6n(-c6ccccc6)c5c4)c4cccc5ccccc45)cc32)cc1 ; c1ccc(N(c2ccccc2)c2cccc3c2-c2ccccc2C32c3ccccc3-c3cc(N(c4ccc5c6ccccc6n(-c6ccccc6)c5c4)c4cccc5ccccc45)ccc32)cc1]

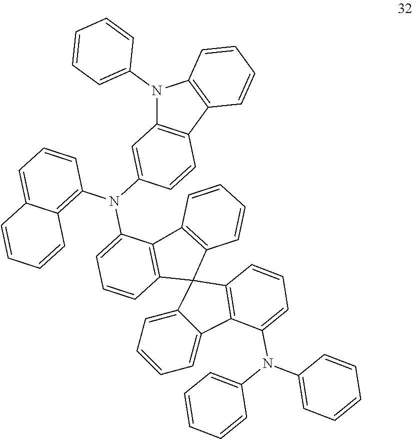 [SMILES: c1ccc(N(c2ccccc2)c2cccc3c2-c2ccccc2C32c3ccccc3-c3c(N(c4ccc5c6ccccc6n(-c6ccccc6)c5c4)c4cccc5ccccc45)cccc32)cc1]

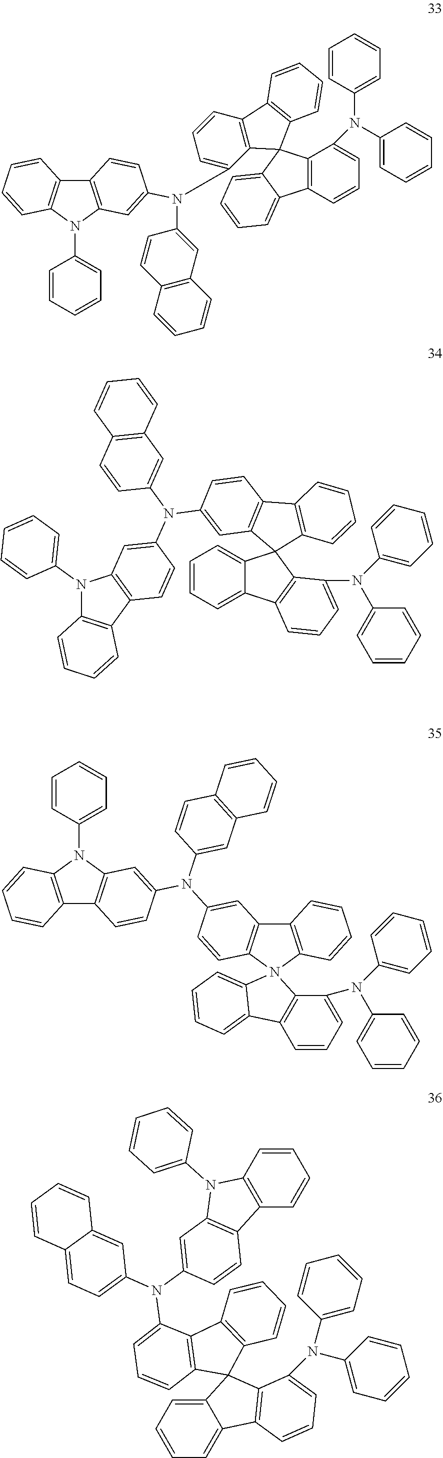

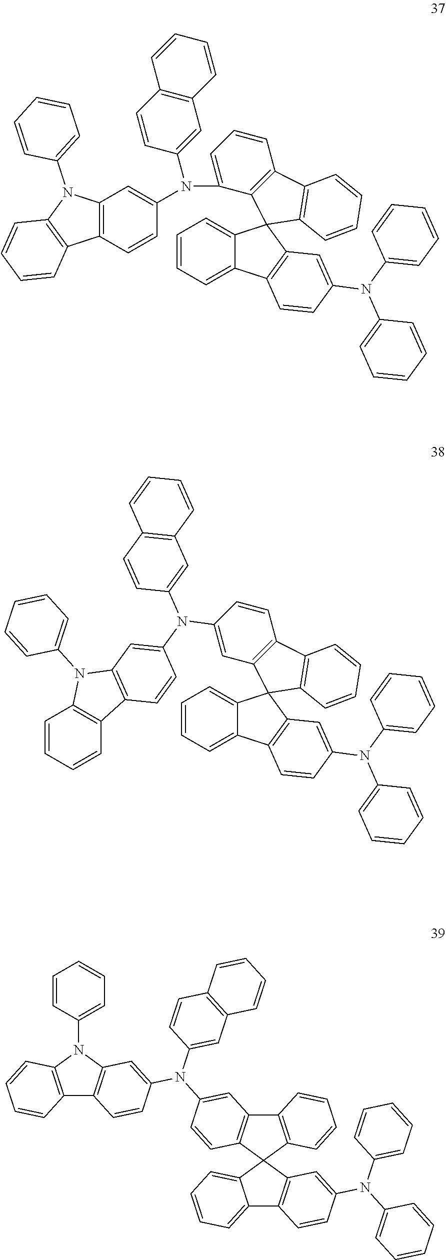 [SMILES: c1ccc(N(c2ccccc2)c2ccc3c(c2)C2(c4ccccc4-3)c3ccccc3-c3cccc(N(c4ccc5ccccc5c4)c4ccc5c6ccccc6n(-c6ccccc6)c5c4)c32)cc1 ; c1ccc(N(c2ccccc2)c2ccc3c(c2)C2(c4ccccc4-3)c3ccccc3-c3ccc(N(c4ccc5ccccc5c4)c4ccc5c6ccccc6n(-c6ccccc6)c5c4)cc32)cc1 ; c1ccc(N(c2ccccc2)c2ccc3c(c2)C2(c4ccccc4-c4cc(N(c5ccc6ccccc6c5)c5ccc6c7ccccc7n(-c7ccccc7)c6c5)ccc42)c2ccccc2-3)cc1]

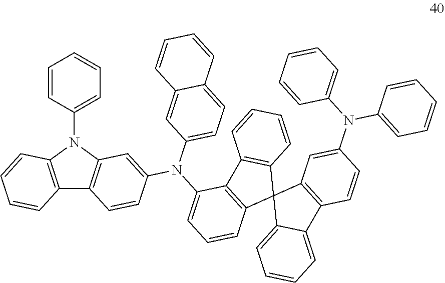 [SMILES: c1ccc(N(c2ccccc2)c2ccc3c(c2)C2(c4ccccc4-3)c3ccccc3-c3c(N(c4ccc5ccccc5c4)c4ccc5c6ccccc6n(-c6ccccc6)c5c4)cccc32)cc1]

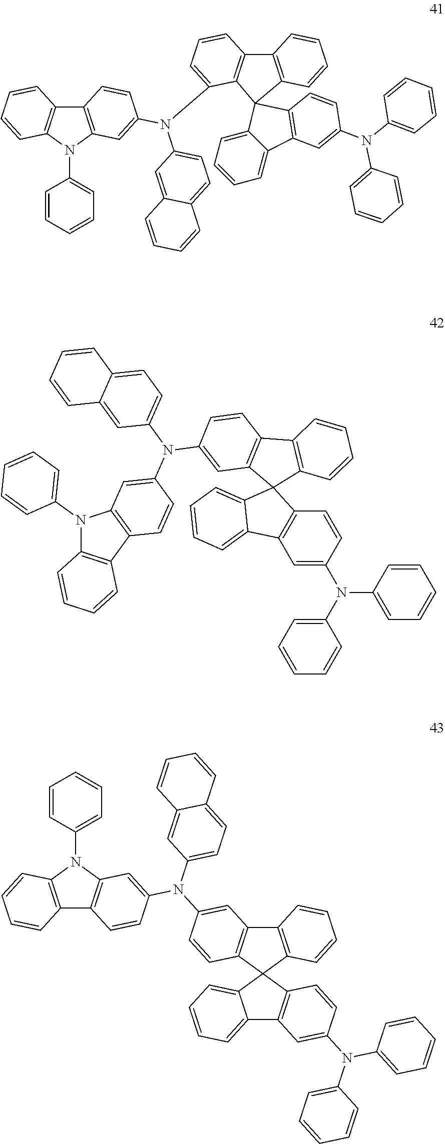 [SMILES: c1ccc(N(c2ccccc2)c2ccc3c(c2)-c2ccccc2C32c3ccccc3-c3cccc(N(c4ccc5ccccc5c4)c4ccc5c6ccccc6n(-c6ccccc6)c5c4)c32)cc1 ; c1ccc(N(c2ccccc2)c2ccc3c(c2)-c2ccccc2C32c3ccccc3-c3ccc(N(c4ccc5ccccc5c4)c4ccc5c6ccccc6n(-c6ccccc6)c5c4)cc32)cc1 ; c1ccc(N(c2ccccc2)c2ccc3c(c2)-c2ccccc2C32c3ccccc3-c3cc(N(c4ccc5ccccc5c4)c4ccc5c6ccccc6n(-c6ccccc6)c5c4)ccc32)cc1]

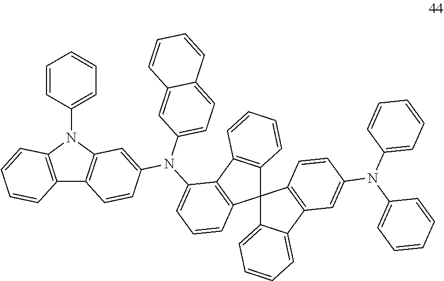 [SMILES: c1ccc(N(c2ccccc2)c2ccc3c(c2)-c2ccccc2C32c3ccccc3-c3c(N(c4ccc5ccccc5c4)c4ccc5c6ccccc6n(-c6ccccc6)c5c4)cccc32)cc1]

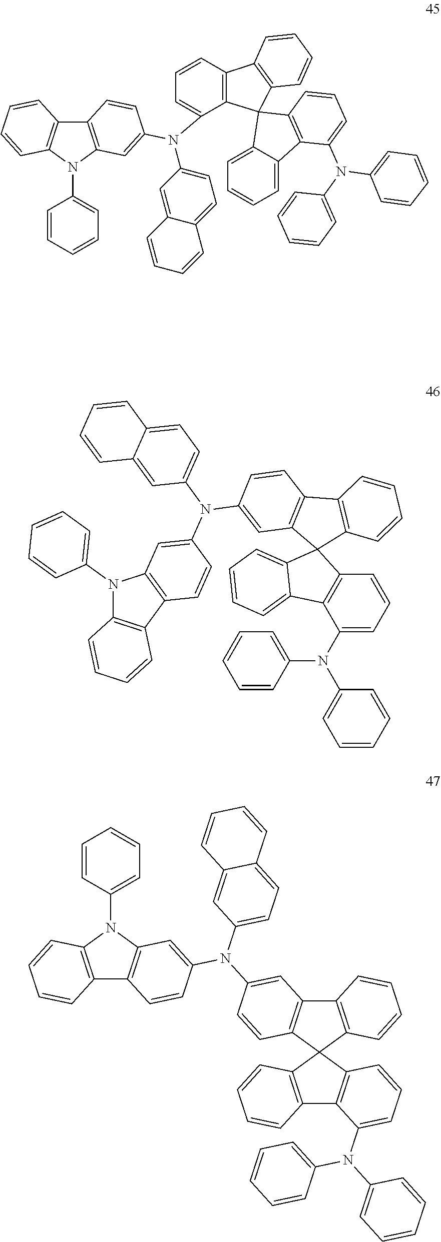 [SMILES: c1ccc(N(c2ccccc2)c2cccc3c2-c2ccccc2C32c3ccccc3-c3cccc(N(c4ccc5ccccc5c4)c4ccc5c6ccccc6n(-c6ccccc6)c5c4)c32)cc1 ; c1ccc(N(c2ccccc2)c2cccc3c2-c2ccccc2C32c3ccccc3-c3ccc(N(c4ccc5ccccc5c4)c4ccc5c6ccccc6n(-c6ccccc6)c5c4)cc32)cc1 ; c1ccc(N(c2ccccc2)c2cccc3c2-c2ccccc2C32c3ccccc3-c3cc(N(c4ccc5ccccc5c4)c4ccc5c6ccccc6n(-c6ccccc6)c5c4)ccc32)cc1]

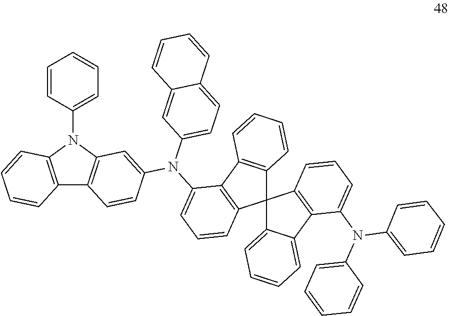 [SMILES: c1ccc(N(c2ccccc2)c2cccc3c2-c2ccccc2C32c3ccccc3-c3c(N(c4ccc5ccccc5c4)c4ccc5c6ccccc6n(-c6ccccc6)c5c4)cccc32)cc1]

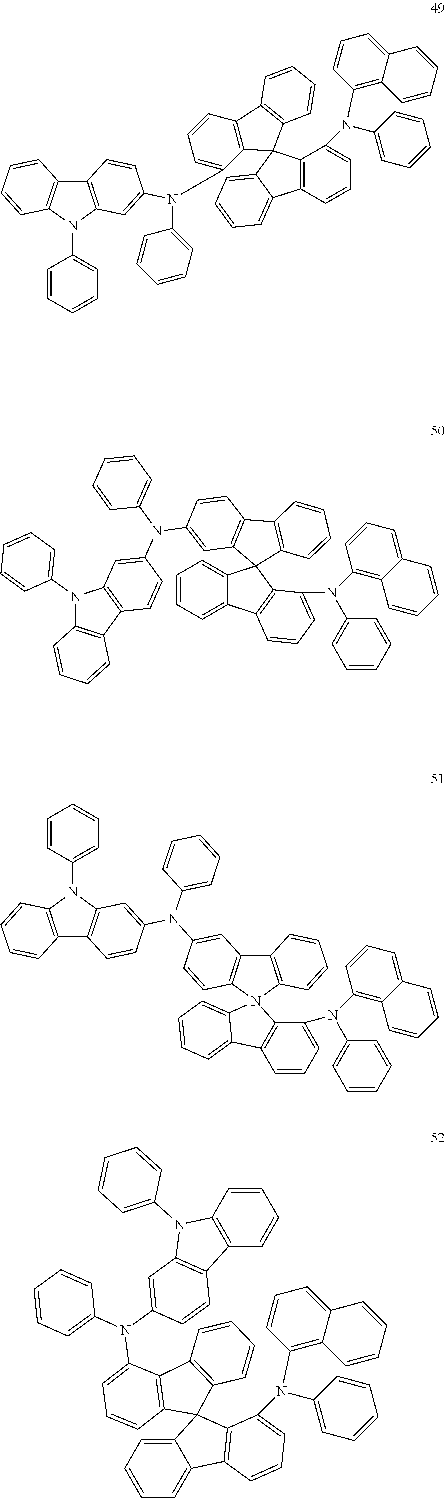

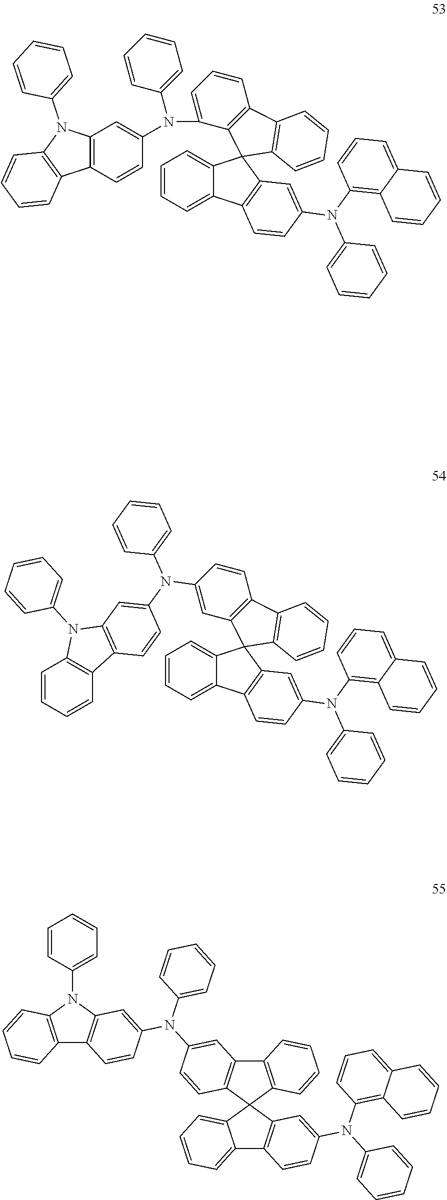 [SMILES: c1ccc(N(c2ccc3c4ccccc4n(-c4ccccc4)c3c2)c2cccc3c2C2(c4ccccc4-c4ccc(N(c5ccccc5)c5cccc6ccccc56)cc42)c2ccccc2-3)cc1 ; c1ccc(N(c2ccc3c(c2)C2(c4ccccc4-3)c3ccccc3-c3ccc(N(c4ccccc4)c4cccc5ccccc45)cc32)c2ccc3c4ccccc4n(-c4ccccc4)c3c2)cc1 ; c1ccc(N(c2ccc3c(c2)-c2ccccc2C32c3ccccc3-c3ccc(N(c4ccccc4)c4cccc5ccccc45)cc32)c2ccc3c4ccccc4n(-c4ccccc4)c3c2)cc1]

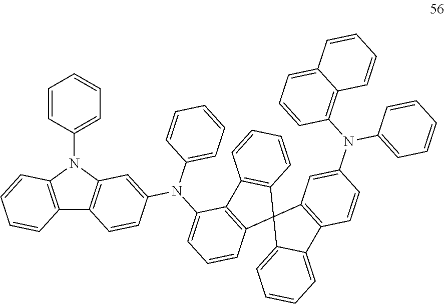 [SMILES: c1ccc(N(c2ccc3c4ccccc4n(-c4ccccc4)c3c2)c2cccc3c2-c2ccccc2C32c3ccccc3-c3ccc(N(c4ccccc4)c4cccc5ccccc45)cc32)cc1]

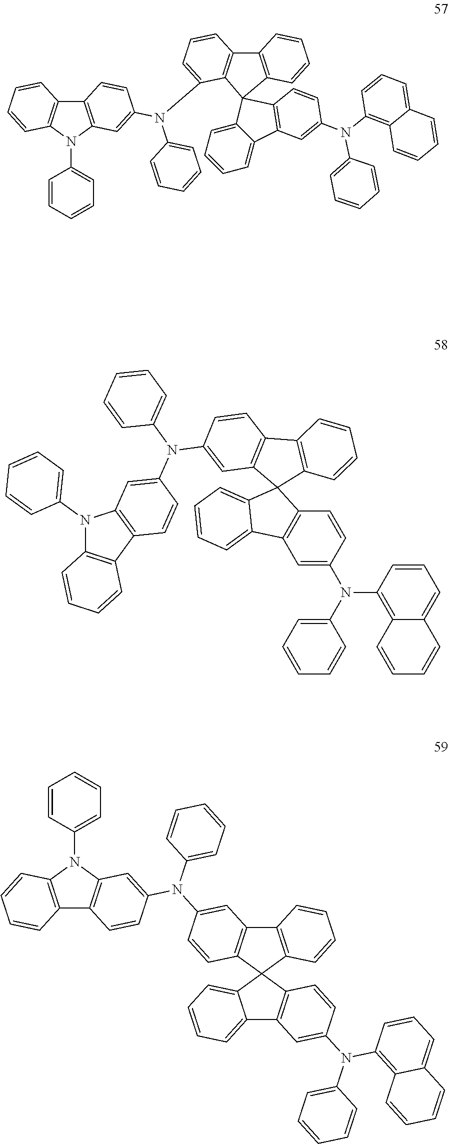 [SMILES: c1ccc(N(c2ccc3c4ccccc4n(-c4ccccc4)c3c2)c2cccc3c2C2(c4ccccc4-c4cc(N(c5ccccc5)c5cccc6ccccc56)ccc42)c2ccccc2-3)cc1 ; c1ccc(N(c2ccc3c(c2)C2(c4ccccc4-c4cc(N(c5ccccc5)c5cccc6ccccc56)ccc42)c2ccccc2-3)c2ccc3c4ccccc4n(-c4ccccc4)c3c2)cc1 ; c1ccc(N(c2ccc3c(c2)-c2ccccc2C32c3ccccc3-c3cc(N(c4ccccc4)c4cccc5ccccc45)ccc32)c2ccc3c4ccccc4n(-c4ccccc4)c3c2)cc1]

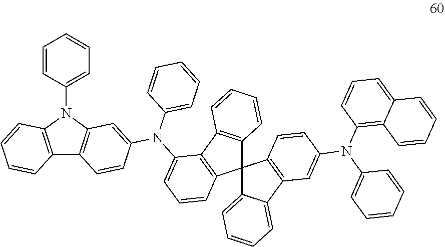 [SMILES: c1ccc(N(c2ccc3c4ccccc4n(-c4ccccc4)c3c2)c2cccc3c2-c2ccccc2C32c3ccccc3-c3cc(N(c4ccccc4)c4cccc5ccccc45)ccc32)cc1]

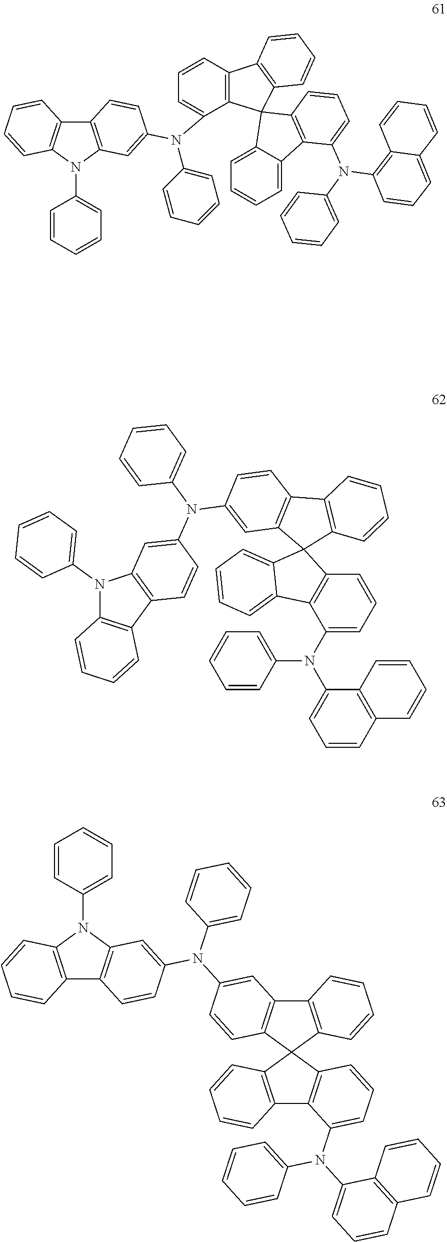 [SMILES: c1ccc(N(c2ccc3c4ccccc4n(-c4ccccc4)c3c2)c2cccc3c2C2(c4ccccc4-3)c3ccccc3-c3c(N(c4ccccc4)c4cccc5ccccc45)cccc32)cc1 ; c1ccc(N(c2ccc3c(c2)C2(c4ccccc4-3)c3ccccc3-c3c(N(c4ccccc4)c4cccc5ccccc45)cccc32)c2ccc3c4ccccc4n(-c4ccccc4)c3c2)cc1 ; c1ccc(N(c2ccc3c(c2)-c2ccccc2C32c3ccccc3-c3c(N(c4ccccc4)c4cccc5ccccc45)cccc32)c2ccc3c4ccccc4n(-c4ccccc4)c3c2)cc1]

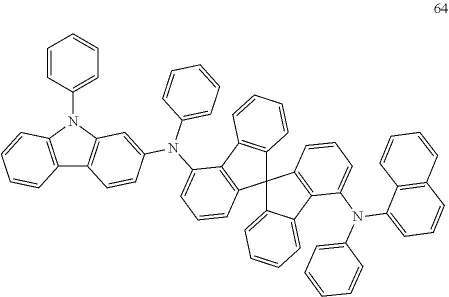 [SMILES: c1ccc(N(c2ccc3c4ccccc4n(-c4ccccc4)c3c2)c2cccc3c2-c2ccccc2C32c3ccccc3-c3c(N(c4ccccc4)c4cccc5ccccc45)cccc32)cc1]

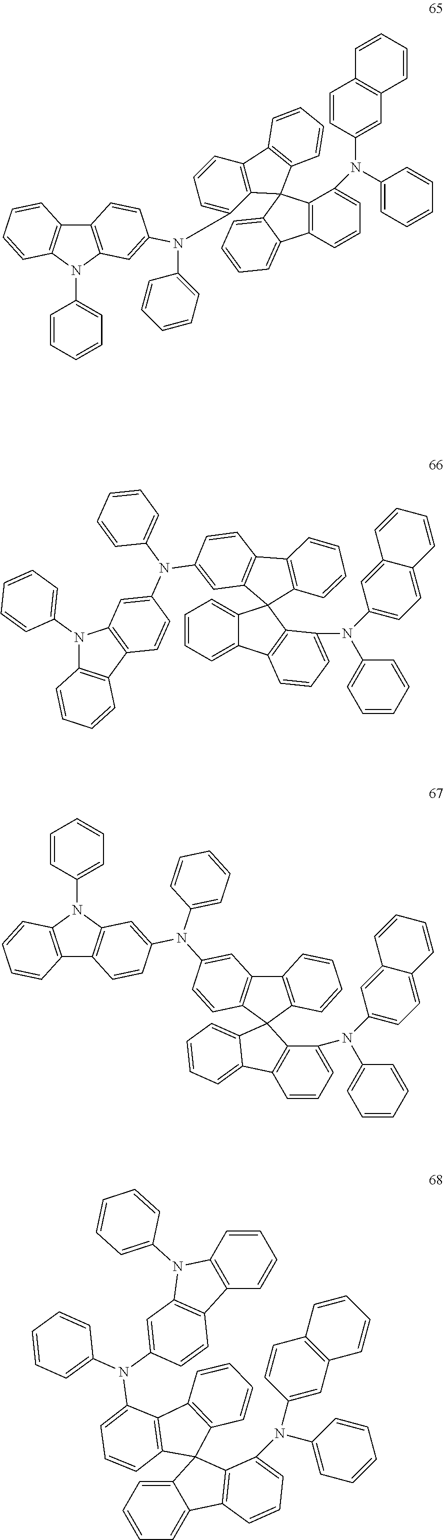 [SMILES: c1ccc(N(c2ccc3ccccc3c2)c2cccc3c2C2(c4ccccc4-3)c3ccccc3-c3cccc(N(c4ccccc4)c4ccc5c6ccccc6n(-c6ccccc6)c5c4)c32)cc1 ; c1ccc(N(c2ccc3c(c2)C2(c4ccccc4-3)c3ccccc3-c3cccc(N(c4ccccc4)c4ccc5ccccc5c4)c32)c2ccc3c4ccccc4n(-c4ccccc4)c3c2)cc1 ; c1ccc(N(c2ccc3c(c2)-c2ccccc2C32c3ccccc3-c3cccc(N(c4ccccc4)c4ccc5ccccc5c4)c32)c2ccc3c4ccccc4n(-c4ccccc4)c3c2)cc1 ; c1ccc(N(c2ccc3c4ccccc4n(-c4ccccc4)c3c2)c2cccc3c2-c2ccccc2C32c3ccccc3-c3cccc(N(c4ccccc4)c4ccc5ccccc5c4)c32)cc1]

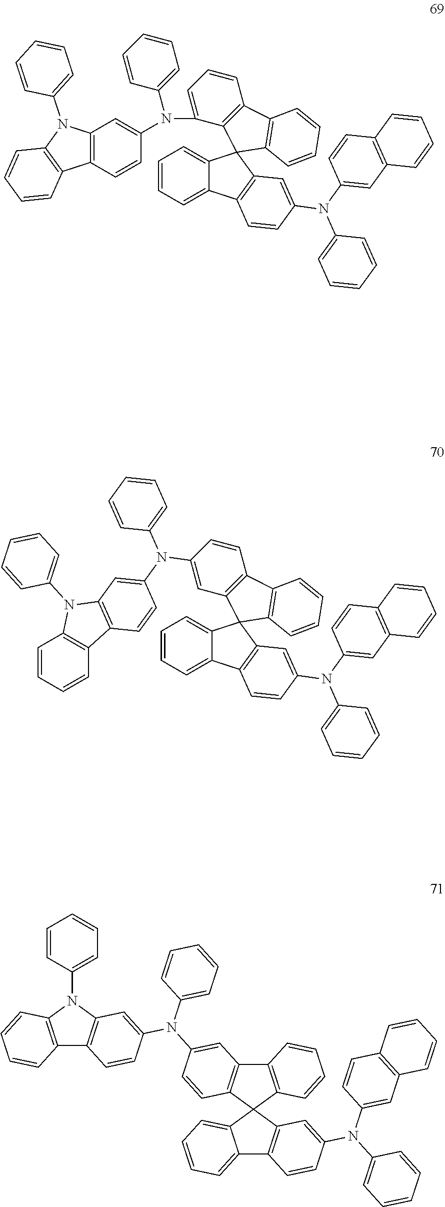 [SMILES: c1ccc(N(c2ccc3c(c2)C2(c4ccccc4-3)c3ccccc3-c3cccc(N(c4ccccc4)c4ccc5c6ccccc6n(-c6ccccc6)c5c4)c32)c2ccc3ccccc3c2)cc1 ; c1ccc(N(c2ccc3c(c2)C2(c4ccccc4-3)c3ccccc3-c3ccc(N(c4ccccc4)c4ccc5c6ccccc6n(-c6ccccc6)c5c4)cc32)c2ccc3ccccc3c2)cc1 ; c1ccc(N(c2ccc3c(c2)C2(c4ccccc4-c4cc(N(c5ccccc5)c5ccc6c7ccccc7n(-c7ccccc7)c6c5)ccc42)c2ccccc2-3)c2ccc3ccccc3c2)cc1]

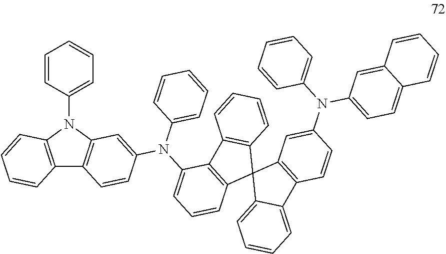 [SMILES: c1ccc(N(c2ccc3c(c2)C2(c4ccccc4-3)c3ccccc3-c3c(N(c4ccccc4)c4ccc5c6ccccc6n(-c6ccccc6)c5c4)cccc32)c2ccc3ccccc3c2)cc1]

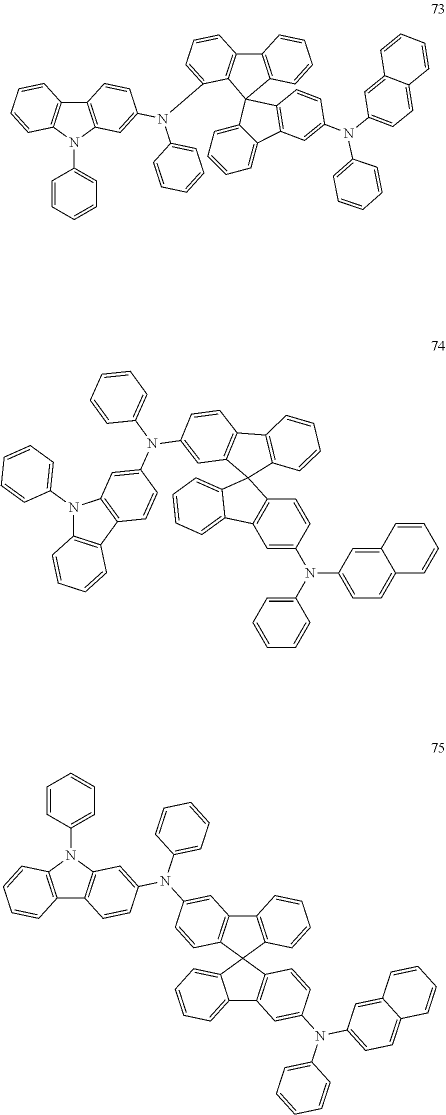 [SMILES: c1ccc(N(c2ccc3c(c2)-c2ccccc2C32c3ccccc3-c3cccc(N(c4ccccc4)c4ccc5c6ccccc6n(-c6ccccc6)c5c4)c32)c2ccc3ccccc3c2)cc1 ; c1ccc(N(c2ccc3c(c2)-c2ccccc2C32c3ccccc3-c3ccc(N(c4ccccc4)c4ccc5c6ccccc6n(-c6ccccc6)c5c4)cc32)c2ccc3ccccc3c2)cc1 ; c1ccc(N(c2ccc3c(c2)-c2ccccc2C32c3ccccc3-c3cc(N(c4ccccc4)c4ccc5c6ccccc6n(-c6ccccc6)c5c4)ccc32)c2ccc3ccccc3c2)cc1]

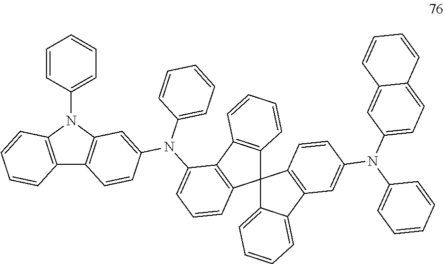 [SMILES: c1ccc(N(c2ccc3c(c2)-c2ccccc2C32c3ccccc3-c3c(N(c4ccccc4)c4ccc5c6ccccc6n(-c6ccccc6)c5c4)cccc32)c2ccc3ccccc3c2)cc1]

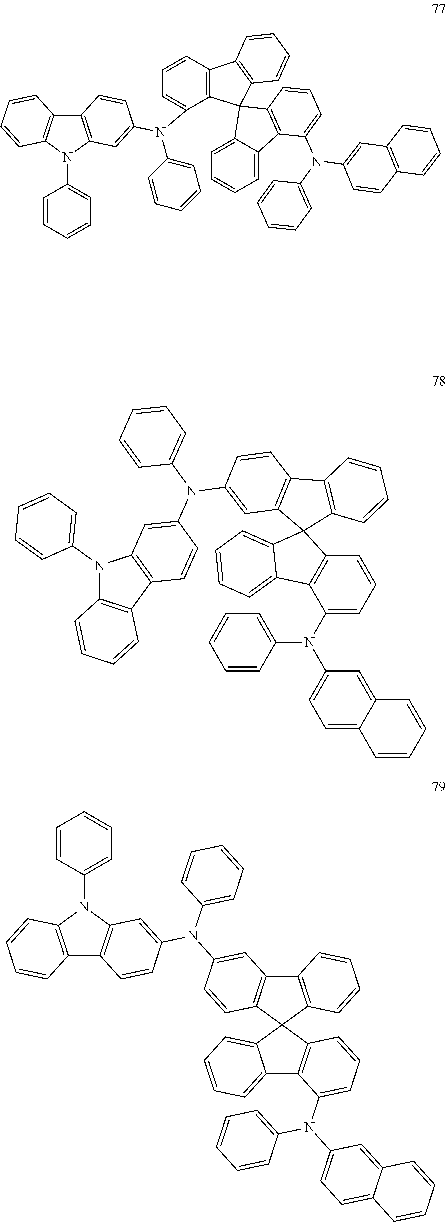 [SMILES: c1ccc(N(c2ccc3ccccc3c2)c2cccc3c2-c2ccccc2C32c3ccccc3-c3cccc(N(c4ccccc4)c4ccc5c6ccccc6n(-c6ccccc6)c5c4)c32)cc1 ; c1ccc(N(c2ccc3c(c2)C2(c4ccccc4-3)c3ccccc3-c3c(N(c4ccccc4)c4ccc5ccccc5c4)cccc32)c2ccc3c4ccccc4n(-c4ccccc4)c3c2)cc1 ; c1ccc(N(c2ccc3c(c2)-c2ccccc2C32c3ccccc3-c3c(N(c4ccccc4)c4ccc5ccccc5c4)cccc32)c2ccc3c4ccccc4n(-c4ccccc4)c3c2)cc1]

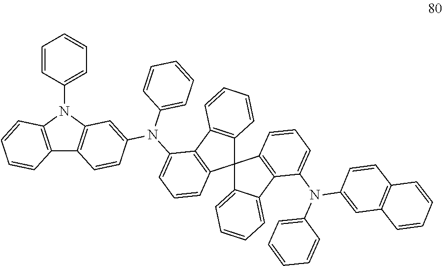 [SMILES: c1ccc(N(c2ccc3ccccc3c2)c2cccc3c2-c2ccccc2C32c3ccccc3-c3c(N(c4ccccc4)c4ccc5c6ccccc6n(-c6ccccc6)c5c4)cccc32)cc1]

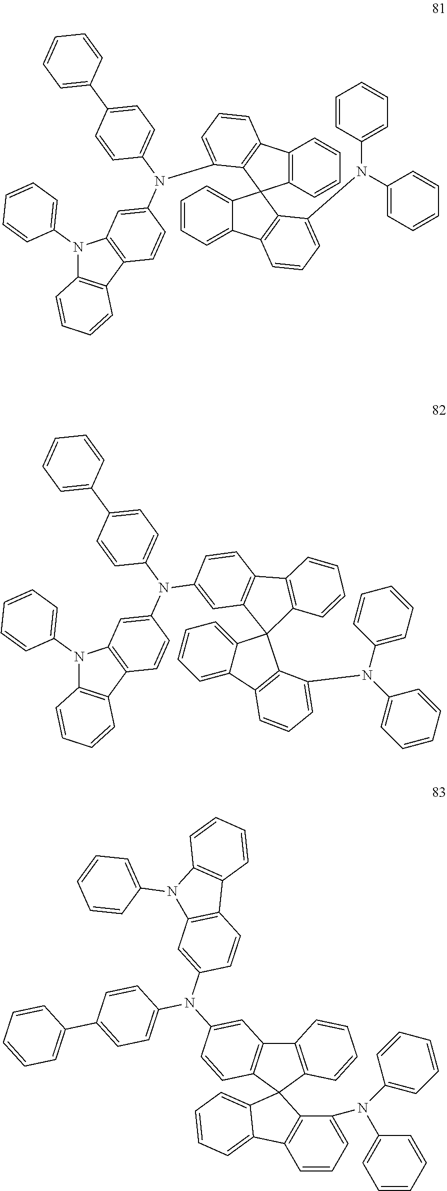 [SMILES: c1ccc(-c2ccc(N(c3ccc4c5ccccc5n(-c5ccccc5)c4c3)c3cccc4c3C3(c5ccccc5-c5cccc(N(c6ccccc6)c6ccccc6)c53)c3ccccc3-4)cc2)cc1 ; c1ccc(-c2ccc(N(c3ccc4c(c3)C3(c5ccccc5-4)c4ccccc4-c4cccc(N(c5ccccc5)c5ccccc5)c43)c3ccc4c5ccccc5n(-c5ccccc5)c4c3)cc2)cc1 ; c1ccc(-c2ccc(N(c3ccc4c(c3)-c3ccccc3C43c4ccccc4-c4cccc(N(c5ccccc5)c5ccccc5)c43)c3ccc4c5ccccc5n(-c5ccccc5)c4c3)cc2)cc1]

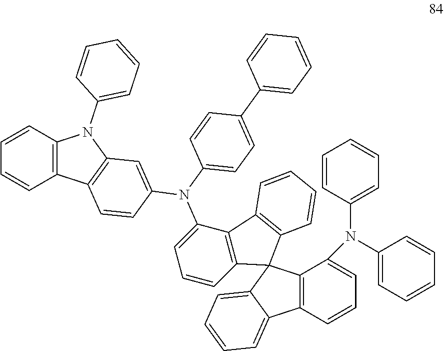 [SMILES: c1ccc(-c2ccc(N(c3ccc4c5ccccc5n(-c5ccccc5)c4c3)c3cccc4c3-c3ccccc3C43c4ccccc4-c4cccc(N(c5ccccc5)c5ccccc5)c43)cc2)cc1]

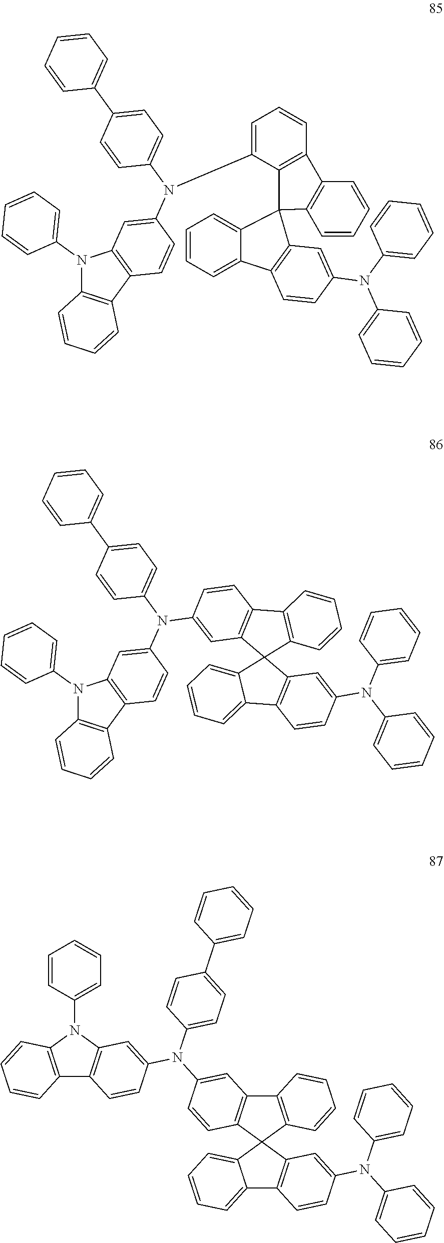 [SMILES: c1ccc(-c2ccc(N(c3ccc4c5ccccc5n(-c5ccccc5)c4c3)c3cccc4c3C3(c5ccccc5-c5ccc(N(c6ccccc6)c6ccccc6)cc53)c3ccccc3-4)cc2)cc1 ; c1ccc(-c2ccc(N(c3ccc4c(c3)C3(c5ccccc5-c5ccc(N(c6ccccc6)c6ccccc6)cc53)c3ccccc3-4)c3ccc4c5ccccc5n(-c5ccccc5)c4c3)cc2)cc1 ; c1ccc(-c2ccc(N(c3ccc4c(c3)-c3ccccc3C43c4ccccc4-c4ccc(N(c5ccccc5)c5ccccc5)cc43)c3ccc4c5ccccc5n(-c5ccccc5)c4c3)cc2)cc1]

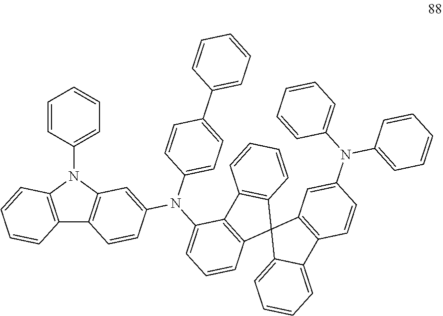 [SMILES: c1ccc(-c2ccc(N(c3ccc4c5ccccc5n(-c5ccccc5)c4c3)c3cccc4c3-c3ccccc3C43c4ccccc4-c4ccc(N(c5ccccc5)c5ccccc5)cc43)cc2)cc1]

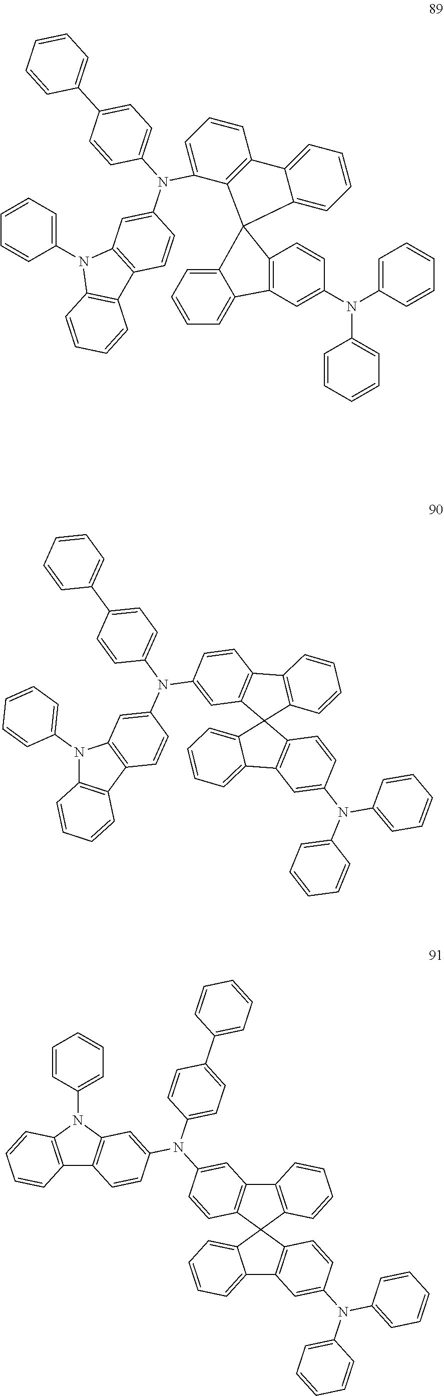 [SMILES: c1ccc(-c2ccc(N(c3ccc4c5ccccc5n(-c5ccccc5)c4c3)c3cccc4c3C3(c5ccccc5-c5cc(N(c6ccccc6)c6ccccc6)ccc53)c3ccccc3-4)cc2)cc1 ; c1ccc(-c2ccc(N(c3ccc4c(c3)C3(c5ccccc5-c5cc(N(c6ccccc6)c6ccccc6)ccc53)c3ccccc3-4)c3ccc4c5ccccc5n(-c5ccccc5)c4c3)cc2)cc1 ; c1ccc(-c2ccc(N(c3ccc4c(c3)-c3ccccc3C43c4ccccc4-c4cc(N(c5ccccc5)c5ccccc5)ccc43)c3ccc4c5ccccc5n(-c5ccccc5)c4c3)cc2)cc1]

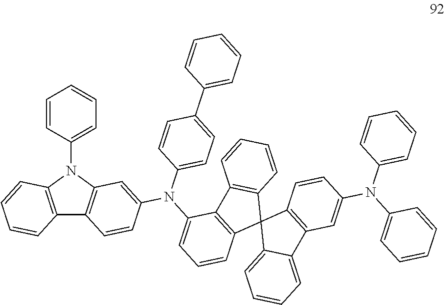 [SMILES: c1ccc(-c2ccc(N(c3ccc4c5ccccc5n(-c5ccccc5)c4c3)c3cccc4c3-c3ccccc3C43c4ccccc4-c4cc(N(c5ccccc5)c5ccccc5)ccc43)cc2)cc1]

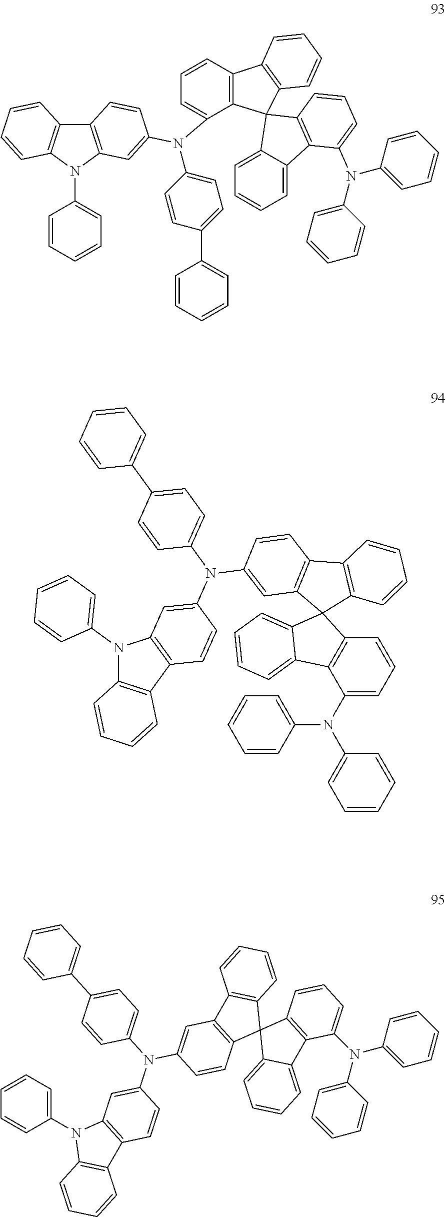 [SMILES: c1ccc(-c2ccc(N(c3ccc4c5ccccc5n(-c5ccccc5)c4c3)c3cccc4c3C3(c5ccccc5-4)c4ccccc4-c4c(N(c5ccccc5)c5ccccc5)cccc43)cc2)cc1 ; c1ccc(-c2ccc(N(c3ccc4c(c3)C3(c5ccccc5-4)c4ccccc4-c4c(N(c5ccccc5)c5ccccc5)cccc43)c3ccc4c5ccccc5n(-c5ccccc5)c4c3)cc2)cc1 ; c1ccc(-c2ccc(N(c3ccc4c(c3)-c3ccccc3C43c4ccccc4-c4c(N(c5ccccc5)c5ccccc5)cccc43)c3ccc4c5ccccc5n(-c5ccccc5)c4c3)cc2)cc1]

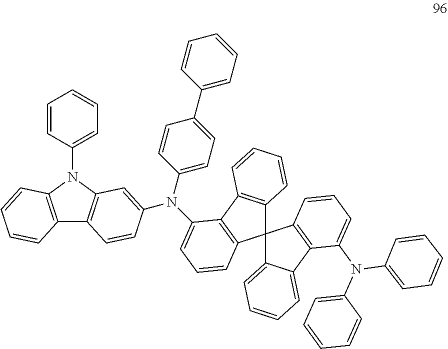 [SMILES: c1ccc(-c2ccc(N(c3ccc4c5ccccc5n(-c5ccccc5)c4c3)c3cccc4c3-c3ccccc3C43c4ccccc4-c4c(N(c5ccccc5)c5ccccc5)cccc43)cc2)cc1]

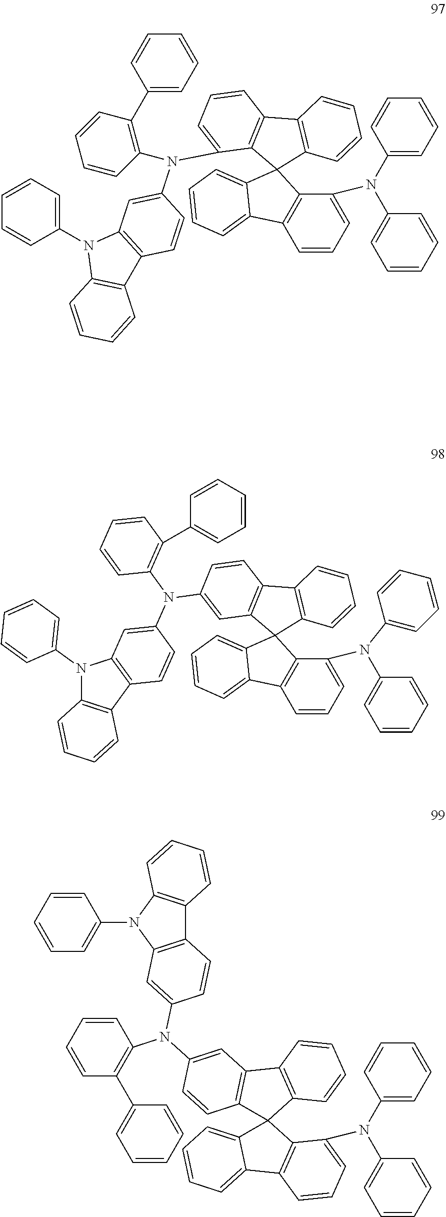 [SMILES: c1ccc(-c2ccccc2N(c2ccc3c4ccccc4n(-c4ccccc4)c3c2)c2cccc3c2C2(c4ccccc4-c4cccc(N(c5ccccc5)c5ccccc5)c42)c2ccccc2-3)cc1 ; c1ccc(-c2ccccc2N(c2ccc3c(c2)C2(c4ccccc4-3)c3ccccc3-c3cccc(N(c4ccccc4)c4ccccc4)c32)c2ccc3c4ccccc4n(-c4ccccc4)c3c2)cc1 ; c1ccc(-c2ccccc2N(c2ccc3c(c2)-c2ccccc2C32c3ccccc3-c3cccc(N(c4ccccc4)c4ccccc4)c32)c2ccc3c4ccccc4n(-c4ccccc4)c3c2)cc1]

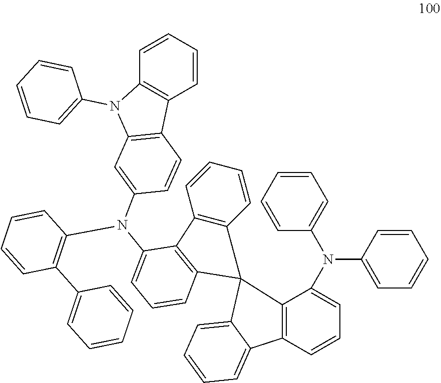 [SMILES: c1ccc(-c2ccccc2N(c2ccc3c4ccccc4n(-c4ccccc4)c3c2)c2cccc3c2-c2ccccc2C32c3ccccc3-c3cccc(N(c4ccccc4)c4ccccc4)c32)cc1]

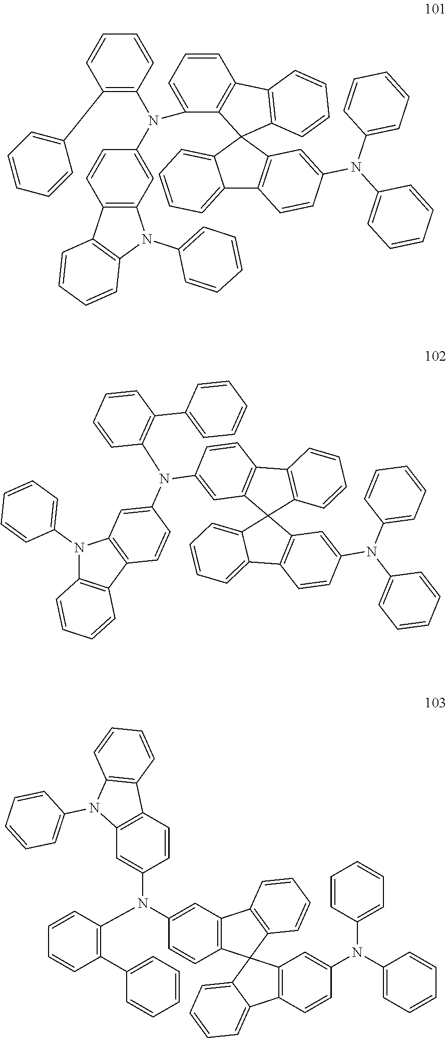 [SMILES: c1ccc(-c2ccccc2N(c2ccc3c4ccccc4n(-c4ccccc4)c3c2)c2cccc3c2C2(c4ccccc4-c4ccc(N(c5ccccc5)c5ccccc5)cc42)c2ccccc2-3)cc1 ; c1ccc(-c2ccccc2N(c2ccc3c(c2)C2(c4ccccc4-c4ccc(N(c5ccccc5)c5ccccc5)cc42)c2ccccc2-3)c2ccc3c4ccccc4n(-c4ccccc4)c3c2)cc1 ; c1ccc(-c2ccccc2N(c2ccc3c(c2)-c2ccccc2C32c3ccccc3-c3ccc(N(c4ccccc4)c4ccccc4)cc32)c2ccc3c4ccccc4n(-c4ccccc4)c3c2)cc1]

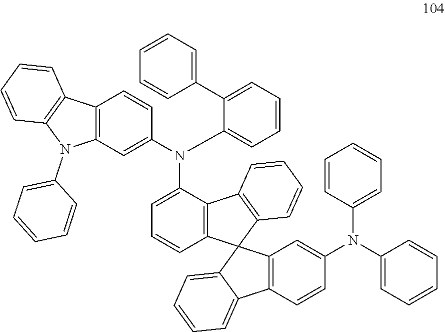 [SMILES: c1ccc(-c2ccccc2N(c2ccc3c4ccccc4n(-c4ccccc4)c3c2)c2cccc3c2-c2ccccc2C32c3ccccc3-c3ccc(N(c4ccccc4)c4ccccc4)cc32)cc1]

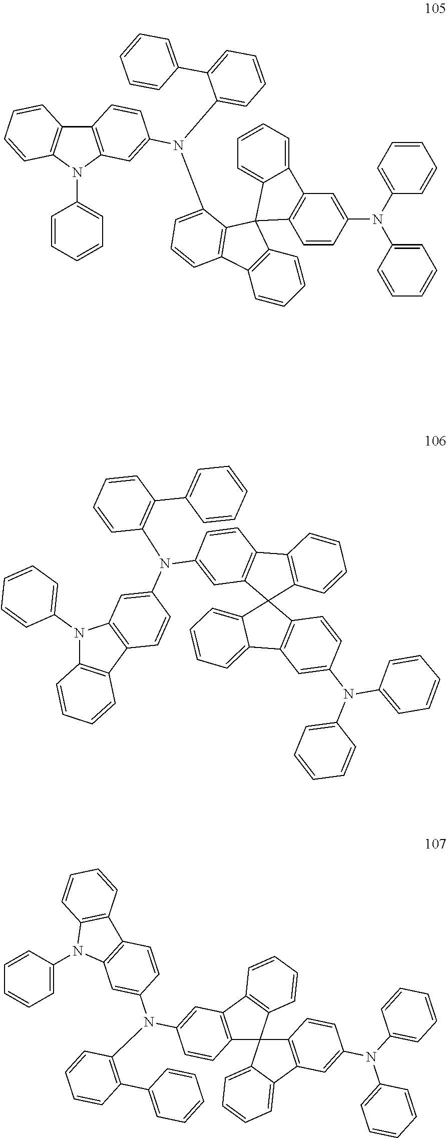 [SMILES: c1ccc(-c2ccccc2N(c2ccc3c4ccccc4n(-c4ccccc4)c3c2)c2cccc3c2C2(c4ccccc4-c4cc(N(c5ccccc5)c5ccccc5)ccc42)c2ccccc2-3)cc1 ; c1ccc(-c2ccccc2N(c2ccc3c(c2)C2(c4ccccc4-c4cc(N(c5ccccc5)c5ccccc5)ccc42)c2ccccc2-3)c2ccc3c4ccccc4n(-c4ccccc4)c3c2)cc1 ; c1ccc(-c2ccccc2N(c2ccc3c(c2)-c2ccccc2C32c3ccccc3-c3cc(N(c4ccccc4)c4ccccc4)ccc32)c2ccc3c4ccccc4n(-c4ccccc4)c3c2)cc1]

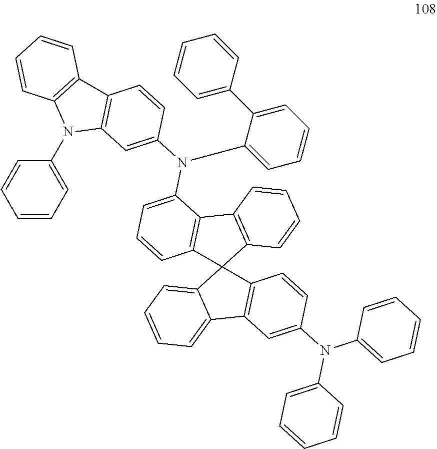 [SMILES: c1ccc(-c2ccccc2N(c2ccc3c4ccccc4n(-c4ccccc4)c3c2)c2cccc3c2-c2ccccc2C32c3ccccc3-c3cc(N(c4ccccc4)c4ccccc4)ccc32)cc1]

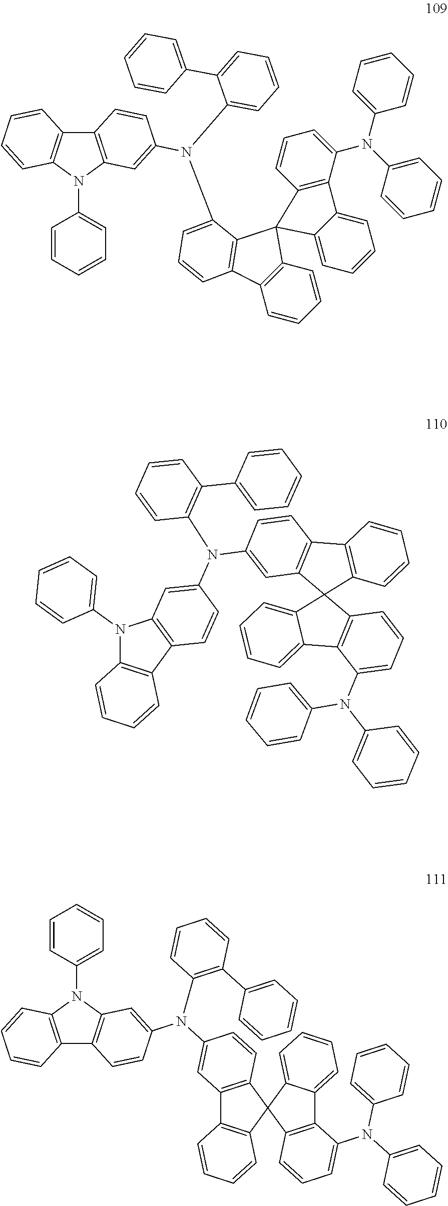 [SMILES: c1ccc(-c2ccccc2N(c2ccc3c4ccccc4n(-c4ccccc4)c3c2)c2cccc3c2C2(c4ccccc4-3)c3ccccc3-c3c(N(c4ccccc4)c4ccccc4)cccc32)cc1 ; c1ccc(-c2ccccc2N(c2ccc3c(c2)C2(c4ccccc4-3)c3ccccc3-c3c(N(c4ccccc4)c4ccccc4)cccc32)c2ccc3c4ccccc4n(-c4ccccc4)c3c2)cc1 ; c1ccc(-c2ccccc2N(c2ccc3c(c2)-c2ccccc2C32c3ccccc3-c3c(N(c4ccccc4)c4ccccc4)cccc32)c2ccc3c4ccccc4n(-c4ccccc4)c3c2)cc1]

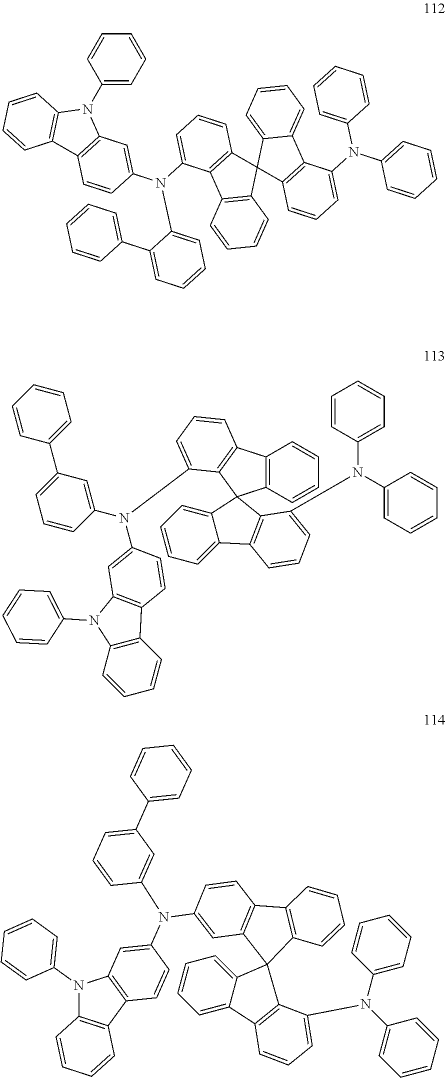 [SMILES: c1ccc(-c2ccccc2N(c2ccc3c4ccccc4n(-c4ccccc4)c3c2)c2cccc3c2-c2ccccc2C32c3ccccc3-c3c(N(c4ccccc4)c4ccccc4)cccc32)cc1 ; c1ccc(-c2cccc(N(c3ccc4c5ccccc5n(-c5ccccc5)c4c3)c3cccc4c3C3(c5ccccc5-c5cccc(N(c6ccccc6)c6ccccc6)c53)c3ccccc3-4)c2)cc1 ; c1ccc(-c2cccc(N(c3ccc4c(c3)C3(c5ccccc5-4)c4ccccc4-c4cccc(N(c5ccccc5)c5ccccc5)c43)c3ccc4c5ccccc5n(-c5ccccc5)c4c3)c2)cc1]

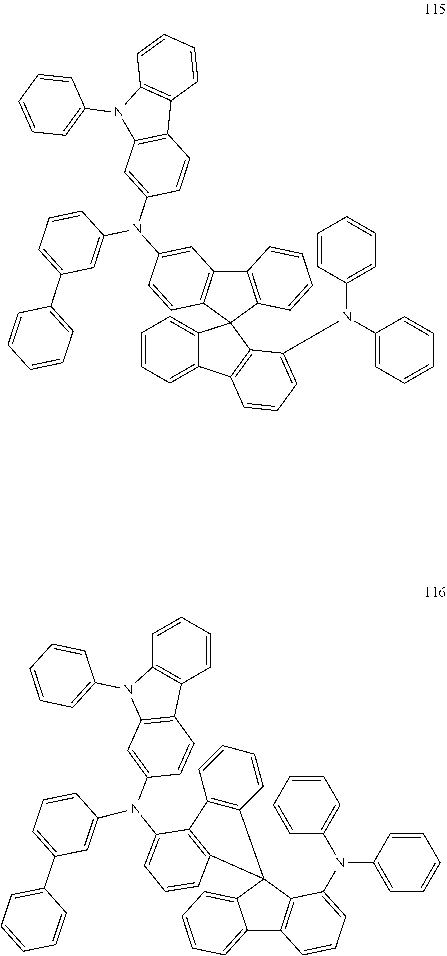 [SMILES: c1ccc(-c2cccc(N(c3ccc4c(c3)-c3ccccc3C43c4ccccc4-c4cccc(N(c5ccccc5)c5ccccc5)c43)c3ccc4c5ccccc5n(-c5ccccc5)c4c3)c2)cc1 ; c1ccc(-c2cccc(N(c3ccc4c5ccccc5n(-c5ccccc5)c4c3)c3cccc4c3-c3ccccc3C43c4ccccc4-c4cccc(N(c5ccccc5)c5ccccc5)c43)c2)cc1]

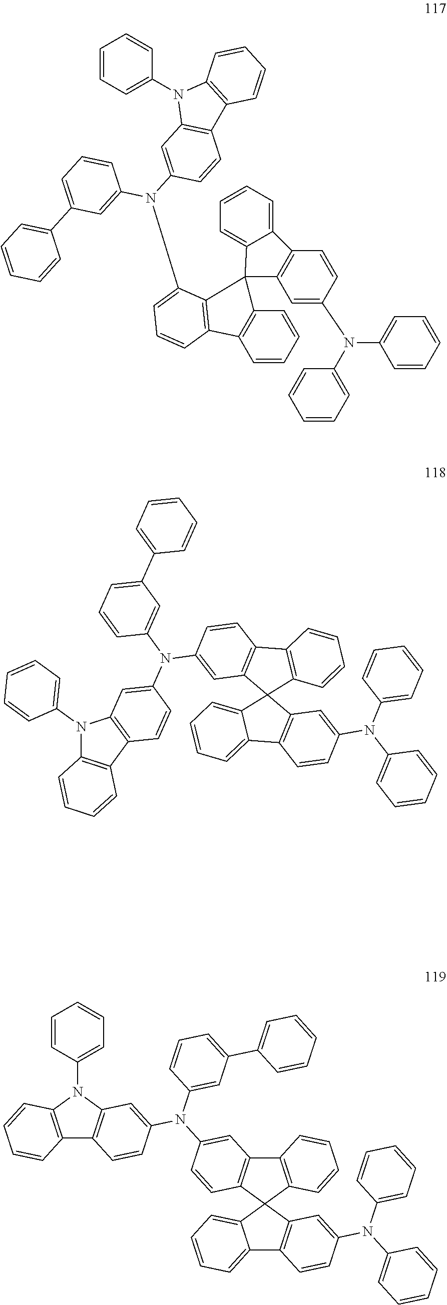 [SMILES: c1ccc(-c2cccc(N(c3ccc4c5ccccc5n(-c5ccccc5)c4c3)c3cccc4c3C3(c5ccccc5-c5ccc(N(c6ccccc6)c6ccccc6)cc53)c3ccccc3-4)c2)cc1 ; c1ccc(-c2cccc(N(c3ccc4c(c3)C3(c5ccccc5-c5ccc(N(c6ccccc6)c6ccccc6)cc53)c3ccccc3-4)c3ccc4c5ccccc5n(-c5ccccc5)c4c3)c2)cc1 ; c1ccc(-c2cccc(N(c3ccc4c(c3)-c3ccccc3C43c4ccccc4-c4ccc(N(c5ccccc5)c5ccccc5)cc43)c3ccc4c5ccccc5n(-c5ccccc5)c4c3)c2)cc1]

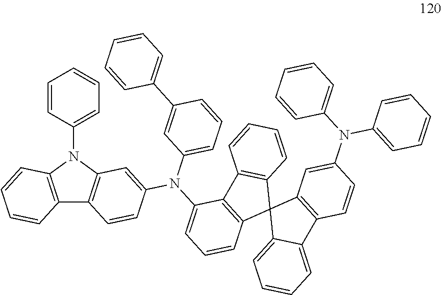 [SMILES: c1ccc(-c2cccc(N(c3ccc4c5ccccc5n(-c5ccccc5)c4c3)c3cccc4c3-c3ccccc3C43c4ccccc4-c4ccc(N(c5ccccc5)c5ccccc5)cc43)c2)cc1]

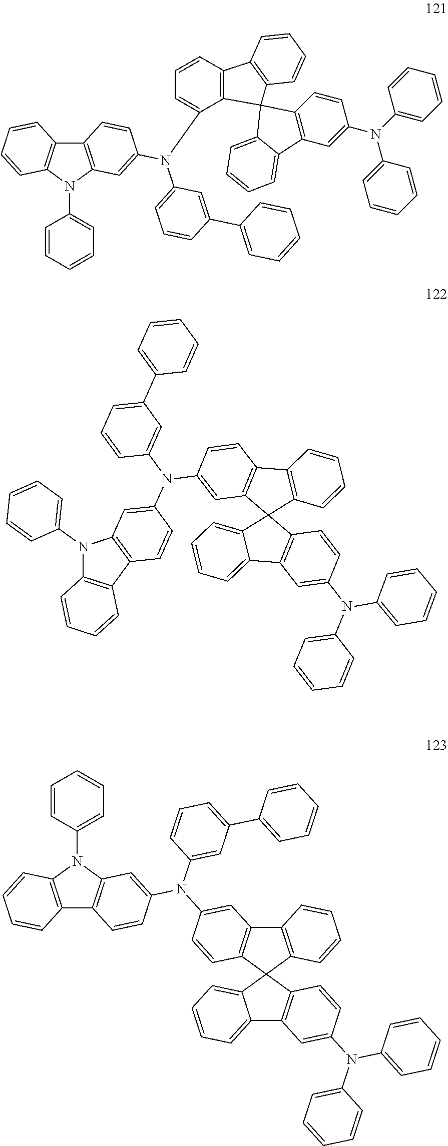 [SMILES: c1ccc(-c2cccc(N(c3ccc4c5ccccc5n(-c5ccccc5)c4c3)c3cccc4c3C3(c5ccccc5-c5cc(N(c6ccccc6)c6ccccc6)ccc53)c3ccccc3-4)c2)cc1 ; c1ccc(-c2cccc(N(c3ccc4c(c3)C3(c5ccccc5-c5cc(N(c6ccccc6)c6ccccc6)ccc53)c3ccccc3-4)c3ccc4c5ccccc5n(-c5ccccc5)c4c3)c2)cc1 ; c1ccc(-c2cccc(N(c3ccc4c(c3)-c3ccccc3C43c4ccccc4-c4cc(N(c5ccccc5)c5ccccc5)ccc43)c3ccc4c5ccccc5n(-c5ccccc5)c4c3)c2)cc1]

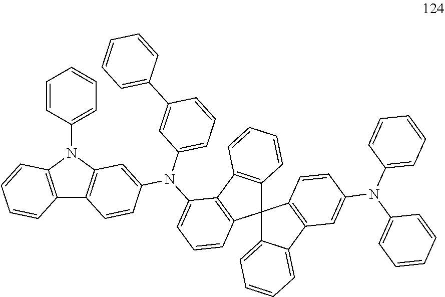 [SMILES: c1ccc(-c2cccc(N(c3ccc4c5ccccc5n(-c5ccccc5)c4c3)c3cccc4c3-c3ccccc3C43c4ccccc4-c4cc(N(c5ccccc5)c5ccccc5)ccc43)c2)cc1]

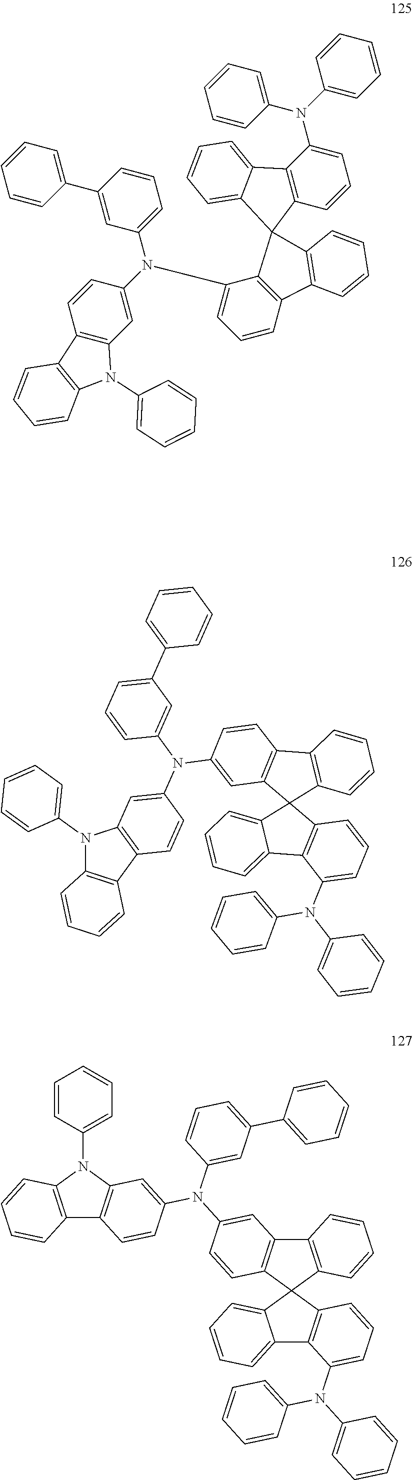 [SMILES: c1ccc(-c2cccc(N(c3ccc4c5ccccc5n(-c5ccccc5)c4c3)c3cccc4c3C3(c5ccccc5-4)c4ccccc4-c4c(N(c5ccccc5)c5ccccc5)cccc43)c2)cc1 ; c1ccc(-c2cccc(N(c3ccc4c(c3)C3(c5ccccc5-4)c4ccccc4-c4c(N(c5ccccc5)c5ccccc5)cccc43)c3ccc4c5ccccc5n(-c5ccccc5)c4c3)c2)cc1 ; c1ccc(-c2cccc(N(c3ccc4c(c3)-c3ccccc3C43c4ccccc4-c4c(N(c5ccccc5)c5ccccc5)cccc43)c3ccc4c5ccccc5n(-c5ccccc5)c4c3)c2)cc1]

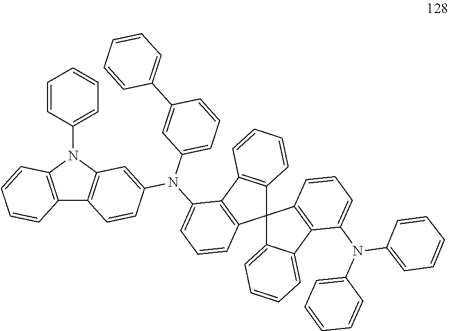 [SMILES: c1ccc(-c2cccc(N(c3ccc4c5ccccc5n(-c5ccccc5)c4c3)c3cccc4c3-c3ccccc3C43c4ccccc4-c4c(N(c5ccccc5)c5ccccc5)cccc43)c2)cc1]

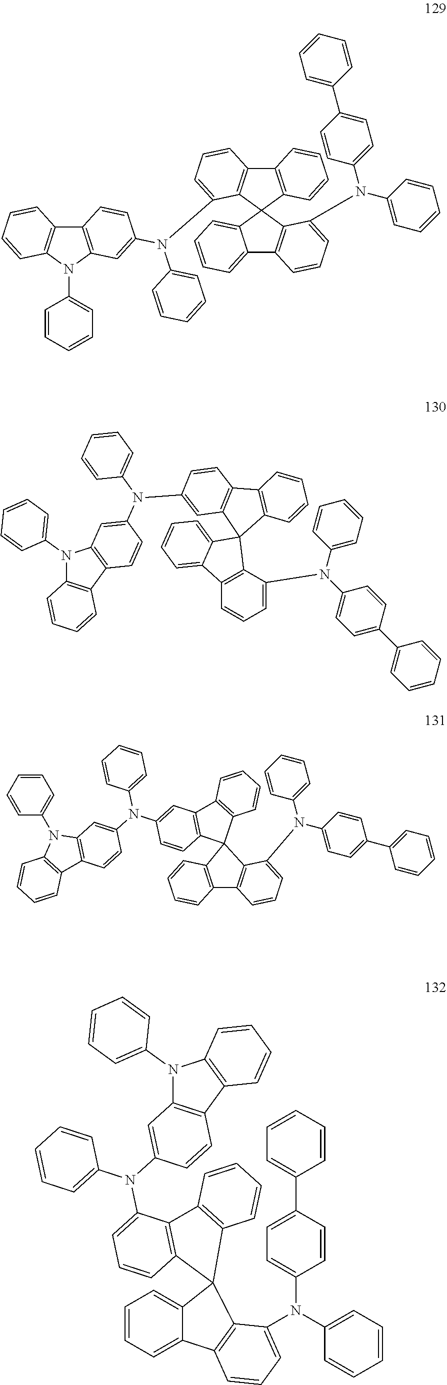 [SMILES: c1ccc(-c2ccc(N(c3ccccc3)c3cccc4c3C3(c5ccccc5-4)c4ccccc4-c4cccc(N(c5ccccc5)c5ccc6c7ccccc7n(-c7ccccc7)c6c5)c43)cc2)cc1 ; c1ccc(-c2ccc(N(c3ccccc3)c3cccc4c3C3(c5ccccc5-c5ccc(N(c6ccccc6)c6ccc7c8ccccc8n(-c8ccccc8)c7c6)cc53)c3ccccc3-4)cc2)cc1 ; c1ccc(-c2ccc(N(c3ccccc3)c3cccc4c3C3(c5ccccc5-c5cc(N(c6ccccc6)c6ccc7c8ccccc8n(-c8ccccc8)c7c6)ccc53)c3ccccc3-4)cc2)cc1 ; c1ccc(-c2ccc(N(c3ccccc3)c3cccc4c3C3(c5ccccc5-4)c4ccccc4-c4c(N(c5ccccc5)c5ccc6c7ccccc7n(-c7ccccc7)c6c5)cccc43)cc2)cc1]

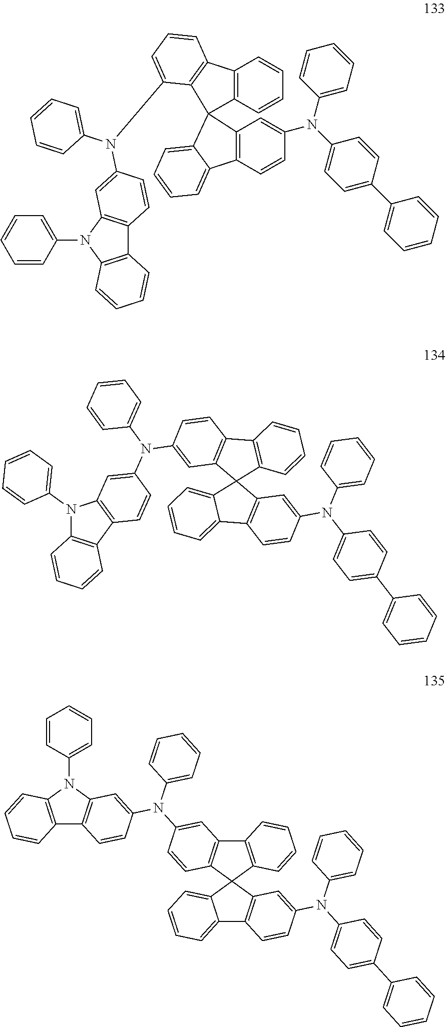 [SMILES: c1ccc(-c2ccc(N(c3ccccc3)c3ccc4c(c3)C3(c5ccccc5-4)c4ccccc4-c4cccc(N(c5ccccc5)c5ccc6c7ccccc7n(-c7ccccc7)c6c5)c43)cc2)cc1 ; c1ccc(-c2ccc(N(c3ccccc3)c3ccc4c(c3)C3(c5ccccc5-4)c4ccccc4-c4ccc(N(c5ccccc5)c5ccc6c7ccccc7n(-c7ccccc7)c6c5)cc43)cc2)cc1 ; c1ccc(-c2ccc(N(c3ccccc3)c3ccc4c(c3)C3(c5ccccc5-c5cc(N(c6ccccc6)c6ccc7c8ccccc8n(-c8ccccc8)c7c6)ccc53)c3ccccc3-4)cc2)cc1]

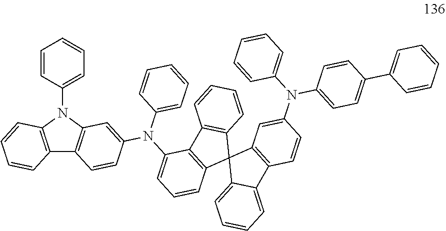 [SMILES: c1ccc(-c2ccc(N(c3ccccc3)c3ccc4c(c3)C3(c5ccccc5-4)c4ccccc4-c4c(N(c5ccccc5)c5ccc6c7ccccc7n(-c7ccccc7)c6c5)cccc43)cc2)cc1]

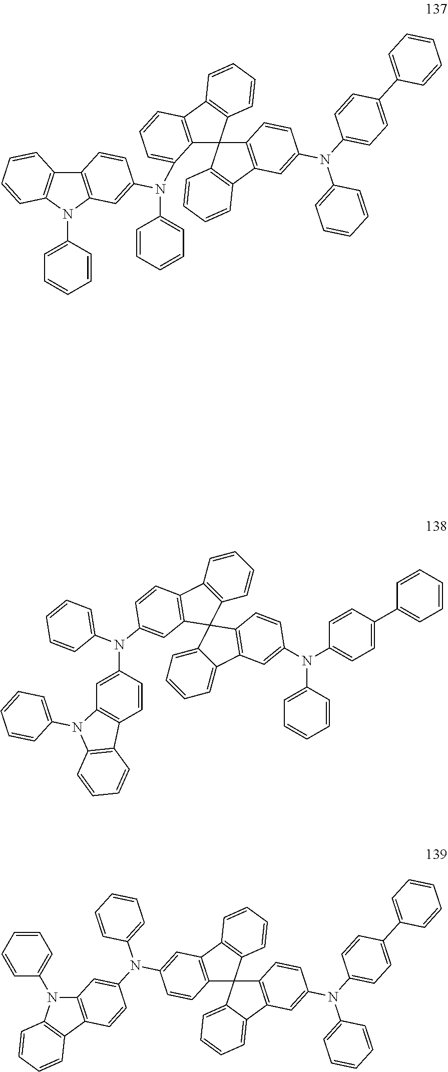 [SMILES: c1ccc(-c2ccc(N(c3ccccc3)c3ccc4c(c3)-c3ccccc3C43c4ccccc4-c4cccc(N(c5ccccc5)c5ccc6c7ccccc7n(-c7ccccc7)c6c5)c43)cc2)cc1 ; c1ccc(-c2ccc(N(c3ccccc3)c3ccc4c(c3)-c3ccccc3C43c4ccccc4-c4ccc(N(c5ccccc5)c5ccc6c7ccccc7n(-c7ccccc7)c6c5)cc43)cc2)cc1 ; c1ccc(-c2ccc(N(c3ccccc3)c3ccc4c(c3)-c3ccccc3C43c4ccccc4-c4cc(N(c5ccccc5)c5ccc6c7ccccc7n(-c7ccccc7)c6c5)ccc43)cc2)cc1]

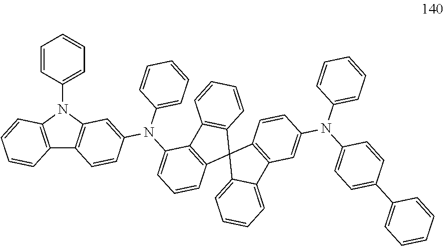 [SMILES: c1ccc(-c2ccc(N(c3ccccc3)c3ccc4c(c3)-c3ccccc3C43c4ccccc4-c4c(N(c5ccccc5)c5ccc6c7ccccc7n(-c7ccccc7)c6c5)cccc43)cc2)cc1]

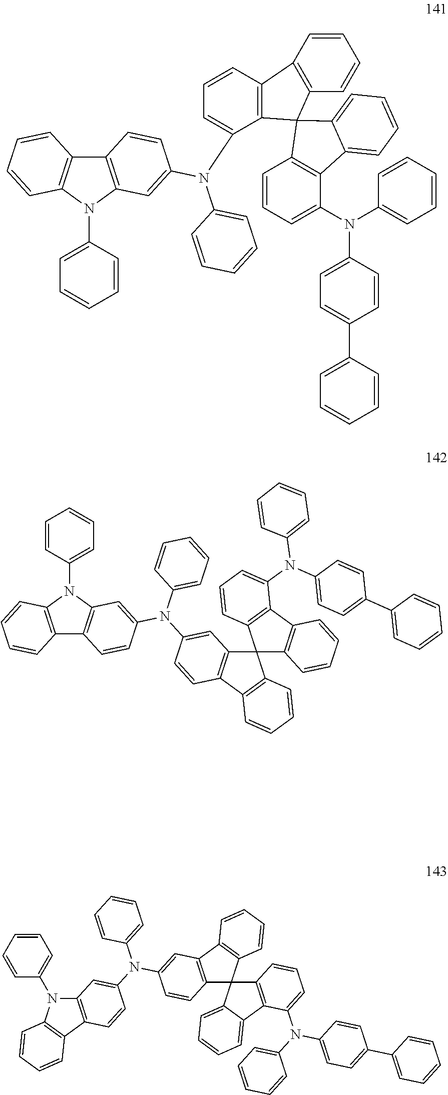 [SMILES: c1ccc(-c2ccc(N(c3ccccc3)c3cccc4c3-c3ccccc3C43c4ccccc4-c4cccc(N(c5ccccc5)c5ccc6c7ccccc7n(-c7ccccc7)c6c5)c43)cc2)cc1 ; c1ccc(-c2ccc(N(c3ccccc3)c3cccc4c3-c3ccccc3C43c4ccccc4-c4ccc(N(c5ccccc5)c5ccc6c7ccccc7n(-c7ccccc7)c6c5)cc43)cc2)cc1 ; c1ccc(-c2ccc(N(c3ccccc3)c3cccc4c3-c3ccccc3C43c4ccccc4-c4cc(N(c5ccccc5)c5ccc6c7ccccc7n(-c7ccccc7)c6c5)ccc43)cc2)cc1]

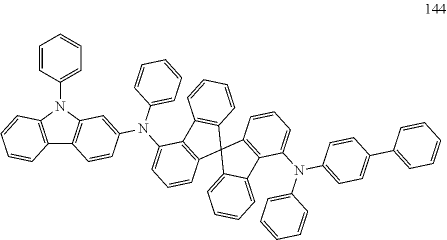 [SMILES: c1ccc(-c2ccc(N(c3ccccc3)c3cccc4c3-c3ccccc3C43c4ccccc4-c4c(N(c5ccccc5)c5ccc6c7ccccc7n(-c7ccccc7)c6c5)cccc43)cc2)cc1]

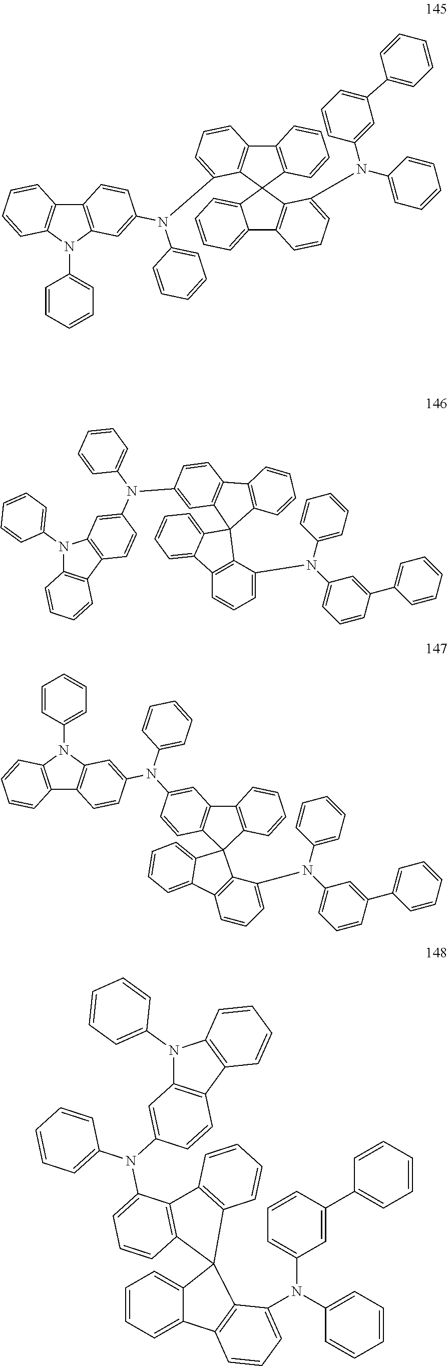 [SMILES: c1ccc(-c2cccc(N(c3ccccc3)c3cccc4c3C3(c5ccccc5-4)c4ccccc4-c4cccc(N(c5ccccc5)c5ccc6c7ccccc7n(-c7ccccc7)c6c5)c43)c2)cc1 ; c1ccc(-c2cccc(N(c3ccccc3)c3cccc4c3C3(c5ccccc5-c5ccc(N(c6ccccc6)c6ccc7c8ccccc8n(-c8ccccc8)c7c6)cc53)c3ccccc3-4)c2)cc1 ; c1ccc(-c2cccc(N(c3ccccc3)c3cccc4c3C3(c5ccccc5-c5cc(N(c6ccccc6)c6ccc7c8ccccc8n(-c8ccccc8)c7c6)ccc53)c3ccccc3-4)c2)cc1 ; c1ccc(-c2cccc(N(c3ccccc3)c3cccc4c3C3(c5ccccc5-4)c4ccccc4-c4c(N(c5ccccc5)c5ccc6c7ccccc7n(-c7ccccc7)c6c5)cccc43)c2)cc1]

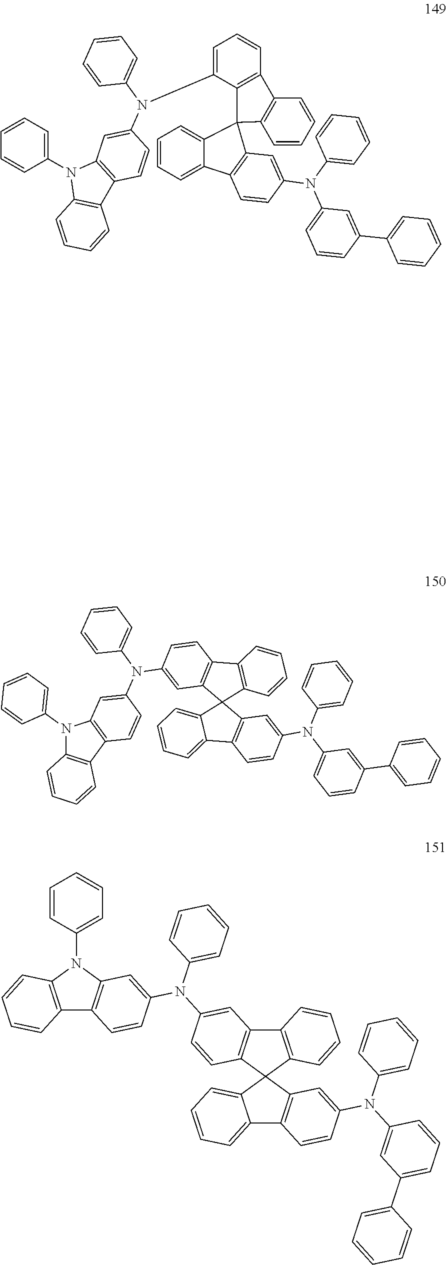 [SMILES: c1ccc(-c2cccc(N(c3ccccc3)c3ccc4c(c3)C3(c5ccccc5-4)c4ccccc4-c4cccc(N(c5ccccc5)c5ccc6c7ccccc7n(-c7ccccc7)c6c5)c43)c2)cc1 ; c1ccc(-c2cccc(N(c3ccccc3)c3ccc4c(c3)C3(c5ccccc5-4)c4ccccc4-c4ccc(N(c5ccccc5)c5ccc6c7ccccc7n(-c7ccccc7)c6c5)cc43)c2)cc1 ; c1ccc(-c2cccc(N(c3ccccc3)c3ccc4c(c3)C3(c5ccccc5-c5cc(N(c6ccccc6)c6ccc7c8ccccc8n(-c8ccccc8)c7c6)ccc53)c3ccccc3-4)c2)cc1]

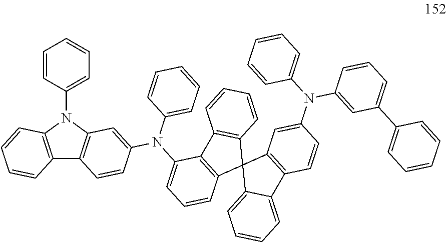 [SMILES: c1ccc(-c2cccc(N(c3ccccc3)c3ccc4c(c3)C3(c5ccccc5-4)c4ccccc4-c4c(N(c5ccccc5)c5ccc6c7ccccc7n(-c7ccccc7)c6c5)cccc43)c2)cc1]

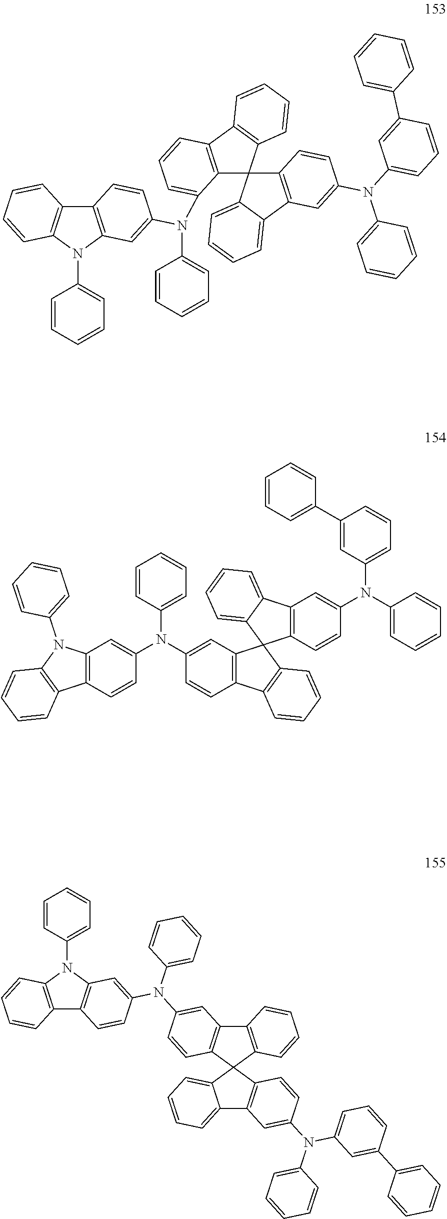 [SMILES: c1ccc(-c2cccc(N(c3ccccc3)c3ccc4c(c3)-c3ccccc3C43c4ccccc4-c4cccc(N(c5ccccc5)c5ccc6c7ccccc7n(-c7ccccc7)c6c5)c43)c2)cc1 ; c1ccc(-c2cccc(N(c3ccccc3)c3ccc4c(c3)-c3ccccc3C43c4ccccc4-c4ccc(N(c5ccccc5)c5ccc6c7ccccc7n(-c7ccccc7)c6c5)cc43)c2)cc1 ; c1ccc(-c2cccc(N(c3ccccc3)c3ccc4c(c3)-c3ccccc3C43c4ccccc4-c4cc(N(c5ccccc5)c5ccc6c7ccccc7n(-c7ccccc7)c6c5)ccc43)c2)cc1]

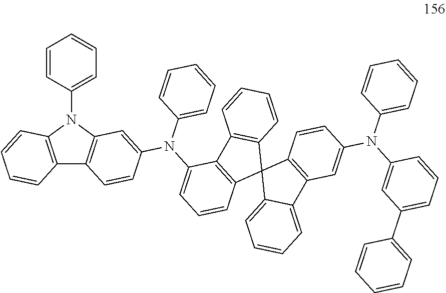 [SMILES: c1ccc(-c2cccc(N(c3ccccc3)c3ccc4c(c3)-c3ccccc3C43c4ccccc4-c4c(N(c5ccccc5)c5ccc6c7ccccc7n(-c7ccccc7)c6c5)cccc43)c2)cc1]

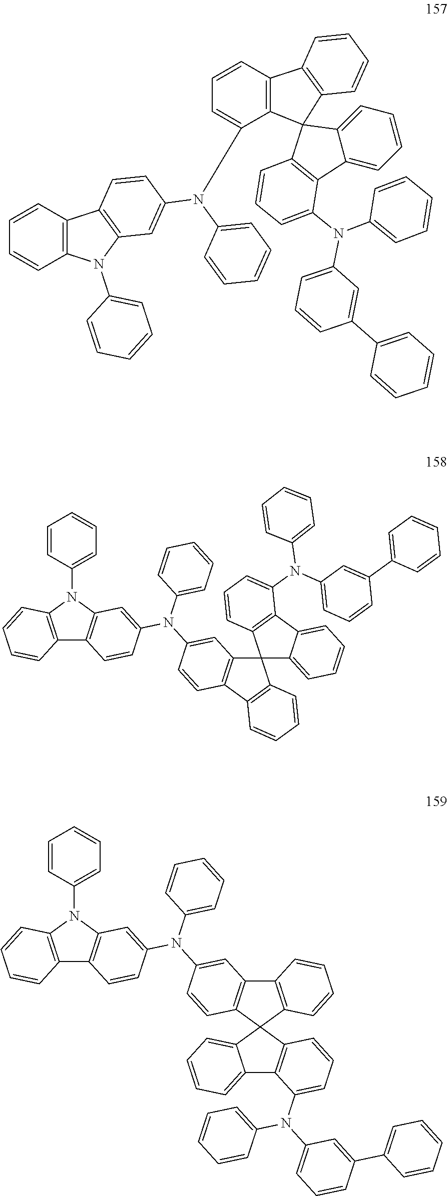 [SMILES: c1ccc(-c2cccc(N(c3ccccc3)c3cccc4c3-c3ccccc3C43c4ccccc4-c4cccc(N(c5ccccc5)c5ccc6c7ccccc7n(-c7ccccc7)c6c5)c43)c2)cc1 ; c1ccc(-c2cccc(N(c3ccccc3)c3cccc4c3-c3ccccc3C43c4ccccc4-c4ccc(N(c5ccccc5)c5ccc6c7ccccc7n(-c7ccccc7)c6c5)cc43)c2)cc1 ; c1ccc(-c2cccc(N(c3ccccc3)c3cccc4c3-c3ccccc3C43c4ccccc4-c4cc(N(c5ccccc5)c5ccc6c7ccccc7n(-c7ccccc7)c6c5)ccc43)c2)cc1]

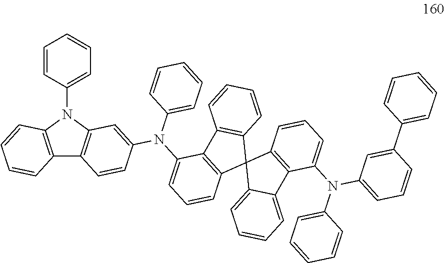 [SMILES: c1ccc(-c2cccc(N(c3ccccc3)c3cccc4c3-c3ccccc3C43c4ccccc4-c4c(N(c5ccccc5)c5ccc6c7ccccc7n(-c7ccccc7)c6c5)cccc43)c2)cc1]

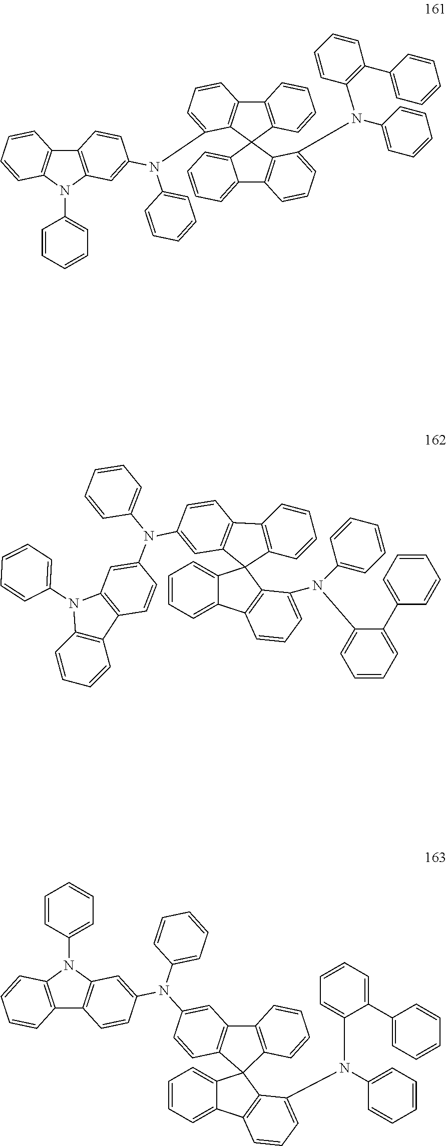 [SMILES: c1ccc(-c2ccccc2N(c2ccccc2)c2cccc3c2C2(c4ccccc4-c4cccc(N(c5ccccc5)c5ccc6c7ccccc7n(-c7ccccc7)c6c5)c42)c2ccccc2-3)cc1 ; c1ccc(-c2ccccc2N(c2ccccc2)c2cccc3c2C2(c4ccccc4-c4ccc(N(c5ccccc5)c5ccc6c7ccccc7n(-c7ccccc7)c6c5)cc42)c2ccccc2-3)cc1 ; c1ccc(-c2ccccc2N(c2ccccc2)c2cccc3c2C2(c4ccccc4-c4cc(N(c5ccccc5)c5ccc6c7ccccc7n(-c7ccccc7)c6c5)ccc42)c2ccccc2-3)cc1]

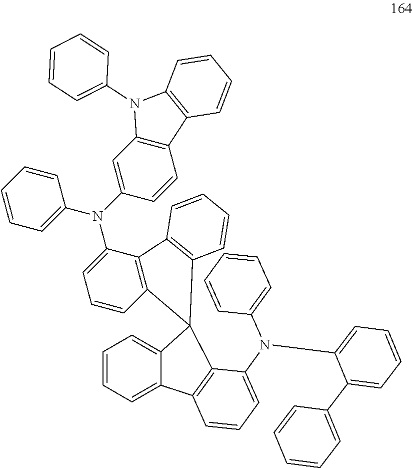 [SMILES: c1ccc(-c2ccccc2N(c2ccccc2)c2cccc3c2C2(c4ccccc4-3)c3ccccc3-c3c(N(c4ccccc4)c4ccc5c6ccccc6n(-c6ccccc6)c5c4)cccc32)cc1]

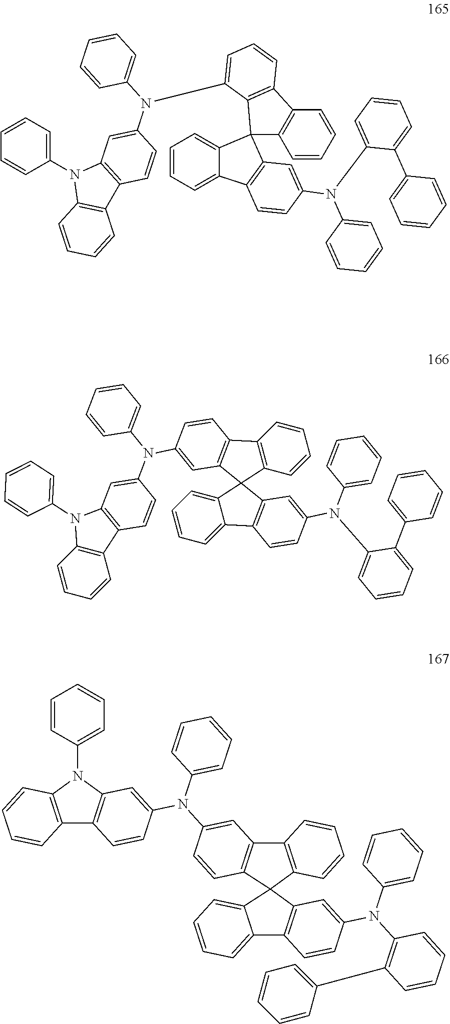 [SMILES: c1ccc(-c2ccccc2N(c2ccccc2)c2ccc3c(c2)C2(c4ccccc4-3)c3ccccc3-c3cccc(N(c4ccccc4)c4ccc5c6ccccc6n(-c6ccccc6)c5c4)c32)cc1 ; c1ccc(-c2ccccc2N(c2ccccc2)c2ccc3c(c2)C2(c4ccccc4-c4ccc(N(c5ccccc5)c5ccc6c7ccccc7n(-c7ccccc7)c6c5)cc42)c2ccccc2-3)cc1 ; c1ccc(-c2ccccc2N(c2ccccc2)c2ccc3c(c2)C2(c4ccccc4-c4cc(N(c5ccccc5)c5ccc6c7ccccc7n(-c7ccccc7)c6c5)ccc42)c2ccccc2-3)cc1]

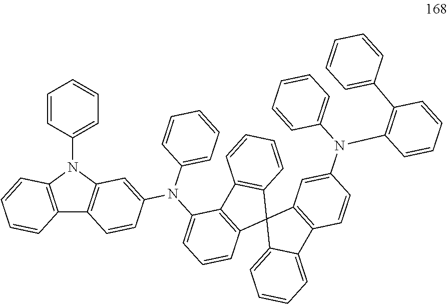 [SMILES: c1ccc(-c2ccccc2N(c2ccccc2)c2ccc3c(c2)C2(c4ccccc4-3)c3ccccc3-c3c(N(c4ccccc4)c4ccc5c6ccccc6n(-c6ccccc6)c5c4)cccc32)cc1]

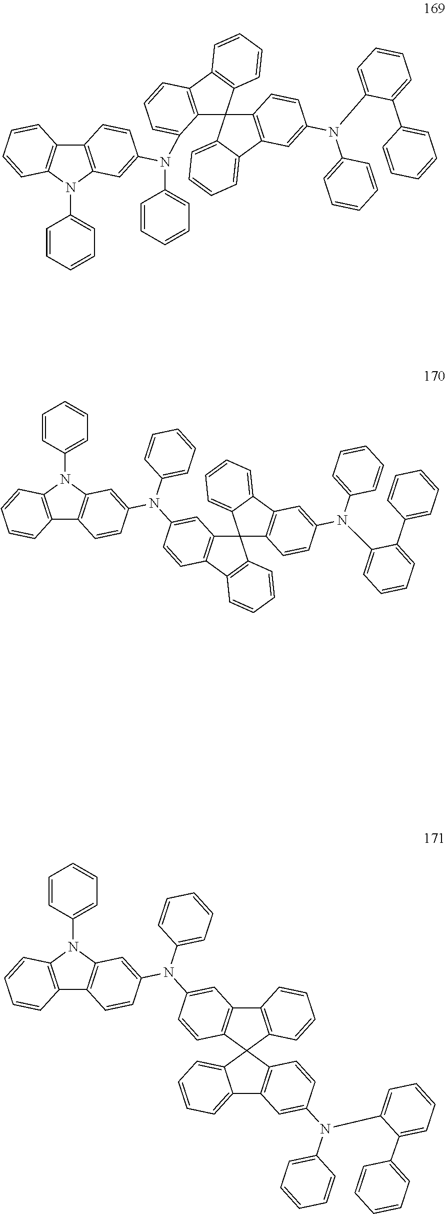 [SMILES: c1ccc(-c2ccccc2N(c2ccccc2)c2ccc3c(c2)-c2ccccc2C32c3ccccc3-c3cccc(N(c4ccccc4)c4ccc5c6ccccc6n(-c6ccccc6)c5c4)c32)cc1 ; c1ccc(-c2ccccc2N(c2ccccc2)c2ccc3c(c2)-c2ccccc2C32c3ccccc3-c3ccc(N(c4ccccc4)c4ccc5c6ccccc6n(-c6ccccc6)c5c4)cc32)cc1 ; c1ccc(-c2ccccc2N(c2ccccc2)c2ccc3c(c2)-c2ccccc2C32c3ccccc3-c3cc(N(c4ccccc4)c4ccc5c6ccccc6n(-c6ccccc6)c5c4)ccc32)cc1]

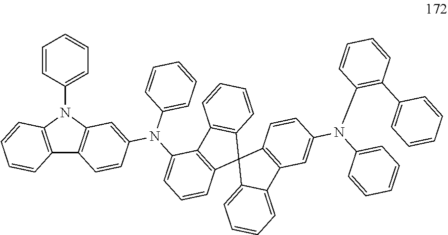 [SMILES: c1ccc(-c2ccccc2N(c2ccccc2)c2ccc3c(c2)-c2ccccc2C32c3ccccc3-c3c(N(c4ccccc4)c4ccc5c6ccccc6n(-c6ccccc6)c5c4)cccc32)cc1]

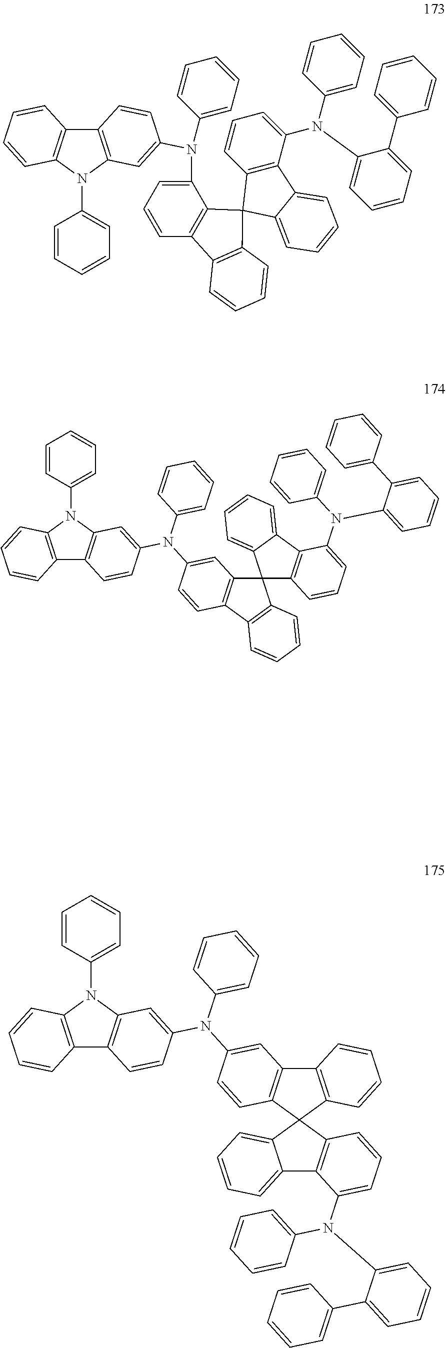 [SMILES: c1ccc(-c2ccccc2N(c2ccccc2)c2cccc3c2-c2ccccc2C32c3ccccc3-c3cccc(N(c4ccccc4)c4ccc5c6ccccc6n(-c6ccccc6)c5c4)c32)cc1 ; c1ccc(-c2ccccc2N(c2ccccc2)c2cccc3c2-c2ccccc2C32c3ccccc3-c3ccc(N(c4ccccc4)c4ccc5c6ccccc6n(-c6ccccc6)c5c4)cc32)cc1 ; c1ccc(-c2ccccc2N(c2ccccc2)c2cccc3c2-c2ccccc2C32c3ccccc3-c3cc(N(c4ccccc4)c4ccc5c6ccccc6n(-c6ccccc6)c5c4)ccc32)cc1]

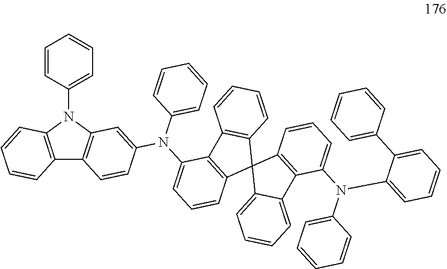 [SMILES: c1ccc(-c2ccccc2N(c2ccccc2)c2cccc3c2-c2ccccc2C32c3ccccc3-c3c(N(c4ccccc4)c4ccc5c6ccccc6n(-c6ccccc6)c5c4)cccc32)cc1]

The amine compound represented by Formula 1 according to an embodiment has a molecular structure in which an amine derivative is bonded to a 9,9′-Spirobi[9H-fluorene] core that is a condensed ring having a spiro structure. More specifically, one fluorene unit of the 9,9′-Spirobi[9H-fluorene] cores includes an amine group containing a carbazole group represented by Formula 2, and the other fluorene unit includes an amine group represented by Formula 3. Accordingly, the amine compound according to an embodiment may have high glass transition temperature and high melting point properties, thereby exhibiting suitable (e.g., excellent) heat resistance and durability characteristics.

In addition, the amine group represented by Formula 2 has a structure in which the nitrogen atom is bonded to the 2-position carbon of the carbazole group. The amine group represented by Formula 2 having such a structure is bonded to Formula 1, and thus may have a relatively high (e.g., deep) highest occupied molecular orbital (HOMO) energy level, thereby further improving hole transport properties. Therefore, when the amine compound of an embodiment is applied in the hole transport region, the hole transport properties may be increased, thus improving recombination probability of holes and electrons in the emission layer, thereby increasing luminous efficiency.

The hole transport region HTR may be formed utilizing various suitable methods such as a vacuum deposition method, a spin coating method, a cast method, a Langmuir-Blodgett (LB) method, an inkjet printing method, a laser printing method, and/or a laser induced thermal imaging (LITI) method.

In one or more embodiments, in the light emitting device ED of an embodiment, the hole transport region HTR may further include a suitable (e.g., known) material.

The hole transport region HTR may include a compound represented by Formula H-1 below:

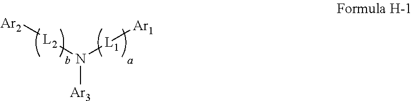

In Formula H-1 above, L₁ and L₂ may be each independently a direct linkage, a substituted or unsubstituted arylene group having 6 to 30 ring-forming carbon atoms, or a substituted or unsubstituted heteroarylene group having 2 to 30 ring-forming carbon atoms. a and b may be each independently an integer of 0 to 10. In one or more embodiments, when a or b is an integer of 2 or greater, a plurality of L₁s and L₂s may be each independently a substituted or unsubstituted arylene group having 6 to 30 ring-forming carbon atoms, or a substituted or unsubstituted heteroarylene group having 2 to 30 ring-forming carbon atoms.

In Formula H-1, Ar₁ and Ar₂ may be each independently a substituted or unsubstituted aryl group having 6 to 30 ring-forming carbon atoms, or a substituted or unsubstituted heteroaryl group having 2 to 30 ring-forming carbon atoms. In addition, in Formula H-1, Ar₃ may be a substituted or unsubstituted aryl group having 6 to 30 ring-forming carbon atoms.

The compound represented by Formula H-1 above may be a monoamine compound. In one or more embodiments, the compound represented by Formula H-1 above may be a diamine compound in which at least one among Ar₁ to Ar₃ includes the amine group as a substituent. In addition, the compound represented by Formula H-1 above may be a carbazole-based compound including a substituted or unsubstituted carbazole group in at least one of Ar₁ or Ar₂, or a fluorene-based compound including a substituted or unsubstituted fluorene group in at least one of Ar₁ or Ar₂.

The compound represented by Formula H-1 may be represented by any one among the compounds of Compound Group H below. However, the compounds listed in Compound Group H below are examples, and the compounds represented by Formula H-1 are not limited to those represented by Compound Group H below:

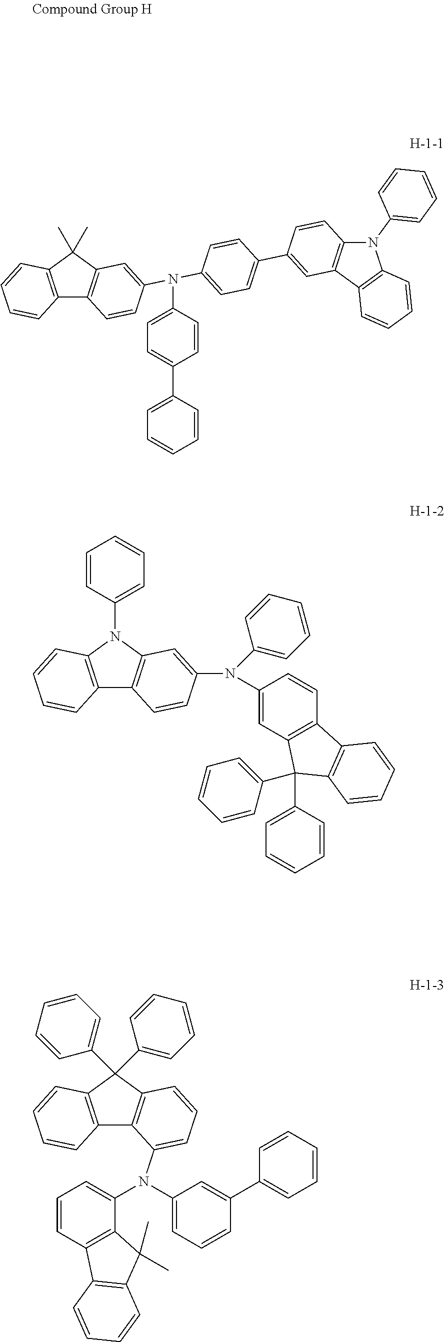 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21 ; c1ccc(N(c2ccc3c(c2)C(c2ccccc2)(c2ccccc2)c2ccccc2-3)c2ccc3c4ccccc4n(-c4ccccc4)c3c2)cc1 ; CC1(C)c2ccccc2-c2cccc(N(c3cccc(-c4ccccc4)c3)c3cccc4c3-c3ccccc3C4(c3ccccc3)c3ccccc3)c21]

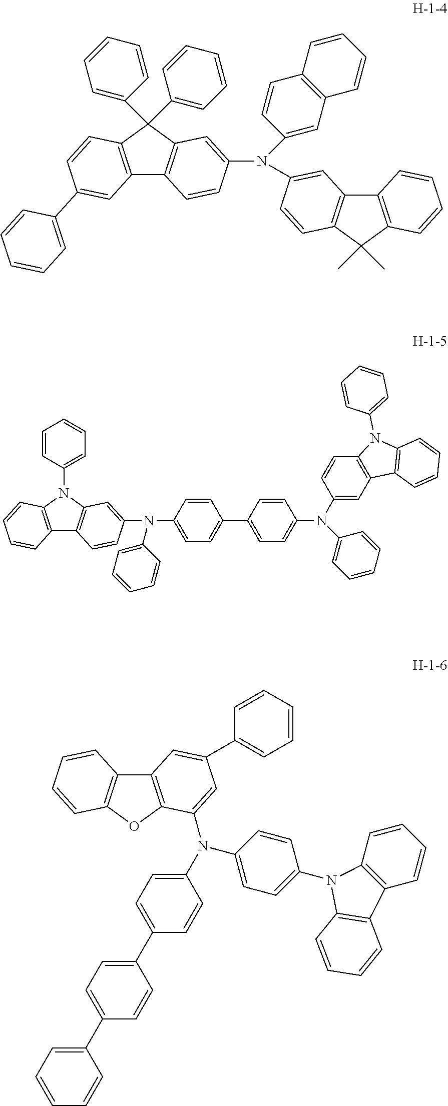 [SMILES: CC1(C)c2ccccc2-c2cc(N(c3ccc4c(c3)C(c3ccccc3)(c3ccccc3)c3ccc(-c5ccccc5)cc3-4)c3ccc4ccccc4c3)ccc21 ; c1ccc(N(c2ccc(-c3ccc(N(c4ccccc4)c4ccc5c6ccccc6n(-c6ccccc6)c5c4)cc3)cc2)c2ccc3c(c2)c2ccccc2n3-c2ccccc2)cc1 ; c1ccc(-c2ccc(-c3ccc(N(c4ccc(-n5c6ccccc6c6ccccc65)cc4)c4cc(-c5ccccc5)cc5c4oc4ccccc45)cc3)cc2)cc1]

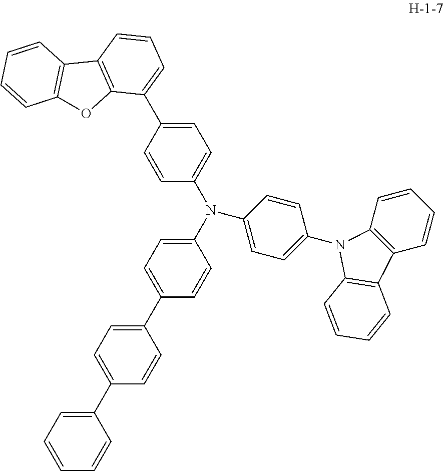 [SMILES: c1ccc(-c2ccc(-c3ccc(N(c4ccc(-c5cccc6c5oc5ccccc56)cc4)c4ccc(-n5c6ccccc6c6ccccc65)cc4)cc3)cc2)cc1]

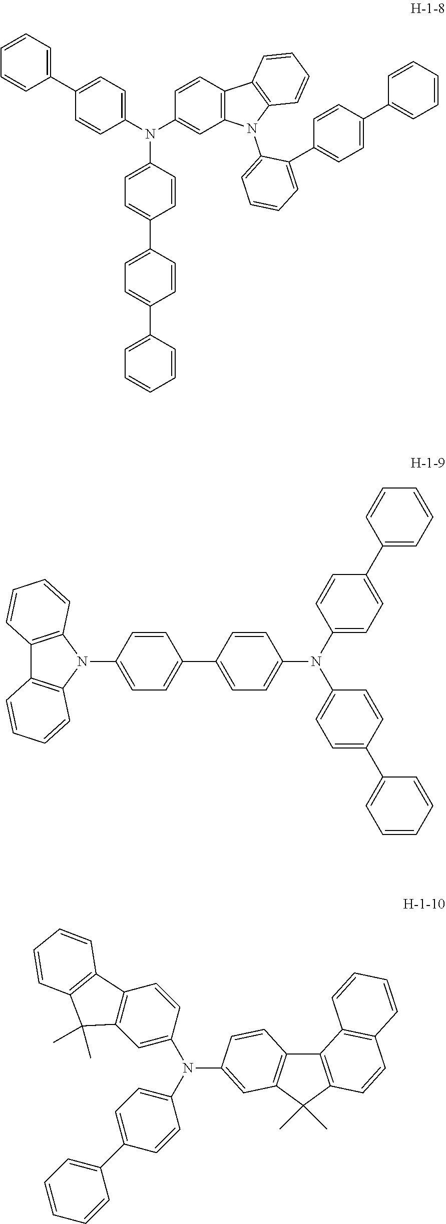 [SMILES: c1ccc(-c2ccc(-c3ccc(N(c4ccc(-c5ccccc5)cc4)c4ccc5c6ccccc6n(-c6ccccc6-c6ccc(-c7ccccc7)cc6)c5c4)cc3)cc2)cc1 ; c1ccc(-c2ccc(N(c3ccc(-c4ccccc4)cc3)c3ccc(-c4ccc(-n5c6ccccc6c6ccccc65)cc4)cc3)cc2)cc1 ; CC1(C)c2ccccc2-c2ccc(N(c3ccc(-c4ccccc4)cc3)c3ccc4c(c3)C(C)(C)c3ccc5ccccc5c3-4)cc21]

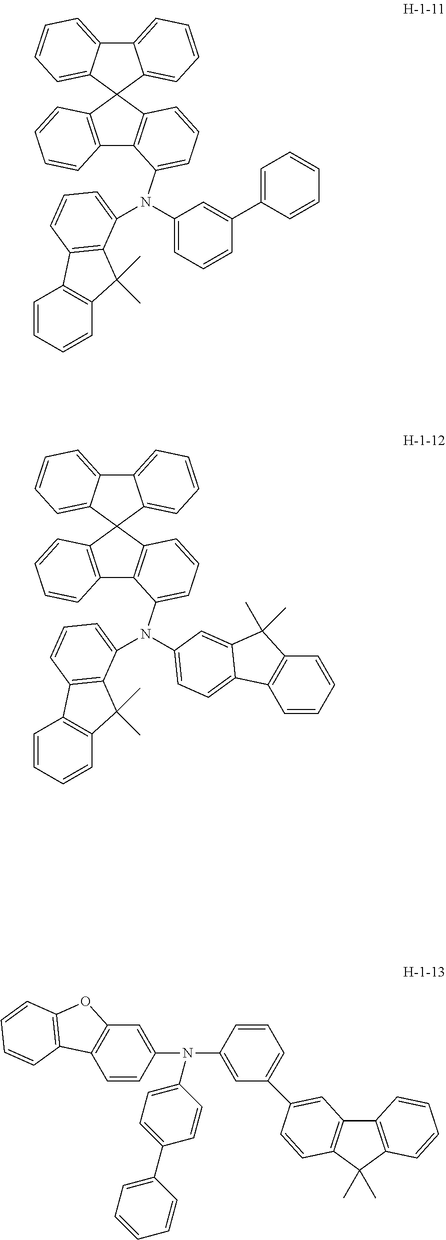 [SMILES: CC1(C)c2ccccc2-c2cccc(N(c3cccc(-c4ccccc4)c3)c3cccc4c3-c3ccccc3C43c4ccccc4-c4ccccc43)c21 ; CC1(C)c2ccccc2-c2ccc(N(c3cccc4c3-c3ccccc3C43c4ccccc4-c4ccccc43)c3cccc4c3C(C)(C)c3ccccc3-4)cc21 ; CC1(C)c2ccccc2-c2cc(-c3cccc(N(c4ccc(-c5ccccc5)cc4)c4ccc5c(c4)oc4ccccc45)c3)ccc21]

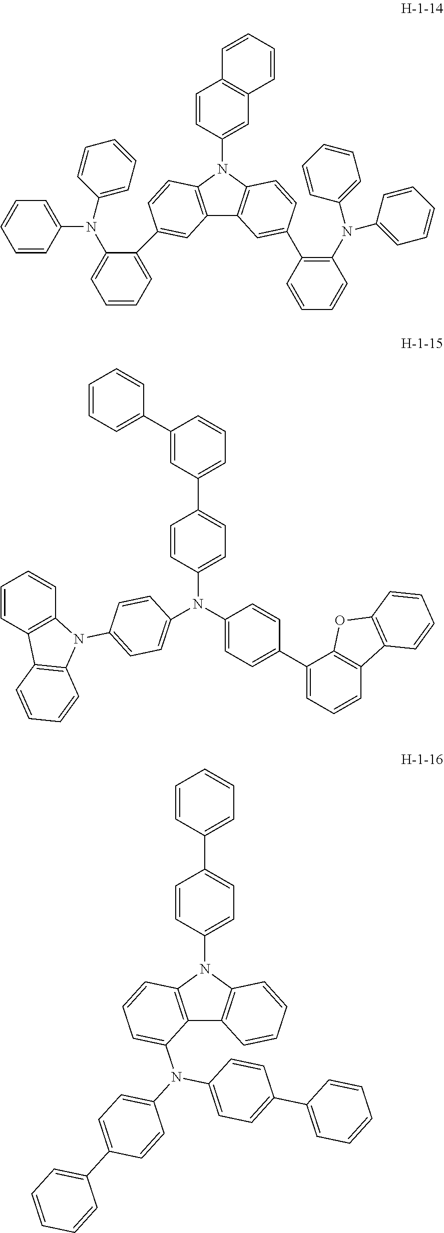 [SMILES: c1ccc(N(c2ccccc2)c2ccccc2-c2ccc3c(c2)c2cc(-c4ccccc4N(c4ccccc4)c4ccccc4)ccc2n3-c2ccc3ccccc3c2)cc1 ; c1ccc(-c2cccc(-c3ccc(N(c4ccc(-c5cccc6c5oc5ccccc56)cc4)c4ccc(-n5c6ccccc6c6ccccc65)cc4)cc3)c2)cc1 ; c1ccc(-c2ccc(N(c3ccc(-c4ccccc4)cc3)c3cccc4c3c3ccccc3n4-c3ccc(-c4ccccc4)cc3)cc2)cc1]

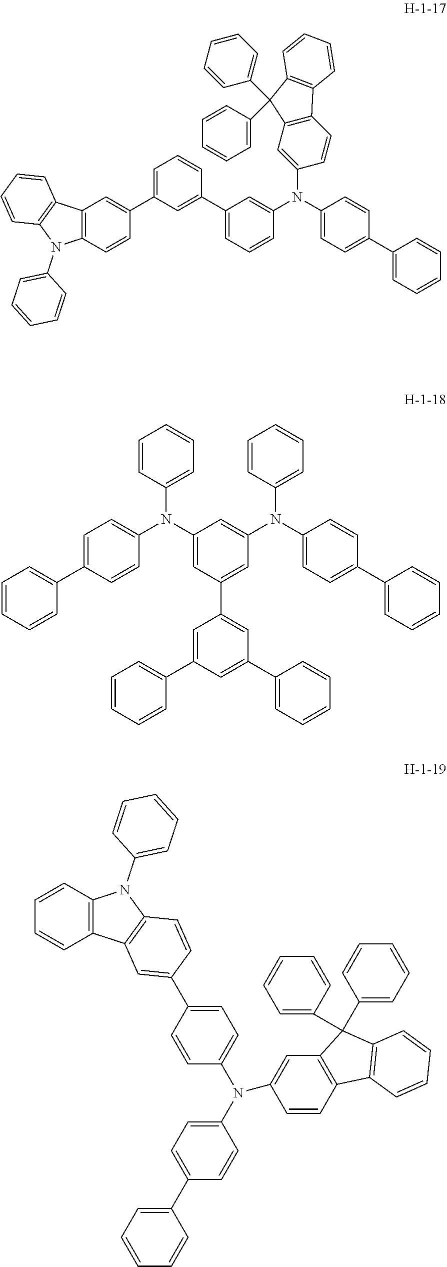 [SMILES: c1ccc(-c2ccc(N(c3cccc(-c4cccc(-c5ccc6c(c5)c5ccccc5n6-c5ccccc5)c4)c3)c3ccc4c(c3)C(c3ccccc3)(c3ccccc3)c3ccccc3-4)cc2)cc1 ; c1ccc(-c2ccc(N(c3ccccc3)c3cc(-c4cc(-c5ccccc5)cc(-c5ccccc5)c4)cc(N(c4ccccc4)c4ccc(-c5ccccc5)cc4)c3)cc2)cc1 ; c1ccc(-c2ccc(N(c3ccc(-c4ccc5c(c4)c4ccccc4n5-c4ccccc4)cc3)c3ccc4c(c3)C(c3ccccc3)(c3ccccc3)c3ccccc3-4)cc2)cc1]

The hole transport region HTR may further include a phthalocyanine compound such as copper phthalocyanine; N¹,N^{1′}-([1,1′-biphenyl]-4,4′-diyl)bis(N¹-phenyl-N⁴,N⁴-di-m-tolylbenzene-1,4-diamine) (DNTPD), 4,4′,4″-[tris(3-methylphenyl)phenylamino]triphenylamine (m-MTDATA), 4,4′4″-tris(N,N-diphenylamino)triphenylamine (TDATA), 4,4′,4″-tris[N(2-naphthyl)-N-phenylamino]-triphenylamine (2-TNATA), poly(3,4-ethylenedioxythiophene)/poly(4-styrenesulfonate) (PEDOT/PSS), polyaniline/dodecylbenzenesulfonic acid (PANI/DBSA), polyaniline/camphor sulfonic acid (PANI/CSA), polyaniline/poly(4-styrenesulfonate) (PANI/PSS), N,N′-di(naphthalene-1-yl)-N,N′-diphenyl-benzidine (NPB), triphenylamine-containing polyetherketone (TPAPEK), 4-isopropyl-4′-methyldiphenyliodonium [tetrakis(pentafluorophenyl)borate], dipyrazino[2,3-f:2′,3′-h]quinoxaline-2,3,6,7,10,11-hexacarbonitrile (HATCN), etc.

The hole transport region HTR may include carbazole derivatives such as N-phenyl carbazole and polyvinyl carbazole, fluorene derivatives, N,N′-bis(3-methylphenyl)-N,N′-diphenyl-[1,1′-biphenyl]-4,4′-diamine (TPD), triphenylamine derivatives such as 4,4′,4″-tris(N-carbazolyl)triphenylamine (TCTA), N,N′-di(naphthalene-1-yl)-N,N′-diphenyl-benzidine (NPB), 4,4′-cyclohexylidene bis[N,N-bis(4-methylphenyl]benzenamine] (TAPC), 4,4′-bis[N,N-(3-tolyl)amino]-3,3′-dimethylbiphenyl (HMTPD), 1,3-bis(N-carbazolyl)benzene (mCP), etc.

In addition, the hole transport region HTR may further include 9-(4-tert-butylphenyl)-3,6-bis(triphenylsilyl)-9H-carbazole (CzSi), 9-phenyl-9H-3,9′-bicarbazole (CCP), 1,3-bis(1,8-dimethyl-9H-carbazol-9-yl)benzene (mDCP), etc.

The hole transport region HTR may include the above-described compound of the hole transport region in at least one of a hole injection layer HIL, a hole transport layer HTL, or an electron blocking layer EBL.

The thickness of the hole transport region HTR may be from about 100 Å to about 10,000 Å, for example, from about 100 Å to about 5,000 Å. When the hole transport region HTR includes the hole injection layer HIL, the hole injection layer HIL may have, for example, a thickness of about 30 Å to about 1,000 Å. When the hole transport region HTR includes the hole transport layer HTL, the hole transport layer HTL may have a thickness of about 30 Å to about 1,000 Å. For example, when the hole transport region HTR includes the electron blocking layer EBL, the electron blocking layer EBL may have a thickness of about 10 Å to about 1,000 Å. When the thicknesses of the hole transport region HTR, the hole injection layer HIL, the hole transport layer HTL and the electron blocking layer EBL satisfy the above-described ranges, satisfactory hole transport properties may be achieved without a substantial increase in a driving voltage.

The hole transport region HTR may further include a charge generating material to increase conductivity in addition to the above-described materials. The charge generating material may be dispersed uniformly or non-uniformly in the hole transport region HTR. The charge generating material may be, for example, a p-dopant. The p-dopant may include at least one of a halogenated metal compound, a quinone derivative, a metal oxide, or a cyano group-containing compound, but the embodiment of the present disclosure is not limited thereto. For example, the p-dopant may include metal halides (such as CuI and/or RbI), quinone derivatives (such as tetracyanoquinodimethane (TCNQ) and/or 2,3,5,6-tetrafluoro-7,7′8,8-tetracyanoquinodimethane (F4-TCNQ)), metal oxides (such as tungsten oxide and/or molybdenum oxide), dipyrazino[2,3-f: 2′,3′-h]quinoxaline-2,3,6,7,10,11-hexacarbonitrile (HATCN), 4-[[2,3-bis[cyano-(4-cyano-2,3,5,6-tetrafluorophenyl)methylidene]cyclopropylidene]-cyanomethyl]-2,3,5,6-tetrafluorobenzonitrile (NDP9), etc., but the embodiment of the present disclosure is not limited thereto.

As described above, the hole transport region HTR may further include at least one of the buffer layer or the electron blocking layer EBL in addition to the hole injection layer HIL and the hole transport layer HTL. The buffer layer may compensate a resonance distance according to the wavelength of light emitted from the emission layer EML and may thus increase light emission efficiency. Materials which may be included in the hole transport region HTR may be utilized as materials to be included in the buffer layer. The electron blocking layer EBL is a layer that serves to prevent or reduce the electron injection from the electron transport region ETR to the hole transport region HTR.

The emission layer EML is provided on the hole transport region HTR. The emission layer EML may have a thickness of, for example, about 100 Å to about 1,000 Å or about 100 Å to about 300 Å. The emission layer EML may have a single layer formed of a single material, a single layer formed of a plurality of different materials, or a multilayer structure having a plurality of layers formed of a plurality of different materials.

In the light emitting device ED of an embodiment, the emission layer EML may include one or more of anthracene derivatives, pyrene derivatives, fluoranthene derivatives, chrysene derivatives, dehydrobenzanthracene derivatives, and/or triphenylene derivatives. In one or more embodiments, the emission layer EML may include one or more anthracene derivatives and/or pyrene derivatives.

In each light emitting device ED of embodiments illustrated in FIGS. 3-6, the emission layer EML may include a host and a dopant, and the emission layer EML may include a compound represented by Formula E-1 below. The compound represented by Formula E-1 below may be utilized as a fluorescence host material.

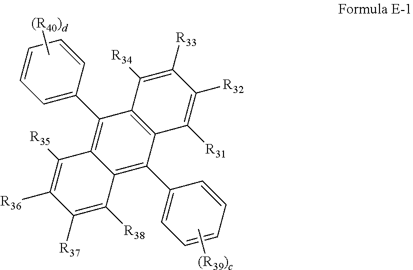

In Formula E-1, R₃₁ to R₄₀ may be each independently a hydrogen atom, a deuterium atom, a halogen atom, a substituted or unsubstituted silyl group, a substituted or unsubstituted thio group, a substituted or unsubstituted oxy group, a substituted or unsubstituted alkyl group having 1 to 10 carbon atoms, a substituted or unsubstituted alkenyl group having 1 to 10 carbon atoms, a substituted or unsubstituted aryl group having 6 to 30 ring-forming carbon atoms, or a substituted or unsubstituted heteroaryl group having 2 to 30 ring-forming carbon atoms, or may be bonded to an adjacent group to form a ring. In one or more embodiments, R₃₁ to R₄₀ may be bonded to an adjacent group to form a saturated hydrocarbon ring or an unsaturated hydrocarbon ring, a saturated heterocycle, or an unsaturated heterocycle.

In Formula E-1, c and d may be each independently an integer of 0 to 5.

Formula E-1 may be represented by any one among Compound E1 to Compound E19 below:

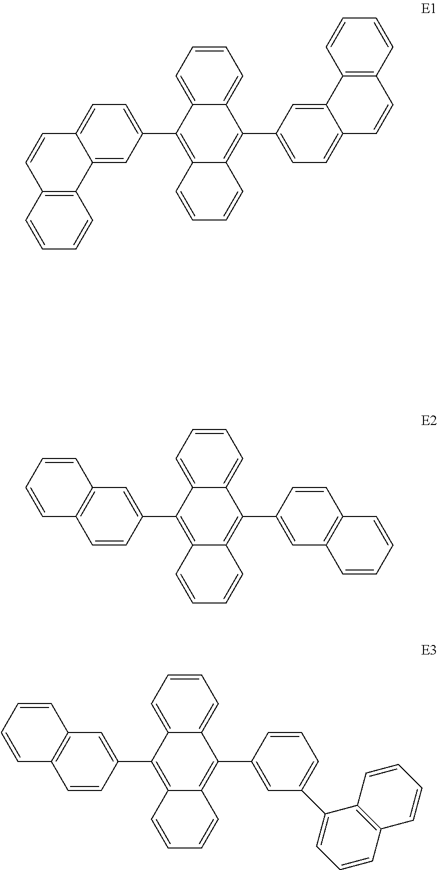

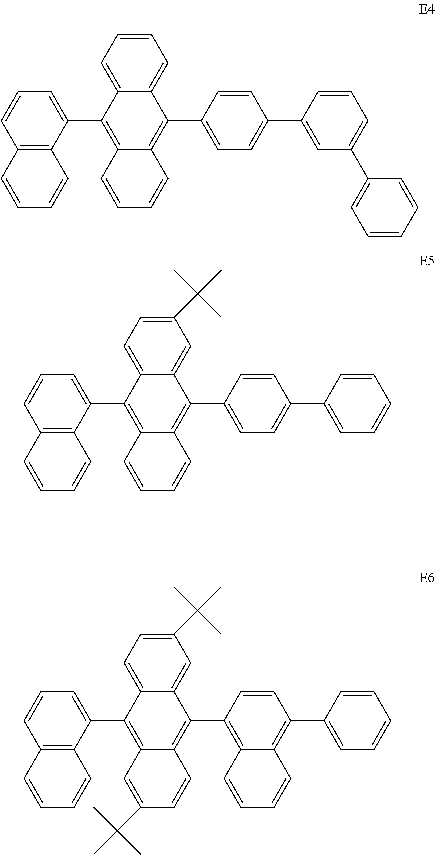

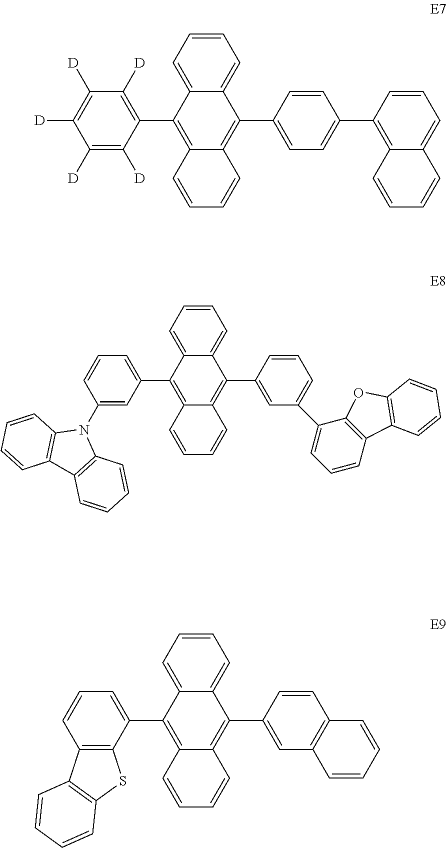 [SMILES: [2H]c1c([2H])c([2H])c(-c2c3ccccc3c(-c3ccc(-c4cccc5ccccc45)cc3)c3ccccc23)c([2H])c1[2H] ; c1cc(-c2c3ccccc3c(-c3cccc(-n4c5ccccc5c5ccccc54)c3)c3ccccc23)cc(-c2cccc3c2oc2ccccc23)c1 ; c1ccc2cc(-c3c4ccccc4c(-c4cccc5c4sc4ccccc45)c4ccccc34)ccc2c1]

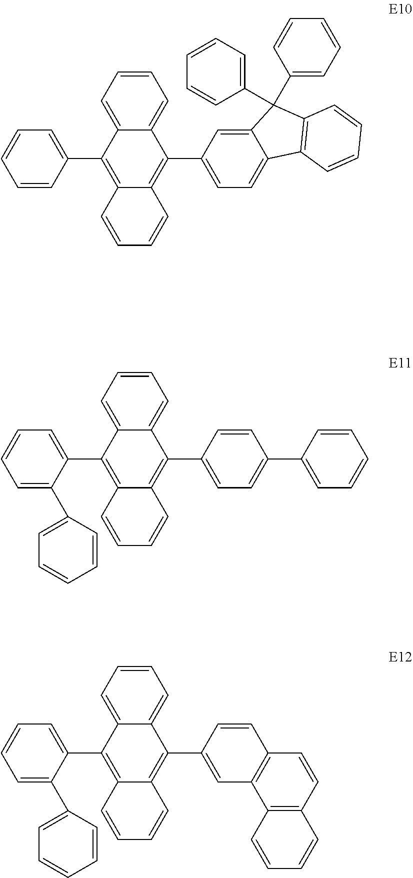

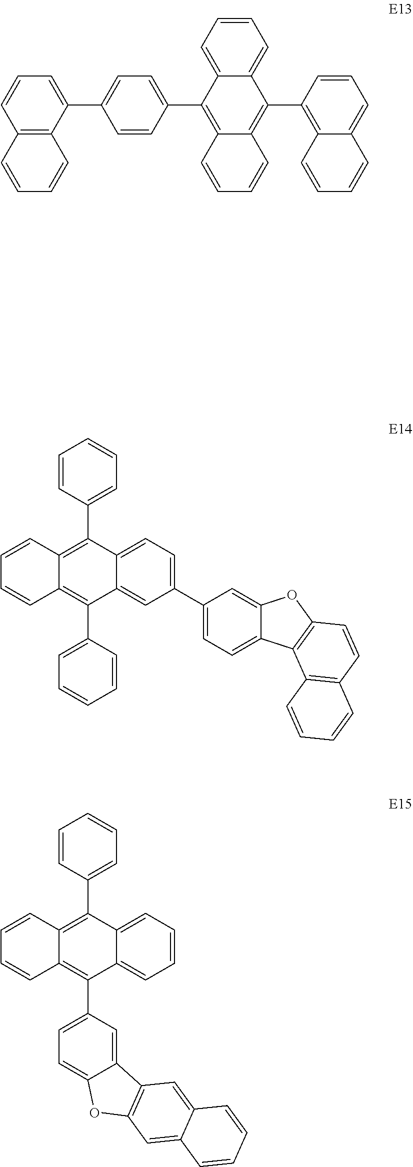

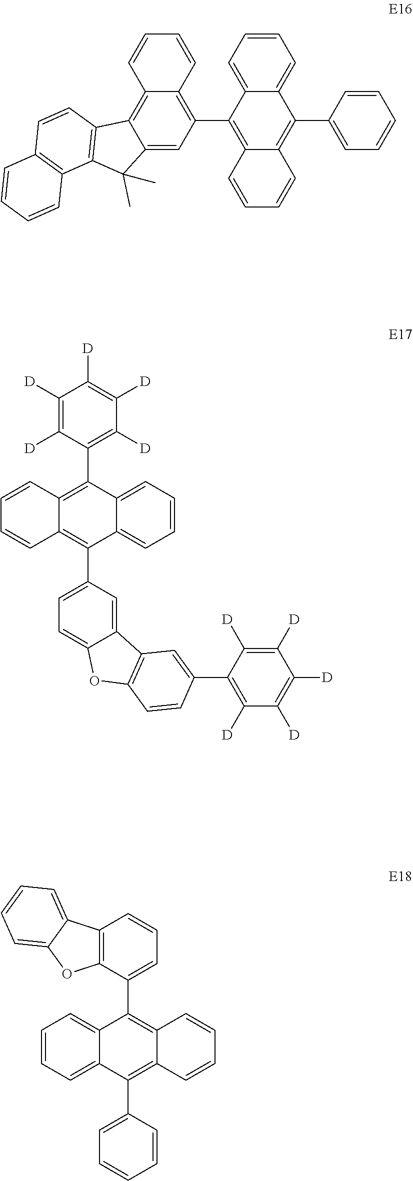

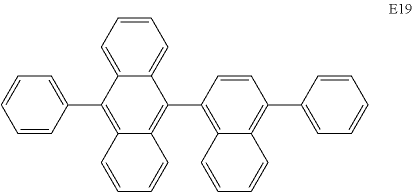 [SMILES: c1ccc(-c2ccc(-c3c4ccccc4c(-c4ccccc4)c4ccccc34)c3ccccc23)cc1]

In an embodiment, the emission layer EML may include a compound represented by Formula E-2a or Formula E-2b below. The compound represented by Formula E-2a or Formula E-2b below may be utilized as a phosphorescence host material.

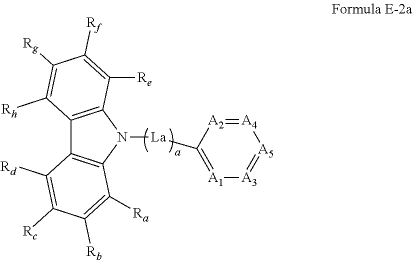

In Formula E-2a, and a may be an integer of 0 to 10, La may be a direct linkage, a substituted or unsubstituted arylene group having 6 to 30 ring-forming carbon atoms, or a substituted or unsubstituted heteroarylene group having 2 to 30 ring-forming carbon atoms. In one or more embodiments, when a is an integer of 2 or more, a plurality of Las may be each independently a substituted or unsubstituted arylene group having 6 to 30 ring-forming carbon atoms, or a substituted or unsubstituted heteroarylene group having 2 to 30 ring-forming carbon atoms.

In addition, in Formula E-2a, A₁ to A₅ may be each independently N or CRᵢ. Rₐ to Rᵢ may be each independently a hydrogen atom, a deuterium atom, a substituted or unsubstituted amine group, a substituted or unsubstituted thio group, a substituted or unsubstituted oxy group, a substituted or unsubstituted alkyl group having 1 to 20 carbon atoms, a substituted or unsubstituted alkenyl group having 2 to 20 carbon atoms, a substituted or unsubstituted aryl group having 6 to 30 ring-forming carbon atoms, or a substituted or unsubstituted heteroaryl group having 2 to 30 ring-forming carbon atoms, or may be bonded to an adjacent group to form a ring. Rₐ to Rᵢ may be bonded to an adjacent group to form a hydrocarbon ring or a heterocycle containing N, O, S, etc. as a ring-forming atom.

In one or more embodiments, in Formula E-2a, two or three groups selected from among A₁ to A₅ may be N, and the remainder (e.g., the rest) may be CRᵢ.

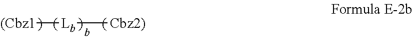

In Formula E-2b, Cbz1 and Cbz2 may be each independently an unsubstituted carbazole group, or a carbazole group substituted with an aryl group having 6 to 30 ring-forming carbon atoms. L_{b} may be a direct linkage, a substituted or unsubstituted arylene group having 6 to 30 ring-forming carbon atoms, or a substituted or unsubstituted heteroarylene group having 2 to 30 ring-forming carbon atoms. b may be an integer of 0 to 10, and when b is an integer of 2 or more, a plurality of L_{b}s may be each independently a substituted or unsubstituted arylene group having 6 to 30 ring-forming carbon atoms, or a substituted or unsubstituted heteroarylene group having 2 to 30 ring-forming carbon atoms.

The compound represented by Formula E-2a or Formula E-2b may be represented by any one among the compounds of Compound Group E-2 below. However, the compounds listed in Compound Group E-2 below are examples, the compound represented by Formula E-2a or Formula E-2b is not limited to those represented by Compound Group E-2 below.

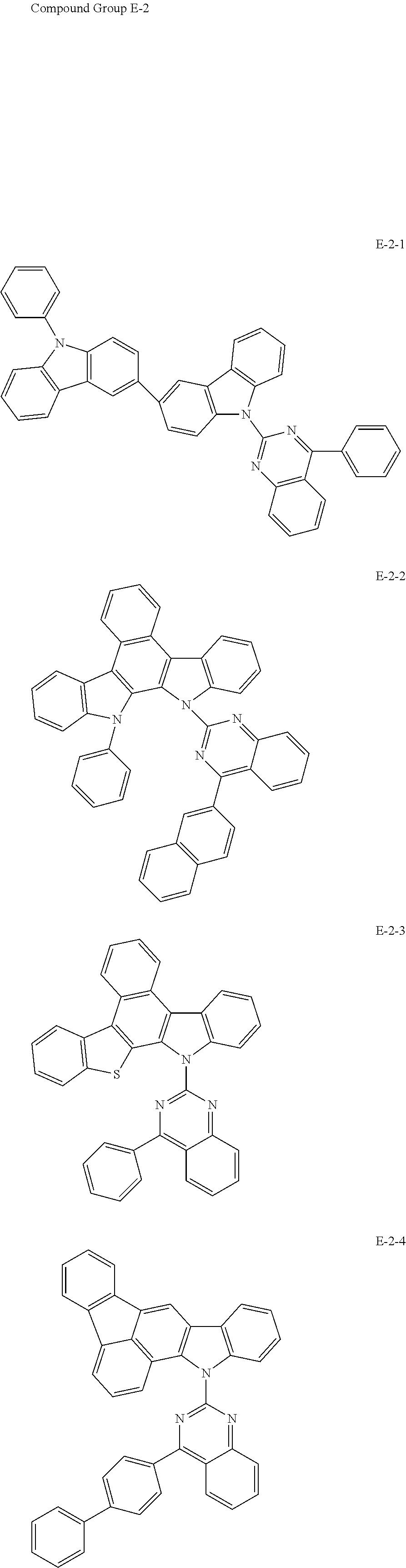 [SMILES: c1ccc(-c2nc(-n3c4ccccc4c4cc(-c5ccc6c(c5)c5ccccc5n6-c5ccccc5)ccc43)nc3ccccc23)cc1 ; c1ccc(-n2c3ccccc3c3c4ccccc4c4c5ccccc5n(-c5nc(-c6ccc7ccccc7c6)c6ccccc6n5)c4c32)cc1 ; c1ccc(-c2nc(-n3c4ccccc4c4c5ccccc5c5c6ccccc6sc5c43)nc3ccccc23)cc1 ; c1ccc(-c2ccc(-c3nc(-n4c5ccccc5c5cc6c7c(cccc7c54)-c4ccccc4-6)nc4ccccc34)cc2)cc1]

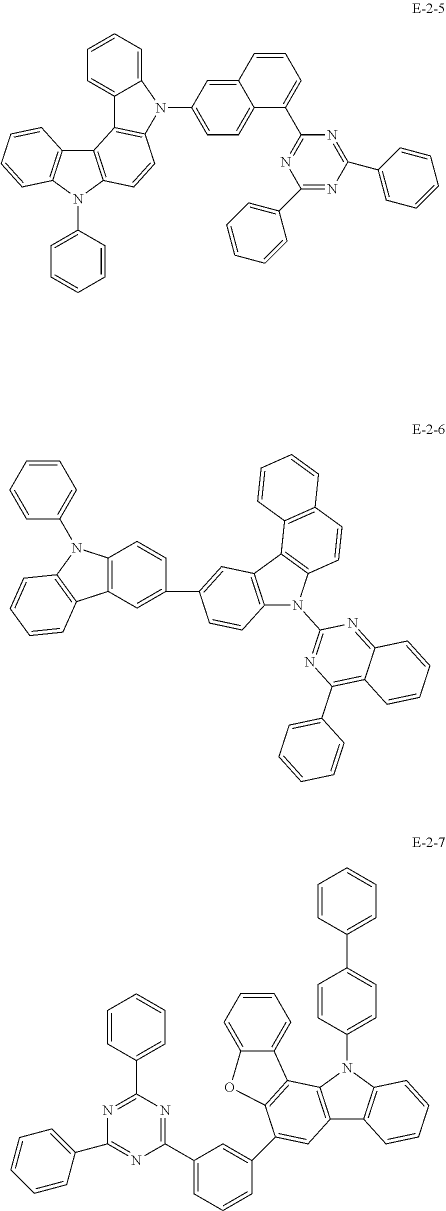 [SMILES: c1ccc(-c2nc(-c3ccccc3)nc(-c3cccc4cc(-n5c6ccccc6c6c7c8ccccc8n(-c8ccccc8)c7ccc65)ccc34)n2)cc1 ; c1ccc(-c2nc(-n3c4ccc(-c5ccc6c(c5)c5ccccc5n6-c5ccccc5)cc4c4c5ccccc5ccc43)nc3ccccc23)cc1 ; c1ccc(-c2ccc(-n3c4ccccc4c4cc(-c5cccc(-c6nc(-c7ccccc7)nc(-c7ccccc7)n6)c5)c5oc6ccccc6c5c43)cc2)cc1]

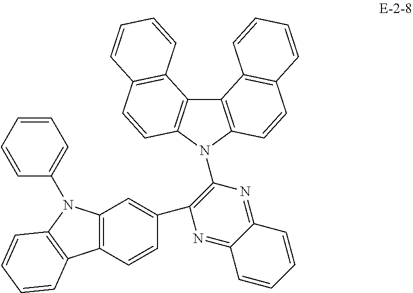 [SMILES: c1ccc(-n2c3ccccc3c3ccc(-c4nc5ccccc5nc4-n4c5ccc6ccccc6c5c5c6ccccc6ccc54)cc32)cc1]

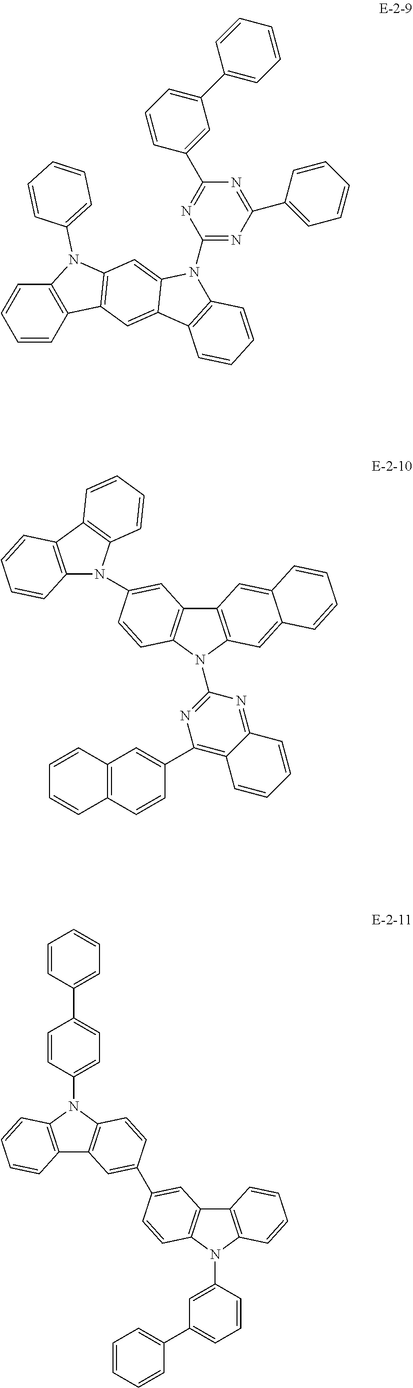 [SMILES: c1ccc(-c2cccc(-c3nc(-c4ccccc4)nc(-n4c5ccccc5c5cc6c7ccccc7n(-c7ccccc7)c6cc54)n3)c2)cc1 ; c1ccc2cc(-c3nc(-n4c5ccc(-n6c7ccccc7c7ccccc76)cc5c5cc6ccccc6cc54)nc4ccccc34)ccc2c1 ; c1ccc(-c2ccc(-n3c4ccccc4c4cc(-c5ccc6c(c5)c5ccccc5n6-c5cccc(-c6ccccc6)c5)ccc43)cc2)cc1]

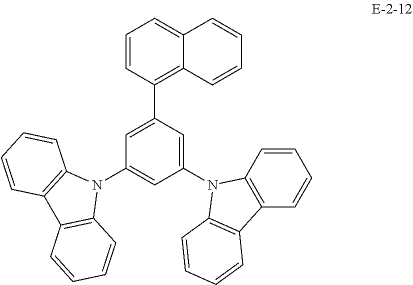 [SMILES: c1ccc2c(-c3cc(-n4c5ccccc5c5ccccc54)cc(-n4c5ccccc5c5ccccc54)c3)cccc2c1]

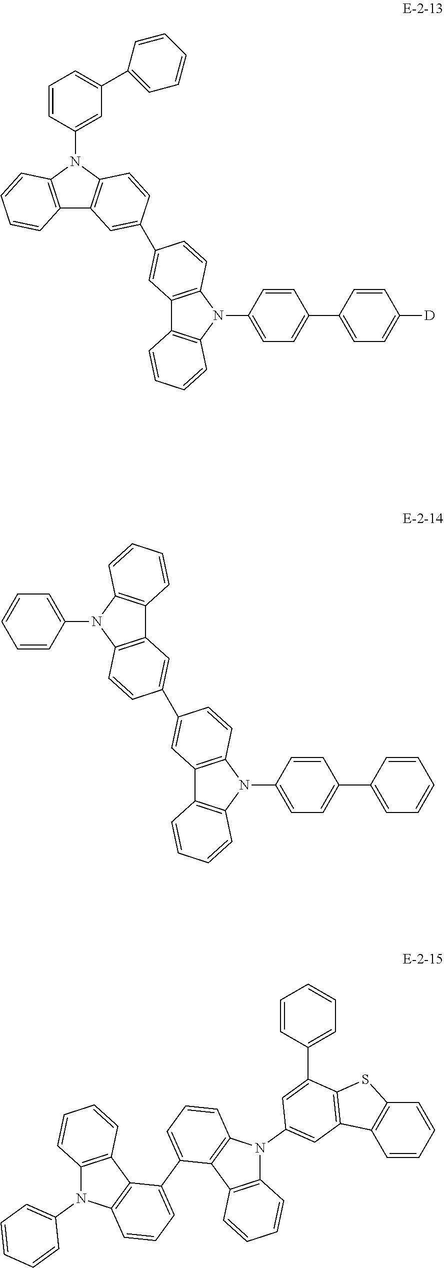 [SMILES: [2H]c1ccc(-c2ccc(-n3c4ccccc4c4cc(-c5ccc6c(c5)c5ccccc5n6-c5cccc(-c6ccccc6)c5)ccc43)cc2)cc1 ; c1ccc(-c2ccc(-n3c4ccccc4c4cc(-c5ccc6c(c5)c5ccccc5n6-c5ccccc5)ccc43)cc2)cc1 ; c1ccc(-c2cc(-n3c4ccccc4c4c(-c5cccc6c5c5ccccc5n6-c5ccccc5)cccc43)cc3c2sc2ccccc23)cc1]

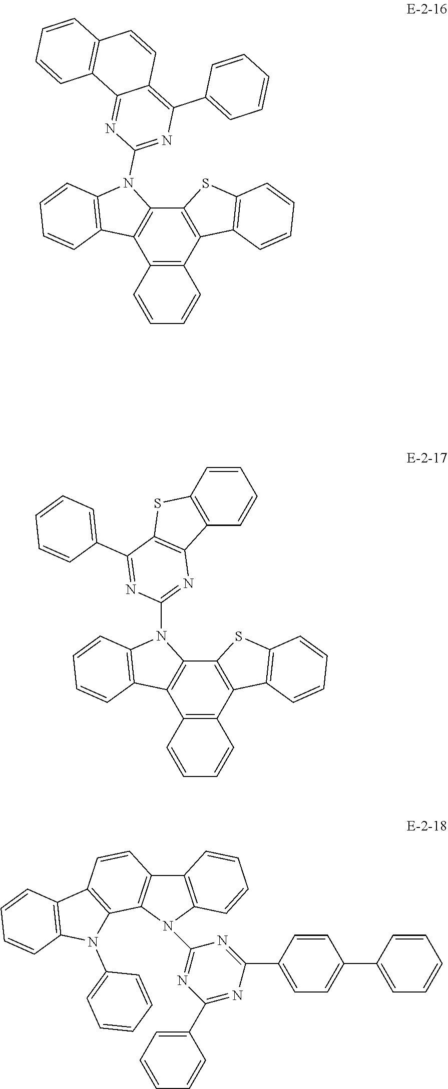 [SMILES: c1ccc(-c2nc(-n3c4ccccc4c4c5ccccc5c5c6ccccc6sc5c43)nc3c2ccc2ccccc23)cc1 ; c1ccc(-c2nc(-n3c4ccccc4c4c5ccccc5c5c6ccccc6sc5c43)nc3c2sc2ccccc23)cc1 ; c1ccc(-c2ccc(-c3nc(-c4ccccc4)nc(-n4c5ccccc5c5ccc6c7ccccc7n(-c7ccccc7)c6c54)n3)cc2)cc1]

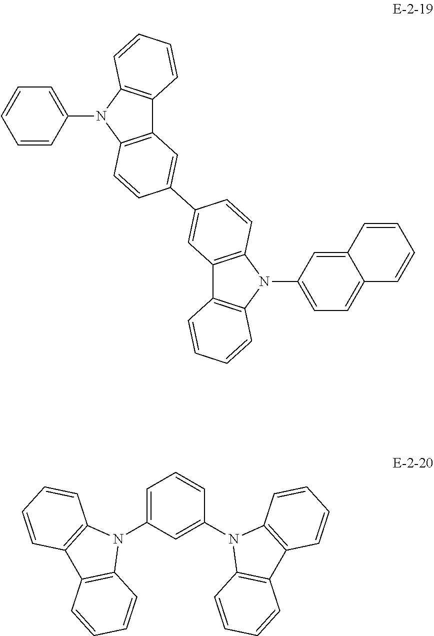 [SMILES: c1ccc(-n2c3ccccc3c3cc(-c4ccc5c(c4)c4ccccc4n5-c4ccc5ccccc5c4)ccc32)cc1 ; c1cc(-n2c3ccccc3c3ccccc32)cc(-n2c3ccccc3c3ccccc32)c1]

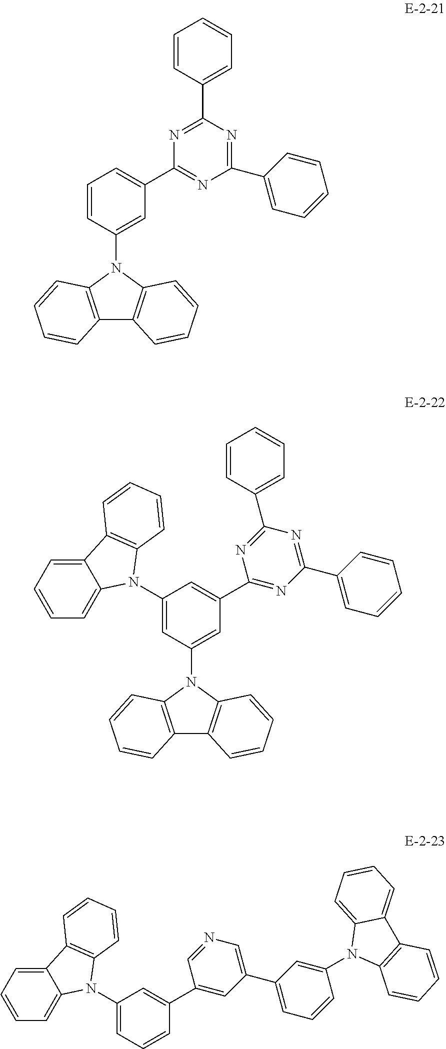 [SMILES: c1ccc(-c2nc(-c3ccccc3)nc(-c3cccc(-n4c5ccccc5c5ccccc54)c3)n2)cc1 ; c1ccc(-c2nc(-c3ccccc3)nc(-c3cc(-n4c5ccccc5c5ccccc54)cc(-n4c5ccccc5c5ccccc54)c3)n2)cc1 ; c1cc(-c2cncc(-c3cccc(-n4c5ccccc5c5ccccc54)c3)c2)cc(-n2c3ccccc3c3ccccc32)c1]

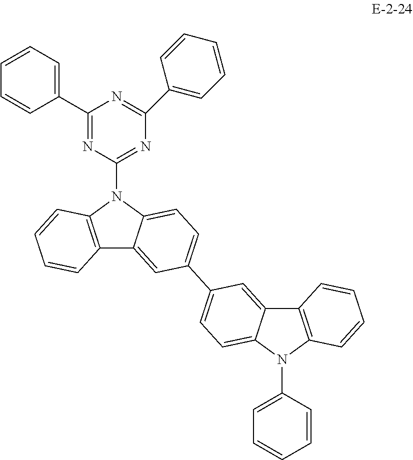 [SMILES: c1ccc(-c2nc(-c3ccccc3)nc(-n3c4ccccc4c4cc(-c5ccc6c(c5)c5ccccc5n6-c5ccccc5)ccc43)n2)cc1]

The emission layer EML may further include a suitable (e.g., a general material known in the art) as a host material. For example, the emission layer EML may include, as a host material, at least one of bis[2-(diphenylphosphino)phenyl]ether oxide (DPEPO), 4,4′-bis(N-carbazolyl)-1,1′-biphenyl (CBP), 1,3-bis(carbazol-9-yl)benzene (mCP), 2,8-bis(diphenylphosphoryl)dibenzo[b,d]furan (PPF), 4,4′,4″-tris(carbazol-9-yl)-triphenylamine (TCTA), or 1,3,5-tris(1-phenyl-1H-benzo[d]imidazole-2-yl)benzene (TPBi). However, the embodiment of the present disclosure is not limited thereto, for example, tris(8-hydroxyquinolino)aluminum (Alq3), 9,10-di(naphthalene-2-yl)anthracene (ADN), 2-tert-butyl-9,10-di(naphth-2-yl)anthracene (TBADN), distyrylarylene (DSA), 4,4′-bis(9-carbazolyl)-2,2′-dimethyl-biphenyl (CDBP), 2-methyl-9,10-bis(naphthalen-2-yl)anthracene (MADN), hexaphenyl cyclotriphosphazene (CP1), 1,4-bis(triphenylsilyl)benzene (UGH2), hexaphenylcyclotrisiloxane (DPSiO₃), octaphenylcyclotetra siloxane (DPSiO₄), etc. may be utilized as the host material.

The emission layer EML may include a compound represented by Formula M-a or Formula M-b below. The compound represented by Formula M-a or Formula M-b below may be utilized as a phosphorescence dopant material.

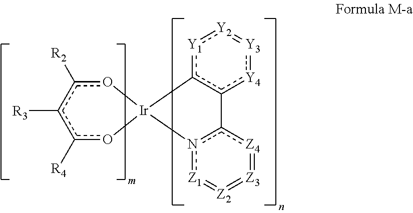

In Formula M-a above, Y₁ to Y₄ and Z₁ to Z₄ may be each independently CR₁ or N, and R₁ to R₄ may be each independently a hydrogen atom, a deuterium atom, a substituted or unsubstituted amine group, a substituted or unsubstituted thio group, a substituted or unsubstituted oxy group, a substituted or unsubstituted alkyl group having 1 to 20 carbon atoms, a substituted or unsubstituted alkenyl group having 2 to 20 carbon atoms, a substituted or unsubstituted aryl group having 6 to 30 ring-forming carbon atoms, or a substituted or unsubstituted heteroaryl group having 2 to 30 ring-forming carbon atoms, or may be bonded to an adjacent group to form a ring. In Formula M-a, m is 0 or 1, and n is 2 or 3. In Formula M-a, when m is 0, n is 3, and when m is 1, n is 2.

The compound represented by Formula M-a may be utilized as a phosphorescence dopant.

The compound represented by Formula M-a may be represented by any one among Compound M-a1 to Compound M-a25 below. However, Compounds M-a1 to M-a25 below are examples, and the compound represented by Formula M-a is not limited to those represented by Compounds M-a1 to M-a25 below.

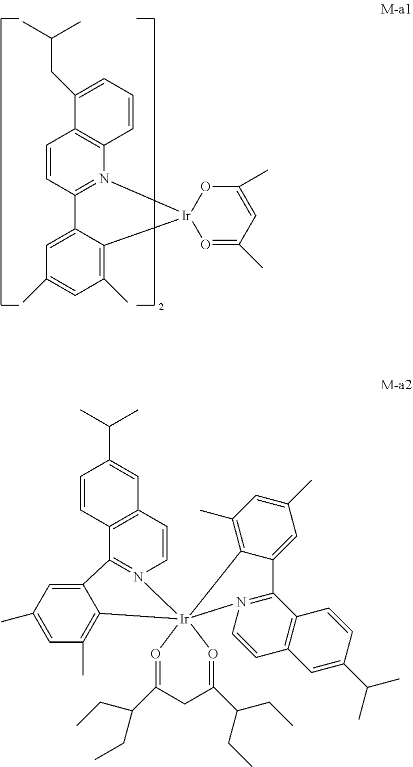

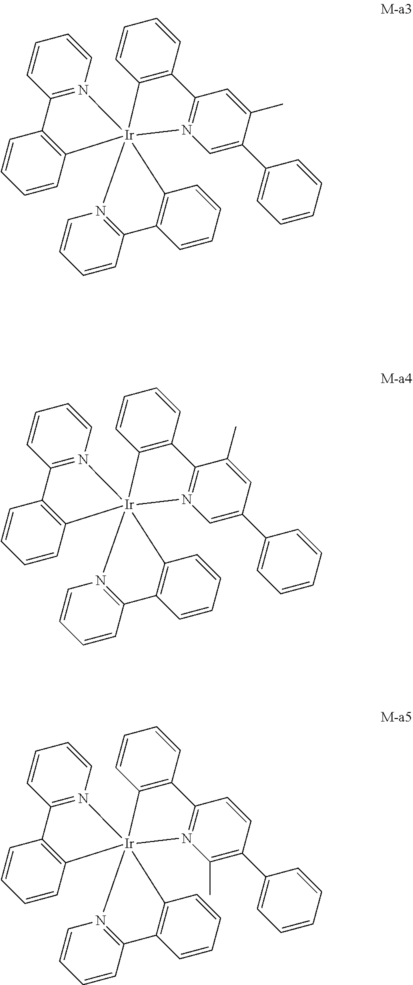

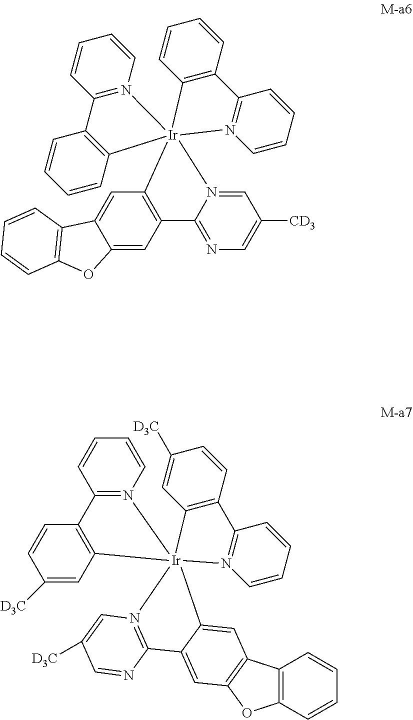

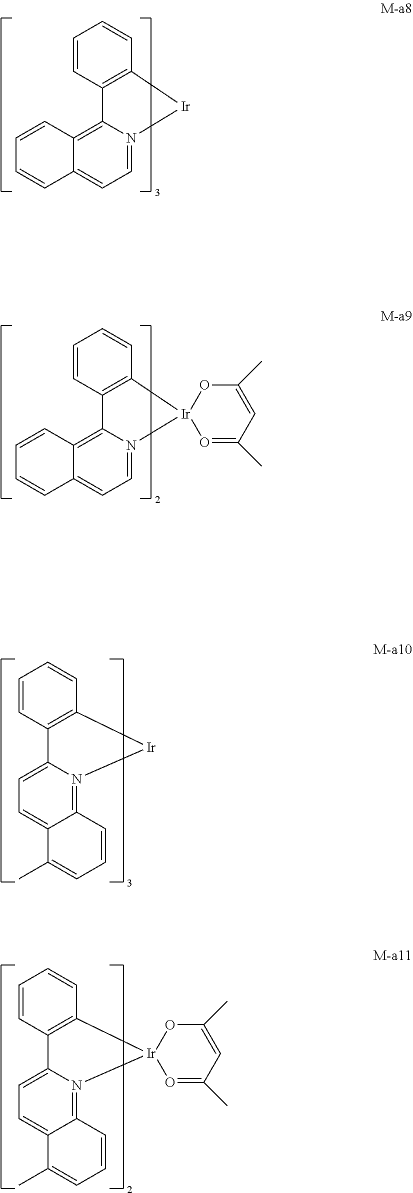

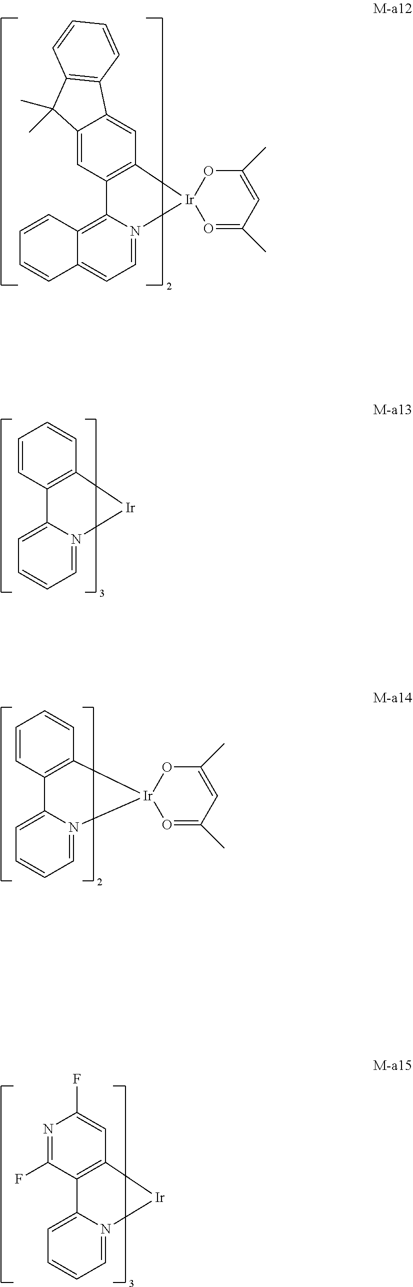

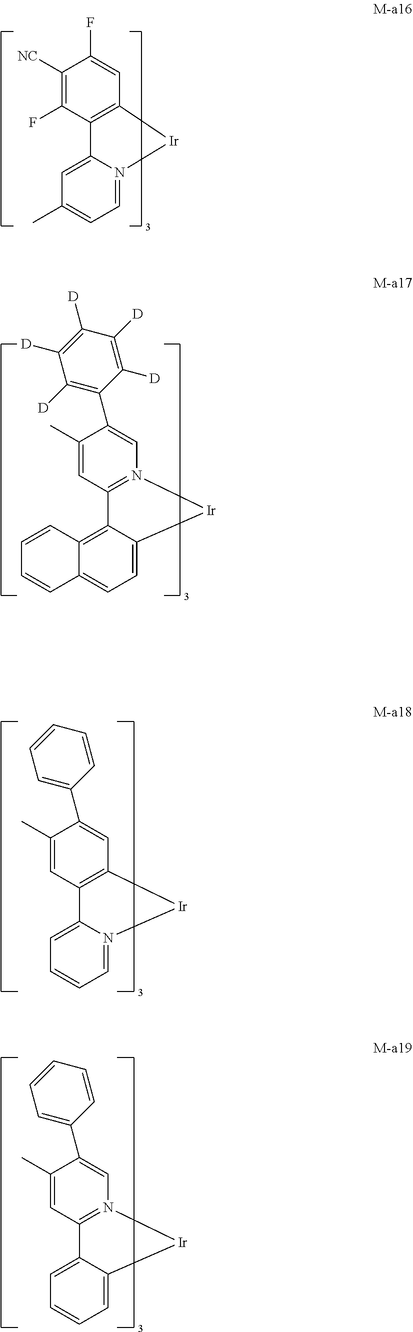

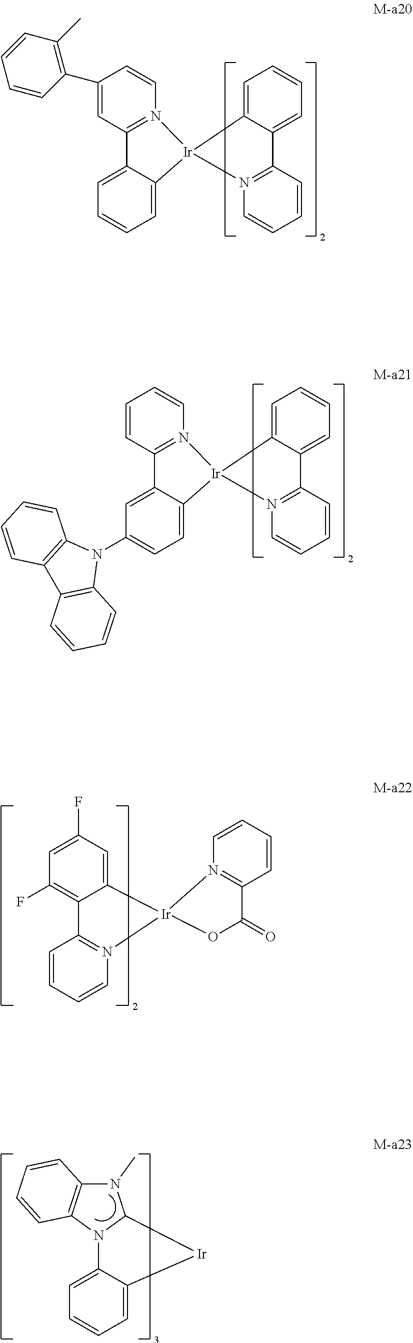

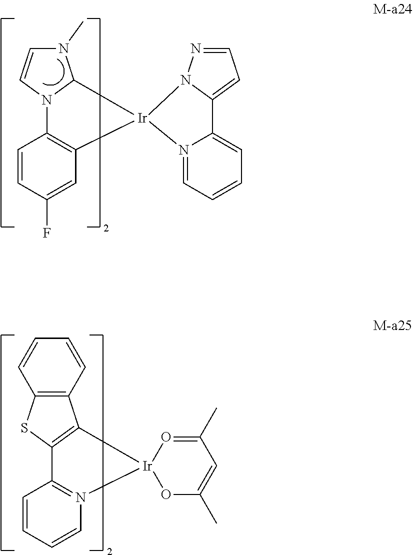

Compound M-a1 and Compound M-a2 may be utilized as a red dopant material, and Compound M-a3 to Compound M-a7 may be utilized as a green dopant material.

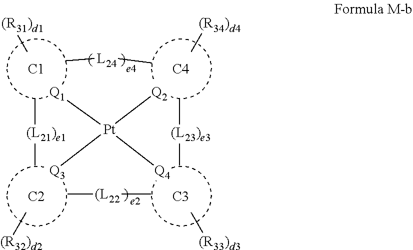

In Formula M-b, Q₁ to Q₄ are each independently C or N, and C1 to C4 are each independently a substituted or unsubstituted hydrocarbon ring having 5 to 30 ring-forming carbon atoms, or a substituted or unsubstituted heterocycle having 2 to 30 ring-forming carbon atoms. L₂₁ to L₂₄ are each independently a direct linkage,

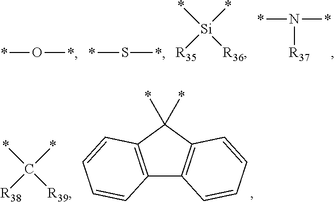

a substituted or unsubstituted divalent alkyl group having 1 to 20 carbon atoms, a substituted or unsubstituted arylene group having 6 to 30 ring-forming carbon atoms, or a substituted or unsubstituted heteroarylene group having 2 to 30 ring-forming carbon atoms, and el to e4 are each independently 0 or 1. R₃₁ to R₃₉ are each independently a hydrogen atom, a deuterium atom, a halogen atom, a cyano group, a substituted or unsubstituted amine group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms, and/or bonded to an adjacent group to form a ring; and d1 to d4 are each independently an integer of 0 to 4.

The compound represented by Formula M-b may be utilized as a blue phosphorescence dopant or a green phosphorescence dopant.

The compound represented by Formula M-b may be represented by any one among the compounds below. However, the compounds below are examples, and the compound represented by Formula M-b is not limited to those represented by the compounds below.

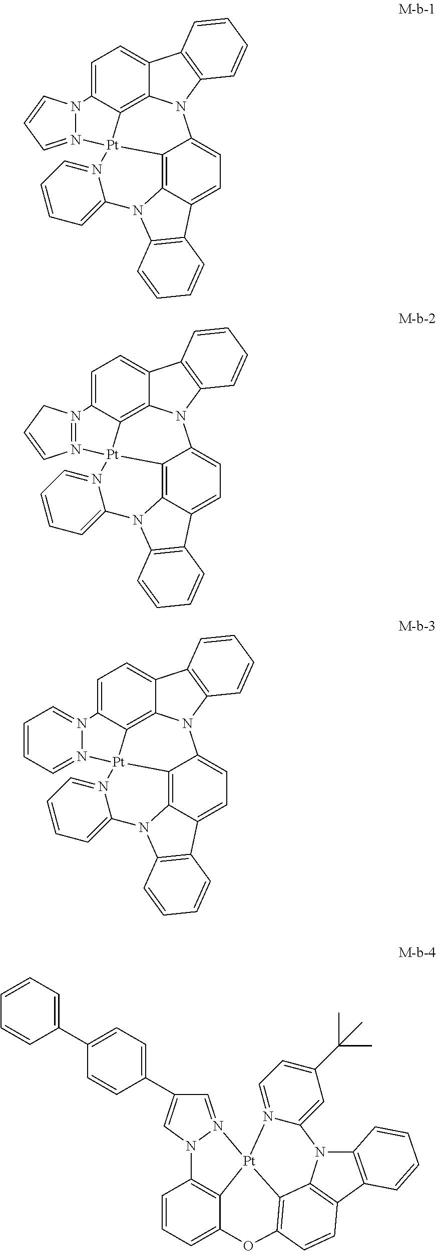

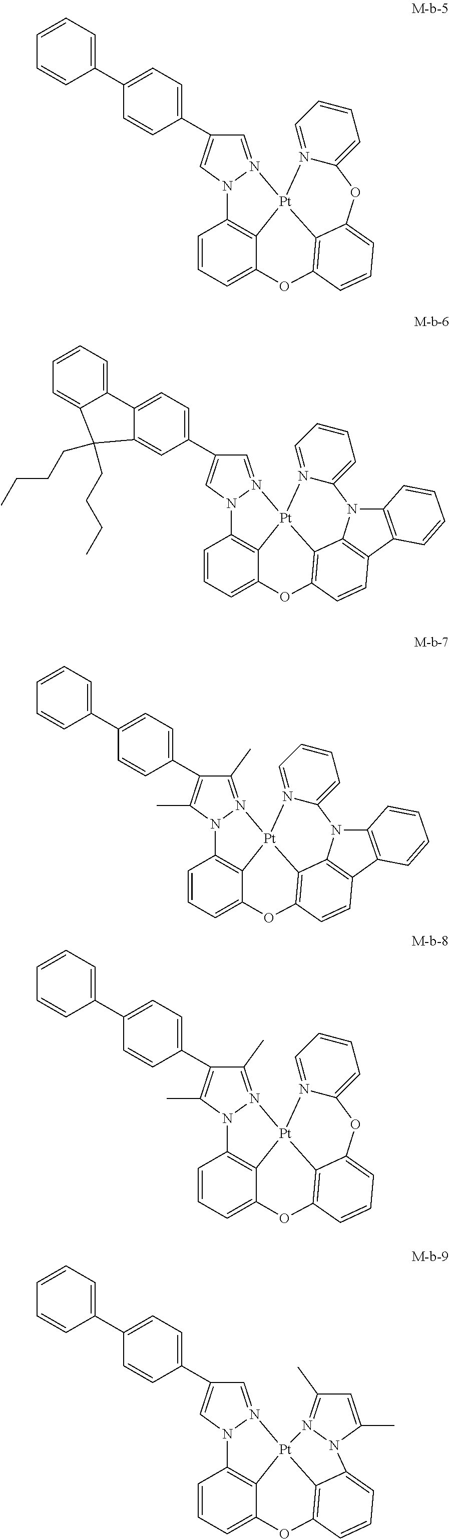

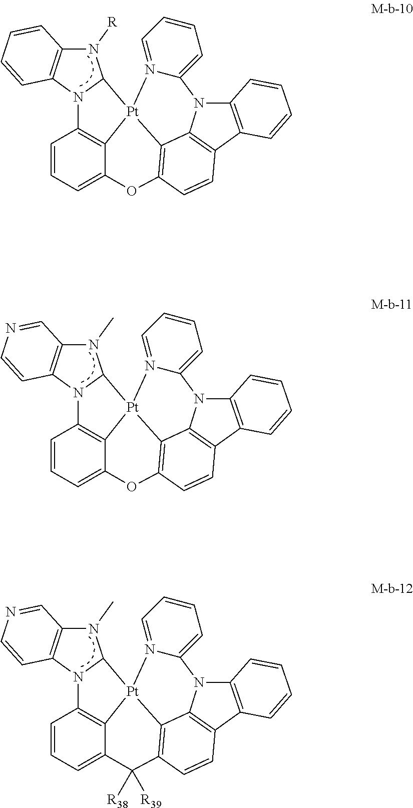

In these compounds, R, R₃₈, and R₃₉ may be each independently a hydrogen atom, a deuterium atom, a halogen atom, a cyano group, a substituted or unsubstituted amine group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. The emission layer EML may include a compound represented by any one among Formula F-a to Formula F-c below. The compounds represented by Formula F-a to Formula F-c below may be utilized as a fluorescence dopant material.

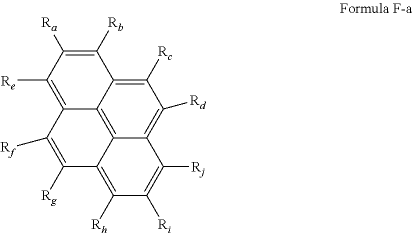

In Formula F-a, two groups selected from among Rₐ to Rⱼ may each independently be substituted with *—NAr₁Ar₂. The others (e.g., remainder groups) among Rₐ to Rⱼ, which are not substituted with *—NAr₁Ar₂, may be each independently a hydrogen atom, a deuterium atom, a halogen atom, a cyano group, a substituted or unsubstituted amine group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. In *—NAr₁Ar₂, Ar₁ and Ar₂ may be each independently a substituted or unsubstituted aryl group having 6 to 30 ring-forming carbon atoms, or a substituted or unsubstituted heteroaryl group having 2 to 30 ring-forming carbon atoms. For example, Ar₁ and/or Ar₂ may be a heteroaryl group containing O or S as a ring-forming atom.

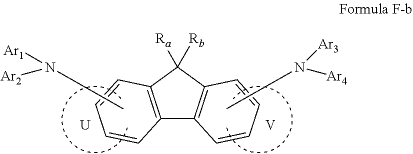

In Formula F-b, Rₐ and R_{b} may be each independently a hydrogen atom, a deuterium atom, a substituted or unsubstituted alkyl group having 1 to 20 carbon atoms, a substituted or unsubstituted alkenyl group having 2 to 20 carbon atoms, a substituted or unsubstituted aryl group having 6 to 30 ring-forming carbon atoms, or a substituted or unsubstituted heteroaryl group having 2 to 30 ring-forming carbon atoms, or may be bonded to an adjacent group to form a ring.

In Formula F-b, U and V may be each independently a substituted or unsubstituted hydrocarbon ring having 5 to 30 ring-forming carbon atoms, or a substituted or unsubstituted heterocycle having 2 to 30 ring-forming carbon atoms.

In Formula F-b, the number of rings represented by U and V may be each independently 0 or 1. For example, in Formula F-b, when the number of U or V is 1, one ring forms a condensed ring at a part described as U or V, and when the number of U or V is 0, a ring described as U or V is not present. For example, when the number of U is 0 and the number of V is 1, or when the number of U is 1 and the number of V is 0, the condensed ring having a fluorene core of Formula F-b may be a four-ring cyclic compound. In addition, when each number of U and V is 0, the condensed ring of Formula F-b may be a three-ring cyclic compound. In addition, when each number of U and V is 1, the condensed ring having a fluorene core of Formula F-b may be a five-ring cyclic compound.

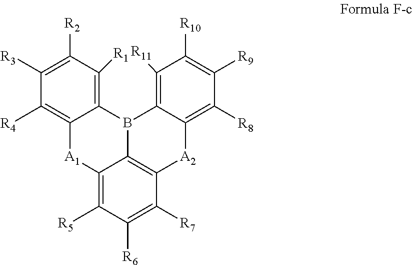

In Formula F-c, A₁ and A₂ may be each independently O, S, Se, or NRₘ, and Rₘ may be a hydrogen atom, a deuterium atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. R₁ to R₁₁ are each independently a hydrogen atom, a deuterium atom, a halogen atom, a cyano group, a substituted or unsubstituted amine group, a substituted or unsubstituted boryl group, a substituted or unsubstituted oxy group, a substituted or unsubstituted thio group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms, or are bonded to an adjacent group to form a ring.

In Formula F-c, A₁ and A₂ may each independently be bonded to substituents of an adjacent ring to form a condensed ring. For example, when A₁ and A₂ are each independently NRₘ, A₁ may be bonded to R₄ or R₅ to form a ring. In addition, A₂ may be bonded to R₇ or R₈ to form a ring.

In an embodiment, the emission layer EML may include, as a suitable (e.g., known) dopant material, styryl derivatives (e.g., 1,4-bis[2-(3-N-ethylcarbazoryl)vinyl]benzene (BCzVB), 4-(di-p-tolylamino)-4′-[(di-p-tolylamino)styryl]stilbene (DPAVB), and/or N-(4-((E)-2-(6-((E)-4-(diphenylamino)styryl)naphthalen-2-yl)vinyl)phenyl)-N-phenylbenzenamine (N-BDAVBi)), 4,4′-bis[2-(4-(N,N-diphenylamino)phenyl)vinyl]biphenyl (DPAVBi)), perylene and the derivatives thereof (e.g., 2,5,8,11-tetra-t-butylperylene (TBP)), pyrene and the derivatives thereof (e.g., 1,1-dipyrene, 1,4-dipyrenylbenzene, and/or 1,4-bis(N,N-diphenylamino)pyrene), etc.

The emission layer EML may further include a suitable (e.g., known) phosphorescence dopant material. For example, a metal complex including iridium (Ir), platinum (Pt), osmium (Os), gold (Au), titanium (Ti), zirconium (Zr), hafnium (Hf), europium (Eu), terbium (Tb), and/or thulium (Tm) may be utilized as a phosphorescence dopant. In one or more embodiments, iridium(III) bis(4,6-difluorophenylpyridinato-N,C2′)picolinate (Flrpic), bis(2,4-difluorophenylpyridinato)-tetrakis(1-pyrazolyl)borate iridium(III) (Fir6), and/or platinum octaethyl porphyrin (PtOEP) may be utilized as a phosphorescence dopant. However, the embodiment of the present disclosure is not limited thereto.

The emission layer EML may include a quantum dot material. The core of the quantum dot may be selected from a Group II-VI compound, a Group III-VI compound, a Group compound, a Group III-V compound, a Group III-II-V compound, a Group IV-VI compound, a Group IV element, a Group IV compound, or a combination thereof.

The Group II-VI compound may be selected from the group consisting of a binary compound selected from the group consisting of CdSe, CdTe, CdS, ZnS, ZnSe, ZnTe, ZnO, HgS, HgSe, HgTe, MgSe, MgS, and a mixture thereof, a ternary compound selected from the group consisting of CdSeS, CdSeTe, CdSTe, ZnSeS, ZnSeTe, ZnSTe, HgSeS, HgSeTe, HgSTe, CdZnS, CdZnSe, CdZnTe, CdHgS, CdHgSe, CdHgTe, HgZnS, HgZnSe, HgZnTe, MgZnSe, MgZnS, and a mixture thereof, and a quaternary compound selected from the group consisting of HgZnTeS, CdZnSeS, CdZnSeTe, CdZnSTe, CdHgSeS, CdHgSeTe, CdHgSTe, HgZnSeS, HgZnSeTe, HgZnSTe, and a mixture thereof.

The Group III-VI compound may include a binary compound such as In₂S₃ and/or In₂Se₃, a ternary compound such as InGaS₃ and/or InGaSe₃, or any combination thereof.

The Group compound may be selected from a ternary compound selected from the group consisting of AgInS, AgInS₂, CuInS, CuInS₂, AgGaS₂, CuGaS₂ CuGaO₂, AgGaO₂, AgAlO₂, and a mixture thereof, and a quaternary compound such as AgInGaS₂ and/or CuInGaS₂.

The Group III-V compound may be selected from the group consisting of a binary compound selected from the group consisting of GaN, GaP, GaAs, GaSb, AlN, AlP, AlAs, AlSb, InN, InP, InAs, InSb, and a mixture thereof, a ternary compound selected from the group consisting of GaNP, GaNAs, GaNSb, GaPAs, GaPSb, AlNP, AlNAs, AlNSb, AlPAs, AlPSb, InGaP, InAlP, InNP, InNAs, InNSb, InPAs, InPSb, and a mixture thereof, and a quaternary compound selected from the group consisting of GaAlNP, GaAlNAs, GaAlNSb, GaAlPAs, GaAlPSb, GaInNP, GaInNAs, GaInNSb, GaInPAs, GaInPSb, InAlNP, InAINAs, InAlNSb, InAlPAs, InAlPSb, and a mixture thereof. In one or more embodiments, the Group III-V compound may further include a Group II metal. For example, InZnP, etc. may be selected as a Group III-II-V compound.

The Group IV-VI compound may be selected from the group consisting of a binary compound selected from the group consisting of SnS, SnSe, SnTe, PbS, PbSe, PbTe, and a mixture thereof, a ternary compound selected from the group consisting of SnSeS, SnSeTe, SnSTe, PbSeS, PbSeTe, PbSTe, SnPbS, SnPbSe, SnPbTe, and a mixture thereof, and a quaternary compound selected from the group consisting of SnPbSSe, SnPbSeTe, SnPbSTe, and a mixture thereof. The Group IV element may be selected from the group consisting of Si, Ge, and a mixture thereof. The Group IV compound may be a binary compound selected from the group consisting of SiC, SiGe, and a mixture thereof.

In one or more embodiments, the binary compound, the ternary compound, or the quaternary compound may be present in particles in a uniform concentration distribution, or may be present in the same particle in a partially different concentration distribution. In addition, the quantum dot may have a core/shell structure in which one quantum dot is around (e.g., surrounds) another quantum dot. In a core/shell structure, the interface of the core and the shell may have a concentration gradient in which the concentration of an element present in the shell becomes lower towards the core.

In some embodiments, a quantum dot may have the above-described core-shell structure including a core containing nanocrystals and a shell around (e.g., surrounding) the core. The shell of the quantum dot may serve as a protection layer to prevent or substantially prevent the chemical deformation of the core so as to maintain semiconductor properties, and/or serve as a charging layer to impart electrophoresis properties to the quantum dot. The shell may be a single layer or a multilayer. An example of the shell of the quantum dot may include a metal or non-metal oxide, a semiconductor compound, or a combination thereof.

For example, the metal or non-metal oxide may be a binary compound such as SiO₂, Al₂O₃, TiO₂, ZnO, MnO, Mn₂O₃, Mn₃O₄, CuO, FeO, Fe₂O₃, Fe₃O₄, CoO, Co₃O₄, and/or NiO, or a ternary compound such as MgAl₂O₄, CoFe₂O₄, NiFe₂O₄, and/or CoMn₂O₄, but the embodiment of the present disclosure is not limited thereto.

Also, the semiconductor compound may be, for example, CdS, CdSe, CdTe, ZnS, ZnSe, ZnTe, ZnSeS, ZnTeS, GaAs, GaP, GaSb, HgS, HgSe, HgTe, InAs, InP, InGaP, InSb, AlAs, AlP, AlSb, etc., but the embodiment of the present disclosure is not limited thereto.

The quantum dot may have a full width of half maximum (FWHM) of a light emission wavelength spectrum of about 45 nm or less, for example, about 40 nm or less, or about 30 nm or less, and color purity or color reproducibility may be improved in the above range. In addition, light emitted through such a quantum dot is emitted in all directions, and thus a wide viewing angle may be obtained (e.g., improved).

In addition, although the form of a quantum dot is not particularly limited as long as it is a form commonly utilized in the related art, in one or more embodiments, a quantum dot in the form of spherical, pyramidal, multi-arm, and/or cubic nanoparticles, nanotubes, nanowires, nanofibers, nanoplates, etc. may be utilized.

The quantum dot may control the color of emitted light according to the particle size thereof, and accordingly, the quantum dot may have various suitable emission colors such as blue, red, and/or green.

In each light emitting device ED of embodiments illustrated in FIGS. 3-6, the electron transport region ETR is provided on the emission layer EML. The electron transport region ETR may include at least one of the hole blocking layer HBL, the electron transport layer ETL, or the electron injection layer EIL, but the embodiment of the present disclosure is not limited thereto.

The electron transport region ETR may have a single layer formed of a single material, a single layer formed of a plurality of different materials, or a multilayer structure including a plurality of layers formed of a plurality of different materials.

For example, the electron transport region ETR may have a single layer structure of the electron injection layer EIL or the electron transport layer ETL, and may have a single layer structure formed of an electron injection material and an electron transport material. In addition, the electron transport region ETR may have a single layer structure formed of a plurality of different materials, or may have a structure in which an electron transport layer ETL/electron injection layer EIL, a hole blocking layer HBL/electron transport layer ETL/electron injection layer EIL are stacked in the respective stated order from the emission layer EML, but the embodiment of the present disclosure is not limited thereto. The electron transport region ETR may have a thickness, for example, from about 1,000 Å to about 1,500 Å.

The electron transport region ETR may be formed by utilizing various suitable methods such as a vacuum deposition method, a spin coating method, a cast method, a Langmuir-Blodgett (LB) method, an inkjet printing method, a laser printing method, a laser induced thermal imaging (LITI) method, etc.

The electron transport region ETR may include a compound represented by Formula ET-1 below:

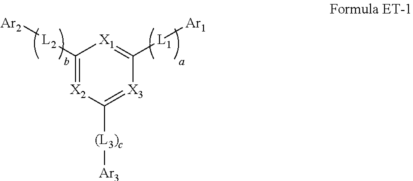

In Formula ET-1, at least one among X₁ to X₃ is N, and the remainder (e.g., the rest) are CRₐ. Rₐ may be a hydrogen atom, a deuterium atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. Ar₁ to Ar₃ may be each independently a hydrogen atom, a deuterium atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms.

In Formula ET-1, a to c may be each independently an integer of 0 to 10. In Formula ET-1, L₁ to L₃ may be each independently a direct linkage, a substituted or unsubstituted arylene group having 6 to 30 ring-forming carbon atoms, or a substituted or unsubstituted heteroarylene group having 2 to 30 ring-forming carbon atoms. In one or more embodiments, when a to c are an integer of 2 or more, L₁ to L₃ may be each independently a substituted or unsubstituted arylene group having 6 to 30 ring-forming carbon atoms, or a substituted or unsubstituted heteroarylene group having 2 to 30 ring-forming carbon atoms.

The electron transport region ETR may include an anthracene-based compound. However, the embodiment of the present disclosure is not limited thereto, and the electron transport region ETR may include, for example, tris(8-hydroxyquinolinato)aluminum (Alq3), 1,3,5-tri[(3-pyridyl)-phen-3-yl]benzene, 2,4,6-tris(3′-(pyridin-3-yl)biphenyl-3-yl)-1,3,5-triazine, 2-(4-(N-phenylbenzoimidazol-1-yl)phenyl)-9,10-dinaphthylanthracene, 1,3,5-tri(1-phenyl-1H-benzo[d]imidazol-2-yl)benzene (TPBi), 2,9-dimethyl-4,7-diphenyl-1,10-phenanthroline (BCP), 4,7-diphenyl-1,10-phenanthroline (Bphen), 3-(4-biphenylyl)-4-phenyl-5-tert-butylphenyl-1,2,4-triazole (TAZ), 4-(naphthalen-1-yl)-3,5-diphenyl-4H-1,2,4-triazole (NTAZ), 2-(4-biphenylyl)-5-(4-tert-butylphenyl)-1,3,4-oxadiazole (tBu-PBD), bis(2-methyl-8-quinolinolato-N1,O8)-(1,1′-biphenyl-4-olato)aluminum (BAlq), berylliumbis(benzoquinolin-10-olate (Bebq₂), 9,10-di(naphthalene-2-yl)anthracene (ADN), 1,3-Bis[3,5-di(pyridin-3-yl)phenyl]benzene (BmPyPhB), or a mixture thereof.

The electron transport region ETR may include at least one among Compound ET1 to Compound ET36 below:

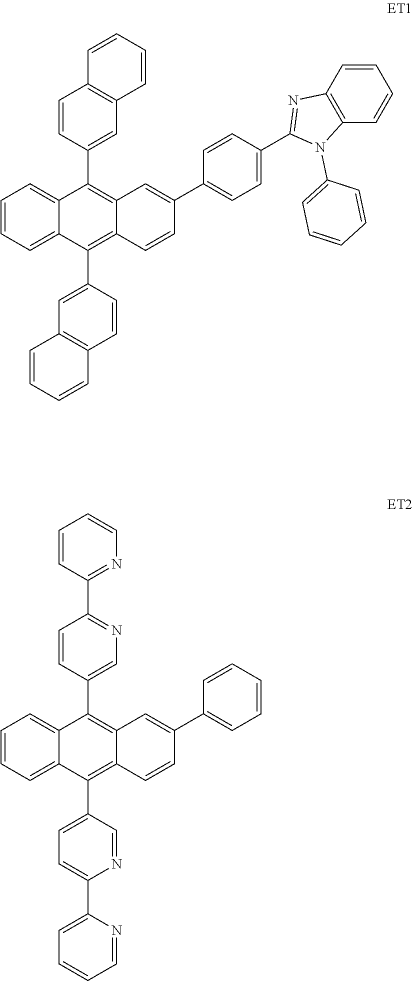 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; c1ccc(-c2ccc3c(-c4ccc(-c5ccccn5)nc4)c4ccccc4c(-c4ccc(-c5ccccn5)nc4)c3c2)cc1]

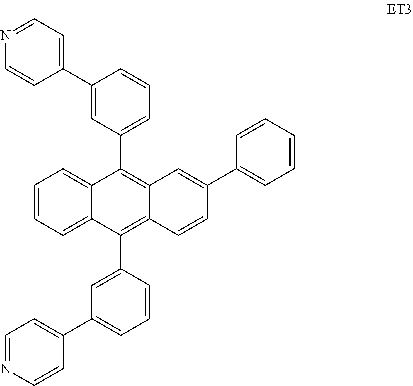 [SMILES: c1ccc(-c2ccc3c(-c4cccc(-c5ccncc5)c4)c4ccccc4c(-c4cccc(-c5ccncc5)c4)c3c2)cc1]

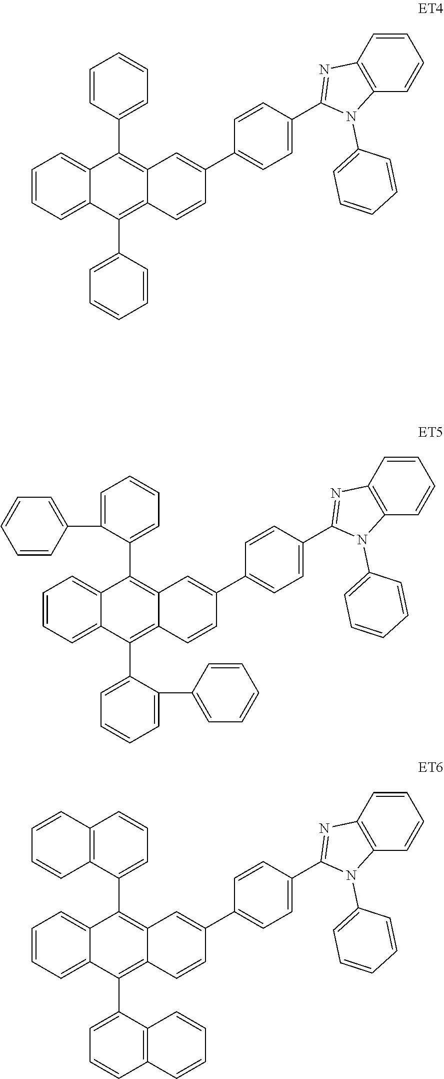 [SMILES: c1ccc(-c2c3ccccc3c(-c3ccccc3)c3cc(-c4ccc(-c5nc6ccccc6n5-c5ccccc5)cc4)ccc23)cc1 ; c1ccc(-c2ccccc2-c2c3ccccc3c(-c3ccccc3-c3ccccc3)c3cc(-c4ccc(-c5nc6ccccc6n5-c5ccccc5)cc4)ccc23)cc1 ; c1ccc(-n2c(-c3ccc(-c4ccc5c(-c6cccc7ccccc67)c6ccccc6c(-c6cccc7ccccc67)c5c4)cc3)nc3ccccc32)cc1]

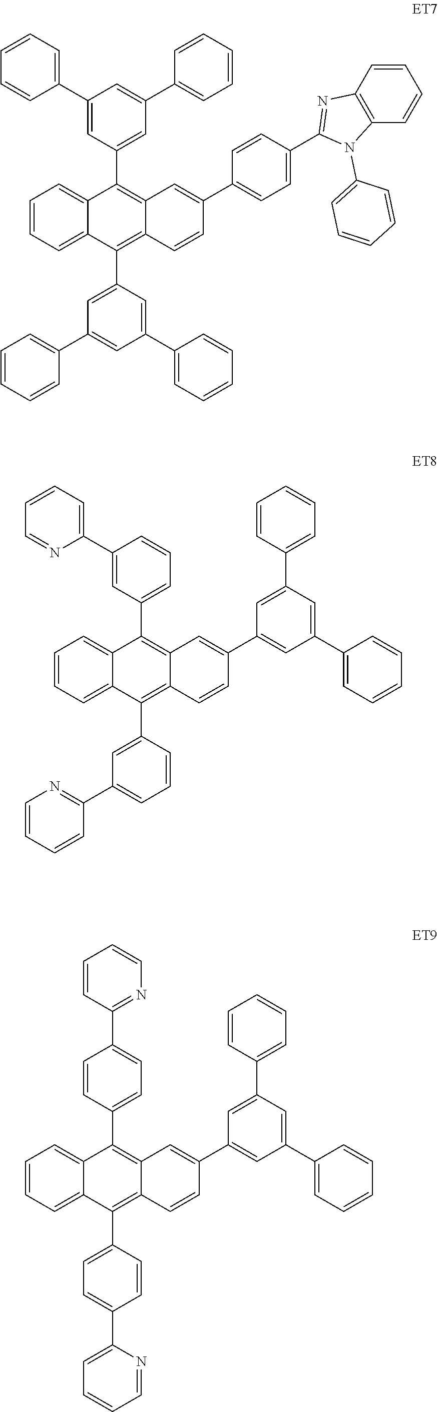 [SMILES: c1ccc(-c2cc(-c3ccccc3)cc(-c3c4ccccc4c(-c4cc(-c5ccccc5)cc(-c5ccccc5)c4)c4cc(-c5ccc(-c6nc7ccccc7n6-c6ccccc6)cc5)ccc34)c2)cc1 ; c1ccc(-c2cc(-c3ccccc3)cc(-c3ccc4c(-c5cccc(-c6ccccn6)c5)c5ccccc5c(-c5cccc(-c6ccccn6)c5)c4c3)c2)cc1 ; c1ccc(-c2cc(-c3ccccc3)cc(-c3ccc4c(-c5ccc(-c6ccccn6)cc5)c5ccccc5c(-c5ccc(-c6ccccn6)cc5)c4c3)c2)cc1]

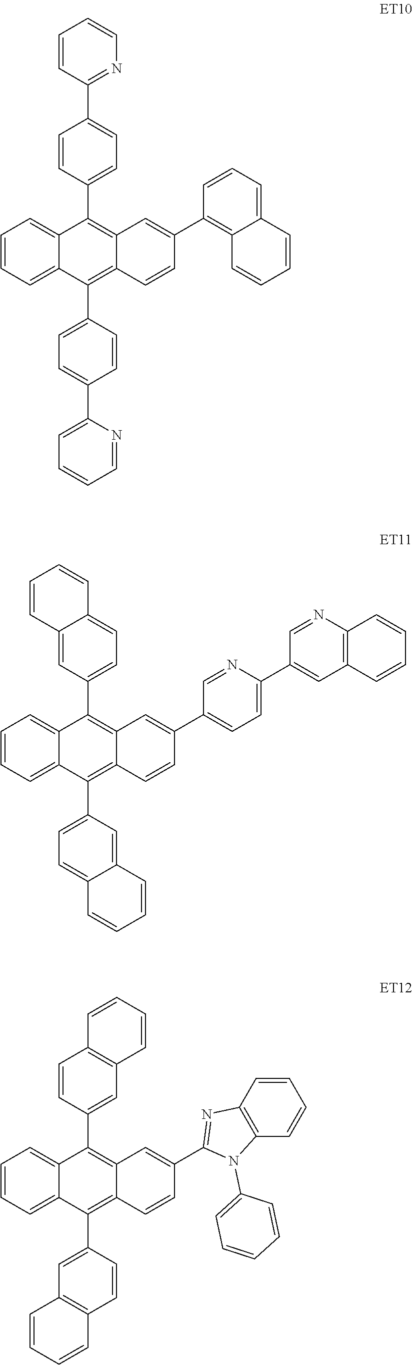 [SMILES: c1ccc(-c2ccc(-c3c4ccccc4c(-c4ccc(-c5ccccn5)cc4)c4cc(-c5cccc6ccccc56)ccc34)cc2)nc1 ; c1ccc2cc(-c3c4ccccc4c(-c4ccc5ccccc5c4)c4cc(-c5ccc(-c6cnc7ccccc7c6)nc5)ccc34)ccc2c1 ; c1ccc(-n2c(-c3ccc4c(-c5ccc6ccccc6c5)c5ccccc5c(-c5ccc6ccccc6c5)c4c3)nc3ccccc32)cc1]

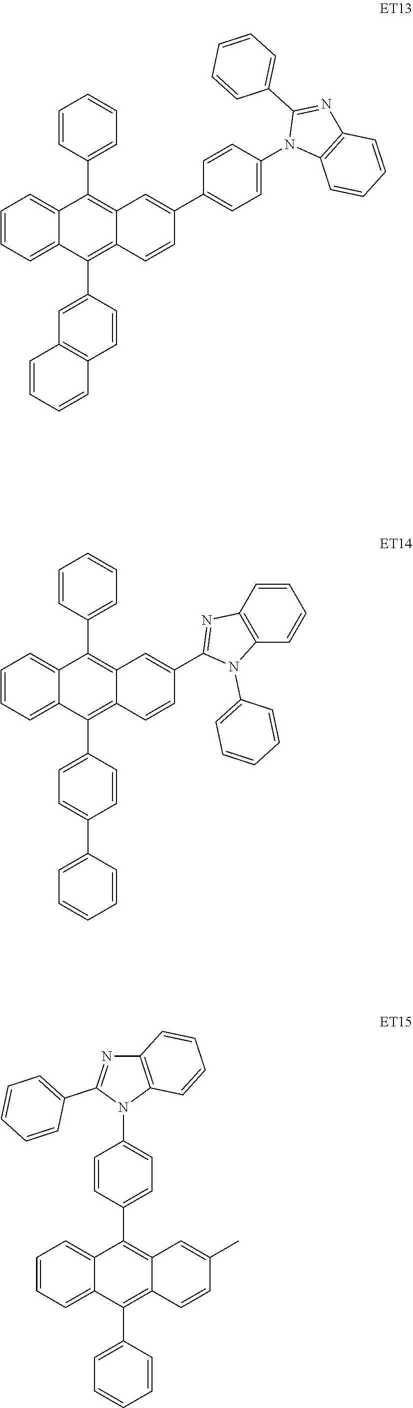 [SMILES: c1ccc(-c2c3ccccc3c(-c3ccc4ccccc4c3)c3ccc(-c4ccc(-n5c(-c6ccccc6)nc6ccccc65)cc4)cc23)cc1 ; c1ccc(-c2ccc(-c3c4ccccc4c(-c4ccccc4)c4cc(-c5nc6ccccc6n5-c5ccccc5)ccc34)cc2)cc1 ; Cc1ccc2c(-c3ccccc3)c3ccccc3c(-c3ccc(-n4c(-c5ccccc5)nc5ccccc54)cc3)c2c1]

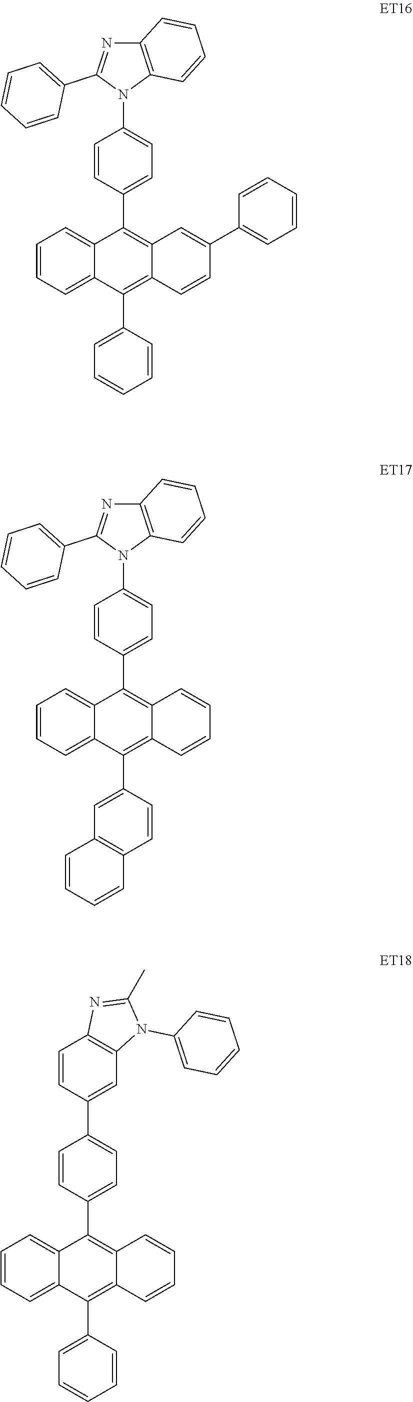 [SMILES: c1ccc(-c2ccc3c(-c4ccccc4)c4ccccc4c(-c4ccc(-n5c(-c6ccccc6)nc6ccccc65)cc4)c3c2)cc1 ; c1ccc(-c2nc3ccccc3n2-c2ccc(-c3c4ccccc4c(-c4ccc5ccccc5c4)c4ccccc34)cc2)cc1 ; Cc1nc2ccc(-c3ccc(-c4c5ccccc5c(-c5ccccc5)c5ccccc45)cc3)cc2n1-c1ccccc1]

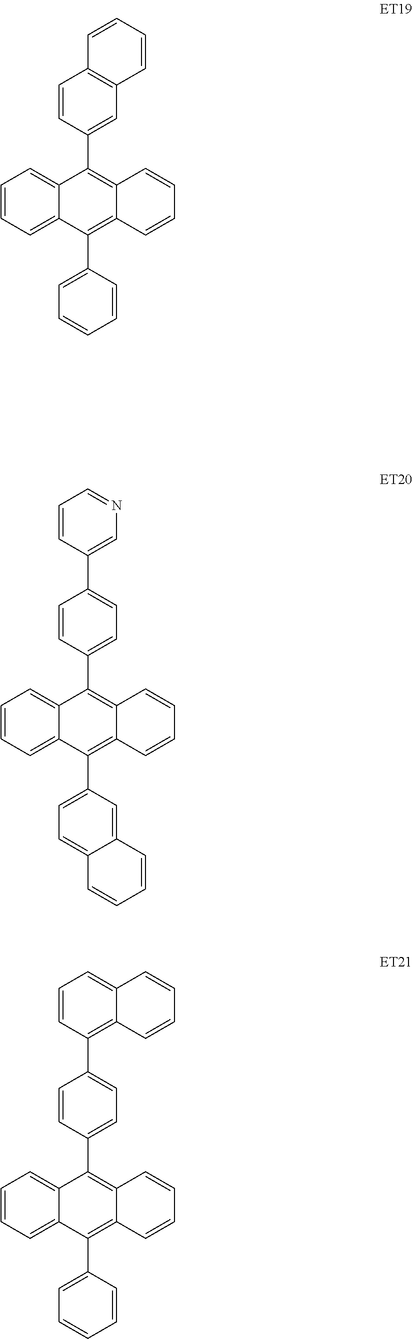

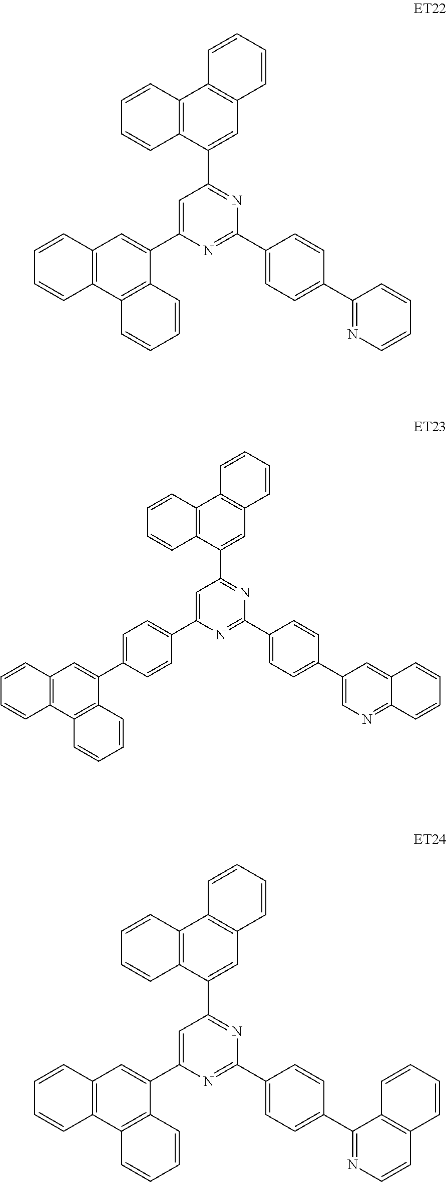 [SMILES: c1ccc(-c2ccc(-c3nc(-c4cc5ccccc5c5ccccc45)cc(-c4cc5ccccc5c5ccccc45)n3)cc2)nc1 ; c1ccc2ncc(-c3ccc(-c4nc(-c5ccc(-c6cc7ccccc7c7ccccc67)cc5)cc(-c5cc6ccccc6c6ccccc56)n4)cc3)cc2c1 ; c1ccc2c(-c3ccc(-c4nc(-c5cc6ccccc6c6ccccc56)cc(-c5cc6ccccc6c6ccccc56)n4)cc3)nccc2c1]

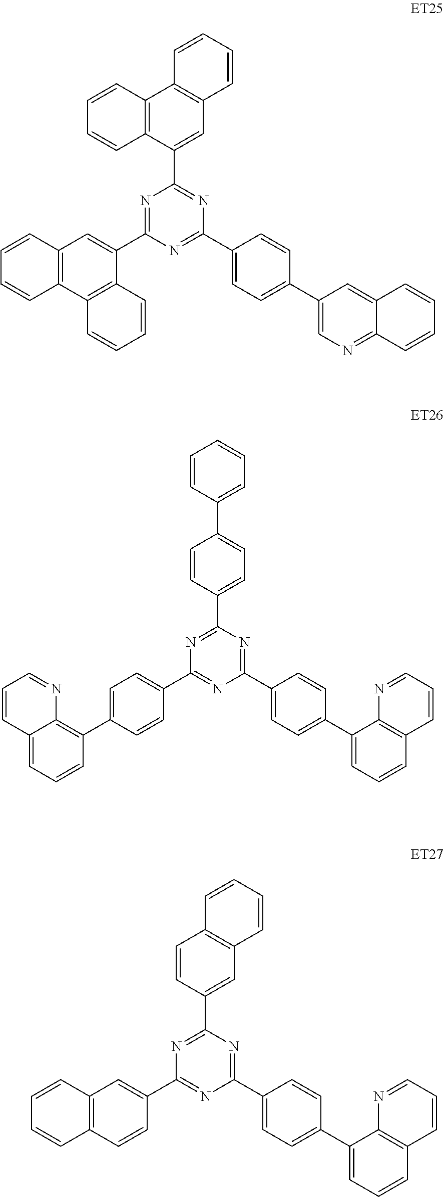 [SMILES: c1ccc2ncc(-c3ccc(-c4nc(-c5cc6ccccc6c6ccccc56)nc(-c5cc6ccccc6c6ccccc56)n4)cc3)cc2c1 ; c1ccc(-c2ccc(-c3nc(-c4ccc(-c5cccc6cccnc56)cc4)nc(-c4ccc(-c5cccc6cccnc56)cc4)n3)cc2)cc1 ; c1ccc2cc(-c3nc(-c4ccc(-c5cccc6cccnc56)cc4)nc(-c4ccc5ccccc5c4)n3)ccc2c1]

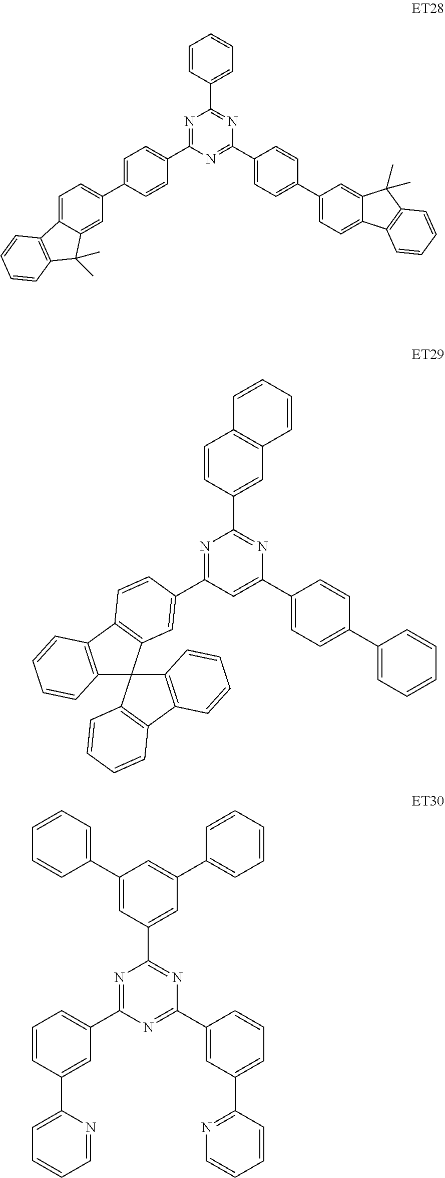 [SMILES: CC1(C)c2ccccc2-c2ccc(-c3ccc(-c4nc(-c5ccccc5)nc(-c5ccc(-c6ccc7c(c6)C(C)(C)c6ccccc6-7)cc5)n4)cc3)cc21 ; c1ccc(-c2ccc(-c3cc(-c4ccc5c(c4)C4(c6ccccc6-c6ccccc64)c4ccccc4-5)nc(-c4ccc5ccccc5c4)n3)cc2)cc1 ; c1ccc(-c2cc(-c3ccccc3)cc(-c3nc(-c4cccc(-c5ccccn5)c4)nc(-c4cccc(-c5ccccn5)c4)n3)c2)cc1]

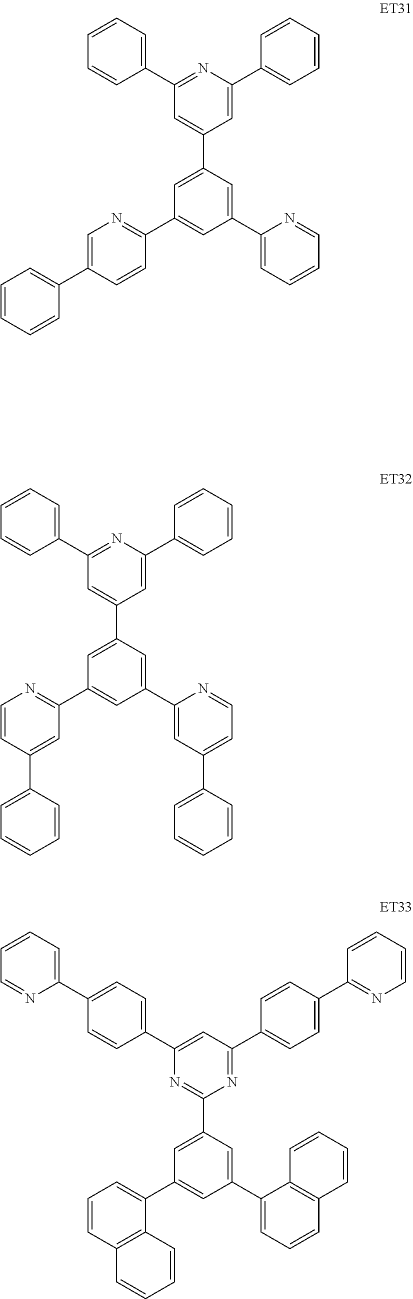 [SMILES: c1ccc(-c2ccc(-c3cc(-c4cc(-c5ccccc5)nc(-c5ccccc5)c4)cc(-c4ccccn4)c3)nc2)cc1 ; c1ccc(-c2ccnc(-c3cc(-c4cc(-c5ccccc5)nc(-c5ccccc5)c4)cc(-c4cc(-c5ccccc5)ccn4)c3)c2)cc1 ; c1ccc(-c2ccc(-c3cc(-c4ccc(-c5ccccn5)cc4)nc(-c4cc(-c5cccc6ccccc56)cc(-c5cccc6ccccc56)c4)n3)cc2)nc1]

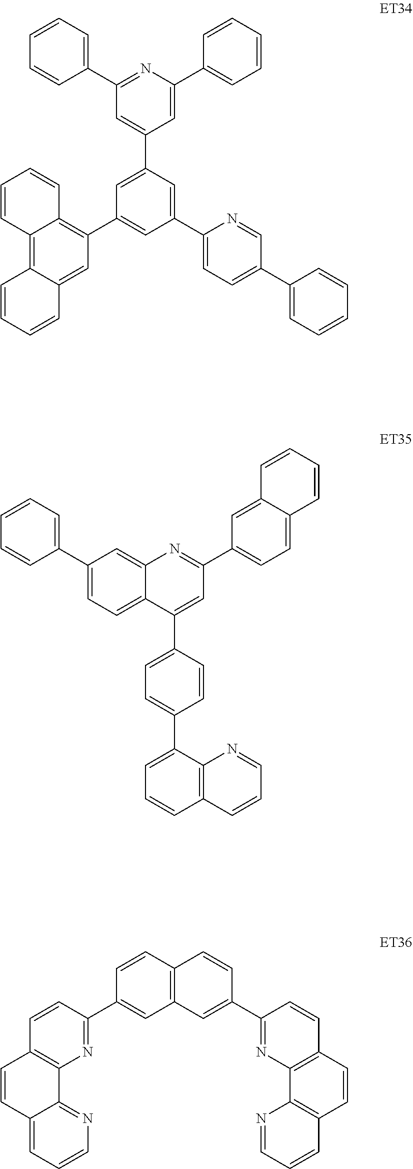 [SMILES: c1ccc(-c2ccc(-c3cc(-c4cc(-c5ccccc5)nc(-c5ccccc5)c4)cc(-c4cc5ccccc5c5ccccc45)c3)nc2)cc1 ; c1ccc(-c2ccc3c(-c4ccc(-c5cccc6cccnc56)cc4)cc(-c4ccc5ccccc5c4)nc3c2)cc1 ; c1cnc2c(c1)ccc1ccc(-c3ccc4ccc(-c5ccc6ccc7cccnc7c6n5)cc4c3)nc12]

In addition, the electron transport region ETR may include a metal halide such as LiF, NaCl, CsF, RbCl, RbI, CuI, and/or KI, a lanthanide metal such as Yb, and a co-deposited material of the metal halide and the lanthanide metal. For example, the electron transport region ETR may include KI:Yb, RbI:Yb, etc., as a co-deposited material. In one or more embodiments, the electron transport region ETR may be formed utilizing a metal oxide such as Li₂O and/or BaO, 8-hydroxyl-lithium quinolate (Liq), etc., but the embodiment of the present disclosure is not limited thereto. The electron transport region ETR may also be formed of a mixture material of an electron transport material and an insulating organometallic salt. The organometallic salt may be a material having an energy band gap of about 4 eV or more. In one or more embodiments, the organometallic salt may include, for example, metal acetates, metal benzoates, metal acetoacetates, metal acetylacetonates, and/or metal stearates.

The electron transport region ETR may further include at least one of 2,9-dimethyl-4,7-diphenyl-1,10-phenanthroline (BCP), or 4,7-diphenyl-1,10-phenanthroline (Bphen) in addition to the above-described materials, but the embodiment of the present disclosure is not limited thereto.

The electron transport region ETR may include the above-described compounds of the hole transport region in at least one of the electron injection layer EIL, the electron transport layer ETL, or the hole blocking layer HBL.

When the electron transport region ETR includes the electron transport layer ETL, the electron transport layer ETL may have a thickness of about 100 Å to about 1,000 Å, for example, about 150 Å to about 500 Å. When the thickness of the electron transport layer ETL satisfies the aforementioned range, satisfactory electron transport characteristics may be obtained without a substantial increase in driving voltage. When the electron transport region ETR includes the electron injection layer EIL, the electron injection layer EIL may have a thickness of about 1 Å to about 100 Å, for example, about 3 Å to about 90 Å. When the thickness of the electron injection layer EIL satisfies the above-described range, satisfactory electron injection characteristics may be obtained without a substantial increase in driving voltage.

The second electrode EL2 is provided on the electron transport region ETR. The second electrode EL2 may be a common electrode. The second electrode EL2 may be a cathode or an anode, but the embodiment of the present disclosure is not limited thereto. For example, when the first electrode EU is an anode, the second electrode EL2 may be a cathode, and when the first electrode EL1 is a cathode, the second electrode EL2 may be an anode.

The second electrode EL2 may be a transmissive electrode, a transflective electrode, or a reflective electrode. When the second electrode EL2 is the transmissive electrode, the second electrode EL2 may be formed of a transparent metal oxide, for example, indium tin oxide (ITO), indium zinc oxide (IZO), zinc oxide (ZnO), indium tin zinc oxide (ITZO), etc.

When the second electrode EL2 is the transflective electrode or the reflective electrode, the second electrode EL2 may include Ag, Mg, Cu, Al, Pt, Pd, Au, Ni, Nd, Ir, Cr, L₁, Ca, LiF/Ca, LiF/Al, Mo, Ti, Yb, W, or a compound or mixture thereof (e.g., AgMg, AgYb, and/or MgAg). In one or more embodiments, the second electrode EL2 may have a multilayer structure including a reflective film or a transflective film formed of the above-described materials, and a transparent conductive film formed of ITO, IZO, ZnO, ITZO, etc. For example, the second electrode EL2 may include the above-described metal materials, combinations of at least two metal materials of the above-described metal materials, oxides of the above-described metal materials, and/or the like.

In one or more embodiments, the second electrode EL2 may be connected with an auxiliary electrode. When the second electrode EL2 is connected with the auxiliary electrode, the resistance of the second electrode EL2 may decrease.

In one or more embodiments, a capping layer CPL may further be on the second electrode EL2 of the light emitting device ED of an embodiment. The capping layer CPL may include a multilayer or a single layer.

In an embodiment, the capping layer CPL may be an organic layer or an inorganic layer. For example, when the capping layer CPL includes an inorganic material, the inorganic material may include an alkali metal compound such as LiF, and/or an alkaline earth metal compound such as MgF₂, SiON, SiNₓ, SiOy, etc.

For example, when the capping layer CPL includes an organic material, the organic material may include α-NPD, NPB, TPD, m-MTDATA, Alq3, CuPc, N4,N4,N4′,N4′-tetra(biphenyl-4-yl)biphenyl-4,4′-diamine (TPD15), 4,4′,4″-tris(carbazol sol-9-yl)triphenylamine (TCTA), etc., an epoxy resin, and/or an acrylate such as methacrylate. However, the embodiment of the present disclosure is not limited thereto, and the capping layer CPL may include at least one among Compounds P1 to P5 below:

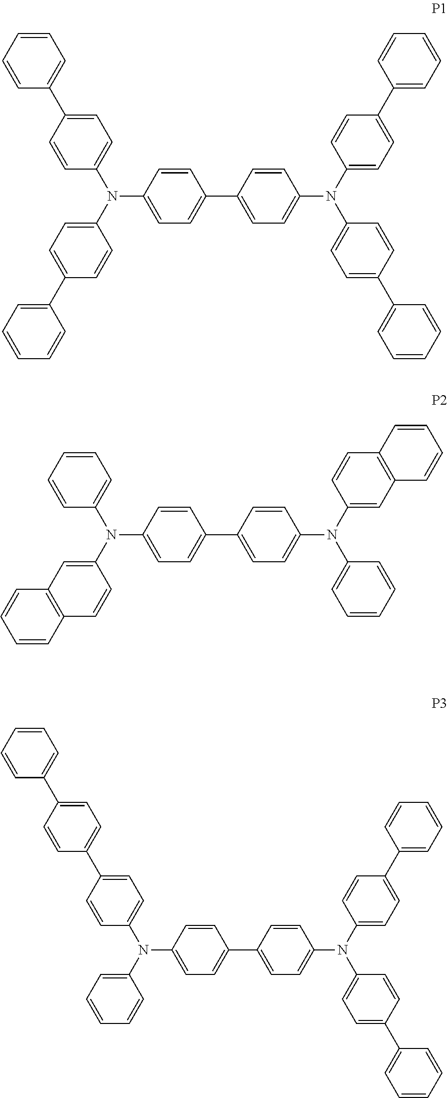 [SMILES: c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1 ; c1ccc(N(c2ccc(-c3ccc(N(c4ccccc4)c4ccc5ccccc5c4)cc3)cc2)c2ccc3ccccc3c2)cc1 ; c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1]

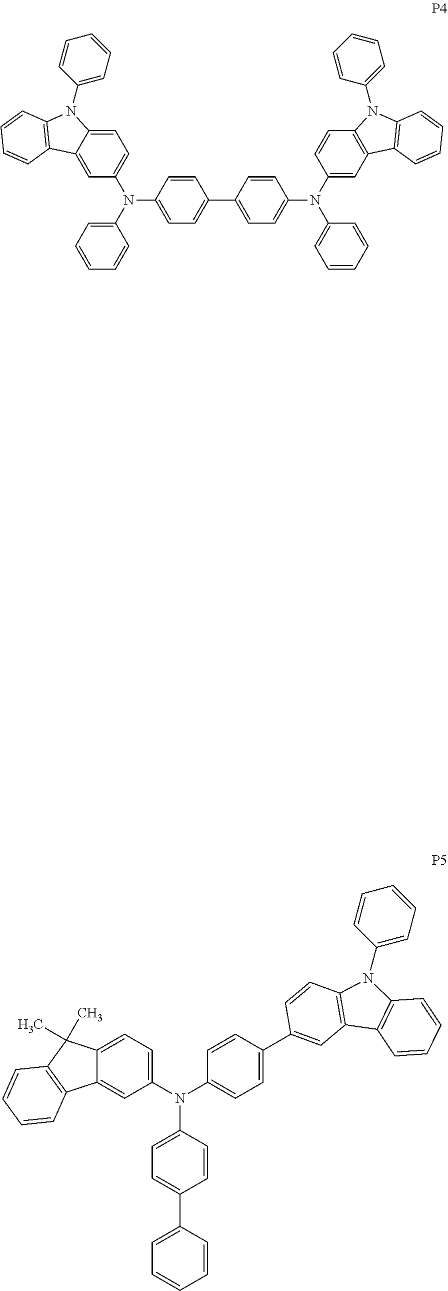 [SMILES: c1ccc(N(c2ccc(-c3ccc(N(c4ccccc4)c4ccc5c(c4)c4ccccc4n5-c4ccccc4)cc3)cc2)c2ccc3c(c2)c2ccccc2n3-c2ccccc2)cc1 ; CC1(C)c2ccccc2-c2cc(N(c3ccc(-c4ccccc4)cc3)c3ccc(-c4ccc5c(c4)c4ccccc4n5-c4ccccc4)cc3)ccc21]

In one or more embodiments, the refractive index of the capping layer CPL may be about 1.6 or more. For example, the refractive index of the capping layer CPL may be about 1.6 or more with respect to light in a wavelength range of about 550 nm to about 660 nm.

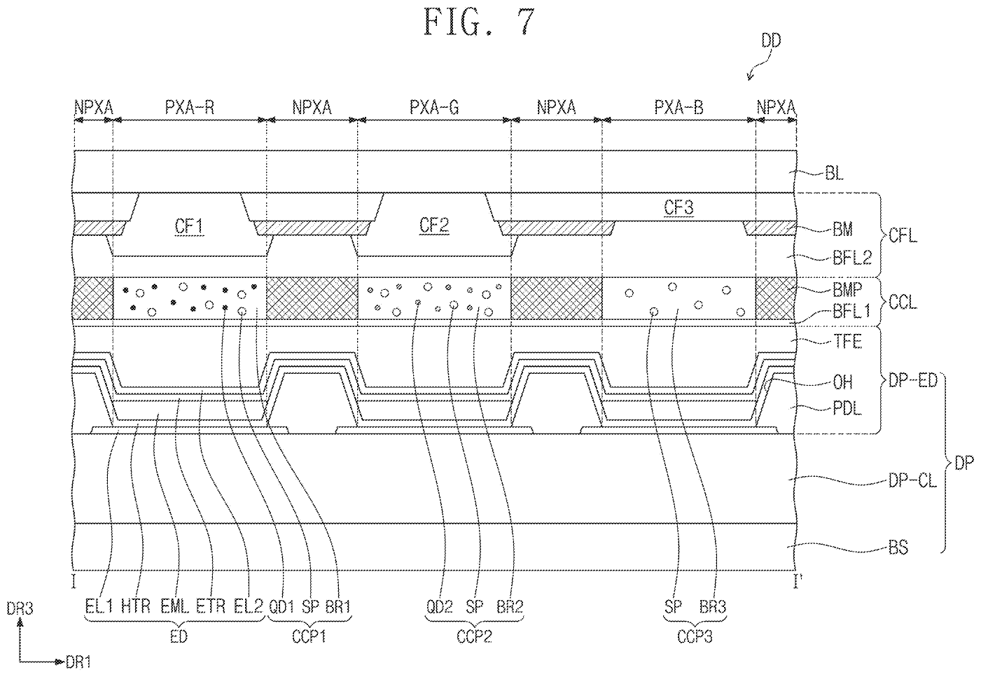

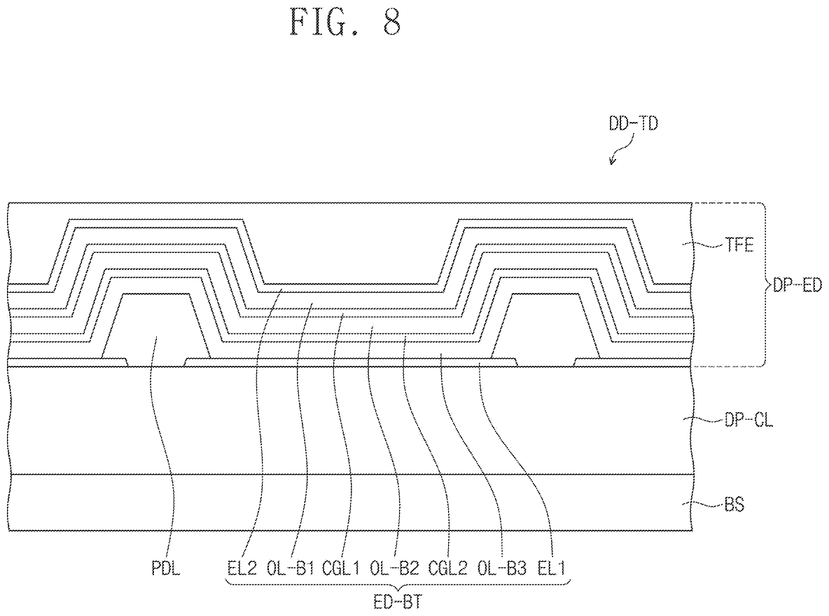

FIGS. 7-8 each are a cross-sectional view of a display apparatus according to an embodiment. Hereinafter, in describing the display apparatus of an embodiment with reference to FIGS. 7-8, the duplicated features which have been described in FIGS. 1-6 are not described again, but their differences will be mainly described.

Referring to FIG. 7, the display apparatus DD according to an embodiment may include a display panel DP including a display device layer DP-ED, a light control layer CCL on the display panel DP, and a color filter layer CFL.

In an embodiment illustrated in FIG. 7, the display panel DP may include a base layer BS, a circuit layer DP-CL provided on the base layer BS, and the display device layer DP-ED, and the display device layer DP-ED may include a light emitting device ED.

The light emitting device ED may include a first electrode EL1, a hole transport region HTR on the first electrode EL1, an emission layer EML on the hole transport region HTR, an electron transport region ETR on the emission layer EML, and a second electrode EL2 on the electron transport region ETR. In one or more embodiments, the structures of the light emitting devices of FIGS. 3-6 as described above may be equally applied to the structure of the light emitting device ED shown in FIG. 7.

Referring to FIG. 7, the emission layer EML may be in an opening OH defined in a pixel defining film PDL. For example, the emission layer EML which is divided by the pixel defining film PDL and provided corresponding to each light emitting regions PXA-R, PXA-G, and PXA-B may emit light in the same wavelength range. In the display apparatus DD of an embodiment, the emission layer EML may emit blue light. In one or more embodiments, unlike what is illustrated, the emission layer EML may be provided as a common layer in the entire light emitting regions PXA-R, PXA-G, and PXA-B.

The light control layer CCL may be on the display panel DP. The light control layer CCL may include a light conversion body. The light conversion body may be a quantum dot, a phosphor, and/or the like. The light conversion body may convert the wavelength of provided light and emit light having a wavelength from the provided light. That is, the light control layer CCL may be a layer containing the quantum dot and/or a layer containing the phosphor.

The light control layer CCL may include a plurality of light control parts CCP1, CCP2 and CCP3. The light control parts CCP1, CCP2, and CCP3 may be spaced apart from one another.

Referring to FIG. 7, divided patterns BMP may be between the light control parts CCP1, CCP2 and CCP3 which are spaced apart from each other, but the embodiment of the present disclosure is not limited thereto. FIG. 7 illustrates that the divided patterns BMP do not overlap the light control parts CCP1, CCP2 and CCP3, but in one or more embodiments, at least a portion of the edges of the light control parts CCP1, CCP2 and CCP3 may overlap the divided patterns BMP.

The light control layer CCL may include a first light control part CCP1 containing a first quantum dot QD1 which converts a first color light provided from the light emitting device ED into a second color light, a second light control part CCP2 containing a second quantum dot QD2 which converts the first color light into a third color light, and a third light control part CCP3 which transmits the first color light.

In an embodiment, the first light control part CCP1 may provide red light as the second color light, and the second light control part CCP2 may provide green light as the third color light. The third light control part CCP3 may provide blue light by transmitting the blue light as the first color light provided in the light emitting device ED. For example, the first quantum dot QD1 may be a red quantum dot (e.g., a red-light emitting quantum dot), and the second quantum dot QD2 may be a green quantum dot (e.g., a green-light emitting quantum dot). The same as described above may be applied with respect to the quantum dots QD1 and QD2.

In addition, the light control layer CCL may further include a scatterer SP. The first light control part CCP1 may include the first quantum dot QD1 and the scatterer SP, the second light control part CCP2 may include the second quantum dot QD2 and the scatterer SP, and the third light control part CCP3 may not include any quantum dot but include the scatterer SP.

The scatterer SP may be inorganic particles. For example, the scatterer SP may include at least one of TiO₂, ZnO, Al₂O₃, SiO₂, or hollow silica. The scatterer SP may include any one of TiO₂, ZnO, Al₂O₃, SiO₂, or hollow silica, or may be a mixture of two or more materials selected from among TiO₂, ZnO, Al₂O₃, SiO₂, and hollow silica.

The first light control part CCP1, the second light control part CCP2, and the third light control part CCP3 each may respectively include base resins BR1, BR2, and BR3 in which the quantum dots QD1 and QD2 and the scatterer SP are dispersed. In an embodiment, the first light control part CCP1 may include the first quantum dot QD1 and the scatterer SP dispersed in a first base resin BR1, the second light control part CCP2 may include the second quantum dot QD2 and the scatterer SP dispersed in a second base resin BR2, and the third light control part CCP3 may include the scatterer SP dispersed in a third base resin BR3. The base resins BR1, BR2, and BR3 are media in which the quantum dots QD1 and QD2 and the scatterer SP are dispersed, and may be formed of various suitable resin compositions, which may be generally referred to as a binder. For example, the base resins BR1, BR2, and BR3 may be acrylic-based resins, urethane-based resins, silicone-based resins, epoxy-based resins, etc. The base resins BR1, BR2, and BR3 may be transparent resins. In an embodiment, the first base resin BR1, the second base resin BR2, and the third base resin BR3 each may be the same as or different from each other.

The light control layer CCL may include a barrier layer BFL1. The barrier layer BFL1 may serve to prevent or reduce the penetration of moisture and/or oxygen (hereinafter, referred to as “moisture/oxygen”). The barrier layer BFL1 may be on the light control parts CCP1, CCP2, and CCP3 to block the light control parts CCP1, CCP2 and CCP3 from being exposed to moisture/oxygen. In one or more embodiments, the barrier layer BFL1 may cover the light control parts CCP1, CCP2, and CCP3. In addition, the barrier layer BFL2 may be provided between the light control parts CCP1, CCP2, and CCP3 and the color filter layer CFL.

The barrier layers BFL1 and BFL2 may include at least one inorganic layer. That is, the barrier layers BFL1 and BFL2 may include an inorganic material. For example, the barrier layers BFL1 and BFL2 may include a silicon nitride, an aluminum nitride, a zirconium nitride, a titanium nitride, a hafnium nitride, a tantalum nitride, a silicon oxide, an aluminum oxide, a titanium oxide, a tin oxide, a cerium oxide, a silicon oxynitride, a metal thin film which secures a transmittance, etc. In one or more embodiments, the barrier layers BFL1 and BFL2 may further include an organic film. The barrier layers BFL1 and BFL2 may be formed of a single layer or a plurality of layers.

In the display apparatus DD of an embodiment, the color filter layer CFL may be on the light control layer CCL. For example, the color filter layer CFL may be directly on the light control layer CCL. In one or more embodiments, the barrier layer BFL2 may be omitted.

The color filter layer CFL may include a light shielding unit BM and filters CF-1, CF-2, and CF-3. The color filter layer CFL may include a first filter CF1 configured to transmit the second color light, a second filter CF2 configured to transmit the third color light, and a third filter CF3 configured to transmit the first color light. For example, the first filter CF1 may be a red filter, the second filter CF2 may be a green filter, and the third filter CF3 may be a blue filter. The filters CF1, CF2, and CF3 may each include a polymeric photosensitive resin and a pigment and/or dye. The first filter CF1 may include a red pigment and/or dye, the second filter CF2 may include a green pigment and/or dye, and the third filter CF3 may include a blue pigment and/or dye. However, the embodiment of the present disclosure is not limited thereto, and the third filter CF3 may not include any pigment or dye. The third filter CF3 may include a polymeric photosensitive resin and may not include a pigment or dye. The third filter CF3 may be transparent. The third filter CF3 may be formed of a transparent photosensitive resin.

Furthermore, in an embodiment, the first filter CF1 and the second filter CF2 may each be a yellow filter. The first filter CF1 and the second filter CF2 may not be separated but be provided as one filter.

The light shielding unit BM may be a black matrix. The light shielding unit BM may include an organic light shielding material or an inorganic light shielding material containing a black pigment and/or dye. The light shielding unit BM may prevent or reduce light leakage, and may separate boundaries between the adjacent filters CF1, CF2, and CF3. In addition, in an embodiment, the light shielding unit BM may be formed of a blue filter.

The first to third filters CF1, CF2, and CF3 may correspond to the red light emitting region PXA-R, the green light emitting region PXA-G, and the blue light emitting region PXA-B, respectively.

A base substrate BL may be on the color filter layer CFL. The base substrate BL may be a member which provides a base surface in which the color filter layer CFL, the light control layer CCL, and/or the like are located. The base substrate BL may be a glass substrate, a metal substrate, a plastic substrate, etc. However, the embodiment of the present disclosure is not limited thereto, and the base substrate BL may be an inorganic layer, an organic layer, or a composite material layer. In addition, unlike what is illustrated, in an embodiment, the base substrate BL may be omitted.

FIG. 8 is a cross-sectional view illustrating a part of a display apparatus according to an embodiment. FIG. 8 illustrates a cross-sectional view of a part corresponding to the display panel DP of FIG. 7. In the display apparatus DD-TD of an embodiment, the light emitting device ED-BT may include a plurality of light emitting structures OL-B1, OL-B2, and OL-B3. The light emitting device ED-BT may include a first electrode EL1 and a second electrode EL2 which face each other (e.g., which are oppositely located), and the plurality of light emitting structures OL-B1, OL-B2, and OL-B3 sequentially stacked in the stated order in the thickness direction between the first electrode EL1 and the second electrode EL2. The light emitting structures OL-B1, OL-B2, and OL-B3 may each include an emission layer EML (FIG. 7) and a hole transport region HTR and an electron transport region ETR with the emission layer EML (FIG. 7) therebetween.

That is, the light emitting device ED-BT included in the display apparatus DD-TD of an embodiment may be a light emitting device having a tandem structure and including a plurality of emission layers.

In an embodiment illustrated in FIG. 8, light respectively emitted from the light emitting structures OL-B1, OL-B2, and OL-B3 may all be blue light. However, the embodiment of the present disclosure is not limited thereto, and the light respectively emitted from the light emitting structures OL-B1, OL-B2, and OL-B3 may have wavelength ranges different from each other. For example, the light emitting device ED-BT including the plurality of light emitting structures OL-B1, OL-B2, and OL-B3, which emit light having wavelength ranges different from each other, may emit white light.

A charge generation layer CGL1, CGL2 may be between the neighboring light emitting structures OL-B1, OL-B2, and OL-B3. The charge generation layer CGL1, CGL2 may include a p-type charge generation layer and/or an n-type charge generation layer.

Hereinafter, with reference to Examples and Comparative Examples, a compound according to an embodiment of the present disclosure and a light emitting device of an embodiment will be described in more detail. In addition, Examples shown below are illustrated only for the understanding of the present disclosure, and the scope of the present disclosure is not limited thereto.

### Examples

### 1. Synthesis of Amine Compound

First, a synthesis method of an amine compound according to the present embodiment will be described in more detail by illustrating the synthesis methods of Compounds 6, 8, 14, 22, 38, 54, 70, 86, 102, 118, 134, 150, and 166. Also, in the following descriptions, the synthesis method of the amine compound is provided as an example, but the synthesis method according to an embodiment of the present disclosure is not limited to Examples below.

### (1) Synthesis of Compound 6

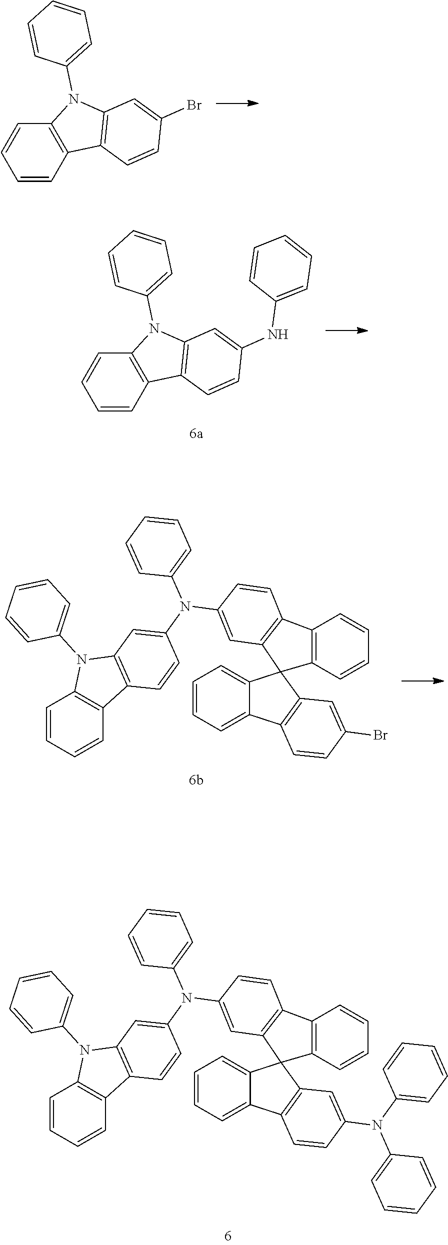 [SMILES: Brc1ccc2c3ccccc3n(-c3ccccc3)c2c1 ; c1ccc(Nc2ccc3c4ccccc4n(-c4ccccc4)c3c2)cc1 ; Brc1ccc2c(c1)C1(c3ccccc3-2)c2ccccc2-c2ccc(N(c3ccccc3)c3ccc4c5ccccc5n(-c5ccccc5)c4c3)cc21 ; c1ccc(N(c2ccccc2)c2ccc3c(c2)C2(c4ccccc4-3)c3ccccc3-c3ccc(N(c4ccccc4)c4ccc5c6ccccc6n(-c6ccccc6)c5c4)cc32)cc1]

### 1-1. (Synthesis of Intermediate 6a)

Aniline (1.0 eq.), 2-bromo-9-phenyl-9H-carbazole (1.1 eq.), tris(dibenzylideneacetone)dipalladium(0) (0.05 eq.), tri-tert-butylphosphine (0.10 eq.), and sodium tert-butoxide (2.0 eq.) were dissolved in toluene, and then the mixture was stirred at about 90° C. for about 2 hours in a nitrogen atmosphere. After cooling, the resulting product was washed three times with ethyl acetate and water, and then an organic layer was obtained. The obtained organic layer was dried over MgSO₄, and then dried under a reduced pressure. The dried organic layer was purified by column chromatography to obtain Intermediate 6a. (yield: 78%)

### 1-2. (Synthesis of Intermediate 6b)

Intermediate 6a (1.0 eq.), 2,2′-dibromo-9,9′-spirobi[fluorene] (2.1 eq.), tris(dibenzylideneacetone)dipalladium(0) (0.02 eq.), tri-tert-butylphosphine (0.04 eq.), and sodium tert-butoxide (1.5 eq.) were dissolved in toluene and then the mixture was stirred at about 80° C. for about 1 hour in a nitrogen atmosphere. After cooling, the resulting product was washed three times with ethyl acetate and water, and then an organic layer was obtained. The obtained organic layer was dried over MgSO₄, and then dried under a reduced pressure. The dried organic layer was purified by column chromatography to obtain Intermediate 6b. (yield: 58%)

### 1-3. Synthesis of Compound 6

Intermediate 6b (1.0 eq.), diphenylamine (1.1 eq.), tris(dibenzylideneacetone)dipalladium(0) (0.02 eq.), tri-tert-butylphosphine (0.04 eq.), and sodium tert-butoxide (1.5 eq.) were dissolved in toluene and then the mixture was stirred at about 80° C. for about 1 hour in a nitrogen atmosphere. After cooling, the resulting product was washed three times with ethyl acetate and water, and then an organic layer was obtained. The obtained organic layer was dried over MgSO₄, and then dried under a reduced pressure. The dried organic layer was purified by column chromatography to obtain Compound 6. (yield: 78%)

By measuring FAB-MS, a mass number of m/z=815.33 was observed by molecular ion peak, thereby identifying Compound 6.

### (2) Synthesis of Compound 8

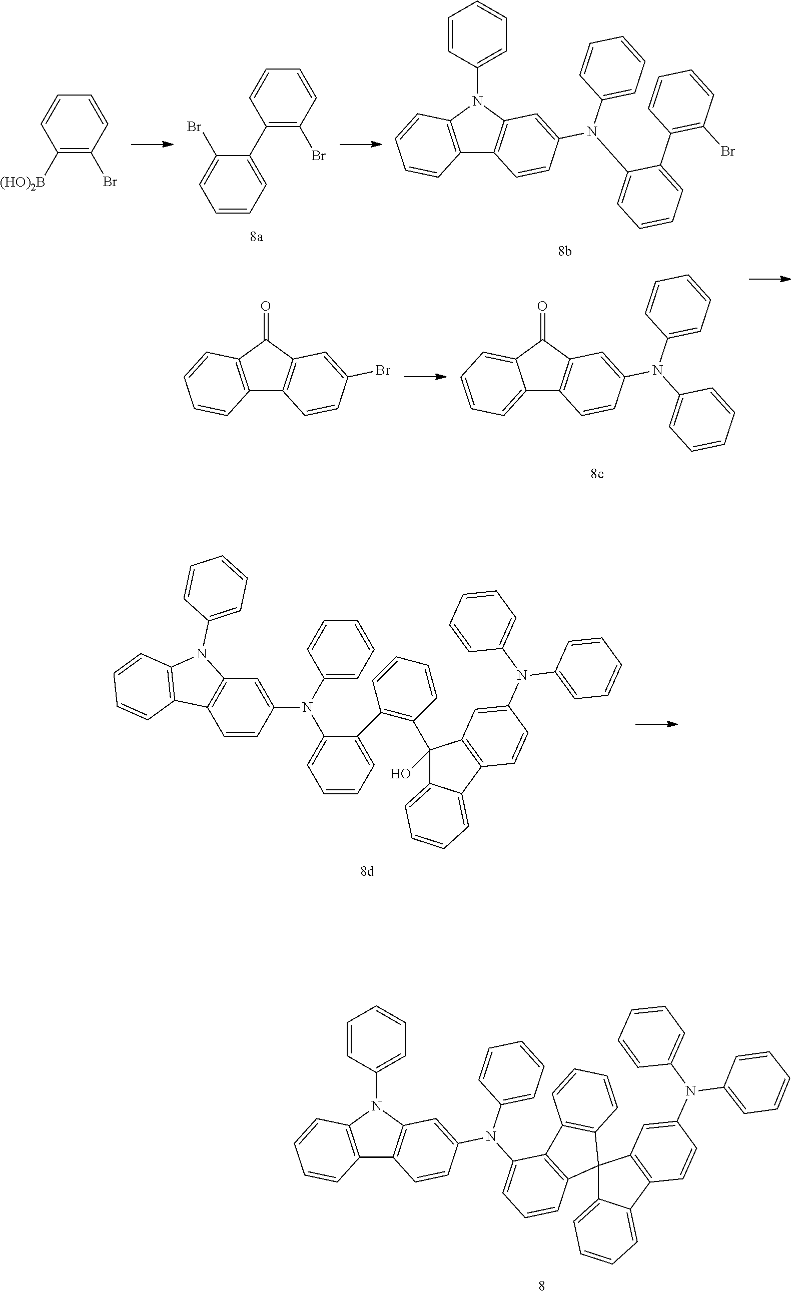

### 2-1. (Synthesis of Intermediate 8a)

(2-bromophenyl)boronic acid (1.0 eq.), 1,2-dibromobenzene (1.2 eq.), tetrakis(triphenylphosphine)palladium (0.05 eq.), and potassium carbonate (2.0 eq.) were dissolved in dioxane:H₂O (which has a volume ratio of about 4:1), and then the mixture was stirred at about 110° C. for about 6 hours in a nitrogen atmosphere. After cooling, the resulting product was washed three times with ethyl acetate and water, and then an organic layer was obtained. The obtained organic layer was dried over MgSO₄, and then dried under a reduced pressure. The dried organic layer was purified by column chromatography to obtain Intermediate 8a. (yield: 52%)

### 2-2. (Synthesis of Intermediate 8b)

Intermediate 6a (1.0 eq.), Intermediate 8a (1.2 eq.), tris(dibenzylideneacetone)dipalladium(0) (0.02 eq.), tri-tert-butylphosphine (0.04 eq.), and sodium tert-butoxide (1.5 eq.) were dissolved in toluene, and then the mixture was stirred at about 80° C. for about 1 hour in a nitrogen atmosphere. After cooling, the resulting product was washed three times with ethyl acetate and water, and then an organic layer was obtained. The obtained organic layer was dried over MgSO₄, and then dried under a reduced pressure. The dried organic layer was purified by column chromatography to obtain Intermediate 8b. (yield: 78%)

### 2-3. (Synthesis of Intermediate 8c)

2-bromo-9H-fluoren-9-one (1.0 eq.), diphenylamine (1.2 eq.), tris(dibenzylideneacetone)dipalladium(0) (0.02 eq.), tri-tert-butylphosphine (0.04 eq.), and sodium tert-butoxide (1.5 eq.) were dissolved in toluene, and then the mixture was stirred at about 80° C. for about 1 hour in a nitrogen atmosphere. After cooling, the resulting product was washed three times with ethyl acetate and water, and then an organic layer was obtained. The obtained organic layer was dried over MgSO₄, and then dried under a reduced pressure. The dried organic layer was purified by column chromatography to obtain Intermediate 8c. (yield: 78%)

### 2-4. (Synthesis of Intermediate 8d)

Anhydrous THF was added dropwise to Intermediate 8b (1.0 eq.), and then the mixture was cooled to about −78° C. in a nitrogen atmosphere. To the cooled solution, n-BuLi (1.1 eq.) was slowly added dropwise, and then the mixture was stirred at about −78° C. for about 1 hour. To this solution, Intermediate 8c (1.1 eq.) was slowly added dropwise, and then the mixture was stirred for about 3 hours. After cooling, the resulting product was washed three times with ethyl acetate and water, and then an organic layer was obtained. The obtained organic layer was dried over MgSO₄, and then dried under a reduced pressure. The dried organic layer was purified by column chromatography to obtain Intermediate 8d. (yield: 82%)

### 2-5. Synthesis of Compound 8

Intermediate 8d (1.0 eq.) was dissolved in acetic acid:hydrochloric acid (which has a volume ratio of about 9:1), and then the mixture was stirred at about 80° C. for about 2 hours in a nitrogen atmosphere. After cooling, the resulting product was washed three times with ethyl acetate and water, and then an organic layer was obtained. The obtained organic layer was dried over MgSO₄, and then dried under a reduced pressure. The dried organic layer was purified by column chromatography to obtain Compound 8. (yield: 78%)

By measuring FAB-MS, a mass number of m/z=815.33 was observed by molecular ion peak, thereby identifying Compound 8.

### (3) Synthesis of Compound 14

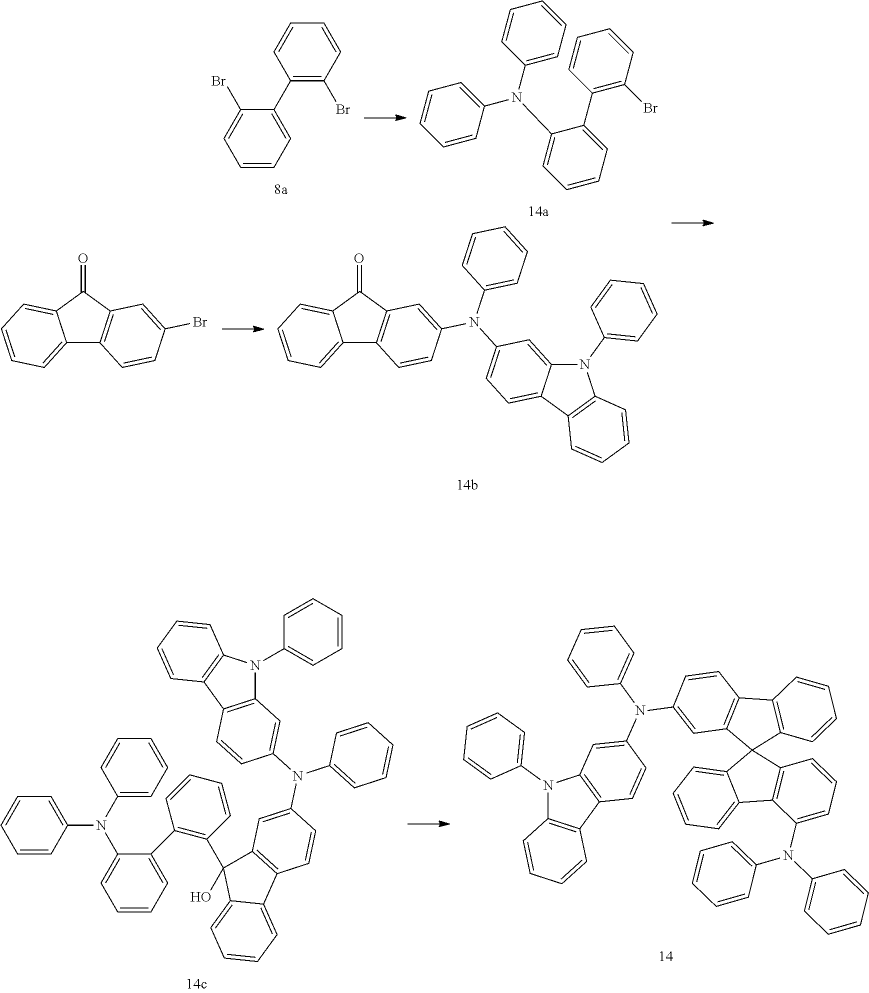 [SMILES: Brc1ccccc1-c1ccccc1Br ; Brc1ccccc1-c1ccccc1N(c1ccccc1)c1ccccc1 ; O=C1c2ccccc2-c2ccc(Br)cc21 ; O=C1c2ccccc2-c2ccc(N(c3ccccc3)c3ccc4c5ccccc5n(-c5ccccc5)c4c3)cc21 ; OC1(c2ccccc2-c2ccccc2N(c2ccccc2)c2ccccc2)c2ccccc2-c2ccc(N(c3ccccc3)c3ccc4c5ccccc5n(-c5ccccc5)c4c3)cc21 ; c1ccc(N(c2ccc3c(c2)C2(c4ccccc4-3)c3ccccc3-c3c(N(c4ccccc4)c4ccccc4)cccc32)c2ccc3c4ccccc4n(-c4ccccc4)c3c2)cc1]

### 3-1. (Synthesis of Intermediate 14a)

Intermediate 8a (1.0 eq.), diphenylamine (1.0 eq.), tris(dibenzylideneacetone)dipalladium(0) (0.02 eq.), tri-tert-butylphosphine (0.04 eq.), and sodium tert-butoxide (1.5 eq.) were dissolved in toluene and then the mixture was stirred at about 80° C. for about 1 hour in a nitrogen atmosphere. After cooling, the resulting product was washed three times with ethyl acetate and water, and then an organic layer was obtained. The obtained organic layer was dried over MgSO₄, and then dried under a reduced pressure. The dried organic layer was purified by column chromatography to obtain Intermediate 14a. (yield: 78%)

### 3-2. (Synthesis of Intermediate 14b)

2-bromo-9H-fluoren-9-one (1.0 eq.), Intermediate 6a (1.1 eq.), tris(dibenzylideneacetone)dipalladium(0) (0.02 eq.), tri-tert-butylphosphine (0.04 eq.), and sodium tert-butoxide (1.5 eq.) were dissolved in toluene, and then the mixture was stirred at about 80° C. for about 1 hour in a nitrogen atmosphere. After cooling, the resulting product was washed three times with ethyl acetate and water, and then an organic layer was obtained. The obtained organic layer was dried over MgSO₄, and then dried under a reduced pressure. The dried organic layer was purified by column chromatography to obtain Intermediate 14b. (yield: 78%)

### 3-3. (Synthesis of Intermediate 14c)

Anhydrous THF was added dropwise to Intermediate 14a (1.0 eq.), and then the mixture was cooled to about −78° C. in a nitrogen atmosphere. To the cooled solution, n-BuLi (1.1 eq.) was slowly added dropwise, and then the mixture was stirred at about −78° C. for about 1 hour. To this solution, Intermediate 14b (1.1 eq.) was slowly added dropwise, and then the mixture was stirred for about 3 hours. After cooling, the resulting product was washed three times with ethyl acetate and water, and then an organic layer was obtained. The obtained organic layer was dried over MgSO₄, and then dried under a reduced pressure. The dried organic layer was purified by column chromatography to obtain Intermediate 14c. (yield: 82%)

### 3-4. Synthesis of Compound 14

Intermediate 14c (1.0 eq.) was dissolved in acetic acid:hydrochloric acid (which has a volume ratio of about 9:1), and then the mixture was stirred at about 80° C. for about 2 hours in a nitrogen atmosphere. After cooling, the resulting product was washed three times with ethyl acetate and water, and then an organic layer was obtained. The obtained organic layer was dried over MgSO₄, and then dried under a reduced pressure. The dried organic layer was purified by column chromatography to obtain Compound 14. (yield: 78%)

By measuring FAB-MS, a mass number of m/z=815.33 was observed by molecular ion peak, thereby identifying Compound 14.

### (4) Synthesis of Compound 22

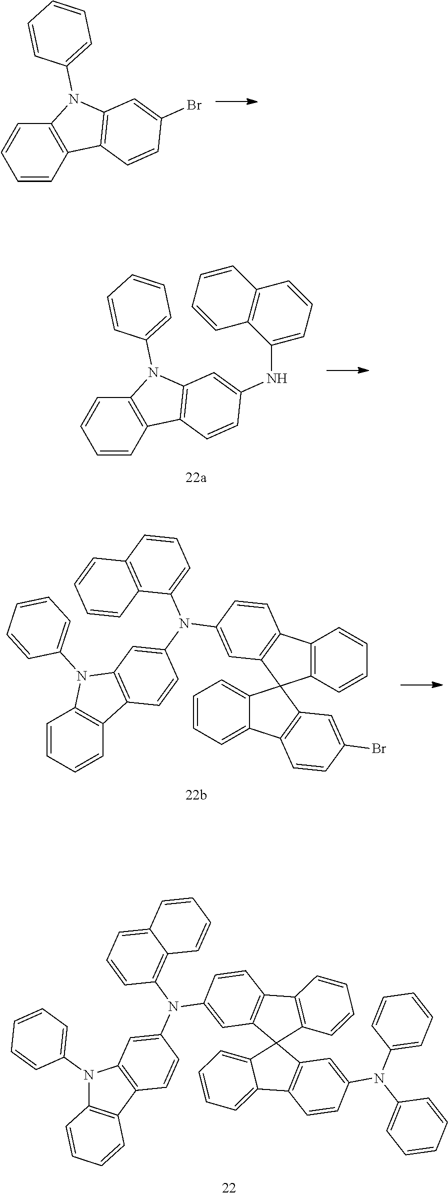 [SMILES: Brc1ccc2c3ccccc3n(-c3ccccc3)c2c1 ; c1ccc(-n2c3ccccc3c3ccc(Nc4cccc5ccccc45)cc32)cc1 ; Brc1ccc2c(c1)C1(c3ccccc3-2)c2ccccc2-c2ccc(N(c3ccc4c5ccccc5n(-c5ccccc5)c4c3)c3cccc4ccccc34)cc21 ; c1ccc(N(c2ccccc2)c2ccc3c(c2)C2(c4ccccc4-3)c3ccccc3-c3ccc(N(c4ccc5c6ccccc6n(-c6ccccc6)c5c4)c4cccc5ccccc45)cc32)cc1]

### 4-1. (Synthesis of Intermediate 22a)

2-bromo-9-phenyl-9H-carbazole (1.0 eq.), 1-aminonaphthalene (1.1 eq.), tris(dibenzylideneacetone)dipalladium(0) (0.02 eq.), tri-tert-butylphosphine (0.04 eq.), and sodium tert-butoxide (1.5 eq.) were dissolved in toluene, and then the mixture was stirred at about 80° C. for about 1 hour in a nitrogen atmosphere. After cooling, the resulting product was washed three times with ethyl acetate and water, and then an organic layer was obtained. The obtained organic layer was dried over MgSO₄, and then dried under a reduced pressure. The dried organic layer was purified by column chromatography to obtain Intermediate 22a. (yield: 78%)

### 4-2. (Synthesis of Intermediate 22b)

Intermediate 22a (1.0 eq.), 2,2′-dibromo-9,9′-spirobi[fluorene] (1.5 eq.), tris(dibenzylideneacetone)dipalladium(0) (0.02 eq.), tri-tert-butylphosphine (0.04 eq.), and sodium tert-butoxide (1.5 eq.) were dissolved in toluene and then the mixture was stirred at about 80° C. for about 1 hour in a nitrogen atmosphere. After cooling, the resulting product was washed three times with ethyl acetate and water, and then an organic layer was obtained. The obtained organic layer was dried over MgSO₄, and then dried under a reduced pressure. The dried organic layer was purified by column chromatography to obtain Intermediate 22b. (yield: 58%)

### 4-3. Synthesis of Compound 22

Intermediate 22b (1.0 eq.), diphenylamine (1.5 eq.), tris(dibenzylideneacetone)dipalladium(0) (0.02 eq.), tri-tert-butylphosphine (0.04 eq.), and sodium tert-butoxide (1.5 eq.) were dissolved in toluene and then the mixture was stirred at about 80° C. for about 1 hour in a nitrogen atmosphere. After cooling, the resulting product was washed three times with ethyl acetate and water, and then an organic layer was obtained. The obtained organic layer was dried over MgSO₄, and then dried under a reduced pressure. The dried organic layer was purified by column chromatography to obtain Compound 22. (yield: 78%)

By measuring FAB-MS, a mass number of m/z=865.35 was observed by molecular ion peak, thereby identifying Compound 22.

### (5) Synthesis of Compound 38

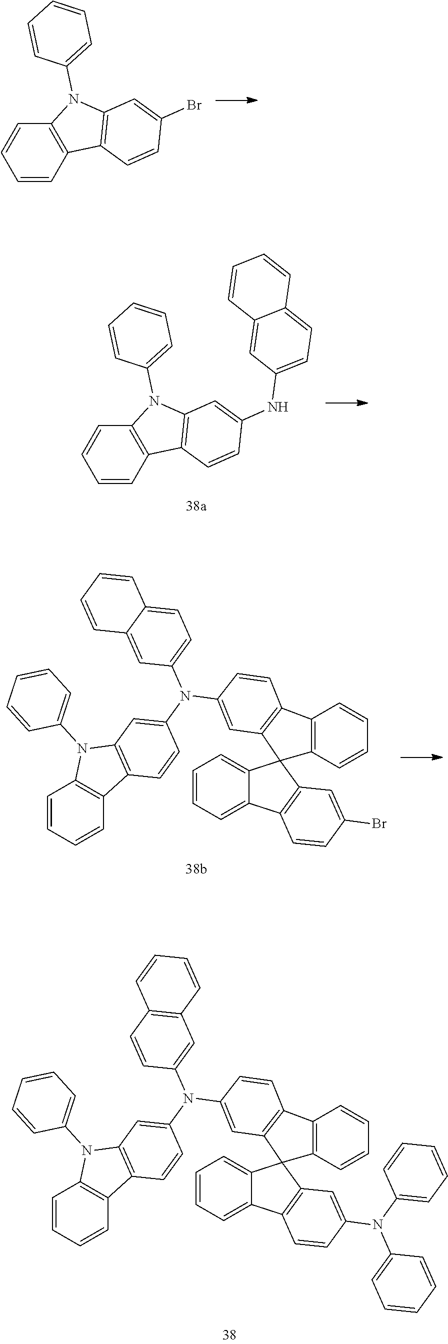 [SMILES: Brc1ccc2c3ccccc3n(-c3ccccc3)c2c1 ; c1ccc(-n2c3ccccc3c3ccc(Nc4ccc5ccccc5c4)cc32)cc1 ; Brc1ccc2c(c1)C1(c3ccccc3-2)c2ccccc2-c2ccc(N(c3ccc4ccccc4c3)c3ccc4c5ccccc5n(-c5ccccc5)c4c3)cc21 ; c1ccc(N(c2ccccc2)c2ccc3c(c2)C2(c4ccccc4-3)c3ccccc3-c3ccc(N(c4ccc5ccccc5c4)c4ccc5c6ccccc6n(-c6ccccc6)c5c4)cc32)cc1]

### 5-1. (Synthesis of Intermediate 38a)

2-bromo-9-phenyl-9H-carbazole (1.0 eq.), 2-aminonaphthalene (1.1 eq.), tris(dibenzylideneacetone)dipalladium(0) (0.02 eq.), tri-tert-butylphosphine (0.04 eq.), and sodium tert-butoxide (1.5 eq.) were dissolved in toluene, and then the mixture was stirred at about 80° C. for about 1 hour in a nitrogen atmosphere. After cooling, the resulting product was washed three times with ethyl acetate and water, and then an organic layer was obtained. The obtained organic layer was dried over MgSO₄, and then dried under a reduced pressure. The dried organic layer was purified by column chromatography to obtain Intermediate 38a. (yield: 78%)

### 5-2. (Synthesis of Intermediate 38b)

Intermediate 38a (1.0 eq.), 2,2′-dibromo-9,9′-spirobi[fluorene] (1.5 eq.), tris(dibenzylideneacetone)dipalladium(0) (0.02 eq.), tri-tert-butylphosphine (0.04 eq.), and sodium tert-butoxide (1.5 eq.) were dissolved in toluene and then the mixture was stirred at about 80° C. for about 1 hour in a nitrogen atmosphere. After cooling, the resulting product was washed three times with ethyl acetate and water, and then an organic layer was obtained. The obtained organic layer was dried over MgSO₄, and then dried under a reduced pressure. The dried organic layer was purified by column chromatography to obtain Intermediate 38b. (yield: 58%)

### 5-3. Synthesis of Compound 38

Intermediate 38b (1.0 eq.), diphenylamine (1.5 eq.), tris(dibenzylideneacetone)dipalladium(0) (0.02 eq.), tri-tert-butylphosphine (0.04 eq.), and sodium tert-butoxide (1.5 eq.) were dissolved in toluene and then the mixture was stirred at about 80° C. for about 1 hour in a nitrogen atmosphere. After cooling, the resulting product was washed three times with ethyl acetate and water, and then an organic layer was obtained. The obtained organic layer was dried over MgSO₄, and then dried under a reduced pressure. The dried organic layer was purified by column chromatography to obtain Compound 38. (yield: 78%)

By measuring FAB-MS, a mass number of m/z=865.35 was observed by molecular ion peak, thereby identifying Compound 38.

### (6) Synthesis of Compound 54

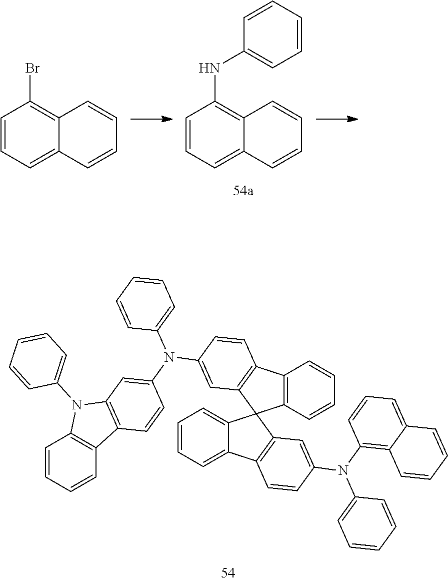 [SMILES: Brc1cccc2ccccc12 ; c1ccc(Nc2cccc3ccccc23)cc1 ; c1ccc(N(c2ccc3c(c2)C2(c4ccccc4-3)c3ccccc3-c3ccc(N(c4ccccc4)c4cccc5ccccc45)cc32)c2ccc3c4ccccc4n(-c4ccccc4)c3c2)cc1]

### 6-1. (Synthesis of Intermediate 54a)

1-bromonaphthalene (1.0 eq.), aniline (1.2 eq.), tris(dibenzylideneacetone)dipalladium(0) (0.02 eq.), tri-tert-butylphosphine (0.04 eq.), and sodium tert-butoxide (1.5 eq.) were dissolved in toluene and then the mixture was stirred at about 80° C. for about 1 hour in a nitrogen atmosphere. After cooling, the resulting product was washed three times with ethyl acetate and water, and then an organic layer was obtained. The obtained organic layer was dried over MgSO₄, and then dried under a reduced pressure. The dried organic layer was purified by column chromatography to obtain Intermediate 54a. (yield: 88%)

### 6-2. Synthesis of Compound 54

Intermediate 54a (1.0 eq.), Intermediate 6b (1.1 eq.), tris(dibenzylideneacetone)dipalladium(0) (0.02 eq.), tri-tert-butylphosphine (0.04 eq.), and sodium tert-butoxide (1.5 eq.) were dissolved in toluene, and then the mixture was stirred at about 80° C. for about 1 hour in a nitrogen atmosphere. After cooling, the resulting product was washed three times with ethyl acetate and water, and then an organic layer was obtained. The obtained organic layer was dried over MgSO₄, and then dried under a reduced pressure. The dried organic layer was purified by column chromatography to obtain Compound 54. (yield: 78%)

By measuring FAB-MS, a mass number of m/z=865.35 was observed by molecular ion peak, thereby identifying Compound 54.

### (7) Synthesis of Compound 70

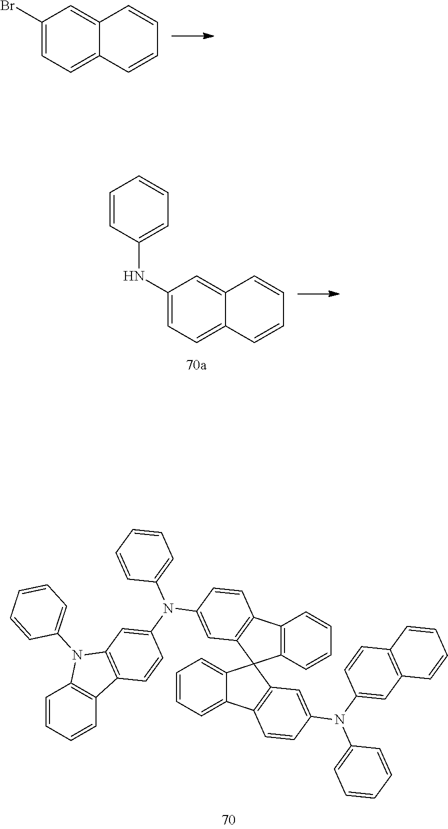 [SMILES: Brc1ccc2ccccc2c1 ; c1ccc(Nc2ccc3ccccc3c2)cc1 ; c1ccc(N(c2ccc3c(c2)C2(c4ccccc4-3)c3ccccc3-c3ccc(N(c4ccccc4)c4ccc5c6ccccc6n(-c6ccccc6)c5c4)cc32)c2ccc3ccccc3c2)cc1]

### 7-1. (Synthesis of Intermediate 70a)

2-bromonaphthalene (1.0 eq.), aniline (1.2 eq.), tris(dibenzylideneacetone)dipalladium(0) (0.02 eq.), tri-tert-butylphosphine (0.04 eq.), and sodium tert-butoxide (1.5 eq.) were dissolved in toluene and then the mixture was stirred at about 80° C. for about 1 hour in a nitrogen atmosphere. After cooling, the resulting product was washed three times with ethyl acetate and water, and then an organic layer was obtained. The obtained organic layer was dried over MgSO₄, and then dried under a reduced pressure. The dried organic layer was purified by column chromatography to obtain Intermediate 70a. (yield: 88%)

### 7-2. Synthesis of Compound 70

Intermediate 70a (1.0 eq.), Intermediate 6b (1.1 eq.), tris(dibenzylideneacetone)dipalladium(0) (0.02 eq.), tri-tert-butylphosphine (0.04 eq.), and sodium tert-butoxide (1.5 eq.) were dissolved in toluene, and then the mixture was stirred at about 80° C. for about 1 hour in a nitrogen atmosphere. After cooling, the resulting product was washed three times with ethyl acetate and water, and then an organic layer was obtained. The obtained organic layer was dried over MgSO₄, and then dried under a reduced pressure. The dried organic layer was purified by column chromatography to obtain Compound 70. (yield: 78%)

By measuring FAB-MS, a mass number of m/z=865.35 was observed by molecular ion peak, thereby identifying Compound 70.

### (8) Synthesis of Compound 86

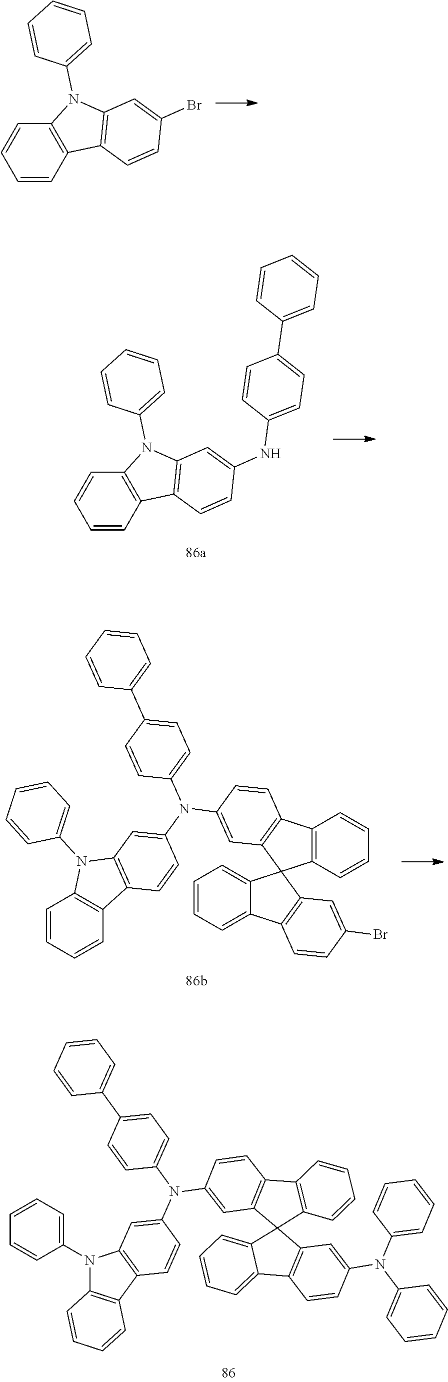 [SMILES: Brc1ccc2c3ccccc3n(-c3ccccc3)c2c1 ; c1ccc(-c2ccc(Nc3ccc4c5ccccc5n(-c5ccccc5)c4c3)cc2)cc1 ; Brc1ccc2c(c1)C1(c3ccccc3-2)c2ccccc2-c2ccc(N(c3ccc(-c4ccccc4)cc3)c3ccc4c5ccccc5n(-c5ccccc5)c4c3)cc21 ; c1ccc(-c2ccc(N(c3ccc4c(c3)C3(c5ccccc5-c5ccc(N(c6ccccc6)c6ccccc6)cc53)c3ccccc3-4)c3ccc4c5ccccc5n(-c5ccccc5)c4c3)cc2)cc1]

### 8-1. (Synthesis of Intermediate 86a)

2-bromo-9-phenyl-9H-carbazole (1.0 eq.), [1,1′-biphenyl] 4-amine (1.1 eq.), tris(dibenzylideneacetone)dipalladium(0) (0.02 eq.), tri-tert-butylphosphine (0.04 eq.), and sodium tert-butoxide (1.5 eq.) were dissolved in toluene, and then the mixture was stirred at about 80° C. for about 1 hour in a nitrogen atmosphere. After cooling, the resulting product was washed three times with ethyl acetate and water, and then an organic layer was obtained. The obtained organic layer was dried over MgSO₄, and then dried under a reduced pressure. The dried organic layer was purified by column chromatography to obtain Intermediate 86a. (yield: 78%)

### 8-2. (Synthesis of Intermediate 86b)

Intermediate 86a (1.0 eq.), 2,2′-dibromo-9,9′-spirobi[fluorene] (1.5 eq.), tris(dibenzylideneacetone)dipalladium(0) (0.02 eq.), tri-tert-butylphosphine (0.04 eq.), and sodium tert-butoxide (1.5 eq.) were dissolved in toluene and then the mixture was stirred at about 80° C. for about 1 hour in a nitrogen atmosphere. After cooling, the resulting product was washed three times with ethyl acetate and water, and then an organic layer was obtained. The obtained organic layer was dried over MgSO₄, and then dried under a reduced pressure. The dried organic layer was purified by column chromatography to obtain Intermediate 86b. (yield: 58%)

### 8-3. Synthesis of Compound 86

Intermediate 86b (1.0 eq.), diphenylamine (1.5 eq.), tris(dibenzylideneacetone)dipalladium(0) (0.02 eq.), tri-tert-butylphosphine (0.04 eq.), and sodium tert-butoxide (1.5 eq.) were dissolved in toluene and then the mixture was stirred at about 80° C. for about 1 hour in a nitrogen atmosphere. After cooling, the resulting product was washed three times with ethyl acetate and water, and then an organic layer was obtained. The obtained organic layer was dried over MgSO₄, and then dried under a reduced pressure. The dried organic layer was purified by column chromatography to obtain Compound 86. (yield: 78%)

By measuring FAB-MS, a mass number of m/z=891.36 was observed by molecular ion peak, thereby identifying Compound 86.

### (9) Synthesis of Compound 102

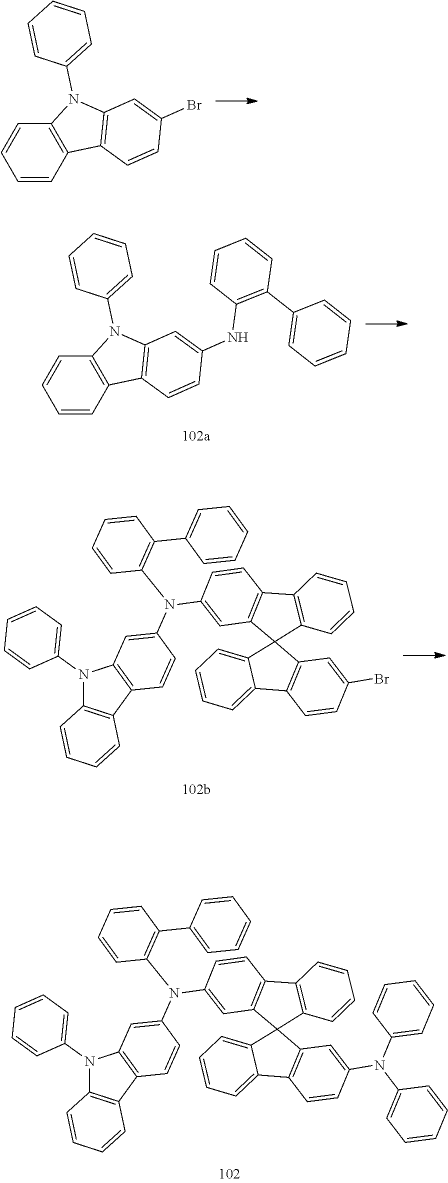 [SMILES: Brc1ccc2c3ccccc3n(-c3ccccc3)c2c1 ; c1ccc(-c2ccccc2Nc2ccc3c4ccccc4n(-c4ccccc4)c3c2)cc1 ; Brc1ccc2c(c1)C1(c3ccccc3-2)c2ccccc2-c2ccc(N(c3ccc4c5ccccc5n(-c5ccccc5)c4c3)c3ccccc3-c3ccccc3)cc21 ; c1ccc(-c2ccccc2N(c2ccc3c(c2)C2(c4ccccc4-c4ccc(N(c5ccccc5)c5ccccc5)cc42)c2ccccc2-3)c2ccc3c4ccccc4n(-c4ccccc4)c3c2)cc1]

### 9-1. (Synthesis of Intermediate 102a)

2-bromo-9-phenyl-9H-carbazole (1.0 eq.), [1,1′-biphenyl]-2-amine (1.1 eq.), tris(dibenzylideneacetone)dipalladium(0) (0.02 eq.), tri-tert-butylphosphine (0.04 eq.), and sodium tert-butoxide (1.5 eq.) were dissolved in toluene, and then the mixture was stirred at about 80° C. for about 1 hour in a nitrogen atmosphere. After cooling, the resulting product was washed three times with ethyl acetate and water, and then an organic layer was obtained. The obtained organic layer was dried over MgSO₄, and then dried under a reduced pressure. The dried organic layer was purified by column chromatography to obtain Intermediate 102a. (yield: 78%)

### 9-2. (Synthesis of Intermediate 102b)

Intermediate 102a (1.0 eq.), 2,2′-dibromo-9,9′-spirobi[fluorene] (1.5 eq.), tris(dibenzylideneacetone)dipalladium(0) (0.02 eq.), tri-tert-butylphosphine (0.04 eq.), and sodium tert-butoxide (1.5 eq.) were dissolved in toluene and then the mixture was stirred at about 80° C. for about 1 hour in a nitrogen atmosphere. After cooling, the resulting product was washed three times with ethyl acetate and water, and then an organic layer was obtained. The obtained organic layer was dried over MgSO₄, and then dried under a reduced pressure. The dried organic layer was purified by column chromatography to obtain Intermediate 102b. (yield: 58%)

### 9-3. Synthesis of Compound 102

Intermediate 102b (1.0 eq.), diphenylamine (1.5 eq.), tris(dibenzylideneacetone)dipalladium(0) (0.02 eq.), tri-tert-butylphosphine (0.04 eq.), and sodium tert-butoxide (1.5 eq.) were dissolved in toluene and then the mixture was stirred at about 80° C. for about 1 hour in a nitrogen atmosphere. After cooling, the resulting product was washed three times with ethyl acetate and water, and then an organic layer was obtained. The obtained organic layer was dried over MgSO₄, and then dried under a reduced pressure. The dried organic layer was purified by column chromatography to obtain Compound 102. (yield: 78%)

By measuring FAB-MS, a mass number of m/z=891.36 was observed by molecular ion peak, thereby identifying Compound 102.

### (10) Synthesis of Compound 118

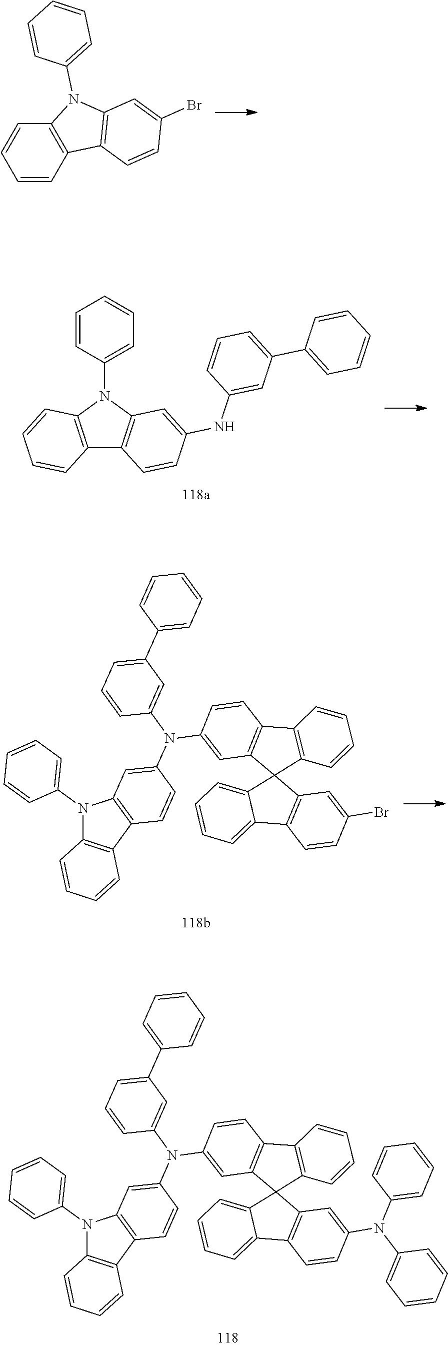 [SMILES: Brc1ccc2c3ccccc3n(-c3ccccc3)c2c1 ; c1ccc(-c2cccc(Nc3ccc4c5ccccc5n(-c5ccccc5)c4c3)c2)cc1 ; Brc1ccc2c(c1)C1(c3ccccc3-2)c2ccccc2-c2ccc(N(c3cccc(-c4ccccc4)c3)c3ccc4c5ccccc5n(-c5ccccc5)c4c3)cc21 ; c1ccc(-c2cccc(N(c3ccc4c(c3)C3(c5ccccc5-c5ccc(N(c6ccccc6)c6ccccc6)cc53)c3ccccc3-4)c3ccc4c5ccccc5n(-c5ccccc5)c4c3)c2)cc1]

### 10-1. (Synthesis of Intermediate 118a)

2-bromo-9-phenyl-9H-carbazole (1.0 eq.), [1,1′-biphenyl]-3-amine (1.1 eq.), tris(dibenzylideneacetone)dipalladium(0) (0.02 eq.), tri-tert-butylphosphine (0.04 eq.), and sodium tert-butoxide (1.5 eq.) were dissolved in toluene, and then the mixture was stirred at about 80° C. for about 1 hour in a nitrogen atmosphere. After cooling, the resulting product was washed three times with ethyl acetate and water, and then an organic layer was obtained. The obtained organic layer was dried over MgSO₄, and then dried under a reduced pressure. The dried organic layer was purified by column chromatography to obtain Intermediate 118a. (yield: 78%)

### 10-2. (Synthesis of Intermediate 118b)

Intermediate 118a (1.0 eq.), 2,2′-dibromo-9,9′-spirobi[fluorene] (1.5 eq.), tris(dibenzylideneacetone)dipalladium(0) (0.02 eq.), tri-tert-butylphosphine (0.04 eq.), and sodium tert-butoxide (1.5 eq.) were dissolved in toluene and then the mixture was stirred at about 80° C. for about 1 hour in a nitrogen atmosphere. After cooling, the resulting product was washed three times with ethyl acetate and water, and then an organic layer was obtained. The obtained organic layer was dried over MgSO₄, and then dried under a reduced pressure. The dried organic layer was purified by column chromatography to obtain Intermediate 118b. (yield: 58%)

### 10-3. Synthesis of Compound 118

Intermediate 118b (1.0 eq.), diphenylamine (1.5 eq.), tris(dibenzylideneacetone)dipalladium(0) (0.02 eq.), tri-tert-butylphosphine (0.04 eq.), and sodium tert-butoxide (1.5 eq.) were dissolved in toluene and then the mixture was stirred at about 80° C. for about 1 hour in a nitrogen atmosphere. After cooling, the resulting product was washed three times with ethyl acetate and water, and then an organic layer was obtained. The obtained organic layer was dried over MgSO₄, and then dried under a reduced pressure. The dried organic layer was purified by column chromatography to obtain Compound 118. (yield: 78%)

By measuring FAB-MS, a mass number of m/z=891.36 was observed by molecular ion peak, thereby identifying Compound 118.

### (11) Synthesis of Compound 134

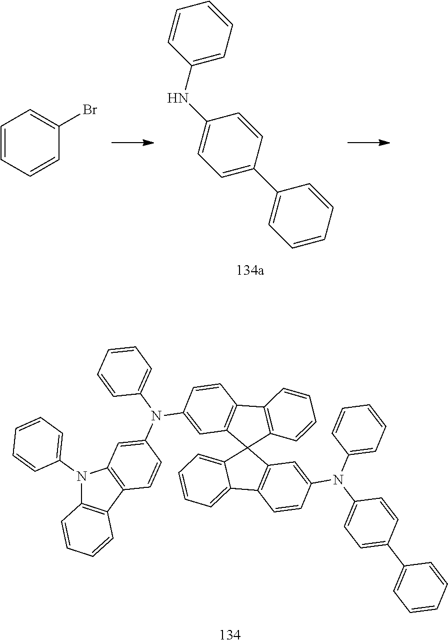 [SMILES: Brc1ccccc1 ; c1ccc(Nc2ccc(-c3ccccc3)cc2)cc1 ; c1ccc(-c2ccc(N(c3ccccc3)c3ccc4c(c3)C3(c5ccccc5-4)c4ccccc4-c4ccc(N(c5ccccc5)c5ccc6c7ccccc7n(-c7ccccc7)c6c5)cc43)cc2)cc1]

### 11-1. (Synthesis of Intermediate 134a)

Bromobenzene (1.0 eq.), [1,1′-biphenyl]-4-amine (1.1 eq.), tris(dibenzylideneacetone)dipalladium(0) (0.02 eq.), tri-tert-butylphosphine (0.04 eq.), and sodium tert-butoxide (1.5 eq.) were dissolved in toluene, and then the mixture was stirred at about 80° C. for about 1 hour in a nitrogen atmosphere. After cooling, the resulting product was washed three times with ethyl acetate and water, and then an organic layer was obtained. The obtained organic layer was dried over MgSO₄, and then dried under a reduced pressure. The dried organic layer was purified by column chromatography to obtain Intermediate 134a. (yield: 88%)

### 11-2. Synthesis of Compound 134

Intermediate 134a (1.0 eq.), Intermediate 6b (1.1 eq.), tris(dibenzylideneacetone)dipalladium(0) (0.02 eq.), tri-tert-butylphosphine (0.04 eq.), and sodium tert-butoxide (1.5 eq.) were dissolved in toluene, and then the mixture was stirred at about 80° C. for about 1 hour in a nitrogen atmosphere. After cooling, the resulting product was washed three times with ethyl acetate and water, and then an organic layer was obtained. The obtained organic layer was dried over MgSO₄, and then dried under a reduced pressure. The dried organic layer was purified by column chromatography to obtain Compound 134. (yield: 78%)

By measuring FAB-MS, a mass number of m/z=891.36 was observed by molecular ion peak, thereby identifying Compound 134.

### (12) Synthesis of Compound 150

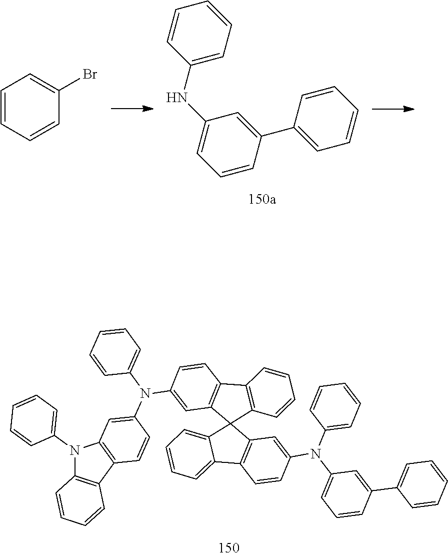 [SMILES: Brc1ccccc1 ; c1ccc(Nc2cccc(-c3ccccc3)c2)cc1 ; c1ccc(-c2cccc(N(c3ccccc3)c3ccc4c(c3)C3(c5ccccc5-4)c4ccccc4-c4ccc(N(c5ccccc5)c5ccc6c7ccccc7n(-c7ccccc7)c6c5)cc43)c2)cc1]

### 12-1. (Synthesis of Intermediate 150a)

Bromobenzene (1.0 eq.), [1,1′-biphenyl]-3-amine (1.1 eq.), tris(dibenzylideneacetone)dipalladium(0) (0.02 eq.), tri-tert-butylphosphine (0.04 eq.), and sodium tert-butoxide (1.5 eq.) were dissolved in toluene, and then the mixture was stirred at about 80° C. for about 1 hour in a nitrogen atmosphere. After cooling, the resulting product was washed three times with ethyl acetate and water, and then an organic layer was obtained. The obtained organic layer was dried over MgSO₄, and then dried under a reduced pressure. The dried organic layer was purified by column chromatography to obtain Intermediate 150a. (yield: 88%)

### 12-2. Synthesis of Compound 150

Intermediate 150a (1.0 eq.), Intermediate 6b (1.1 eq.), tris(dibenzylideneacetone)dipalladium(0) (0.02 eq.), tri-tert-butylphosphine (0.04 eq.), and sodium tert-butoxide (1.5 eq.) were dissolved in toluene, and then the mixture was stirred at about 80° C. for about 1 hour in a nitrogen atmosphere. After cooling, the resulting product was washed three times with ethyl acetate and water, and then an organic layer was obtained. The obtained organic layer was dried over MgSO₄, and then dried under a reduced pressure. The dried organic layer was purified by column chromatography to obtain Compound 150. (yield: 78%)

By measuring FAB-MS, a mass number of m/z=891.36 was observed by molecular ion peak, thereby identifying Compound 150.

### (13) Synthesis of Compound 166

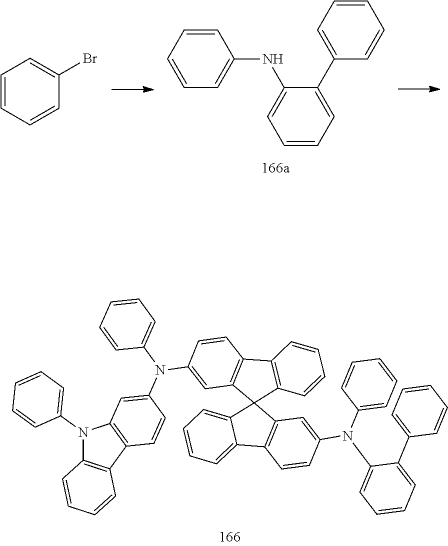 [SMILES: Brc1ccccc1 ; c1ccc(Nc2ccccc2-c2ccccc2)cc1 ; c1ccc(-c2ccccc2N(c2ccccc2)c2ccc3c(c2)C2(c4ccccc4-c4ccc(N(c5ccccc5)c5ccc6c7ccccc7n(-c7ccccc7)c6c5)cc42)c2ccccc2-3)cc1]

### 13-1. (Synthesis of Intermediate 166a)

Bromobenzene (1.0 eq.), [1,1′-biphenyl]-2-amine (1.1 eq.), tris(dibenzylideneacetone)dipalladium(0) (0.02 eq.), tri-tert-butylphosphine (0.04 eq.), and sodium tert-butoxide (1.5 eq.) were dissolved in toluene, and then the mixture was stirred at about 80° C. for about 1 hour in a nitrogen atmosphere. After cooling, the resulting product was washed three times with ethyl acetate and water, and then an organic layer was obtained. The obtained organic layer was dried over MgSO₄, and then dried under a reduced pressure. The dried organic layer was purified by column chromatography to obtain Intermediate 166a. (yield: 88%)

### 13-2. Synthesis of Compound 166

Intermediate 166a (1.0 eq.), Intermediate 6b (1.1 eq.), tris(dibenzylideneacetone)dipalladium(0) (0.02 eq.), tri-tert-butylphosphine (0.04 eq.), and sodium tert-butoxide (1.5 eq.) were dissolved in toluene, and then the mixture was stirred at about 80° C. for about 1 hour in a nitrogen atmosphere. After cooling, the resulting product was washed three times with ethyl acetate and water, and then an organic layer was obtained. The obtained organic layer was dried over MgSO₄, and then dried under a reduced pressure. The dried organic layer was purified by column chromatography to obtain Compound 166. (yield: 78%)

By measuring FAB-MS, a mass number of m/z=891.36 was observed by molecular ion peak, thereby identifying Compound 166.

### 1. Manufacture and Evaluation of Light Emitting Device Including Amine Compound

### Manufacture of Light Emitting Device

Light emitting devices were manufactured utilizing Example Compounds and Comparative Example Compounds below as a hole transport region material:

### Example Compounds

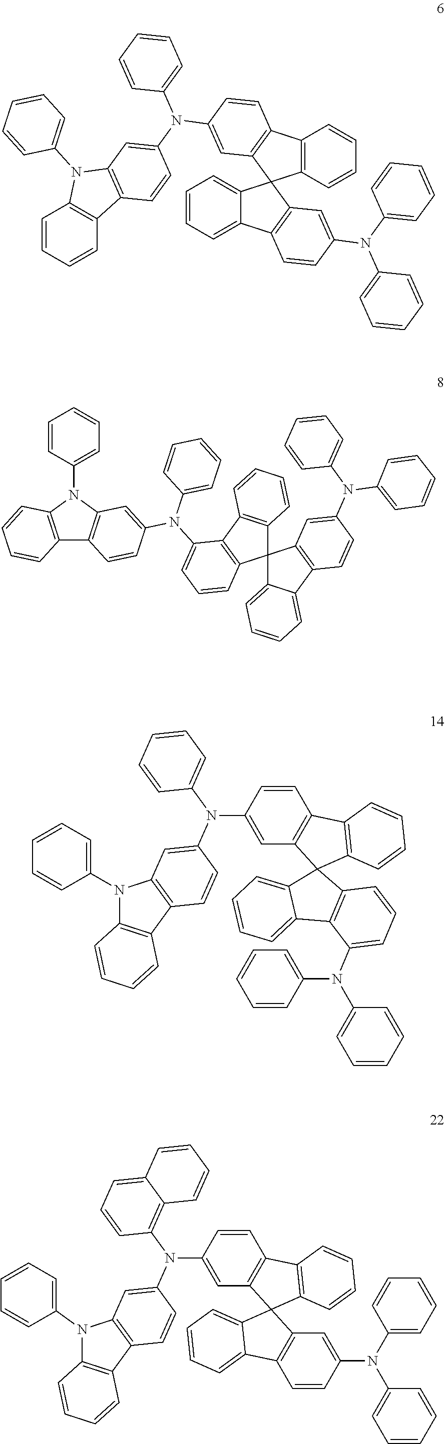 [SMILES: c1ccc(N(c2ccccc2)c2ccc3c(c2)C2(c4ccccc4-3)c3ccccc3-c3ccc(N(c4ccccc4)c4ccc5c6ccccc6n(-c6ccccc6)c5c4)cc32)cc1 ; c1ccc(N(c2ccccc2)c2ccc3c(c2)C2(c4ccccc4-3)c3ccccc3-c3c(N(c4ccccc4)c4ccc5c6ccccc6n(-c6ccccc6)c5c4)cccc32)cc1 ; c1ccc(N(c2ccc3c(c2)C2(c4ccccc4-3)c3ccccc3-c3c(N(c4ccccc4)c4ccccc4)cccc32)c2ccc3c4ccccc4n(-c4ccccc4)c3c2)cc1 ; c1ccc(N(c2ccccc2)c2ccc3c(c2)C2(c4ccccc4-3)c3ccccc3-c3ccc(N(c4ccc5c6ccccc6n(-c6ccccc6)c5c4)c4cccc5ccccc45)cc32)cc1]

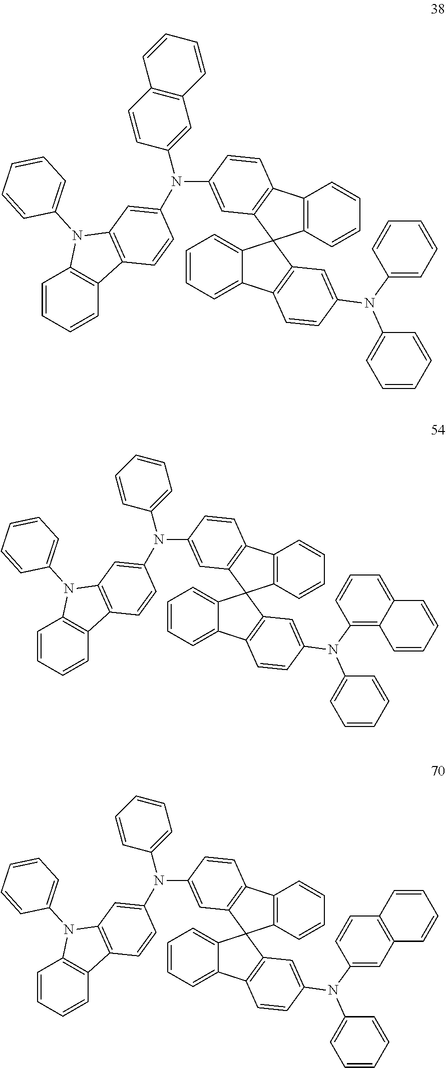 [SMILES: c1ccc(N(c2ccccc2)c2ccc3c(c2)C2(c4ccccc4-3)c3ccccc3-c3ccc(N(c4ccc5ccccc5c4)c4ccc5c6ccccc6n(-c6ccccc6)c5c4)cc32)cc1 ; c1ccc(N(c2ccc3c(c2)C2(c4ccccc4-3)c3ccccc3-c3ccc(N(c4ccccc4)c4cccc5ccccc45)cc32)c2ccc3c4ccccc4n(-c4ccccc4)c3c2)cc1 ; c1ccc(N(c2ccc3c(c2)C2(c4ccccc4-3)c3ccccc3-c3ccc(N(c4ccccc4)c4ccc5c6ccccc6n(-c6ccccc6)c5c4)cc32)c2ccc3ccccc3c2)cc1]

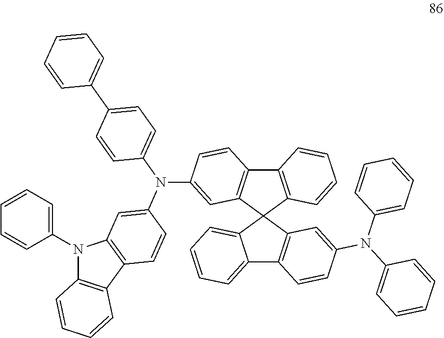 [SMILES: c1ccc(-c2ccc(N(c3ccc4c(c3)C3(c5ccccc5-c5ccc(N(c6ccccc6)c6ccccc6)cc53)c3ccccc3-4)c3ccc4c5ccccc5n(-c5ccccc5)c4c3)cc2)cc1]

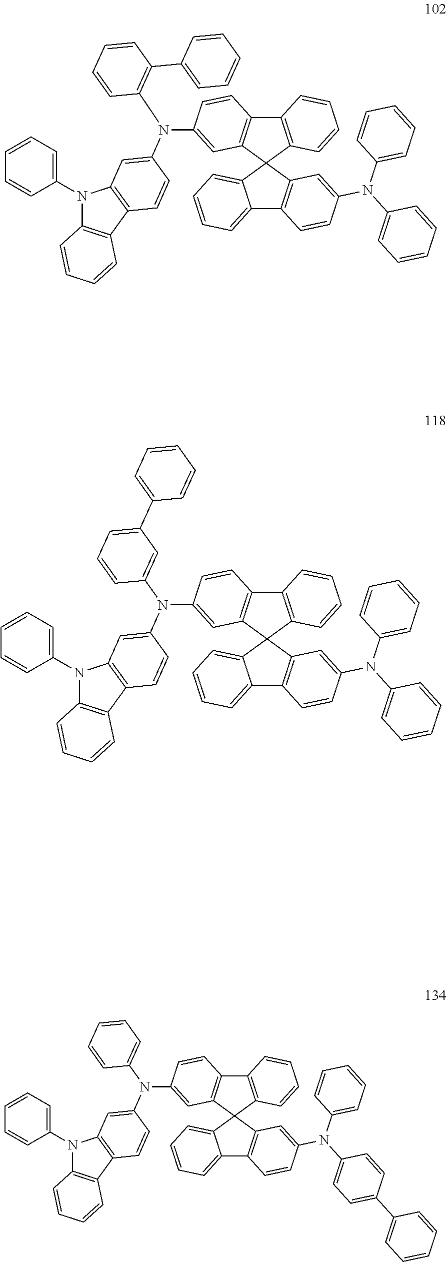 [SMILES: c1ccc(-c2ccccc2N(c2ccc3c(c2)C2(c4ccccc4-c4ccc(N(c5ccccc5)c5ccccc5)cc42)c2ccccc2-3)c2ccc3c4ccccc4n(-c4ccccc4)c3c2)cc1 ; c1ccc(-c2cccc(N(c3ccc4c(c3)C3(c5ccccc5-c5ccc(N(c6ccccc6)c6ccccc6)cc53)c3ccccc3-4)c3ccc4c5ccccc5n(-c5ccccc5)c4c3)c2)cc1 ; c1ccc(-c2ccc(N(c3ccccc3)c3ccc4c(c3)C3(c5ccccc5-4)c4ccccc4-c4ccc(N(c5ccccc5)c5ccc6c7ccccc7n(-c7ccccc7)c6c5)cc43)cc2)cc1]

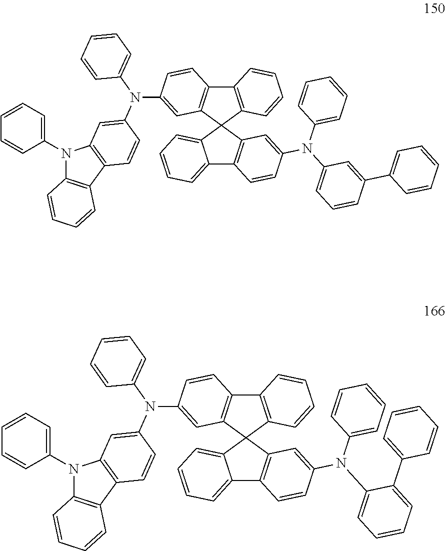 [SMILES: c1ccc(-c2cccc(N(c3ccccc3)c3ccc4c(c3)C3(c5ccccc5-4)c4ccccc4-c4ccc(N(c5ccccc5)c5ccc6c7ccccc7n(-c7ccccc7)c6c5)cc43)c2)cc1 ; c1ccc(-c2ccccc2N(c2ccccc2)c2ccc3c(c2)C2(c4ccccc4-c4ccc(N(c5ccccc5)c5ccc6c7ccccc7n(-c7ccccc7)c6c5)cc42)c2ccccc2-3)cc1]

### Comparative Example Compounds

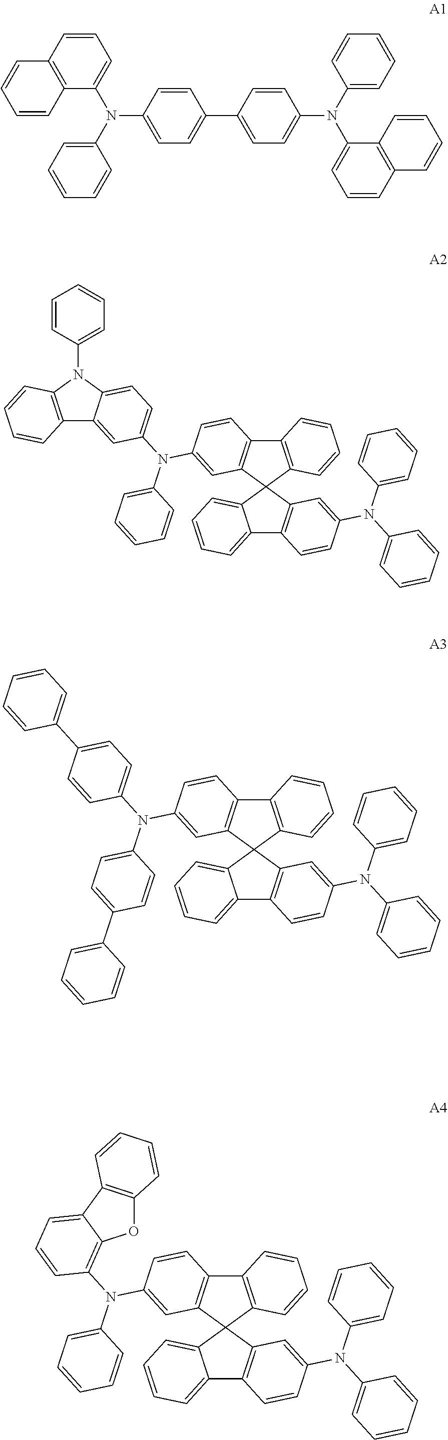 [SMILES: c1ccc(N(c2ccc(-c3ccc(N(c4ccccc4)c4cccc5ccccc45)cc3)cc2)c2cccc3ccccc23)cc1 ; c1ccc(N(c2ccccc2)c2ccc3c(c2)C2(c4ccccc4-3)c3ccccc3-c3ccc(N(c4ccccc4)c4ccc5c(c4)c4ccccc4n5-c4ccccc4)cc32)cc1 ; c1ccc(-c2ccc(N(c3ccc(-c4ccccc4)cc3)c3ccc4c(c3)C3(c5ccccc5-c5ccc(N(c6ccccc6)c6ccccc6)cc53)c3ccccc3-4)cc2)cc1 ; c1ccc(N(c2ccccc2)c2ccc3c(c2)C2(c4ccccc4-3)c3ccccc3-c3ccc(N(c4ccccc4)c4cccc5c4oc4ccccc45)cc32)cc1]

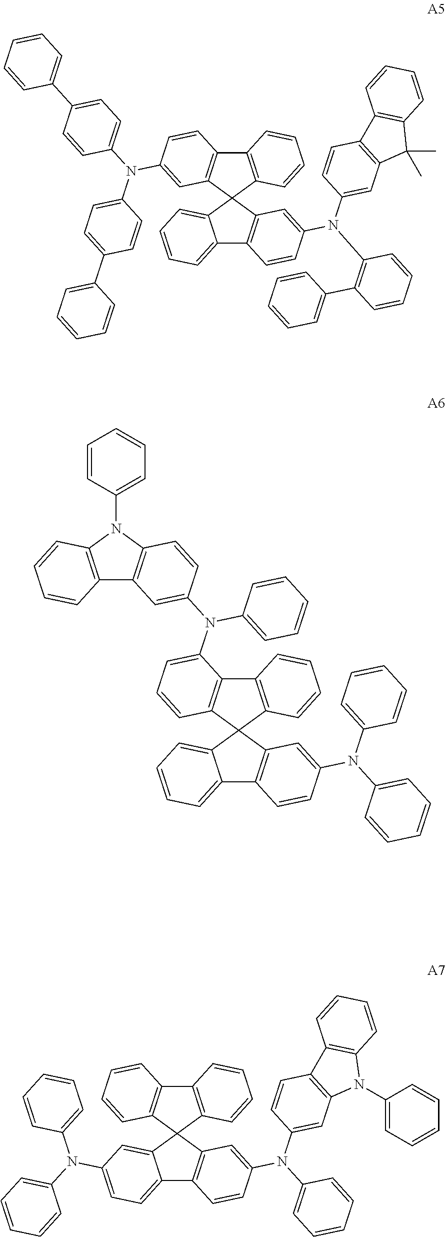 [SMILES: CC1(C)c2ccccc2-c2ccc(N(c3ccc4c(c3)C3(c5ccccc5-c5ccc(N(c6ccc(-c7ccccc7)cc6)c6ccc(-c7ccccc7)cc6)cc53)c3ccccc3-4)c3ccccc3-c3ccccc3)cc21 ; c1ccc(N(c2ccccc2)c2ccc3c(c2)C2(c4ccccc4-3)c3ccccc3-c3c(N(c4ccccc4)c4ccc5c(c4)c4ccccc4n5-c4ccccc4)cccc32)cc1 ; c1ccc(N(c2ccccc2)c2ccc3c(c2)C2(c4ccccc4-c4ccccc42)c2cc(N(c4ccccc4)c4ccc5c6ccccc6n(-c6ccccc6)c5c4)ccc2-3)cc1]

The light emitting devices of Examples and Comparative Examples were manufactured by the following method. For an anode, an ITO glass substrate of about 15 Ω/cm² (about 1,200 Å) made by Corning Co. was cut to a size of 50 mm×50 mm×0.7 mm, cleansed by ultrasonic waves utilizing isopropyl alcohol and pure water for about 5 minutes each, and then irradiated with ultraviolet rays for about 30 minutes and exposed to ozone and cleansed. The glass substrate was installed on a vacuum deposition apparatus.

On the upper portion of the substrate, 2-TNATA, a suitable (e.g., known) material, was deposited in vacuum to form a 600 Å-thick hole injection layer, and then a respective Example or Comparative Example Compound was deposited in vacuum to form a 300 Å-thick hole transport layer.

On the upper portion of the hole transport layer, 9,10-di(naphthalen-2-yl)anthracene (DNA), which is a suitable (e.g., known) compound, as a blue fluorescent host and 4,4′-bis[2-(4-(N,N-diphenylamino)phenyl)vinyl]biphenyl (DPAVBi), which is a suitable (e.g., known) compound, as a blue fluorescent dopant were co-deposited at a weight ratio of 98:2 to form a 300 Å-thick emission layer.

Then, Alq3 was deposited on the upper portion of the emission layer to form a 300 Å-thick electron transport layer, LiF, which is an alkali metal halide, was deposited on the upper portion of the electron transport layer to form a 10 Å-thick electron injection layer, and Al was deposited in vacuum to form a 3,000 Å-thick LiF/Al electrode (negative electrode), thereby manufacturing a light emitting device.

### Evaluation of Light Emitting Device Characteristics

Evaluation results of the light emitting devices of each of Examples 1 to 13, and Comparative Examples 1 to 7 are listed in Table 1. Driving voltage, brightness, luminous efficiency and a half service life of each of the light emitting devices are listed in comparison in Table 1.

In the evaluation results of the characteristics of each of the Examples and Comparative Examples listed in Table 1, voltage and current density were each measured by utilizing SourceMeter (Keithley Instruments, Inc., 2400 series), and external quantum efficiency (EQE) was measured by utilizing an external quantum efficiency measurement apparatus, C9920-12, manufactured by Hamamatsu Photonics, co., Japan. The luminous efficiency represents a current efficiency value with respect to a current density of 50 mA/cm².

TABLE 1

| | | Driving | | | | Half service life |
|---|---|---|---|---|---|---|
| | Hole transport | voltage | Brightness | Efficiency | Luminous | (hr @100 |
| | material | (V) | (cd/m²) | (cd/A) | color | mA/cm²) |
| Example 1 | Compound 6 | 4.15 | 3300 | 7.45 | Blue | 495 |
| Example 2 | Compound 8 | 4.17 | 3250 | 7.25 | Blue | 485 |
| Example 3 | Compound 14 | 4.18 | 3230 | 7.4 | Blue | 490 |
| Example 4 | Compound 22 | 4.07 | 3270 | 7.5 | Blue | 410 |
| Example 5 | Compound 38 | 4.09 | 3280 | 7.35 | Blue | 405 |
| Example 6 | Compound 54 | 4.1 | 3150 | 7.3 | Blue | 430 |
| Example 7 | Compound 70 | 4.13 | 3180 | 7.15 | Blue | 415 |
| Example 8 | Compound 86 | 4.18 | 3075 | 7.25 | Blue | 420 |
| Example 9 | Compound 102 | 4.2 | 3010 | 7.5 | Blue | 410 |
| Example 10 | Compound 118 | 4.22 | 3050 | 7.3 | Blue | 415 |
| Example 11 | Compound 134 | 4.18 | 3160 | 7.2 | Blue | 445 |
| Example 12 | Compound 150 | 4.25 | 3145 | 7.45 | Blue | 430 |
| Example 13 | Compound 166 | 4.3 | 3125 | 7.5 | Blue | 405 |
| Comparative | Compound A1 | 7.01 | 2645 | 5.29 | Blue | 258 |
| Example 1 | | | | | | |
| Comparative | Compound A2 | 4.35 | 3010 | 6.9 | Blue | 397 |
| Example 2 | | | | | | |
| Comparative | Compound A3 | 4.4 | 3030 | 7.1 | Blue | 365 |
| Example 3 | | | | | | |
| Comparative | Compound A4 | 4.36 | 3065 | 6.94 | Blue | 277 |
| Example 4 | | | | | | |
| Comparative | Compound A5 | 4.4 | 3105 | 7.05 | Blue | 279 |
| Example 5 | | | | | | |
| Comparative | Compound A6 | 4.46 | 3055 | 6.92 | Blue | 295 |
| Example 6 | | | | | | |
| Comparative | Compound A7 | 4.5 | 3110 | 6.92 | Blue | 250 |
| Example 7 | | | | | | |

Referring to the results of Table 1, it may be seen that Examples of the light emitting devices utilizing the amine compound according to an embodiment of the present disclosure as a hole transport layer material each exhibit low driving voltage, relatively higher brightness and luminous efficiency, and a longer service life while emitting the same blue light compared to each of Comparative Examples. The amine compound according to an embodiment includes 9,9′-Spirobi[9H-fluorene] as a core. The spiro molecule may have a rigid center and a non-coplanar structure in which two fluorene molecules are linked by sharing the 9 (9′)-position carbon, which is a spa-carbon, thereby being twisted at the 9 (9′)-position carbon. Accordingly, because stacking due to the intermolecular interaction may be reduced or minimized, a stable thin film can not only be formed, but the amine compound according to an embodiment may also have high glass transition temperature and high melting point properties due to improved rigidity at the center, thereby exhibiting suitable (e.g., excellent) heat resistance and durability characteristics.

In addition, the two fluorene units include the amine group containing a carbazole group represented by Formula 2 and the amine group represented by Formula 3, respectively, having 9,9′-Spirobi[9H-fluorene] as a center, and thus may have an elongated conjugation structure, thereby improving hole transport properties. In particular, the amine group represented by Formula 2 has a structure in which the nitrogen atom is regioselectively bonded to the 2-position carbon of the carbazole group, and thus may exhibit efficient resonance properties, therefore, when the amine group represented by Formula 2 is bonded to Formula 1, hole transport properties may be further improved. Therefore, when the amine compound of an embodiment is applied in the hole transport region, the hole transport properties may be increased, thus improving recombination probability of holes and electrons in the emission layer, thereby increasing the luminous efficiency.

Comparative Example 1 does not include 9,9′-Spirobi[9H-fluorene] as a core, and thus exhibits low thermal stability. Accordingly, Comparative Example 1 does not provide sufficient morphological stability during the operation of the device or the manufacture process, and thus the luminous efficiency and service life are reduced compared to each of Examples.

Comparative Example 2 and Comparative Example 6 include 9,9′-Spirobi[9H-fluorene] as a central core, and has a structure in which the amine group containing a carbazole group is substituted at one fluorene unit and a diphenyl amine group is substituted at the other fluorene unit, but the device efficiencies and service lives are reduced because the nitrogen atom of the amine group is connected not at the 2-position carbon of the carbazole group but at the 3-position carbon, and thus the highest occupied molecular orbital (HOMO) energy level becomes relatively low (e.g., shallow).

Comparative Example 3 and Comparative Example 5 each have a structure in which two amine groups are substituted at a central core, 9,9′-Spirobi[9H-fluorene], and have a biphenyl group as a substituent of an amine group, and thus exhibit higher luminous efficiencies than other Comparative Examples due to the characteristics of the elongated conjugation structure, but do not include a carbazole group as a substituent, and thus electron donating effects are relatively reduced compared to each of Examples, so that the luminous efficiencies and service lives are reduced.

Comparative Example 4 includes a dibenzofuran group as a substituent of an amine group, not a carbazole group, and thus both the luminous efficiency and service life are reduced. It is believed that the oxygen of the dibenzofuran group has lower electron donating effects than the nitrogen of the carbazole group, and thus the resonance effects are relatively reduced.

Comparative Example 7 has a structure in which two amine groups are substituted at a central core, 9,9′-Spirobi[9H-fluorene], and one amine group of the two is connected to the 2-position carbon, but both amine groups are substituted at one (e.g., the same) fluorene unit, and thus the luminous efficiency and service life are reduced.

Comparing Comparative Example 7 with Examples, Examples each have a non-conjugation structure due to the spa-carbon of the center because one amine group is substituted at each of the two fluorene units. Accordingly, the HOMO energy level may not only be appropriately controlled, but high hole mobility may also be exhibited because the difference between the HOMO energy level and the HOMO-1 energy level becomes relatively narrow (e.g., small). On the other hand, Comparative Example 7 has a structure in which two amine groups interpose one fluorene unit therebetween and conjugated. Accordingly, it is believed that because the difference between the HOMO energy level and the HOMO-1 energy level becomes relatively broader (e.g., bigger) than those of Example Compounds, the hole mobility is reduced, and thus the luminous efficiency and service life of the device are reduced.

The light emitting device of an embodiment may exhibit improved device characteristics with a low driving voltage, high efficiency, and a long service life.

The amine compound of an embodiment may be included in a hole transport region of the light emitting device to contribute to high efficiency and a long service life of the light emitting device.

Expressions such as “at least one of” or “at least one selected from” when preceding a list of elements, modify the entire list of elements and do not modify the individual elements of the list. Further, the use of “may” when describing embodiments of the present invention refers to “one or more embodiments of the present invention.” As used herein, the term “substantially,” “about,” and similar terms are used as terms of approximation and not as terms of degree, and are intended to account for the inherent deviations in measured or calculated values that would be recognized by those of ordinary skill in the art. Moreover, any numerical range recited herein is intended to include all sub-ranges of the same numerical precision subsumed within the recited range. For example, a range of “1.0 to 10.0” is intended to include all subranges between (and including) the recited minimum value of 1.0 and the recited maximum value of 10.0, that is, having a minimum value equal to or greater than 1.0 and a maximum value equal to or less than 10.0, such as, for example, 2.4 to 7.6. Any maximum numerical limitation recited herein is intended to include all lower numerical limitations subsumed therein and any minimum numerical limitation recited in this specification is intended to include all higher numerical limitations subsumed therein. Accordingly, Applicant reserves the right to amend this specification, including the claims, to expressly recite any sub-range subsumed within the ranges expressly recited herein. All such ranges are intended to be inherently described in this specification such that amending to expressly recite any such subranges would comply with the requirements of 35 U.S.C. § 112(a), and 35 U.S.C. § 132(a).

Although the embodiments of the present disclosure are described, those with ordinary skill in the technical field to which the present disclosure pertains will understood that the present disclosure may be carried out in other specific forms without changing the technical idea or essential features. Therefore, the above-described embodiments are to be understood in all aspects as illustrative and not restrictive.

What is claimed is:

1. A light emitting device comprising:
a first electrode;
a second electrode facing the first electrode; and
an organic layer between the first electrode and the second electrode,
wherein the organic layer comprises an amine compound represented by Formula 1: 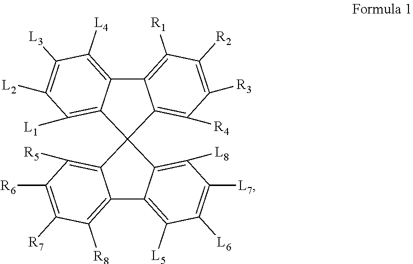
wherein, in Formula 1,
R₁ to R₈ are each independently a hydrogen atom, a deuterium atom, a halogen atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms,
L₁ to L₈ are each independently a hydrogen atom, a deuterium atom, a halogen atom, a substituted or unsubstituted amine group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms,
at least one among L₁ to L₄ is a group represented by Formula 2, and
at least one among L₅ to L₈ is a group represented by Formula 3: 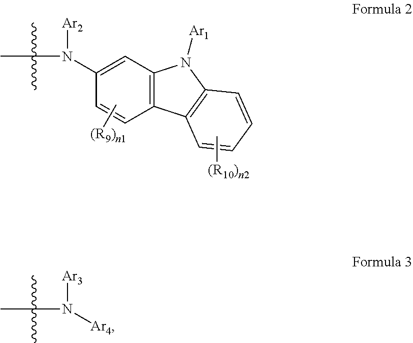
wherein, in Formula 2 and Formula 3,
R₉ and R₁₀ are each independently a hydrogen atom, a deuterium atom, a halogen atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms,
Ar₁ to Ar₄ are each independently a substituted or unsubstituted aryl group having 6 to 30 ring-forming carbon atoms, or a substituted or unsubstituted heteroaryl group having 2 to 30 ring-forming carbon atoms,
n1 is an integer of 0 to 3, and
n2 is an integer of 0 to 4.

2. The light emitting device of claim 1, wherein the organic layer comprises:
a hole transport region on the first electrode;
an emission layer on the hole transport region; and
an electron transport region on the emission layer, and
the hole transport region comprises the amine compound represented by Formula 1.

3. The light emitting device of claim 2, wherein the hole transport region comprises:
a hole injection layer on the first electrode; and
a hole transport layer on the hole injection layer, and
the hole injection layer or the hole transport layer comprises the amine compound represented by Formula 1.

4. The light emitting device of claim 2, wherein the hole transport region comprises:
a hole transport layer on the first electrode; and
an electron blocking layer on the hole transport layer, and
the electron blocking layer comprises the amine compound represented by Formula 1.

5. The light emitting device of claim 1, wherein the amine compound represented by Formula 1 is a diamine compound.

6. The light emitting device of claim 1, wherein the amine compound represented by Formula 1 is represented by any one among Formula 4-1 to Formula 4-4: 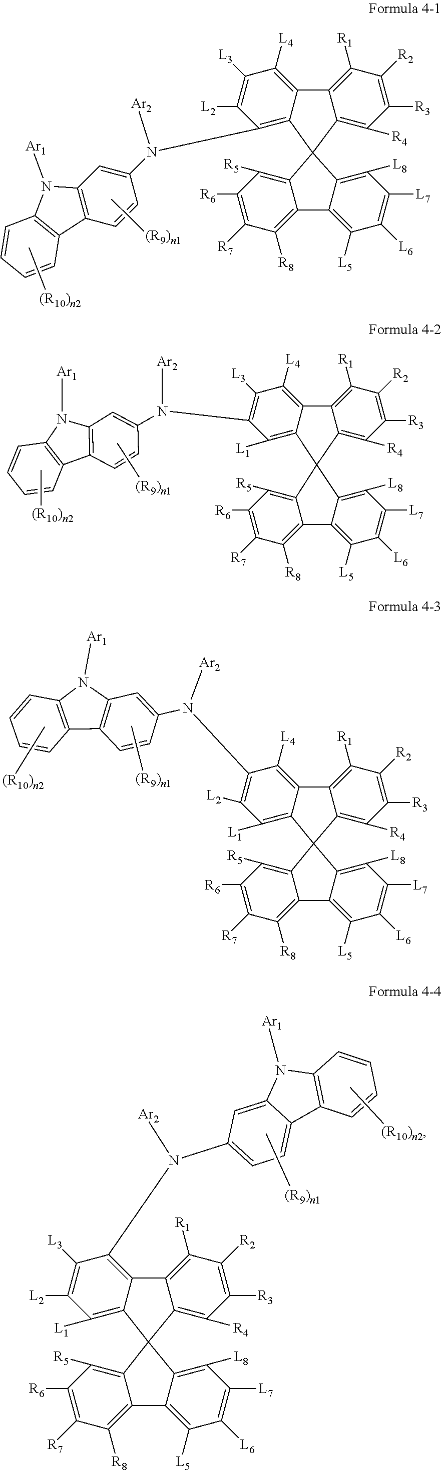
wherein, in Formula 4-1 to Formula 4-4,
R₁ to R₁₀, L₁ to L₈, Ar₁, Ar₂, n1 and n2 are the same as respectively defined in connection with Formula 1 and Formula 2.

7. The light emitting device of claim 1, wherein the amine compound represented by Formula 1 is represented by any one among Formula 5-1 to Formula 5-4: 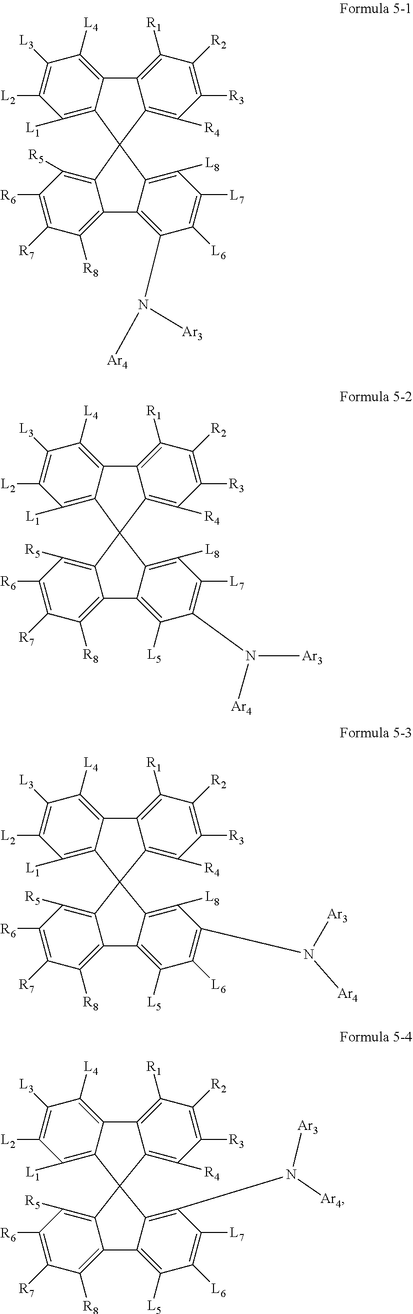
wherein, in Formula 5-1 to Formula 5-4,
R₁ to R₈, L₁ to L₈, Ara, and Ar₄ are the same as respectively defined in connection with Formula 1 and Formula 3.

8. The light emitting device of claim 1, wherein R₁ to R₁₀ are each independently a hydrogen atom or a deuterium atom.

9. The light emitting device of claim 1, wherein Ar₁ to Ar₄ are each independently a substituted or unsubstituted phenyl group, a substituted or unsubstituted naphthyl group, or a substituted or unsubstituted biphenyl group.

10. The light emitting device of claim 1, wherein the group represented by Formula 2 is represented by Formula 2-1: 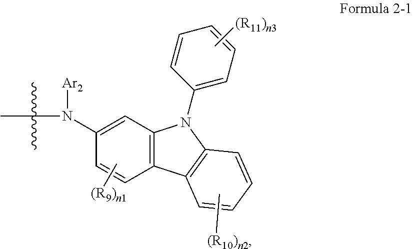
wherein, in Formula 2-1,
R₁₁ is a hydrogen atom, a deuterium atom, a halogen atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms,
n3 is an integer of 0 to 5, and
R₉, R₁₀, Ar₂, n1, and n2 are the same as respectively defined in connection with Formula 2.

11. The light emitting device of claim 1, wherein Ar₁ to Ar₄ are each independently represented by any one among Formula 6-1 to Formula 6-6: 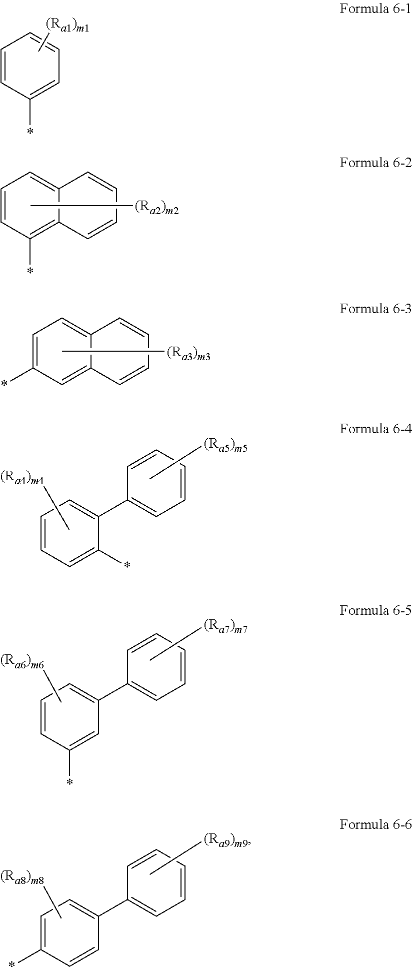
wherein, in Formula 6-1 to Formula 6-6,
Rₐ₁ to Rₐ₉ are each independently a hydrogen atom, a deuterium atom, a halogen atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms,
m1, m5, m7 and m9 are each independently an integer of 0 to 5,
m2 and m3 are each independently an integer of 0 to 7, and
m4, m6 and m8 are each independently an integer of 0 to 4.

12. The light emitting device of claim 1, wherein the amine compound represented by Formula 1 is any one among compounds represented by Compound Group 1:

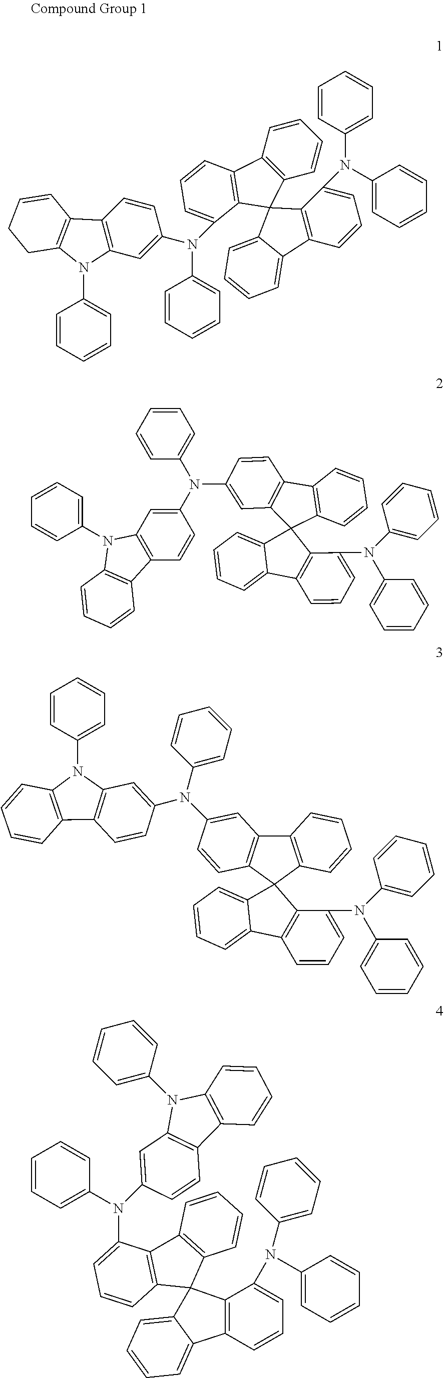 [SMILES: C1=Cc2c(n(-c3ccccc3)c3cc(N(c4ccccc4)c4cccc5c4C4(c6ccccc6-c6cccc(N(c7ccccc7)c7ccccc7)c64)c4ccccc4-5)ccc23)CC1 ; c1ccc(N(c2ccc3c(c2)C2(c4ccccc4-3)c3ccccc3-c3cccc(N(c4ccccc4)c4ccccc4)c32)c2ccc3c4ccccc4n(-c4ccccc4)c3c2)cc1 ; c1ccc(N(c2ccc3c(c2)-c2ccccc2C32c3ccccc3-c3cccc(N(c4ccccc4)c4ccccc4)c32)c2ccc3c4ccccc4n(-c4ccccc4)c3c2)cc1 ; c1ccc(N(c2ccc3c4ccccc4n(-c4ccccc4)c3c2)c2cccc3c2-c2ccccc2C32c3ccccc3-c3cccc(N(c4ccccc4)c4ccccc4)c32)cc1]

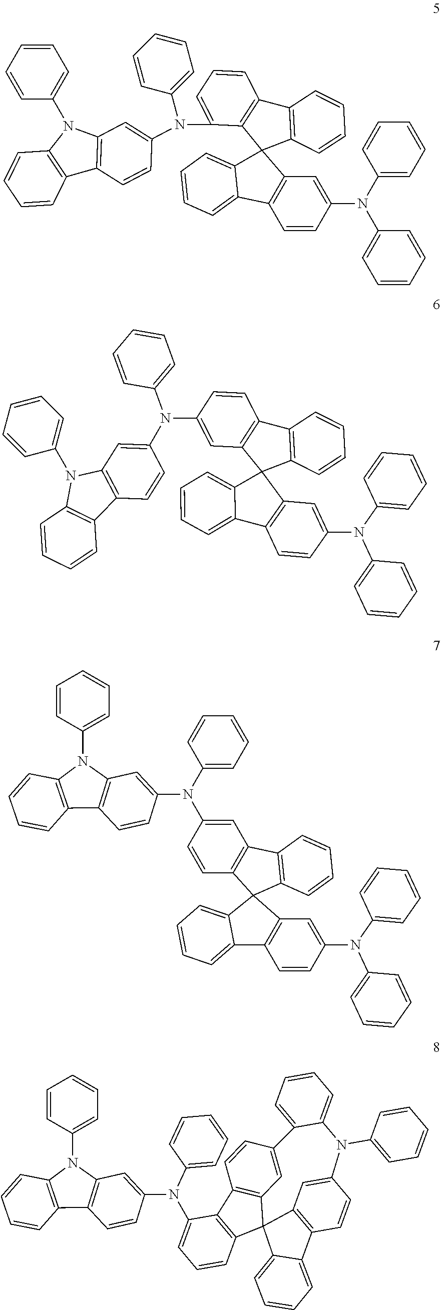 [SMILES: c1ccc(N(c2ccccc2)c2ccc3c(c2)C2(c4ccccc4-3)c3ccccc3-c3cccc(N(c4ccccc4)c4ccc5c6ccccc6n(-c6ccccc6)c5c4)c32)cc1 ; c1ccc(N(c2ccccc2)c2ccc3c(c2)C2(c4ccccc4-3)c3ccccc3-c3ccc(N(c4ccccc4)c4ccc5c6ccccc6n(-c6ccccc6)c5c4)cc32)cc1 ; c1ccc(N(c2ccccc2)c2ccc3c(c2)C2(c4ccccc4-c4cc(N(c5ccccc5)c5ccc6c7ccccc7n(-c7ccccc7)c6c5)ccc42)c2ccccc2-3)cc1 ; c1ccc(N2c3ccc4c(c3)C3(c5ccccc5-4)c4cc(ccc4-c4c(N(c5ccccc5)c5ccc6c7ccccc7n(-c7ccccc7)c6c5)cccc43)-c3ccccc32)cc1]

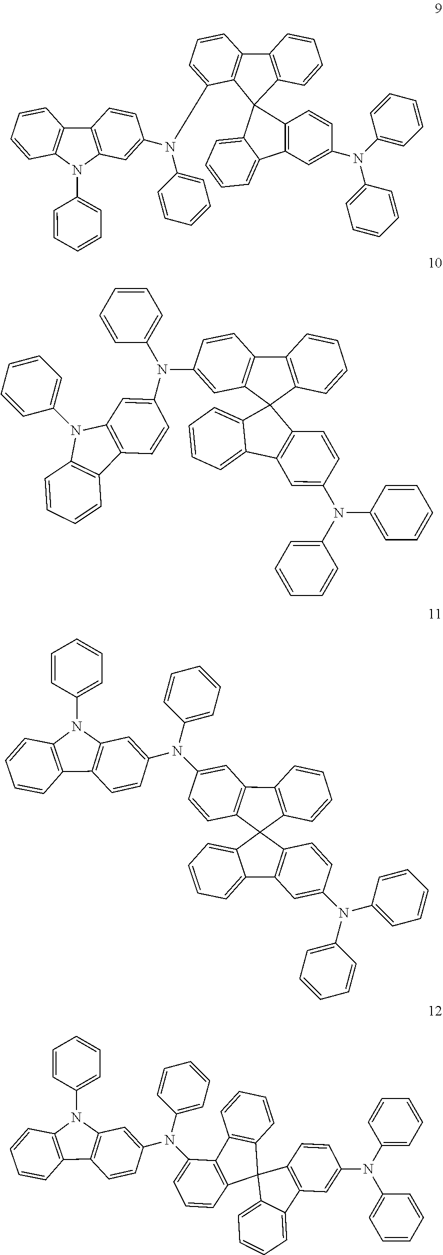 [SMILES: c1ccc(N(c2ccccc2)c2ccc3c(c2)-c2ccccc2C32c3ccccc3-c3cccc(N(c4ccccc4)c4ccc5c6ccccc6n(-c6ccccc6)c5c4)c32)cc1 ; c1ccc(N(c2ccccc2)c2ccc3c(c2)-c2ccccc2C32c3ccccc3-c3ccc(N(c4ccccc4)c4ccc5c6ccccc6n(-c6ccccc6)c5c4)cc32)cc1 ; c1ccc(N(c2ccccc2)c2ccc3c(c2)-c2ccccc2C32c3ccccc3-c3cc(N(c4ccccc4)c4ccc5c6ccccc6n(-c6ccccc6)c5c4)ccc32)cc1 ; c1ccc(N(c2ccccc2)c2ccc3c(c2)-c2ccccc2C32c3ccccc3-c3c(N(c4ccccc4)c4ccc5c6ccccc6n(-c6ccccc6)c5c4)cccc32)cc1]

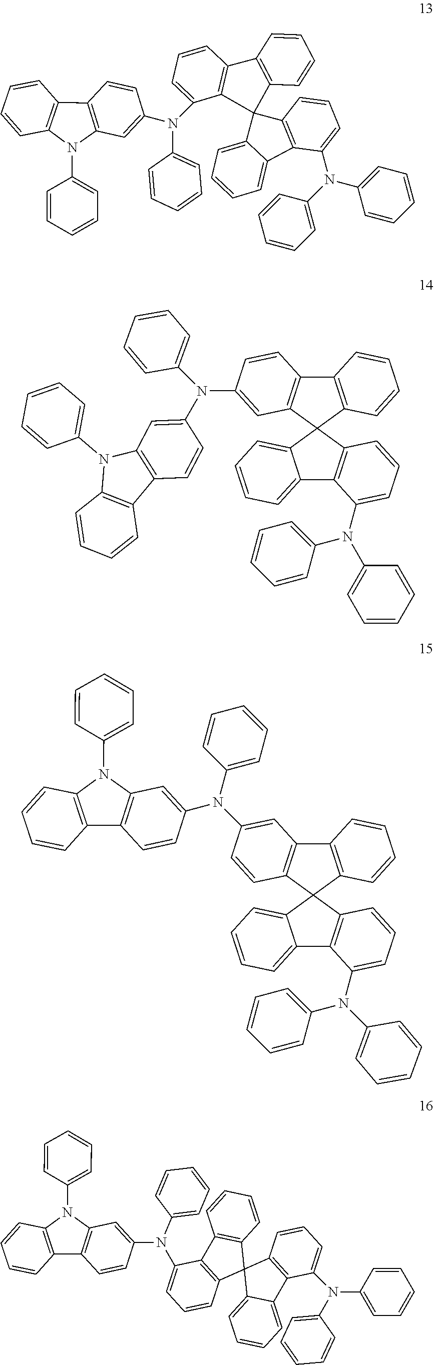 [SMILES: c1ccc(N(c2ccccc2)c2cccc3c2-c2ccccc2C32c3ccccc3-c3cccc(N(c4ccccc4)c4ccc5c6ccccc6n(-c6ccccc6)c5c4)c32)cc1 ; c1ccc(N(c2ccc3c(c2)C2(c4ccccc4-3)c3ccccc3-c3c(N(c4ccccc4)c4ccccc4)cccc32)c2ccc3c4ccccc4n(-c4ccccc4)c3c2)cc1 ; c1ccc(N(c2ccc3c(c2)-c2ccccc2C32c3ccccc3-c3c(N(c4ccccc4)c4ccccc4)cccc32)c2ccc3c4ccccc4n(-c4ccccc4)c3c2)cc1 ; c1ccc(N(c2ccccc2)c2cccc3c2-c2ccccc2C32c3ccccc3-c3c(N(c4ccccc4)c4ccc5c6ccccc6n(-c6ccccc6)c5c4)cccc32)cc1]

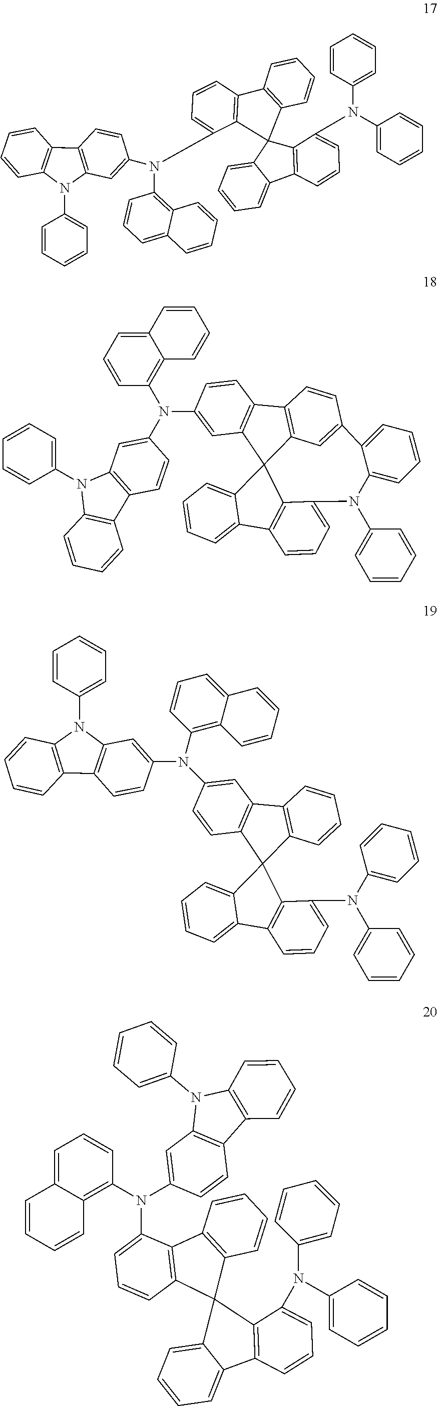 [SMILES: c1ccc(N(c2ccccc2)c2cccc3c2C2(c4ccccc4-3)c3ccccc3-c3cccc(N(c4ccc5c6ccccc6n(-c6ccccc6)c5c4)c4cccc5ccccc45)c32)cc1 ; c1ccc(N2c3ccccc3-c3ccc4c(c3)C3(c5cc(N(c6ccc7c8ccccc8n(-c8ccccc8)c7c6)c6cccc7ccccc67)ccc5-4)c4ccccc4-c4cccc2c43)cc1 ; c1ccc(N(c2ccccc2)c2cccc3c2C2(c4ccccc4-c4cc(N(c5ccc6c7ccccc7n(-c7ccccc7)c6c5)c5cccc6ccccc56)ccc42)c2ccccc2-3)cc1 ; c1ccc(N(c2ccccc2)c2cccc3c2C2(c4ccccc4-3)c3ccccc3-c3c(N(c4ccc5c6ccccc6n(-c6ccccc6)c5c4)c4cccc5ccccc45)cccc32)cc1]

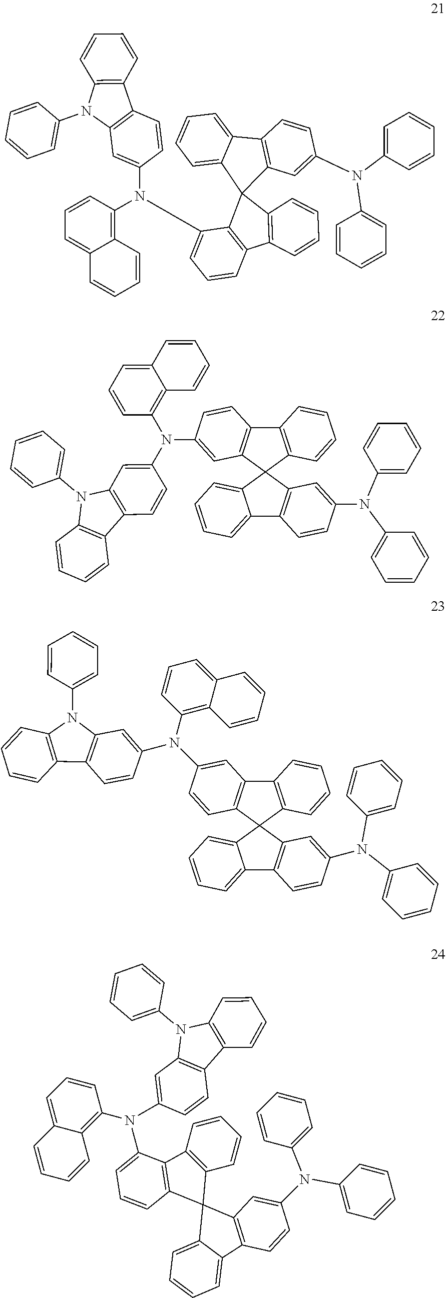 [SMILES: c1ccc(N(c2ccccc2)c2ccc3c(c2)C2(c4ccccc4-3)c3ccccc3-c3cccc(N(c4ccc5c6ccccc6n(-c6ccccc6)c5c4)c4cccc5ccccc45)c32)cc1 ; c1ccc(N(c2ccccc2)c2ccc3c(c2)C2(c4ccccc4-3)c3ccccc3-c3ccc(N(c4ccc5c6ccccc6n(-c6ccccc6)c5c4)c4cccc5ccccc45)cc32)cc1 ; c1ccc(N(c2ccccc2)c2ccc3c(c2)C2(c4ccccc4-c4cc(N(c5ccc6c7ccccc7n(-c7ccccc7)c6c5)c5cccc6ccccc56)ccc42)c2ccccc2-3)cc1 ; c1ccc(N(c2ccccc2)c2ccc3c(c2)C2(c4ccccc4-3)c3ccccc3-c3c(N(c4ccc5c6ccccc6n(-c6ccccc6)c5c4)c4cccc5ccccc45)cccc32)cc1]

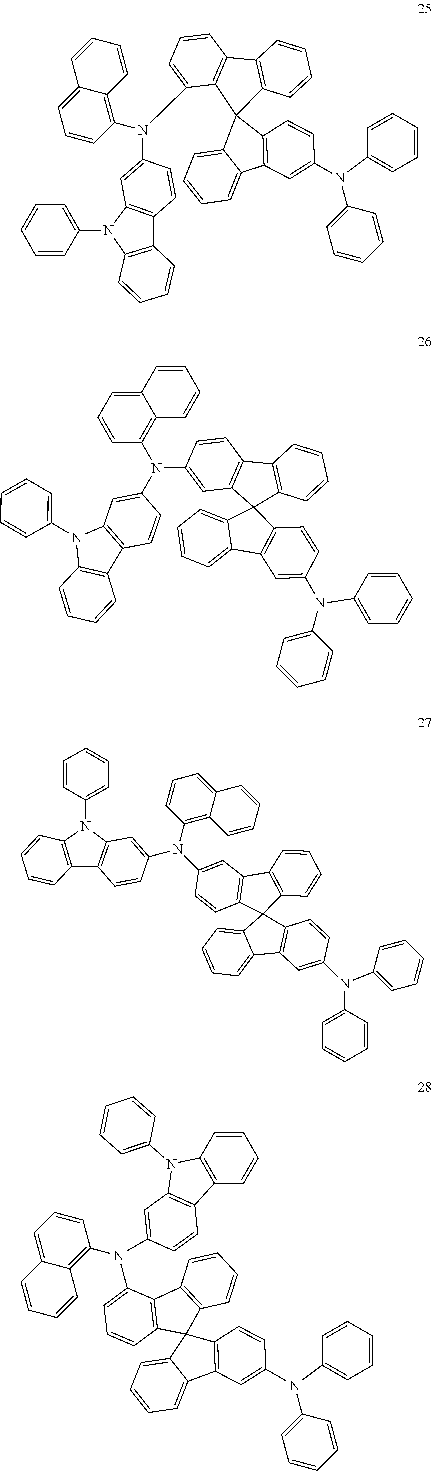 [SMILES: c1ccc(N(c2ccccc2)c2ccc3c(c2)-c2ccccc2C32c3ccccc3-c3cccc(N(c4ccc5c6ccccc6n(-c6ccccc6)c5c4)c4cccc5ccccc45)c32)cc1 ; c1ccc(N(c2ccccc2)c2ccc3c(c2)-c2ccccc2C32c3ccccc3-c3ccc(N(c4ccc5c6ccccc6n(-c6ccccc6)c5c4)c4cccc5ccccc45)cc32)cc1 ; c1ccc(N(c2ccccc2)c2ccc3c(c2)-c2ccccc2C32c3ccccc3-c3cc(N(c4ccc5c6ccccc6n(-c6ccccc6)c5c4)c4cccc5ccccc45)ccc32)cc1 ; c1ccc(N(c2ccccc2)c2ccc3c(c2)-c2ccccc2C32c3ccccc3-c3c(N(c4ccc5c6ccccc6n(-c6ccccc6)c5c4)c4cccc5ccccc45)cccc32)cc1]

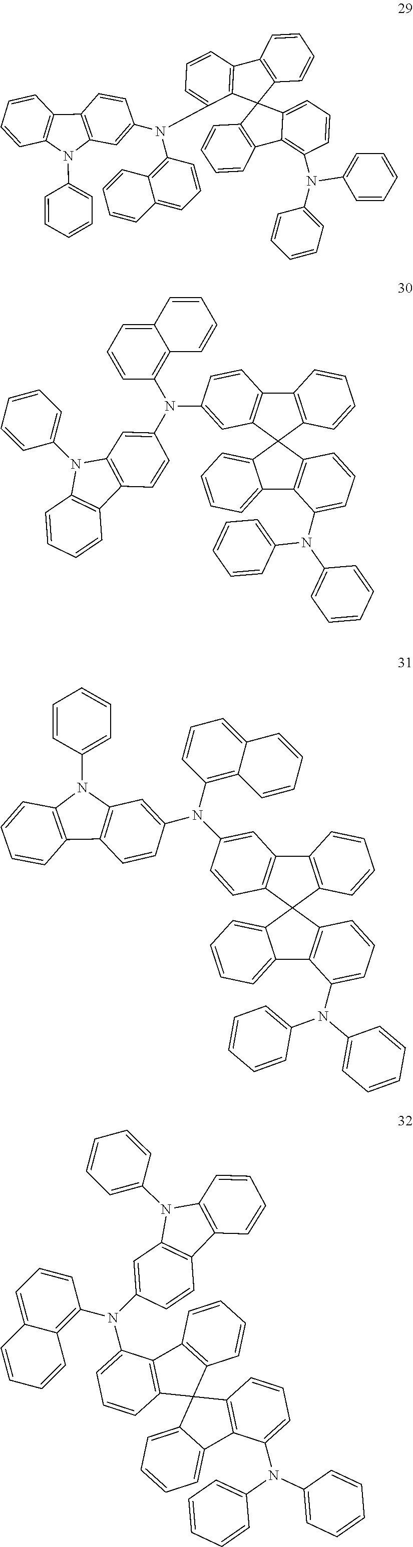 [SMILES: c1ccc(N(c2ccccc2)c2cccc3c2-c2ccccc2C32c3ccccc3-c3cccc(N(c4ccc5c6ccccc6n(-c6ccccc6)c5c4)c4cccc5ccccc45)c32)cc1 ; c1ccc(N(c2ccccc2)c2cccc3c2-c2ccccc2C32c3ccccc3-c3ccc(N(c4ccc5c6ccccc6n(-c6ccccc6)c5c4)c4cccc5ccccc45)cc32)cc1 ; c1ccc(N(c2ccccc2)c2cccc3c2-c2ccccc2C32c3ccccc3-c3cc(N(c4ccc5c6ccccc6n(-c6ccccc6)c5c4)c4cccc5ccccc45)ccc32)cc1 ; c1ccc(N(c2ccccc2)c2cccc3c2-c2ccccc2C32c3ccccc3-c3c(N(c4ccc5c6ccccc6n(-c6ccccc6)c5c4)c4cccc5ccccc45)cccc32)cc1]

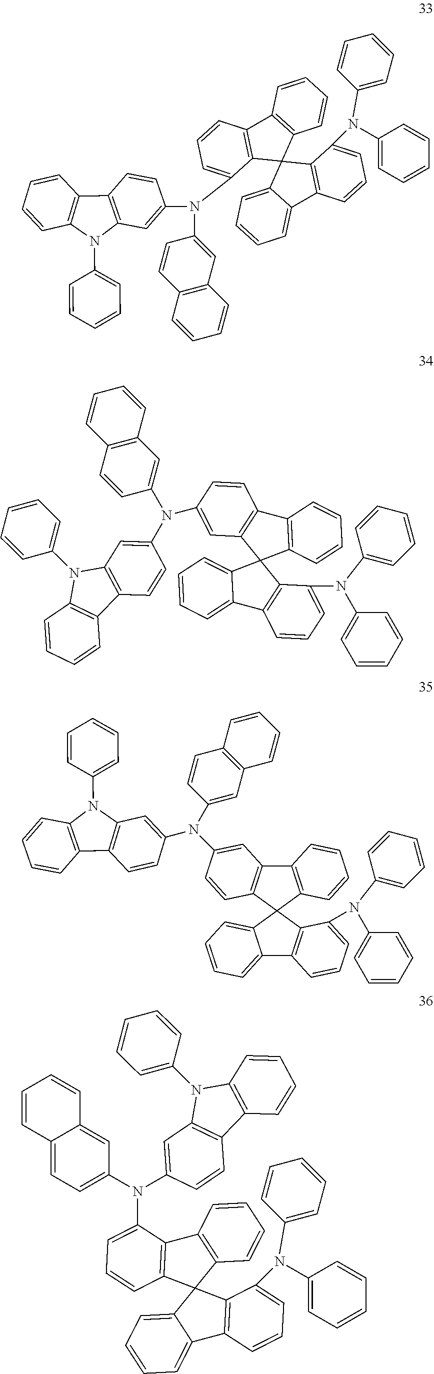 [SMILES: c1ccc(N(c2ccccc2)c2cccc3c2C2(c4ccccc4-3)c3ccccc3-c3cccc(N(c4ccc5ccccc5c4)c4ccc5c6ccccc6n(-c6ccccc6)c5c4)c32)cc1 ; c1ccc(N(c2ccccc2)c2cccc3c2C2(c4ccccc4-c4ccc(N(c5ccc6ccccc6c5)c5ccc6c7ccccc7n(-c7ccccc7)c6c5)cc42)c2ccccc2-3)cc1 ; c1ccc(N(c2ccccc2)c2cccc3c2C2(c4ccccc4-c4cc(N(c5ccc6ccccc6c5)c5ccc6c7ccccc7n(-c7ccccc7)c6c5)ccc42)c2ccccc2-3)cc1 ; c1ccc(N(c2ccccc2)c2cccc3c2C2(c4ccccc4-3)c3ccccc3-c3c(N(c4ccc5ccccc5c4)c4ccc5c6ccccc6n(-c6ccccc6)c5c4)cccc32)cc1]

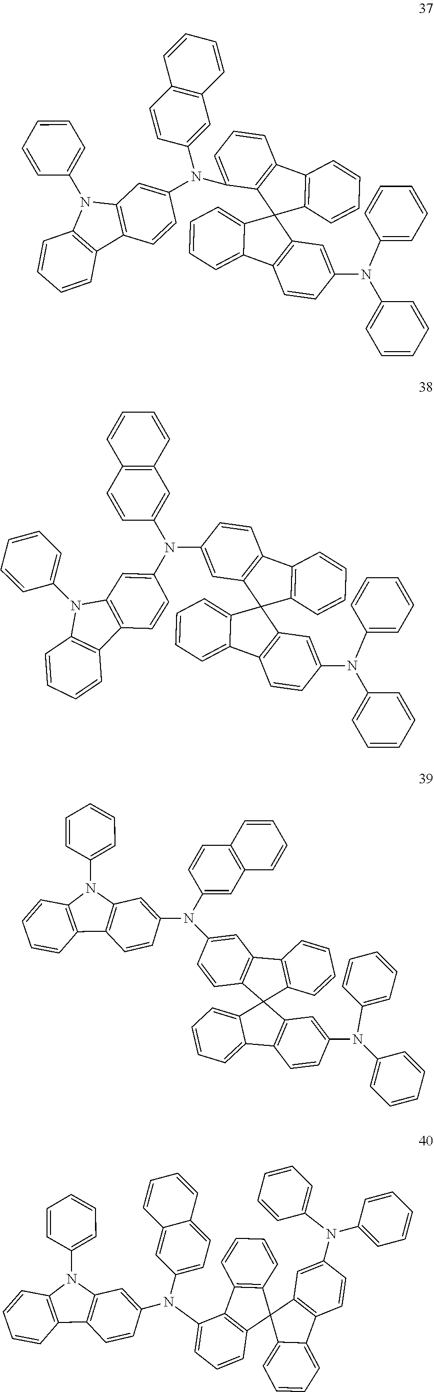 [SMILES: c1ccc(N(c2ccccc2)c2ccc3c(c2)C2(c4ccccc4-3)c3ccccc3-c3cccc(N(c4ccc5ccccc5c4)c4ccc5c6ccccc6n(-c6ccccc6)c5c4)c32)cc1 ; c1ccc(N(c2ccccc2)c2ccc3c(c2)C2(c4ccccc4-3)c3ccccc3-c3ccc(N(c4ccc5ccccc5c4)c4ccc5c6ccccc6n(-c6ccccc6)c5c4)cc32)cc1 ; c1ccc(N(c2ccccc2)c2ccc3c(c2)C2(c4ccccc4-c4cc(N(c5ccc6ccccc6c5)c5ccc6c7ccccc7n(-c7ccccc7)c6c5)ccc42)c2ccccc2-3)cc1 ; c1ccc(N(c2ccccc2)c2ccc3c(c2)C2(c4ccccc4-3)c3ccccc3-c3c(N(c4ccc5ccccc5c4)c4ccc5c6ccccc6n(-c6ccccc6)c5c4)cccc32)cc1]

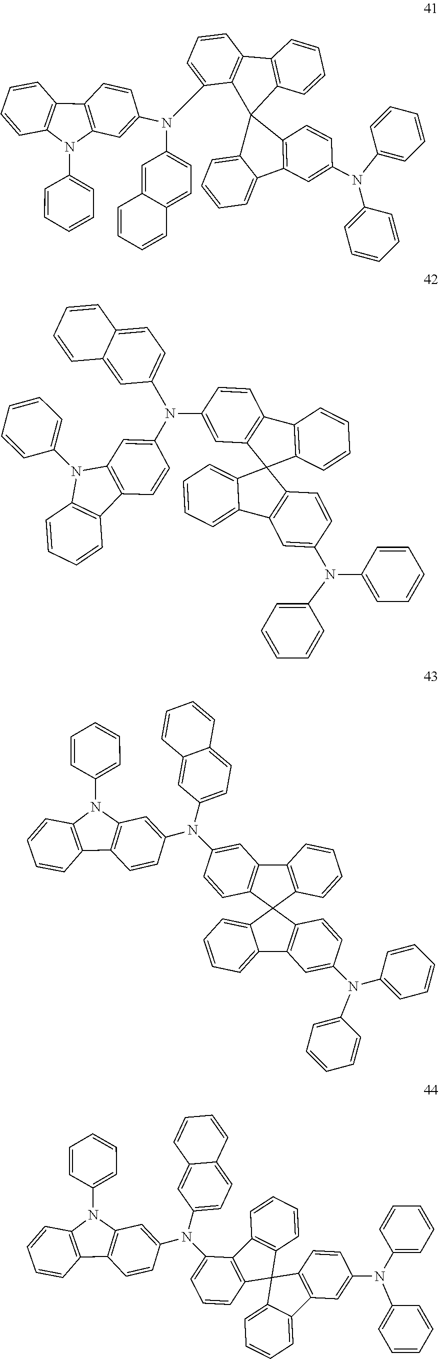 [SMILES: c1ccc(N(c2ccccc2)c2ccc3c(c2)-c2ccccc2C32c3ccccc3-c3cccc(N(c4ccc5ccccc5c4)c4ccc5c6ccccc6n(-c6ccccc6)c5c4)c32)cc1 ; c1ccc(N(c2ccccc2)c2ccc3c(c2)-c2ccccc2C32c3ccccc3-c3ccc(N(c4ccc5ccccc5c4)c4ccc5c6ccccc6n(-c6ccccc6)c5c4)cc32)cc1 ; c1ccc(N(c2ccccc2)c2ccc3c(c2)-c2ccccc2C32c3ccccc3-c3cc(N(c4ccc5ccccc5c4)c4ccc5c6ccccc6n(-c6ccccc6)c5c4)ccc32)cc1 ; c1ccc(N(c2ccccc2)c2ccc3c(c2)-c2ccccc2C32c3ccccc3-c3c(N(c4ccc5ccccc5c4)c4ccc5c6ccccc6n(-c6ccccc6)c5c4)cccc32)cc1]

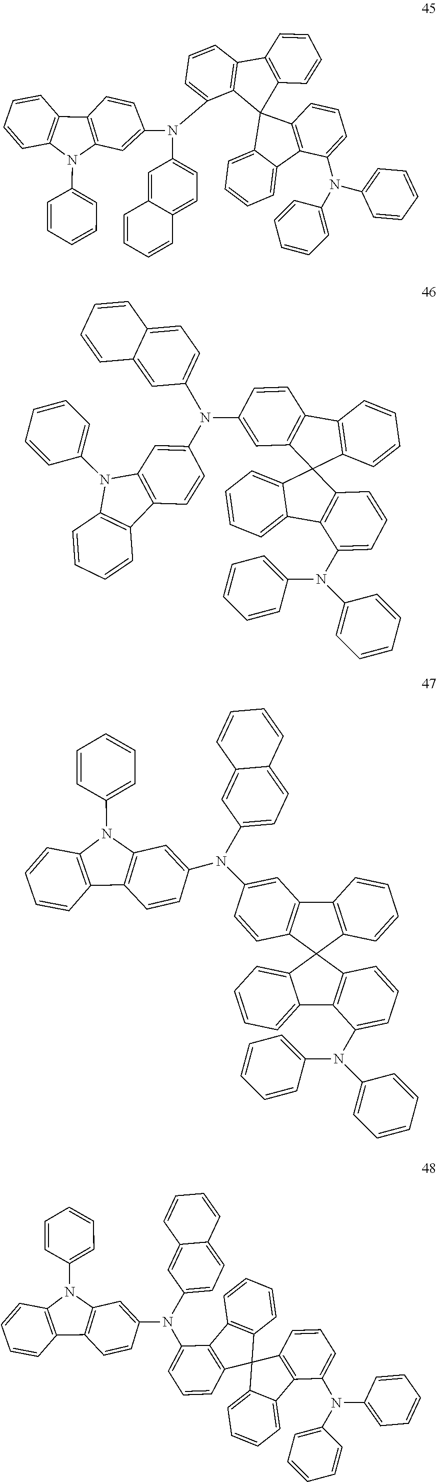 [SMILES: c1ccc(N(c2ccccc2)c2cccc3c2-c2ccccc2C32c3ccccc3-c3cccc(N(c4ccc5ccccc5c4)c4ccc5c6ccccc6n(-c6ccccc6)c5c4)c32)cc1 ; c1ccc(N(c2ccccc2)c2cccc3c2-c2ccccc2C32c3ccccc3-c3ccc(N(c4ccc5ccccc5c4)c4ccc5c6ccccc6n(-c6ccccc6)c5c4)cc32)cc1 ; c1ccc(N(c2ccccc2)c2cccc3c2-c2ccccc2C32c3ccccc3-c3cc(N(c4ccc5ccccc5c4)c4ccc5c6ccccc6n(-c6ccccc6)c5c4)ccc32)cc1 ; c1ccc(N(c2ccccc2)c2cccc3c2-c2ccccc2C32c3ccccc3-c3c(N(c4ccc5ccccc5c4)c4ccc5c6ccccc6n(-c6ccccc6)c5c4)cccc32)cc1]

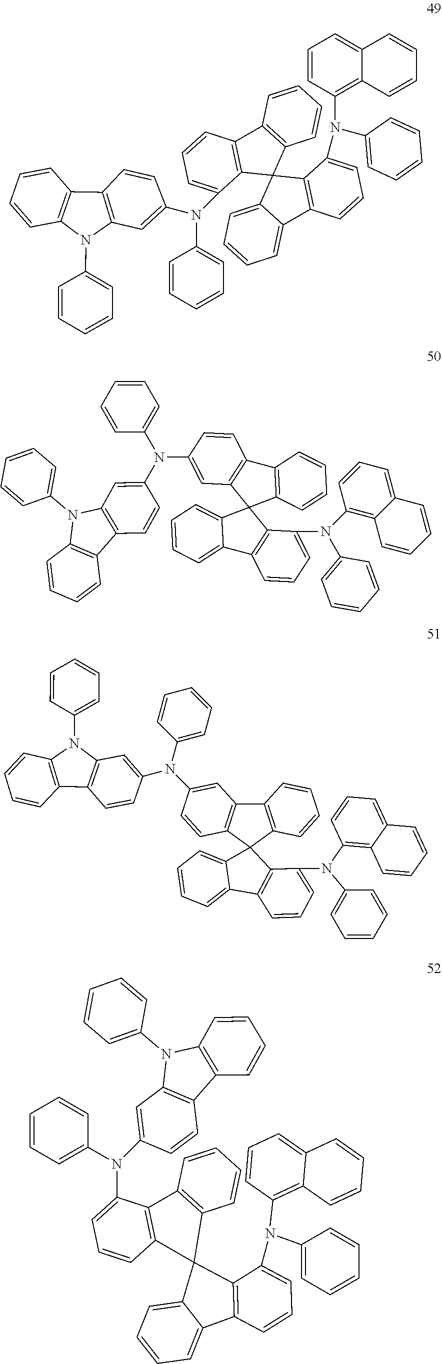 [SMILES: c1ccc(N(c2ccc3c4ccccc4n(-c4ccccc4)c3c2)c2cccc3c2C2(c4ccccc4-3)c3ccccc3-c3cccc(N(c4ccccc4)c4cccc5ccccc45)c32)cc1 ; c1ccc(N(c2ccc3c(c2)C2(c4ccccc4-3)c3ccccc3-c3cccc(N(c4ccccc4)c4cccc5ccccc45)c32)c2ccc3c4ccccc4n(-c4ccccc4)c3c2)cc1 ; c1ccc(N(c2ccc3c(c2)-c2ccccc2C32c3ccccc3-c3cccc(N(c4ccccc4)c4cccc5ccccc45)c32)c2ccc3c4ccccc4n(-c4ccccc4)c3c2)cc1 ; c1ccc(N(c2ccc3c4ccccc4n(-c4ccccc4)c3c2)c2cccc3c2-c2ccccc2C32c3ccccc3-c3cccc(N(c4ccccc4)c4cccc5ccccc45)c32)cc1]

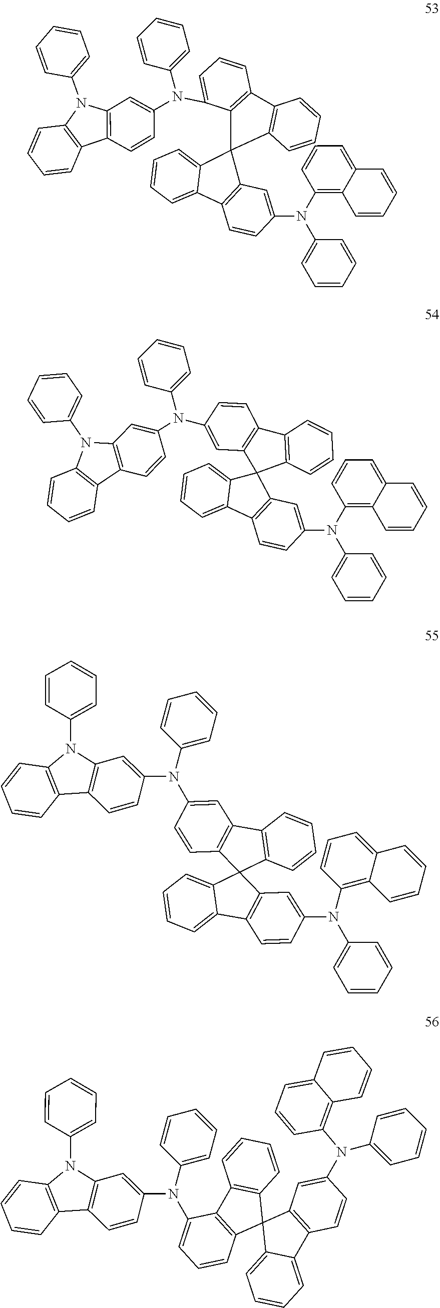 [SMILES: c1ccc(N(c2ccc3c4ccccc4n(-c4ccccc4)c3c2)c2cccc3c2C2(c4ccccc4-c4ccc(N(c5ccccc5)c5cccc6ccccc56)cc42)c2ccccc2-3)cc1 ; c1ccc(N(c2ccc3c(c2)C2(c4ccccc4-3)c3ccccc3-c3ccc(N(c4ccccc4)c4cccc5ccccc45)cc32)c2ccc3c4ccccc4n(-c4ccccc4)c3c2)cc1 ; c1ccc(N(c2ccc3c(c2)-c2ccccc2C32c3ccccc3-c3ccc(N(c4ccccc4)c4cccc5ccccc45)cc32)c2ccc3c4ccccc4n(-c4ccccc4)c3c2)cc1 ; c1ccc(N(c2ccc3c4ccccc4n(-c4ccccc4)c3c2)c2cccc3c2-c2ccccc2C32c3ccccc3-c3ccc(N(c4ccccc4)c4cccc5ccccc45)cc32)cc1]

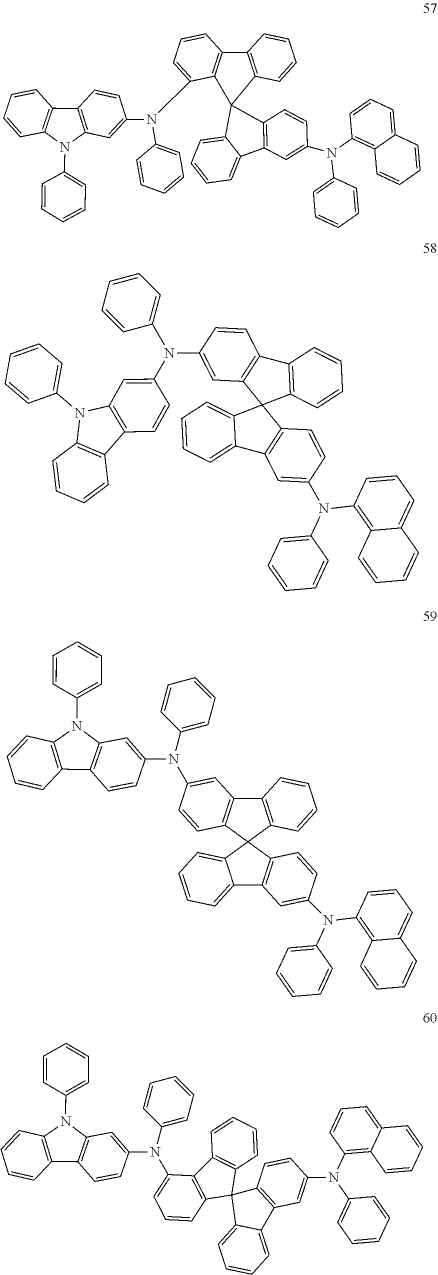 [SMILES: c1ccc(N(c2ccc3c4ccccc4n(-c4ccccc4)c3c2)c2cccc3c2C2(c4ccccc4-c4cc(N(c5ccccc5)c5cccc6ccccc56)ccc42)c2ccccc2-3)cc1 ; c1ccc(N(c2ccc3c(c2)C2(c4ccccc4-c4cc(N(c5ccccc5)c5cccc6ccccc56)ccc42)c2ccccc2-3)c2ccc3c4ccccc4n(-c4ccccc4)c3c2)cc1 ; c1ccc(N(c2ccc3c(c2)-c2ccccc2C32c3ccccc3-c3cc(N(c4ccccc4)c4cccc5ccccc45)ccc32)c2ccc3c4ccccc4n(-c4ccccc4)c3c2)cc1 ; c1ccc(N(c2ccc3c4ccccc4n(-c4ccccc4)c3c2)c2cccc3c2-c2ccccc2C32c3ccccc3-c3cc(N(c4ccccc4)c4cccc5ccccc45)ccc32)cc1]

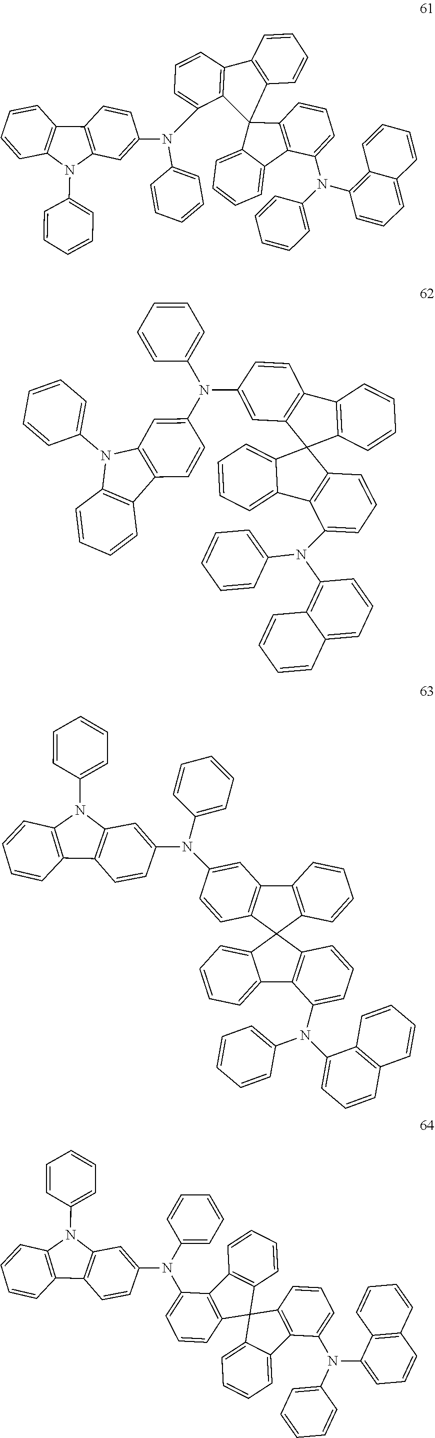 [SMILES: c1ccc(N(c2ccc3c4ccccc4n(-c4ccccc4)c3c2)c2cccc3c2C2(c4ccccc4-3)c3ccccc3-c3c(N(c4ccccc4)c4cccc5ccccc45)cccc32)cc1 ; c1ccc(N(c2ccc3c(c2)C2(c4ccccc4-3)c3ccccc3-c3c(N(c4ccccc4)c4cccc5ccccc45)cccc32)c2ccc3c4ccccc4n(-c4ccccc4)c3c2)cc1 ; c1ccc(N(c2ccc3c(c2)-c2ccccc2C32c3ccccc3-c3c(N(c4ccccc4)c4cccc5ccccc45)cccc32)c2ccc3c4ccccc4n(-c4ccccc4)c3c2)cc1 ; c1ccc(N(c2ccc3c4ccccc4n(-c4ccccc4)c3c2)c2cccc3c2-c2ccccc2C32c3ccccc3-c3c(N(c4ccccc4)c4cccc5ccccc45)cccc32)cc1]

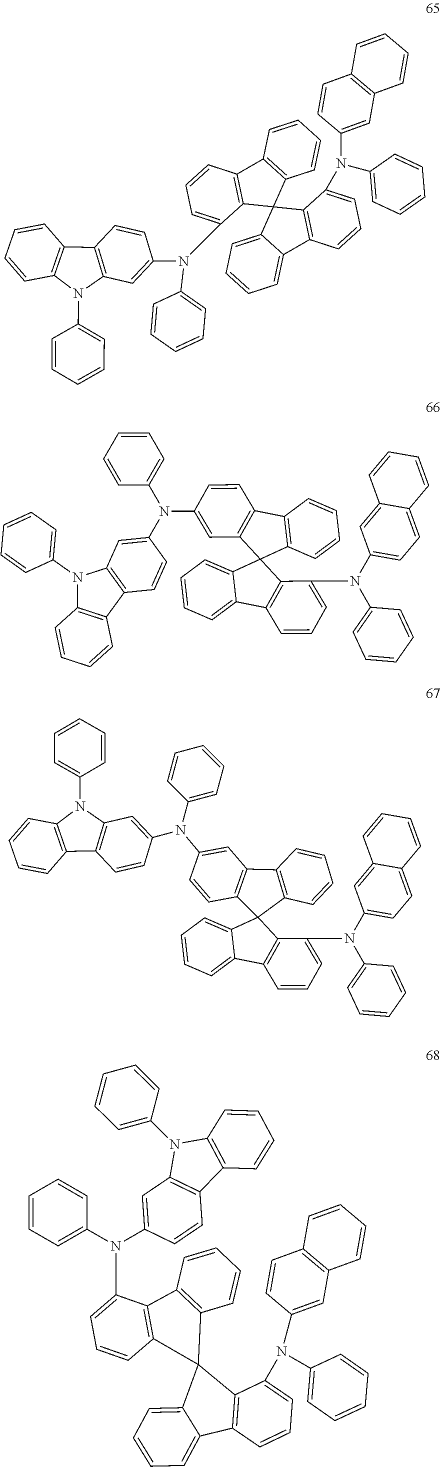 [SMILES: c1ccc(N(c2ccc3ccccc3c2)c2cccc3c2C2(c4ccccc4-3)c3ccccc3-c3cccc(N(c4ccccc4)c4ccc5c6ccccc6n(-c6ccccc6)c5c4)c32)cc1 ; c1ccc(N(c2ccc3c(c2)C2(c4ccccc4-3)c3ccccc3-c3cccc(N(c4ccccc4)c4ccc5ccccc5c4)c32)c2ccc3c4ccccc4n(-c4ccccc4)c3c2)cc1 ; c1ccc(N(c2ccc3c(c2)-c2ccccc2C32c3ccccc3-c3cccc(N(c4ccccc4)c4ccc5ccccc5c4)c32)c2ccc3c4ccccc4n(-c4ccccc4)c3c2)cc1 ; c1ccc(N(c2ccc3c4ccccc4n(-c4ccccc4)c3c2)c2cccc3c2-c2ccccc2C32c3ccccc3-c3cccc(N(c4ccccc4)c4ccc5ccccc5c4)c32)cc1]

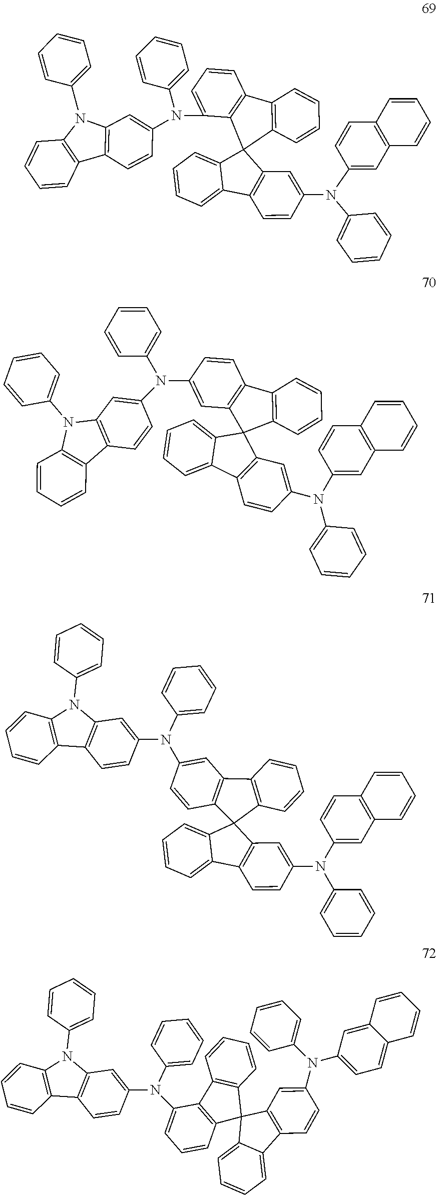 [SMILES: c1ccc(N(c2ccc3c(c2)C2(c4ccccc4-3)c3ccccc3-c3cccc(N(c4ccccc4)c4ccc5c6ccccc6n(-c6ccccc6)c5c4)c32)c2ccc3ccccc3c2)cc1 ; c1ccc(N(c2ccc3c(c2)C2(c4ccccc4-3)c3ccccc3-c3ccc(N(c4ccccc4)c4ccc5c6ccccc6n(-c6ccccc6)c5c4)cc32)c2ccc3ccccc3c2)cc1 ; c1ccc(N(c2ccc3c(c2)C2(c4ccccc4-c4cc(N(c5ccccc5)c5ccc6c7ccccc7n(-c7ccccc7)c6c5)ccc42)c2ccccc2-3)c2ccc3ccccc3c2)cc1 ; c1ccc(N(c2ccc3c(c2)C2(c4ccccc4-3)c3ccccc3-c3c(N(c4ccccc4)c4ccc5c6ccccc6n(-c6ccccc6)c5c4)cccc32)c2ccc3ccccc3c2)cc1]

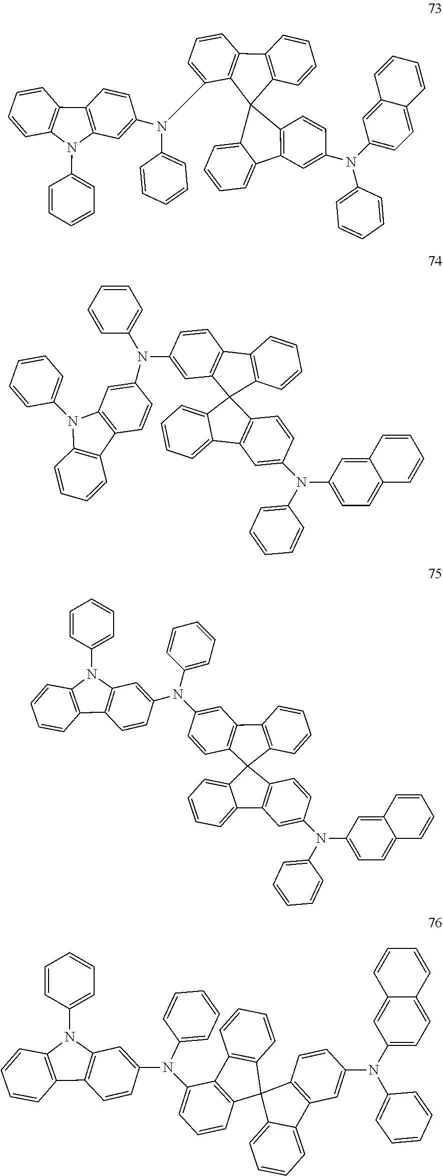 [SMILES: c1ccc(N(c2ccc3c(c2)-c2ccccc2C32c3ccccc3-c3cccc(N(c4ccccc4)c4ccc5c6ccccc6n(-c6ccccc6)c5c4)c32)c2ccc3ccccc3c2)cc1 ; c1ccc(N(c2ccc3c(c2)-c2ccccc2C32c3ccccc3-c3ccc(N(c4ccccc4)c4ccc5c6ccccc6n(-c6ccccc6)c5c4)cc32)c2ccc3ccccc3c2)cc1 ; c1ccc(N(c2ccc3c(c2)-c2ccccc2C32c3ccccc3-c3cc(N(c4ccccc4)c4ccc5c6ccccc6n(-c6ccccc6)c5c4)ccc32)c2ccc3ccccc3c2)cc1 ; c1ccc(N(c2ccc3c(c2)-c2ccccc2C32c3ccccc3-c3c(N(c4ccccc4)c4ccc5c6ccccc6n(-c6ccccc6)c5c4)cccc32)c2ccc3ccccc3c2)cc1]

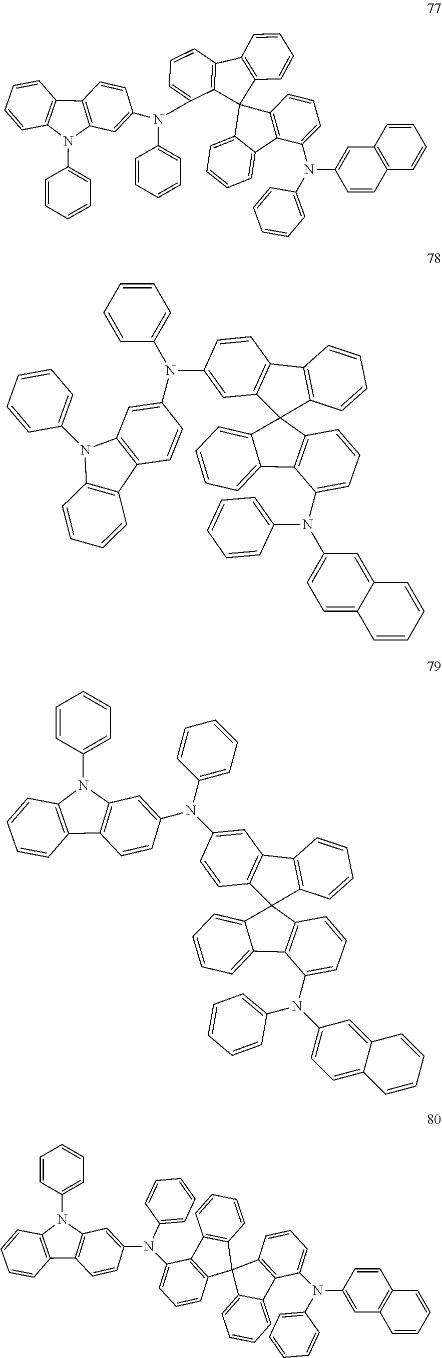 [SMILES: c1ccc(N(c2ccc3ccccc3c2)c2cccc3c2-c2ccccc2C32c3ccccc3-c3cccc(N(c4ccccc4)c4ccc5c6ccccc6n(-c6ccccc6)c5c4)c32)cc1 ; c1ccc(N(c2ccc3c(c2)C2(c4ccccc4-3)c3ccccc3-c3c(N(c4ccccc4)c4ccc5ccccc5c4)cccc32)c2ccc3c4ccccc4n(-c4ccccc4)c3c2)cc1 ; c1ccc(N(c2ccc3c(c2)-c2ccccc2C32c3ccccc3-c3c(N(c4ccccc4)c4ccc5ccccc5c4)cccc32)c2ccc3c4ccccc4n(-c4ccccc4)c3c2)cc1 ; c1ccc(N(c2ccc3ccccc3c2)c2cccc3c2-c2ccccc2C32c3ccccc3-c3c(N(c4ccccc4)c4ccc5c6ccccc6n(-c6ccccc6)c5c4)cccc32)cc1]

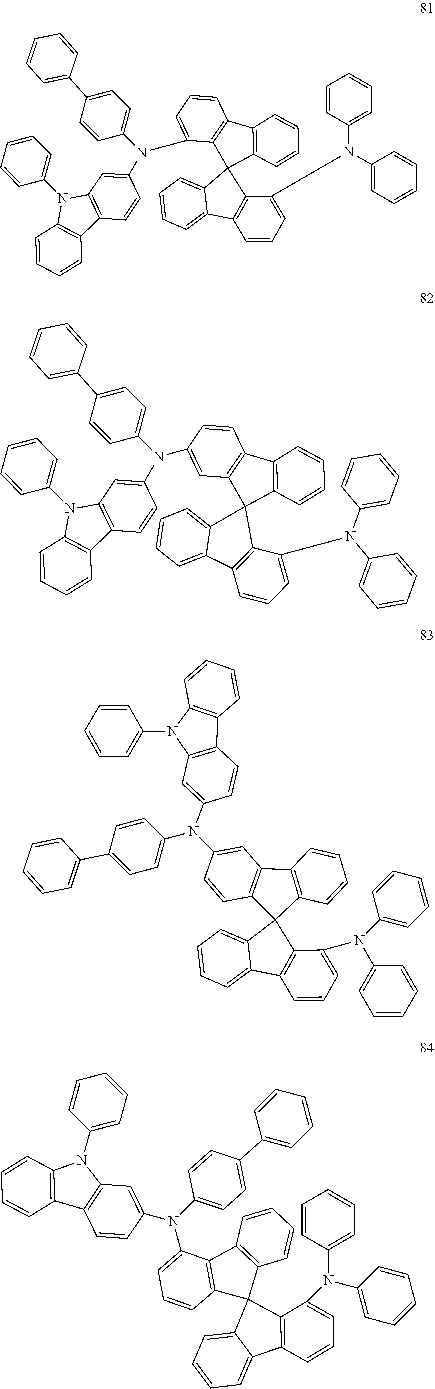 [SMILES: c1ccc(-c2ccc(N(c3ccc4c5ccccc5n(-c5ccccc5)c4c3)c3cccc4c3C3(c5ccccc5-c5cccc(N(c6ccccc6)c6ccccc6)c53)c3ccccc3-4)cc2)cc1 ; c1ccc(-c2ccc(N(c3ccc4c(c3)C3(c5ccccc5-4)c4ccccc4-c4cccc(N(c5ccccc5)c5ccccc5)c43)c3ccc4c5ccccc5n(-c5ccccc5)c4c3)cc2)cc1 ; c1ccc(-c2ccc(N(c3ccc4c(c3)-c3ccccc3C43c4ccccc4-c4cccc(N(c5ccccc5)c5ccccc5)c43)c3ccc4c5ccccc5n(-c5ccccc5)c4c3)cc2)cc1 ; c1ccc(-c2ccc(N(c3ccc4c5ccccc5n(-c5ccccc5)c4c3)c3cccc4c3-c3ccccc3C43c4ccccc4-c4cccc(N(c5ccccc5)c5ccccc5)c43)cc2)cc1]

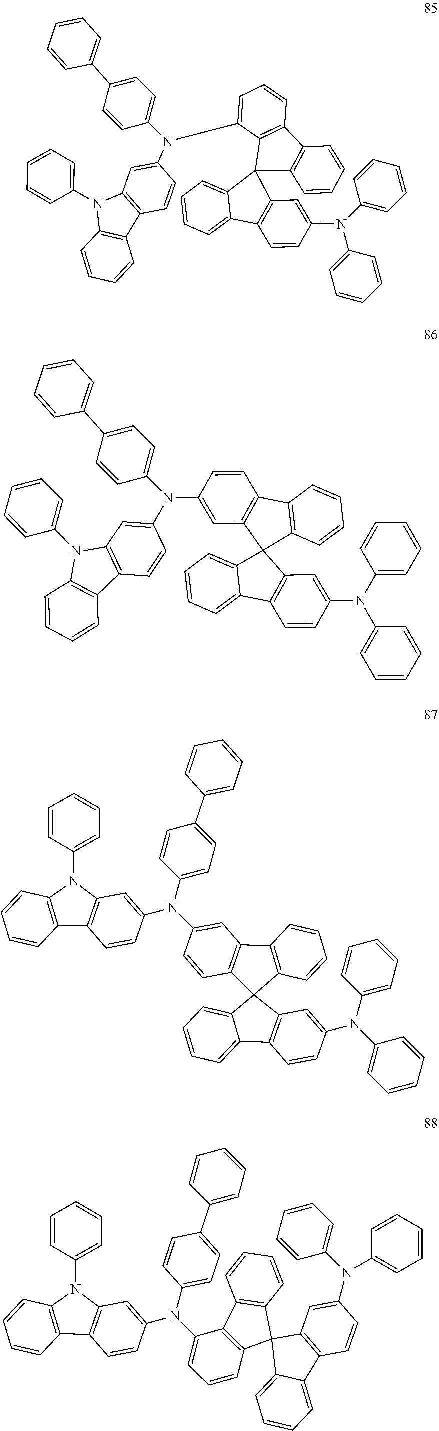 [SMILES: c1ccc(-c2ccc(N(c3ccc4c5ccccc5n(-c5ccccc5)c4c3)c3cccc4c3C3(c5ccccc5-c5ccc(N(c6ccccc6)c6ccccc6)cc53)c3ccccc3-4)cc2)cc1 ; c1ccc(-c2ccc(N(c3ccc4c(c3)C3(c5ccccc5-c5ccc(N(c6ccccc6)c6ccccc6)cc53)c3ccccc3-4)c3ccc4c5ccccc5n(-c5ccccc5)c4c3)cc2)cc1 ; c1ccc(-c2ccc(N(c3ccc4c(c3)-c3ccccc3C43c4ccccc4-c4ccc(N(c5ccccc5)c5ccccc5)cc43)c3ccc4c5ccccc5n(-c5ccccc5)c4c3)cc2)cc1 ; c1ccc(-c2ccc(N(c3ccc4c5ccccc5n(-c5ccccc5)c4c3)c3cccc4c3-c3ccccc3C43c4ccccc4-c4ccc(N(c5ccccc5)c5ccccc5)cc43)cc2)cc1]

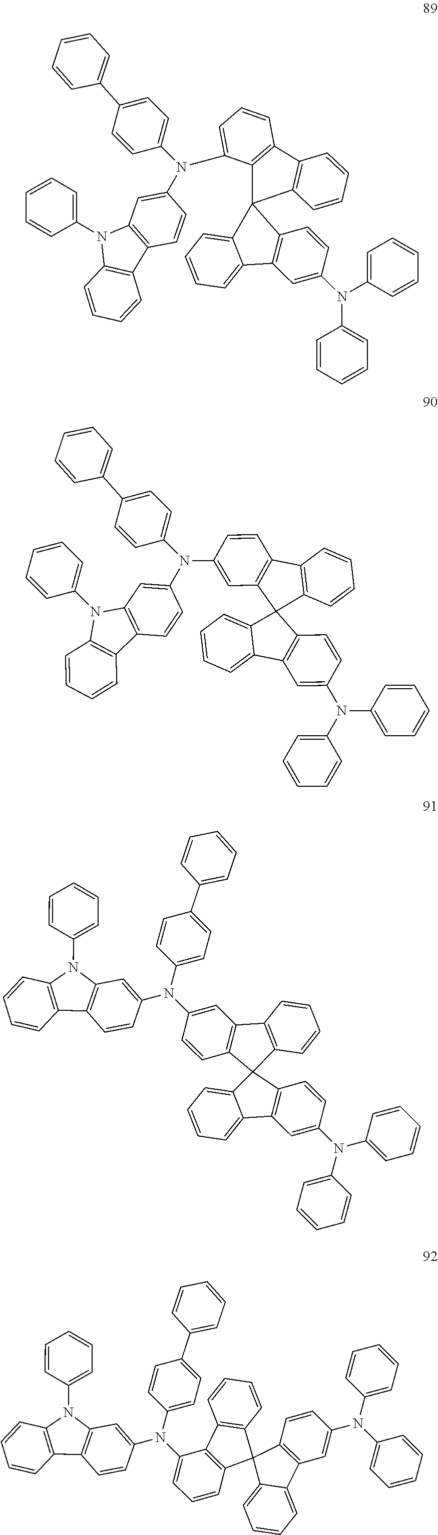 [SMILES: c1ccc(-c2ccc(N(c3ccc4c5ccccc5n(-c5ccccc5)c4c3)c3cccc4c3C3(c5ccccc5-c5cc(N(c6ccccc6)c6ccccc6)ccc53)c3ccccc3-4)cc2)cc1 ; c1ccc(-c2ccc(N(c3ccc4c(c3)C3(c5ccccc5-c5cc(N(c6ccccc6)c6ccccc6)ccc53)c3ccccc3-4)c3ccc4c5ccccc5n(-c5ccccc5)c4c3)cc2)cc1 ; c1ccc(-c2ccc(N(c3ccc4c(c3)-c3ccccc3C43c4ccccc4-c4cc(N(c5ccccc5)c5ccccc5)ccc43)c3ccc4c5ccccc5n(-c5ccccc5)c4c3)cc2)cc1 ; c1ccc(-c2ccc(N(c3ccc4c5ccccc5n(-c5ccccc5)c4c3)c3cccc4c3-c3ccccc3C43c4ccccc4-c4cc(N(c5ccccc5)c5ccccc5)ccc43)cc2)cc1]

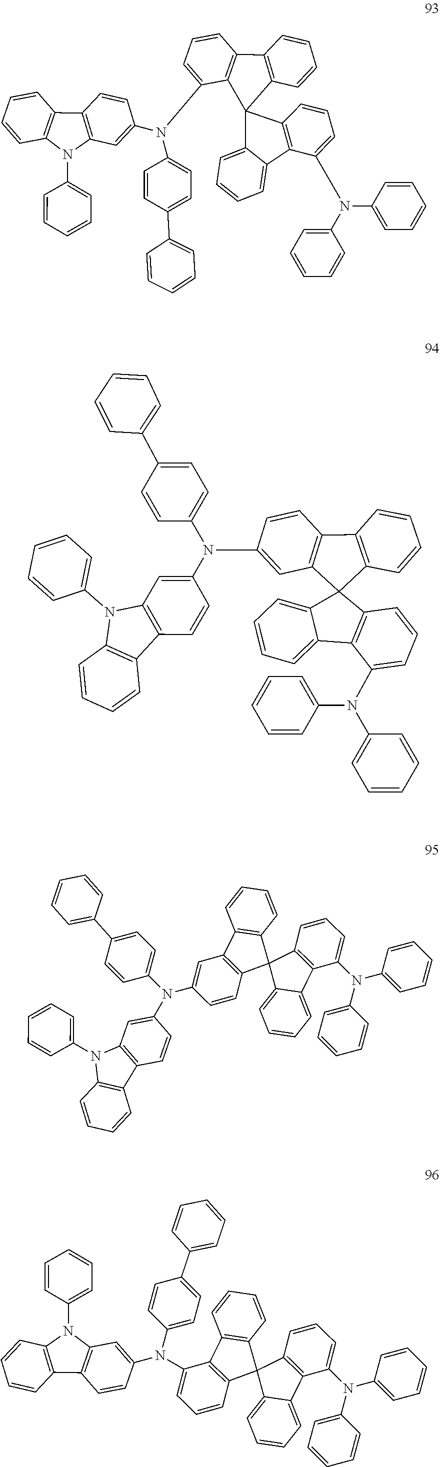 [SMILES: c1ccc(-c2ccc(N(c3ccc4c5ccccc5n(-c5ccccc5)c4c3)c3cccc4c3C3(c5ccccc5-4)c4ccccc4-c4c(N(c5ccccc5)c5ccccc5)cccc43)cc2)cc1 ; c1ccc(-c2ccc(N(c3ccc4c(c3)C3(c5ccccc5-4)c4ccccc4-c4c(N(c5ccccc5)c5ccccc5)cccc43)c3ccc4c5ccccc5n(-c5ccccc5)c4c3)cc2)cc1 ; c1ccc(-c2ccc(N(c3ccc4c(c3)-c3ccccc3C43c4ccccc4-c4c(N(c5ccccc5)c5ccccc5)cccc43)c3ccc4c5ccccc5n(-c5ccccc5)c4c3)cc2)cc1 ; c1ccc(-c2ccc(N(c3ccc4c5ccccc5n(-c5ccccc5)c4c3)c3cccc4c3-c3ccccc3C43c4ccccc4-c4c(N(c5ccccc5)c5ccccc5)cccc43)cc2)cc1]

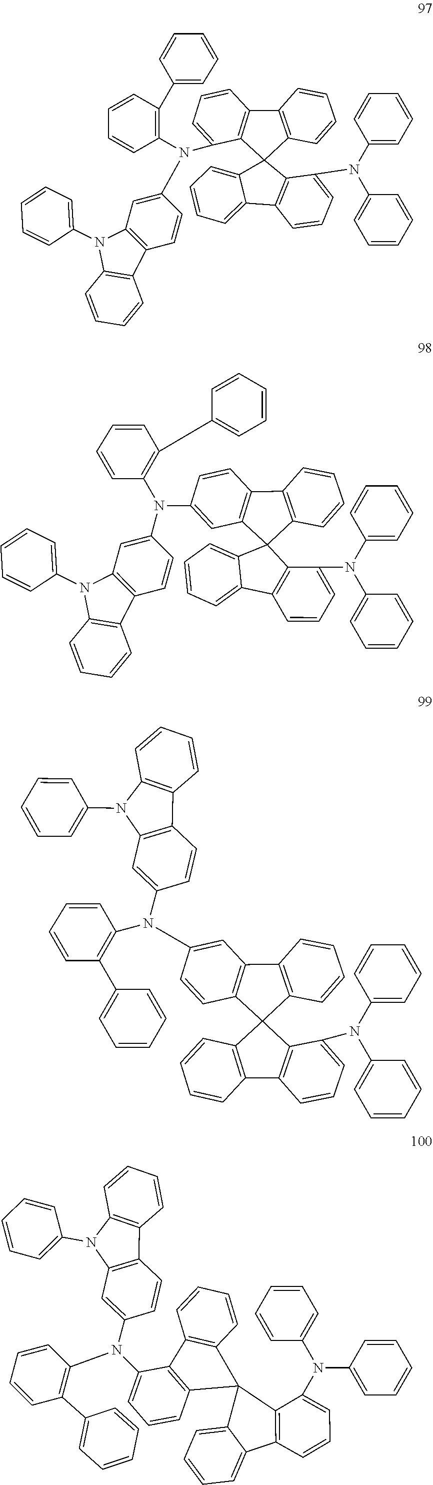 [SMILES: c1ccc(-c2ccccc2N(c2ccc3c4ccccc4n(-c4ccccc4)c3c2)c2cccc3c2C2(c4ccccc4-c4cccc(N(c5ccccc5)c5ccccc5)c42)c2ccccc2-3)cc1 ; c1ccc(-c2ccccc2N(c2ccc3c(c2)C2(c4ccccc4-3)c3ccccc3-c3cccc(N(c4ccccc4)c4ccccc4)c32)c2ccc3c4ccccc4n(-c4ccccc4)c3c2)cc1 ; c1ccc(-c2ccccc2N(c2ccc3c(c2)-c2ccccc2C32c3ccccc3-c3cccc(N(c4ccccc4)c4ccccc4)c32)c2ccc3c4ccccc4n(-c4ccccc4)c3c2)cc1 ; c1ccc(-c2ccccc2N(c2ccc3c4ccccc4n(-c4ccccc4)c3c2)c2cccc3c2-c2ccccc2C32c3ccccc3-c3cccc(N(c4ccccc4)c4ccccc4)c32)cc1]

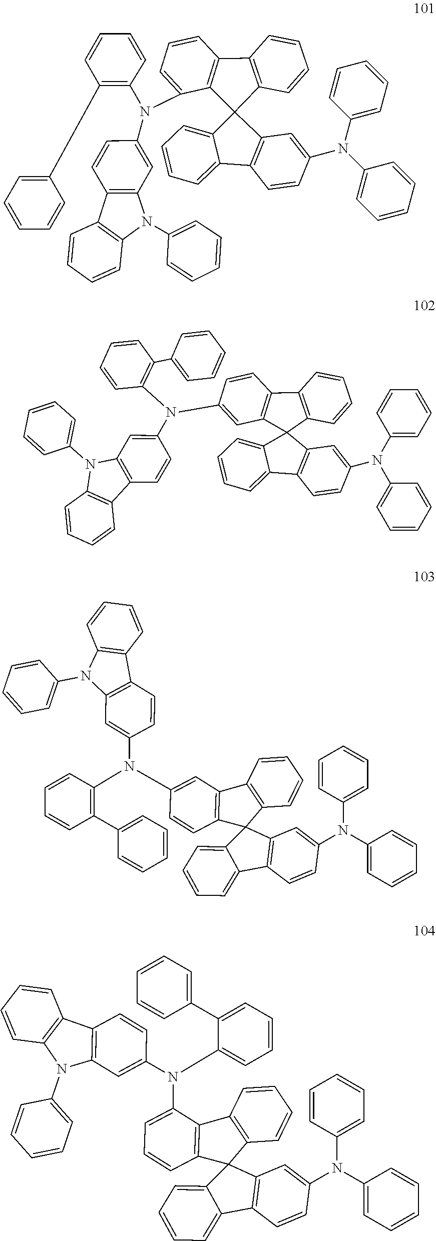 [SMILES: c1ccc(-c2ccccc2N(c2ccc3c4ccccc4n(-c4ccccc4)c3c2)c2cccc3c2C2(c4ccccc4-c4ccc(N(c5ccccc5)c5ccccc5)cc42)c2ccccc2-3)cc1 ; c1ccc(-c2ccccc2N(c2ccc3c(c2)C2(c4ccccc4-c4ccc(N(c5ccccc5)c5ccccc5)cc42)c2ccccc2-3)c2ccc3c4ccccc4n(-c4ccccc4)c3c2)cc1 ; c1ccc(-c2ccccc2N(c2ccc3c(c2)-c2ccccc2C32c3ccccc3-c3ccc(N(c4ccccc4)c4ccccc4)cc32)c2ccc3c4ccccc4n(-c4ccccc4)c3c2)cc1 ; c1ccc(-c2ccccc2N(c2ccc3c4ccccc4n(-c4ccccc4)c3c2)c2cccc3c2-c2ccccc2C32c3ccccc3-c3ccc(N(c4ccccc4)c4ccccc4)cc32)cc1]

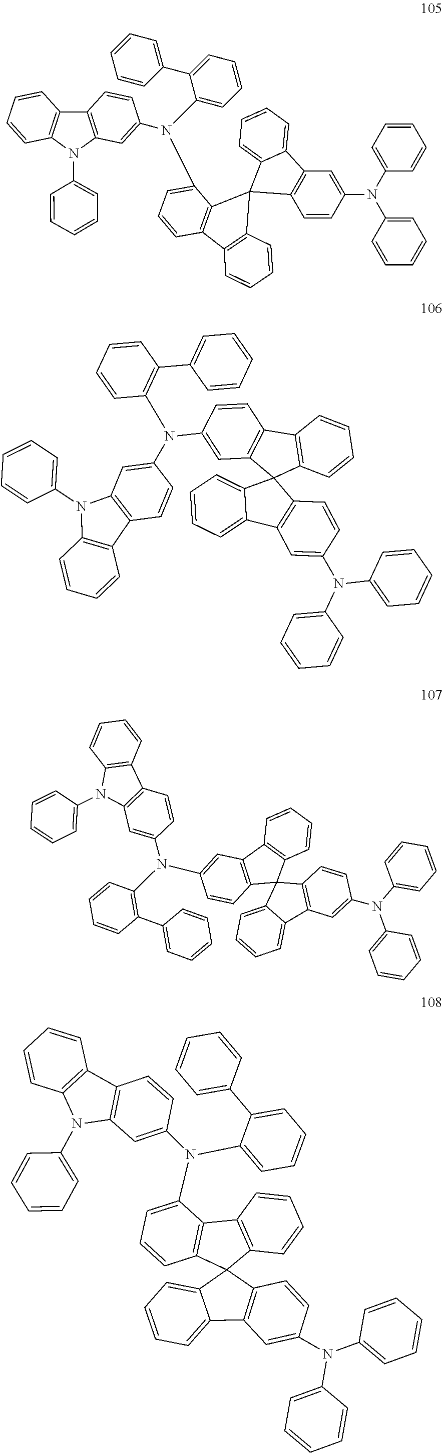 [SMILES: c1ccc(-c2ccccc2N(c2ccc3c4ccccc4n(-c4ccccc4)c3c2)c2cccc3c2C2(c4ccccc4-c4cc(N(c5ccccc5)c5ccccc5)ccc42)c2ccccc2-3)cc1 ; c1ccc(-c2ccccc2N(c2ccc3c(c2)C2(c4ccccc4-c4cc(N(c5ccccc5)c5ccccc5)ccc42)c2ccccc2-3)c2ccc3c4ccccc4n(-c4ccccc4)c3c2)cc1 ; c1ccc(-c2ccccc2N(c2ccc3c(c2)-c2ccccc2C32c3ccccc3-c3cc(N(c4ccccc4)c4ccccc4)ccc32)c2ccc3c4ccccc4n(-c4ccccc4)c3c2)cc1 ; c1ccc(-c2ccccc2N(c2ccc3c4ccccc4n(-c4ccccc4)c3c2)c2cccc3c2-c2ccccc2C32c3ccccc3-c3cc(N(c4ccccc4)c4ccccc4)ccc32)cc1]

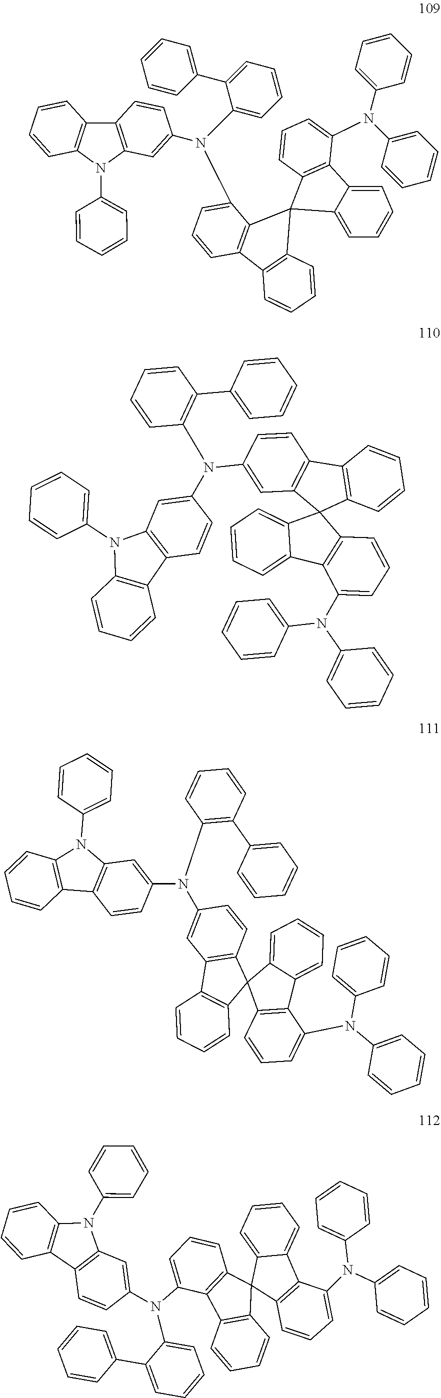 [SMILES: c1ccc(-c2ccccc2N(c2ccc3c4ccccc4n(-c4ccccc4)c3c2)c2cccc3c2C2(c4ccccc4-3)c3ccccc3-c3c(N(c4ccccc4)c4ccccc4)cccc32)cc1 ; c1ccc(-c2ccccc2N(c2ccc3c(c2)C2(c4ccccc4-3)c3ccccc3-c3c(N(c4ccccc4)c4ccccc4)cccc32)c2ccc3c4ccccc4n(-c4ccccc4)c3c2)cc1 ; c1ccc(-c2ccccc2N(c2ccc3c(c2)-c2ccccc2C32c3ccccc3-c3c(N(c4ccccc4)c4ccccc4)cccc32)c2ccc3c4ccccc4n(-c4ccccc4)c3c2)cc1 ; c1ccc(-c2ccccc2N(c2ccc3c4ccccc4n(-c4ccccc4)c3c2)c2cccc3c2-c2ccccc2C32c3ccccc3-c3c(N(c4ccccc4)c4ccccc4)cccc32)cc1]

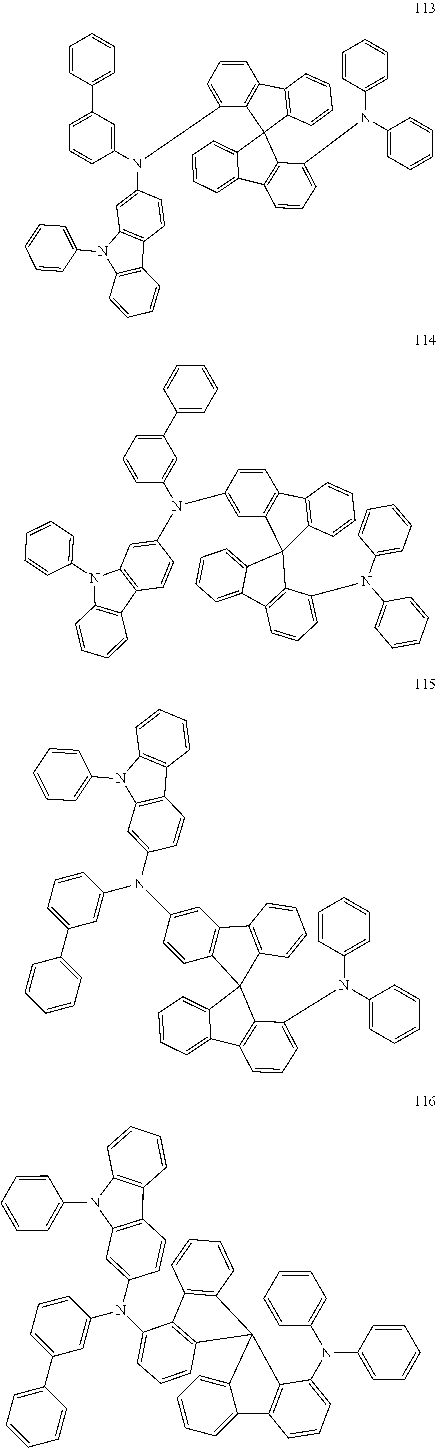 [SMILES: c1ccc(-c2cccc(N(c3ccc4c5ccccc5n(-c5ccccc5)c4c3)c3cccc4c3C3(c5ccccc5-c5cccc(N(c6ccccc6)c6ccccc6)c53)c3ccccc3-4)c2)cc1 ; c1ccc(-c2cccc(N(c3ccc4c(c3)C3(c5ccccc5-4)c4ccccc4-c4cccc(N(c5ccccc5)c5ccccc5)c43)c3ccc4c5ccccc5n(-c5ccccc5)c4c3)c2)cc1 ; c1ccc(-c2cccc(N(c3ccc4c(c3)-c3ccccc3C43c4ccccc4-c4cccc(N(c5ccccc5)c5ccccc5)c43)c3ccc4c5ccccc5n(-c5ccccc5)c4c3)c2)cc1 ; c1ccc(-c2cccc(N(c3ccc4c5ccccc5n(-c5ccccc5)c4c3)c3cccc4c3-c3ccccc3C43c4ccccc4-c4cccc(N(c5ccccc5)c5ccccc5)c43)c2)cc1]

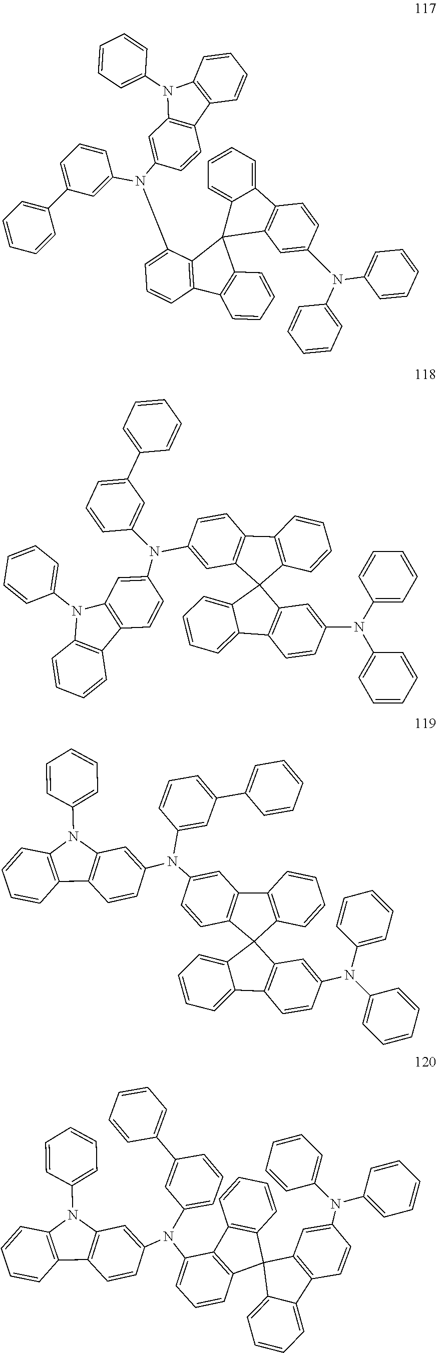 [SMILES: c1ccc(-c2cccc(N(c3ccc4c5ccccc5n(-c5ccccc5)c4c3)c3cccc4c3C3(c5ccccc5-c5ccc(N(c6ccccc6)c6ccccc6)cc53)c3ccccc3-4)c2)cc1 ; c1ccc(-c2cccc(N(c3ccc4c(c3)C3(c5ccccc5-c5ccc(N(c6ccccc6)c6ccccc6)cc53)c3ccccc3-4)c3ccc4c5ccccc5n(-c5ccccc5)c4c3)c2)cc1 ; c1ccc(-c2cccc(N(c3ccc4c(c3)-c3ccccc3C43c4ccccc4-c4ccc(N(c5ccccc5)c5ccccc5)cc43)c3ccc4c5ccccc5n(-c5ccccc5)c4c3)c2)cc1 ; c1ccc(-c2cccc(N(c3ccc4c5ccccc5n(-c5ccccc5)c4c3)c3cccc4c3-c3ccccc3C43c4ccccc4-c4ccc(N(c5ccccc5)c5ccccc5)cc43)c2)cc1]

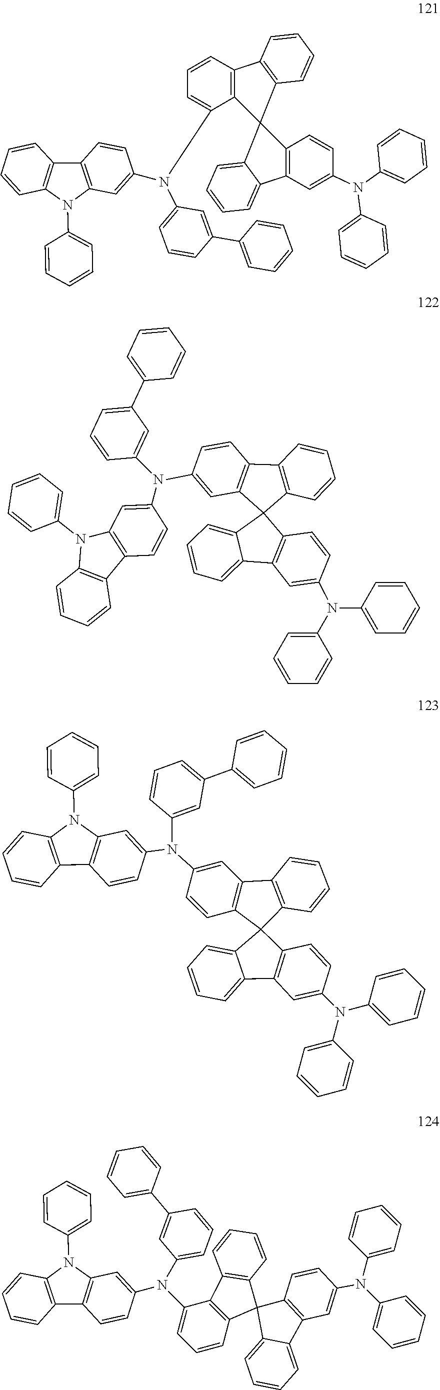 [SMILES: c1ccc(-c2cccc(N(c3ccc4c5ccccc5n(-c5ccccc5)c4c3)c3cccc4c3C3(c5ccccc5-c5cc(N(c6ccccc6)c6ccccc6)ccc53)c3ccccc3-4)c2)cc1 ; c1ccc(-c2cccc(N(c3ccc4c(c3)C3(c5ccccc5-c5cc(N(c6ccccc6)c6ccccc6)ccc53)c3ccccc3-4)c3ccc4c5ccccc5n(-c5ccccc5)c4c3)c2)cc1 ; c1ccc(-c2cccc(N(c3ccc4c(c3)-c3ccccc3C43c4ccccc4-c4cc(N(c5ccccc5)c5ccccc5)ccc43)c3ccc4c5ccccc5n(-c5ccccc5)c4c3)c2)cc1 ; c1ccc(-c2cccc(N(c3ccc4c5ccccc5n(-c5ccccc5)c4c3)c3cccc4c3-c3ccccc3C43c4ccccc4-c4cc(N(c5ccccc5)c5ccccc5)ccc43)c2)cc1]

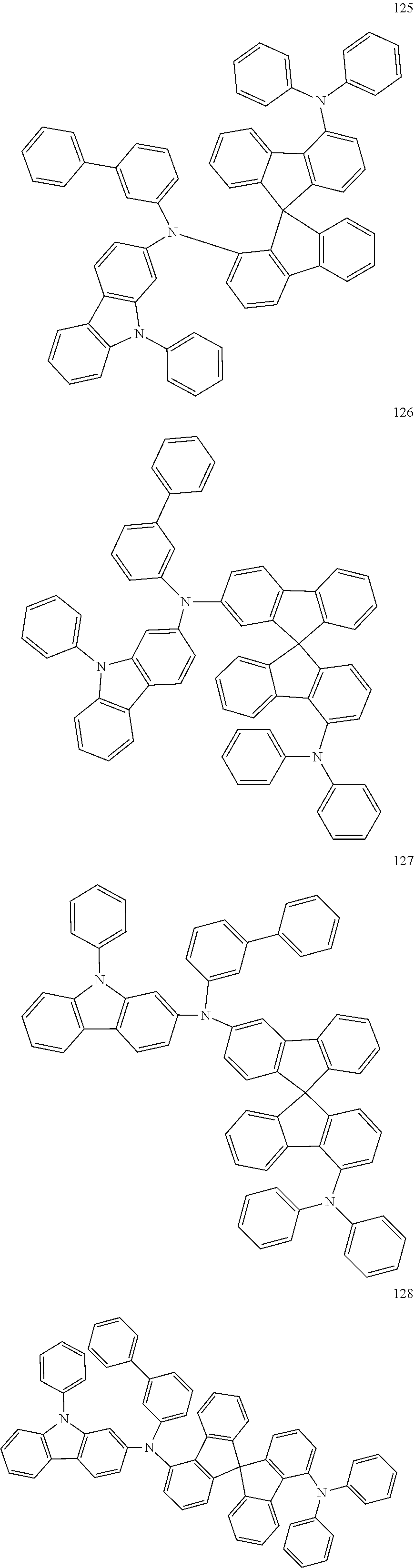

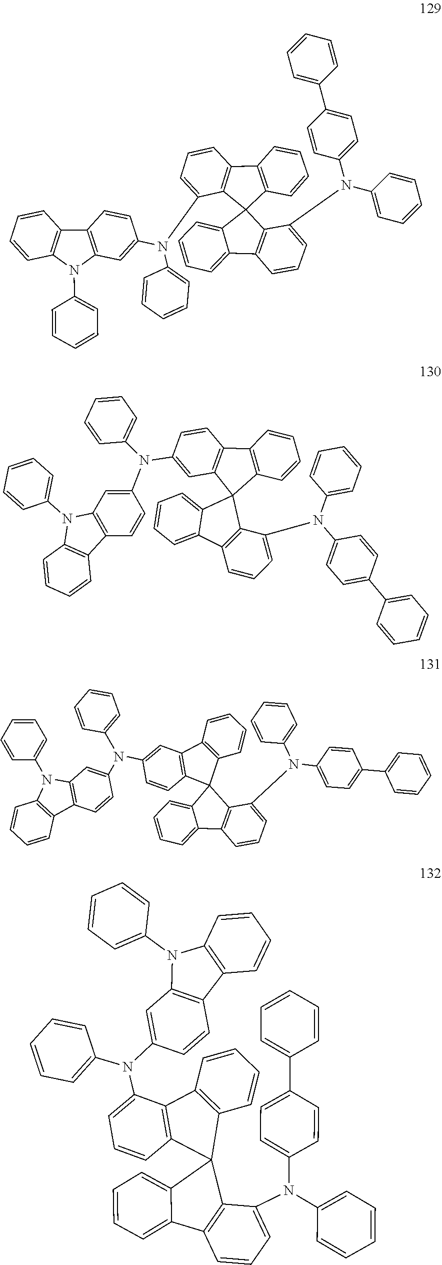 [SMILES: c1ccc(-c2ccc(N(c3ccccc3)c3cccc4c3C3(c5ccccc5-4)c4ccccc4-c4cccc(N(c5ccccc5)c5ccc6c7ccccc7n(-c7ccccc7)c6c5)c43)cc2)cc1 ; c1ccc(-c2ccc(N(c3ccccc3)c3cccc4c3C3(c5ccccc5-c5ccc(N(c6ccccc6)c6ccc7c8ccccc8n(-c8ccccc8)c7c6)cc53)c3ccccc3-4)cc2)cc1 ; c1ccc(-c2ccc(N(c3ccccc3)c3cccc4c3C3(c5ccccc5-c5cc(N(c6ccccc6)c6ccc7c8ccccc8n(-c8ccccc8)c7c6)ccc53)c3ccccc3-4)cc2)cc1 ; c1ccc(-c2ccc(N(c3ccccc3)c3cccc4c3C3(c5ccccc5-4)c4ccccc4-c4c(N(c5ccccc5)c5ccc6c7ccccc7n(-c7ccccc7)c6c5)cccc43)cc2)cc1]

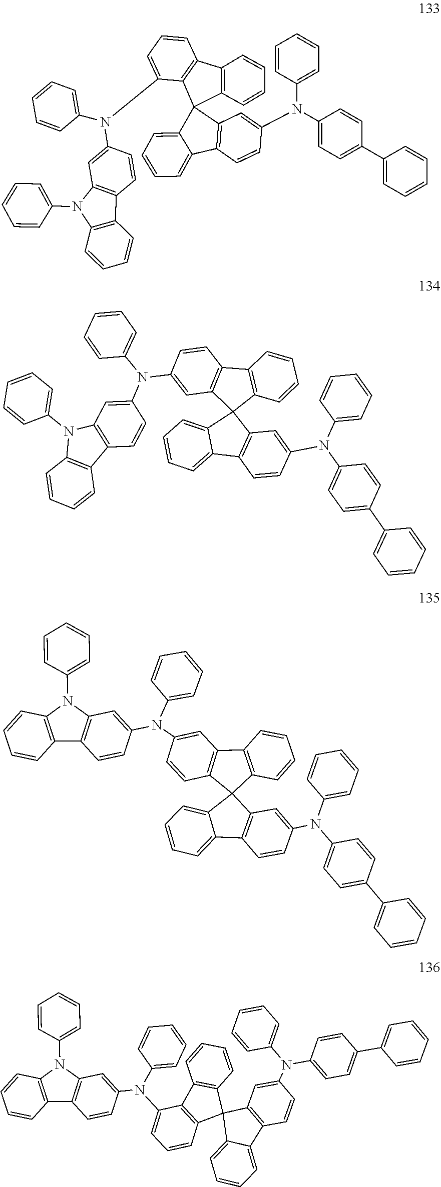 [SMILES: c1ccc(-c2ccc(N(c3ccccc3)c3ccc4c(c3)C3(c5ccccc5-4)c4ccccc4-c4cccc(N(c5ccccc5)c5ccc6c7ccccc7n(-c7ccccc7)c6c5)c43)cc2)cc1 ; c1ccc(-c2ccc(N(c3ccccc3)c3ccc4c(c3)C3(c5ccccc5-4)c4ccccc4-c4ccc(N(c5ccccc5)c5ccc6c7ccccc7n(-c7ccccc7)c6c5)cc43)cc2)cc1 ; c1ccc(-c2ccc(N(c3ccccc3)c3ccc4c(c3)C3(c5ccccc5-c5cc(N(c6ccccc6)c6ccc7c8ccccc8n(-c8ccccc8)c7c6)ccc53)c3ccccc3-4)cc2)cc1 ; c1ccc(-c2ccc(N(c3ccccc3)c3ccc4c(c3)C3(c5ccccc5-4)c4ccccc4-c4c(N(c5ccccc5)c5ccc6c7ccccc7n(-c7ccccc7)c6c5)cccc43)cc2)cc1]

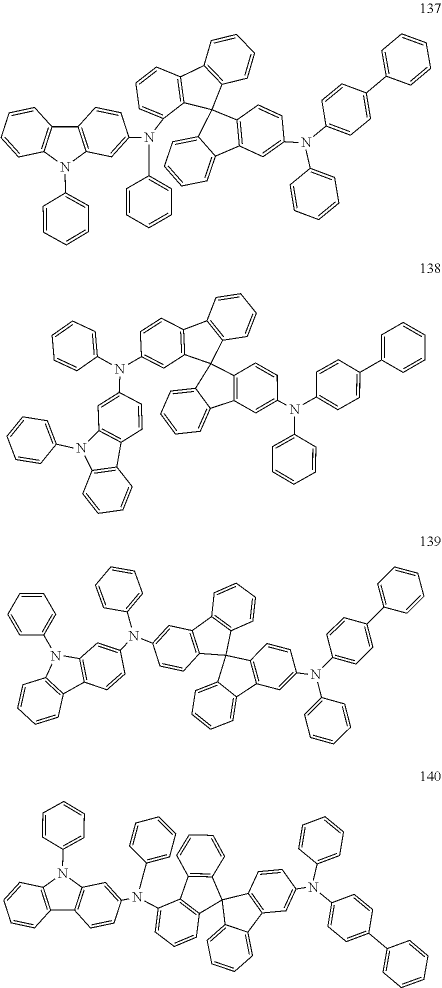 [SMILES: c1ccc(-c2ccc(N(c3ccccc3)c3ccc4c(c3)-c3ccccc3C43c4ccccc4-c4cccc(N(c5ccccc5)c5ccc6c7ccccc7n(-c7ccccc7)c6c5)c43)cc2)cc1 ; c1ccc(-c2ccc(N(c3ccccc3)c3ccc4c(c3)-c3ccccc3C43c4ccccc4-c4ccc(N(c5ccccc5)c5ccc6c7ccccc7n(-c7ccccc7)c6c5)cc43)cc2)cc1 ; c1ccc(-c2ccc(N(c3ccccc3)c3ccc4c(c3)-c3ccccc3C43c4ccccc4-c4cc(N(c5ccccc5)c5ccc6c7ccccc7n(-c7ccccc7)c6c5)ccc43)cc2)cc1 ; c1ccc(-c2ccc(N(c3ccccc3)c3ccc4c(c3)-c3ccccc3C43c4ccccc4-c4c(N(c5ccccc5)c5ccc6c7ccccc7n(-c7ccccc7)c6c5)cccc43)cc2)cc1]

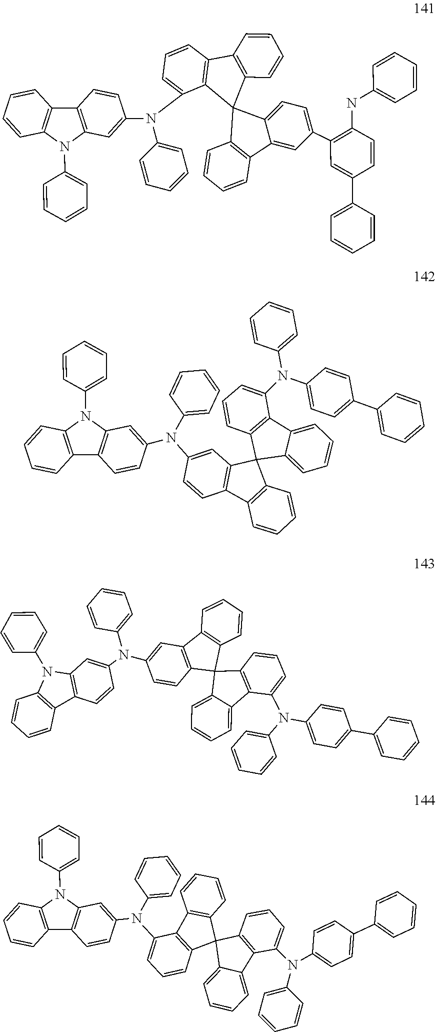 [SMILES: c1ccc(Nc2ccc(-c3ccccc3)cc2-c2ccc3c(c2)-c2ccccc2C32c3ccccc3-c3cccc(N(c4ccccc4)c4ccc5c6ccccc6n(-c6ccccc6)c5c4)c32)cc1 ; c1ccc(-c2ccc(N(c3ccccc3)c3cccc4c3-c3ccccc3C43c4ccccc4-c4ccc(N(c5ccccc5)c5ccc6c7ccccc7n(-c7ccccc7)c6c5)cc43)cc2)cc1 ; c1ccc(-c2ccc(N(c3ccccc3)c3cccc4c3-c3ccccc3C43c4ccccc4-c4cc(N(c5ccccc5)c5ccc6c7ccccc7n(-c7ccccc7)c6c5)ccc43)cc2)cc1 ; c1ccc(-c2ccc(N(c3ccccc3)c3cccc4c3-c3ccccc3C43c4ccccc4-c4c(N(c5ccccc5)c5ccc6c7ccccc7n(-c7ccccc7)c6c5)cccc43)cc2)cc1]

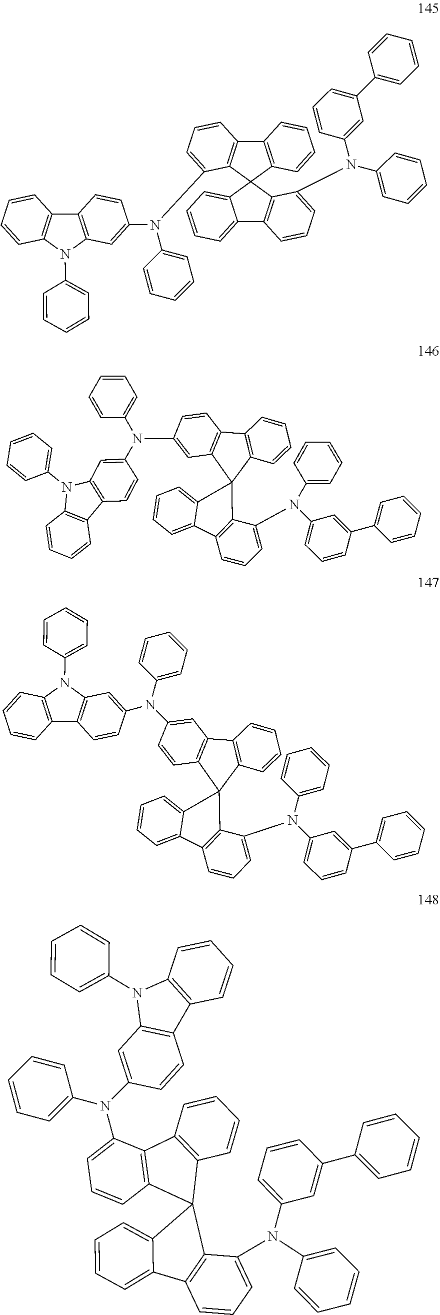 [SMILES: c1ccc(-c2cccc(N(c3ccccc3)c3cccc4c3C3(c5ccccc5-4)c4ccccc4-c4cccc(N(c5ccccc5)c5ccc6c7ccccc7n(-c7ccccc7)c6c5)c43)c2)cc1 ; c1ccc(-c2cccc(N(c3ccccc3)c3cccc4c3C3(c5ccccc5-c5ccc(N(c6ccccc6)c6ccc7c8ccccc8n(-c8ccccc8)c7c6)cc53)c3ccccc3-4)c2)cc1 ; c1ccc(-c2cccc(N(c3ccccc3)c3cccc4c3C3(c5ccccc5-c5cc(N(c6ccccc6)c6ccc7c8ccccc8n(-c8ccccc8)c7c6)ccc53)c3ccccc3-4)c2)cc1 ; c1ccc(-c2cccc(N(c3ccccc3)c3cccc4c3C3(c5ccccc5-4)c4ccccc4-c4c(N(c5ccccc5)c5ccc6c7ccccc7n(-c7ccccc7)c6c5)cccc43)c2)cc1]

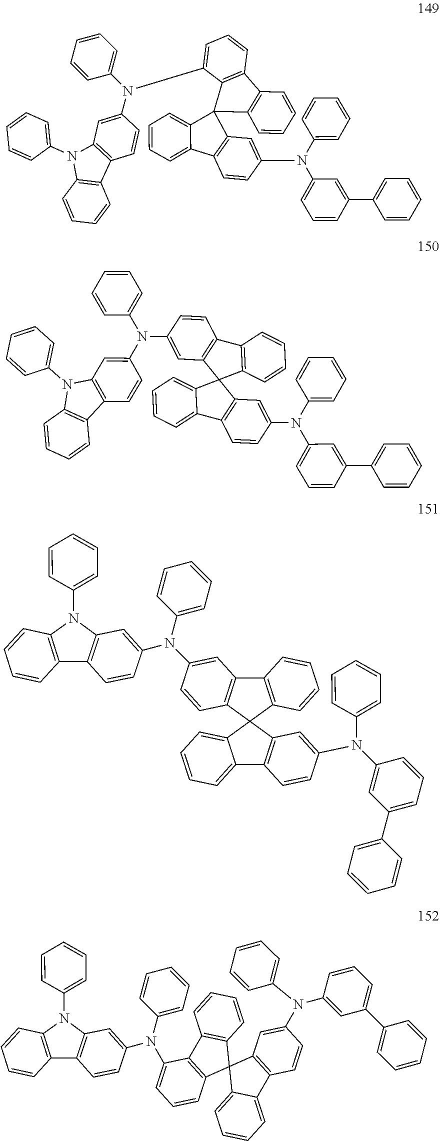 [SMILES: c1ccc(-c2cccc(N(c3ccccc3)c3ccc4c(c3)C3(c5ccccc5-4)c4ccccc4-c4cccc(N(c5ccccc5)c5ccc6c7ccccc7n(-c7ccccc7)c6c5)c43)c2)cc1 ; c1ccc(-c2cccc(N(c3ccccc3)c3ccc4c(c3)C3(c5ccccc5-4)c4ccccc4-c4ccc(N(c5ccccc5)c5ccc6c7ccccc7n(-c7ccccc7)c6c5)cc43)c2)cc1 ; c1ccc(-c2cccc(N(c3ccccc3)c3ccc4c(c3)C3(c5ccccc5-c5cc(N(c6ccccc6)c6ccc7c8ccccc8n(-c8ccccc8)c7c6)ccc53)c3ccccc3-4)c2)cc1 ; c1ccc(-c2cccc(N(c3ccccc3)c3ccc4c(c3)C3(c5ccccc5-4)c4ccccc4-c4c(N(c5ccccc5)c5ccc6c7ccccc7n(-c7ccccc7)c6c5)cccc43)c2)cc1]

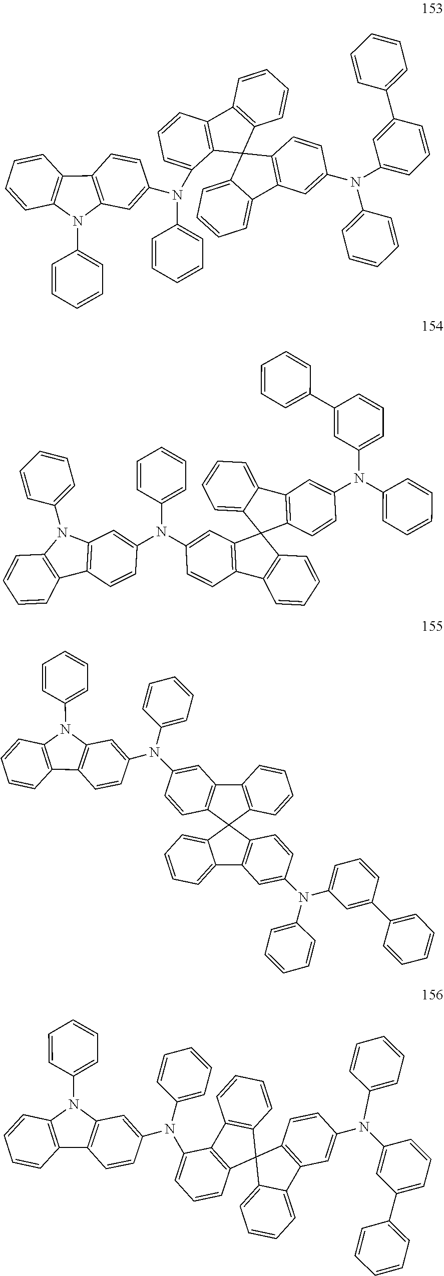 [SMILES: c1ccc(-c2cccc(N(c3ccccc3)c3ccc4c(c3)-c3ccccc3C43c4ccccc4-c4cccc(N(c5ccccc5)c5ccc6c7ccccc7n(-c7ccccc7)c6c5)c43)c2)cc1 ; c1ccc(-c2cccc(N(c3ccccc3)c3ccc4c(c3)-c3ccccc3C43c4ccccc4-c4ccc(N(c5ccccc5)c5ccc6c7ccccc7n(-c7ccccc7)c6c5)cc43)c2)cc1 ; c1ccc(-c2cccc(N(c3ccccc3)c3ccc4c(c3)-c3ccccc3C43c4ccccc4-c4cc(N(c5ccccc5)c5ccc6c7ccccc7n(-c7ccccc7)c6c5)ccc43)c2)cc1 ; c1ccc(-c2cccc(N(c3ccccc3)c3ccc4c(c3)-c3ccccc3C43c4ccccc4-c4c(N(c5ccccc5)c5ccc6c7ccccc7n(-c7ccccc7)c6c5)cccc43)c2)cc1]

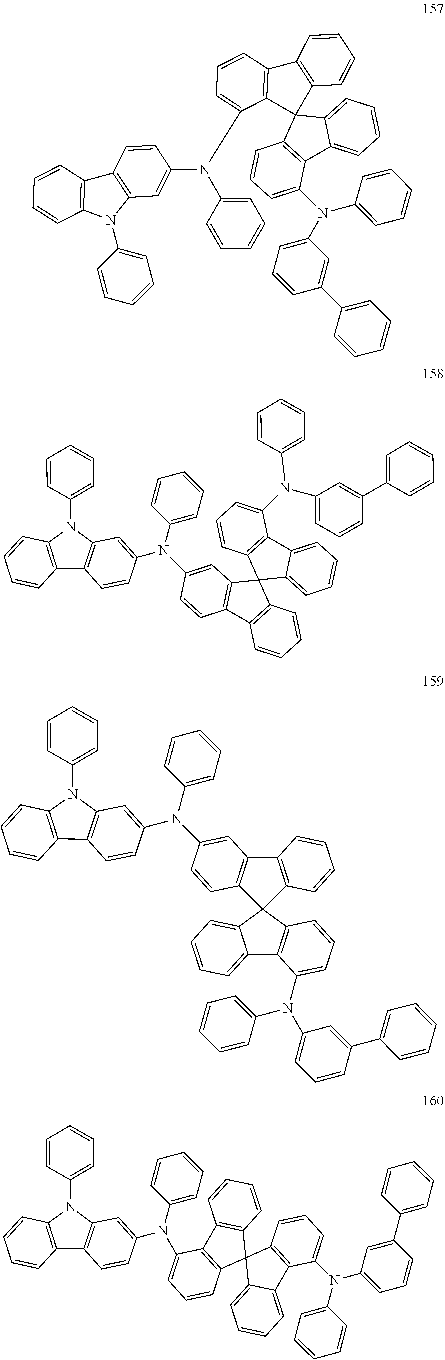 [SMILES: c1ccc(-c2cccc(N(c3ccccc3)c3cccc4c3-c3ccccc3C43c4ccccc4-c4cccc(N(c5ccccc5)c5ccc6c7ccccc7n(-c7ccccc7)c6c5)c43)c2)cc1 ; c1ccc(-c2cccc(N(c3ccccc3)c3cccc4c3-c3ccccc3C43c4ccccc4-c4ccc(N(c5ccccc5)c5ccc6c7ccccc7n(-c7ccccc7)c6c5)cc43)c2)cc1 ; c1ccc(-c2cccc(N(c3ccccc3)c3cccc4c3-c3ccccc3C43c4ccccc4-c4cc(N(c5ccccc5)c5ccc6c7ccccc7n(-c7ccccc7)c6c5)ccc43)c2)cc1 ; c1ccc(-c2cccc(N(c3ccccc3)c3cccc4c3-c3ccccc3C43c4ccccc4-c4c(N(c5ccccc5)c5ccc6c7ccccc7n(-c7ccccc7)c6c5)cccc43)c2)cc1]

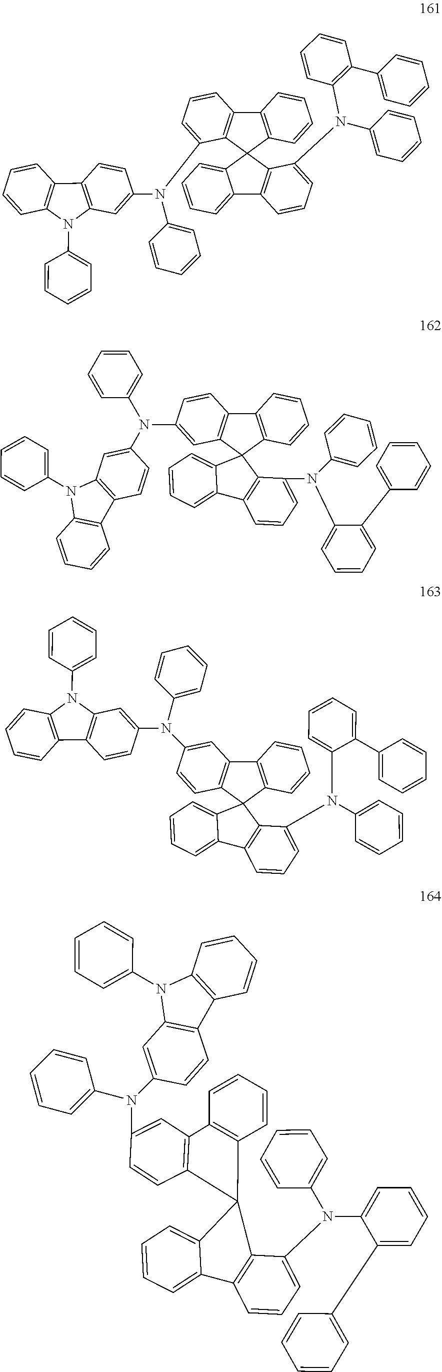 [SMILES: c1ccc(-c2ccccc2N(c2ccccc2)c2cccc3c2C2(c4ccccc4-c4cccc(N(c5ccccc5)c5ccc6c7ccccc7n(-c7ccccc7)c6c5)c42)c2ccccc2-3)cc1 ; c1ccc(-c2ccccc2N(c2ccccc2)c2cccc3c2C2(c4ccccc4-c4ccc(N(c5ccccc5)c5ccc6c7ccccc7n(-c7ccccc7)c6c5)cc42)c2ccccc2-3)cc1 ; c1ccc(-c2ccccc2N(c2ccccc2)c2cccc3c2C2(c4ccccc4-c4cc(N(c5ccccc5)c5ccc6c7ccccc7n(-c7ccccc7)c6c5)ccc42)c2ccccc2-3)cc1 ; c1ccc(-c2ccccc2N(c2ccccc2)c2cccc3c2C2(c4ccccc4-c4cc(N(c5ccccc5)c5ccc6c7ccccc7n(-c7ccccc7)c6c5)ccc42)c2ccccc2-3)cc1]

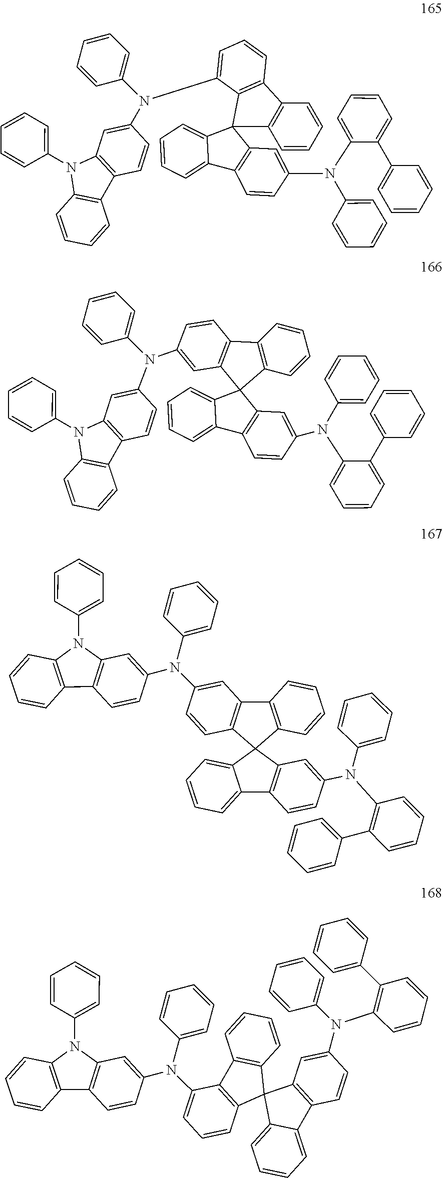 [SMILES: c1ccc(-c2ccccc2N(c2ccccc2)c2ccc3c(c2)C2(c4ccccc4-3)c3ccccc3-c3cccc(N(c4ccccc4)c4ccc5c6ccccc6n(-c6ccccc6)c5c4)c32)cc1 ; c1ccc(-c2ccccc2N(c2ccccc2)c2ccc3c(c2)C2(c4ccccc4-c4ccc(N(c5ccccc5)c5ccc6c7ccccc7n(-c7ccccc7)c6c5)cc42)c2ccccc2-3)cc1 ; c1ccc(-c2ccccc2N(c2ccccc2)c2ccc3c(c2)C2(c4ccccc4-c4cc(N(c5ccccc5)c5ccc6c7ccccc7n(-c7ccccc7)c6c5)ccc42)c2ccccc2-3)cc1 ; c1ccc(-c2ccccc2N(c2ccccc2)c2ccc3c(c2)C2(c4ccccc4-3)c3ccccc3-c3c(N(c4ccccc4)c4ccc5c6ccccc6n(-c6ccccc6)c5c4)cccc32)cc1]

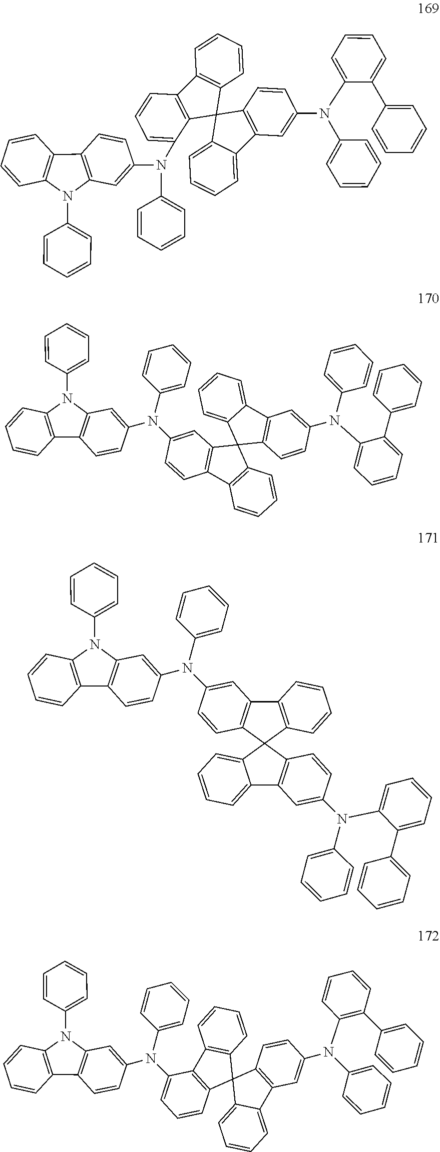 [SMILES: c1ccc(-c2ccccc2N(c2ccccc2)c2ccc3c(c2)-c2ccccc2C32c3ccccc3-c3cccc(N(c4ccccc4)c4ccc5c6ccccc6n(-c6ccccc6)c5c4)c32)cc1 ; c1ccc(-c2ccccc2N(c2ccccc2)c2ccc3c(c2)-c2ccccc2C32c3ccccc3-c3ccc(N(c4ccccc4)c4ccc5c6ccccc6n(-c6ccccc6)c5c4)cc32)cc1 ; c1ccc(-c2ccccc2N(c2ccccc2)c2ccc3c(c2)-c2ccccc2C32c3ccccc3-c3cc(N(c4ccccc4)c4ccc5c6ccccc6n(-c6ccccc6)c5c4)ccc32)cc1 ; c1ccc(-c2ccccc2N(c2ccccc2)c2ccc3c(c2)-c2ccccc2C32c3ccccc3-c3c(N(c4ccccc4)c4ccc5c6ccccc6n(-c6ccccc6)c5c4)cccc32)cc1]

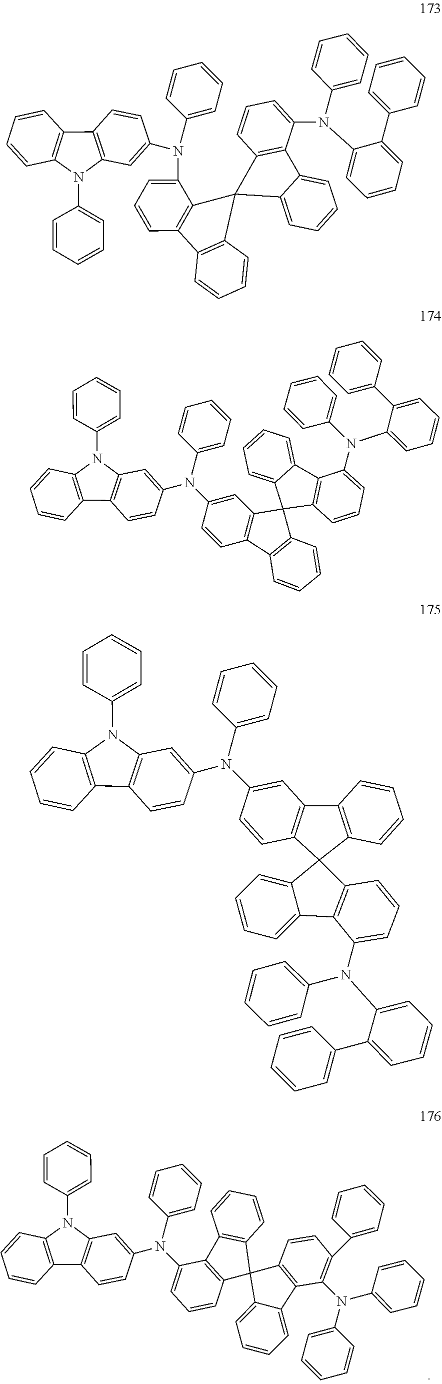 [SMILES: c1ccc(-c2ccccc2N(c2ccccc2)c2cccc3c2-c2ccccc2C32c3ccccc3-c3cccc(N(c4ccccc4)c4ccc5c6ccccc6n(-c6ccccc6)c5c4)c32)cc1 ; c1ccc(-c2ccccc2N(c2ccccc2)c2cccc3c2-c2ccccc2C32c3ccccc3-c3ccc(N(c4ccccc4)c4ccc5c6ccccc6n(-c6ccccc6)c5c4)cc32)cc1 ; c1ccc(-c2ccccc2N(c2ccccc2)c2cccc3c2-c2ccccc2C32c3ccccc3-c3cc(N(c4ccccc4)c4ccc5c6ccccc6n(-c6ccccc6)c5c4)ccc32)cc1 ; c1ccc(-c2ccc3c(c2N(c2ccccc2)c2ccccc2)-c2ccccc2C32c3ccccc3-c3c(N(c4ccccc4)c4ccc5c6ccccc6n(-c6ccccc6)c5c4)cccc32)cc1]

13. An amine compound represented by Formula 1: 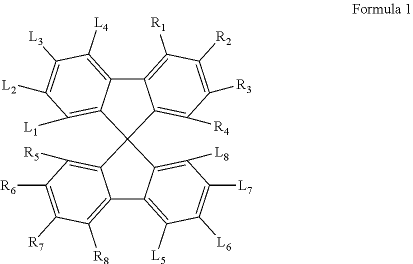
wherein, in Formula 1,
R₁ to R₈ are each independently a hydrogen atom, a deuterium atom, a halogen atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms,
L₁ to L₈ are each independently a hydrogen atom, a deuterium atom, a halogen atom, a substituted or unsubstituted amine group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms,
at least one among L₁ to L₄ is a group represented by Formula 2, and
at least one among L₅ to L₈ is a group represented by Formula 3: 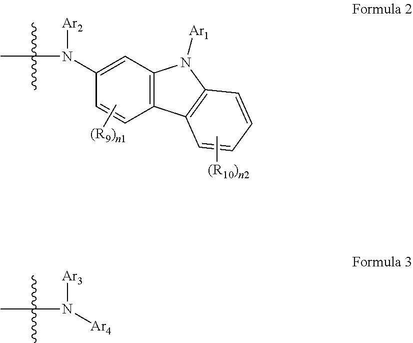
wherein, in Formula 2 and Formula 3,
R₉ and R₁₀ are each independently a hydrogen atom, a deuterium atom, a halogen atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms,
Ar₁ to Ar₄ are each independently a substituted or unsubstituted aryl group having 6 to 30 ring-forming carbon atoms, or a substituted or unsubstituted heteroaryl group having 2 to 30 ring-forming carbon atoms,
n1 is an integer of 0 to 3, and
n2 is an integer of 0 to 4.

14. The amine compound of claim 13, wherein the amine compound represented by Formula 1 is a diamine compound.

15. The amine compound of claim 13, wherein the amine compound represented by Formula 1 is represented by any one among Formula 4-1 to Formula 4-4: 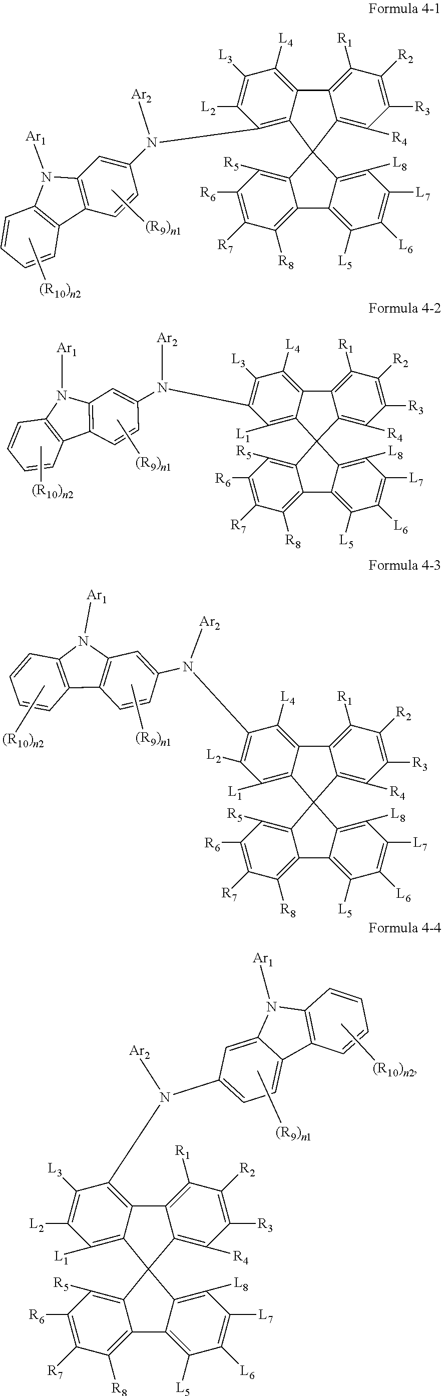
wherein, in Formula 4-1 to Formula 4-4,
R₁ to R₁₀, L₁ to L₈, Ar₁, Ar₂, n1 and n2 are the same as respectively defined in connection with Formula 1 and Formula 2.

16. The amine compound of claim 13, wherein the amine compound represented by Formula 1 is represented by any one among Formula 5-1 to Formula 5-4: 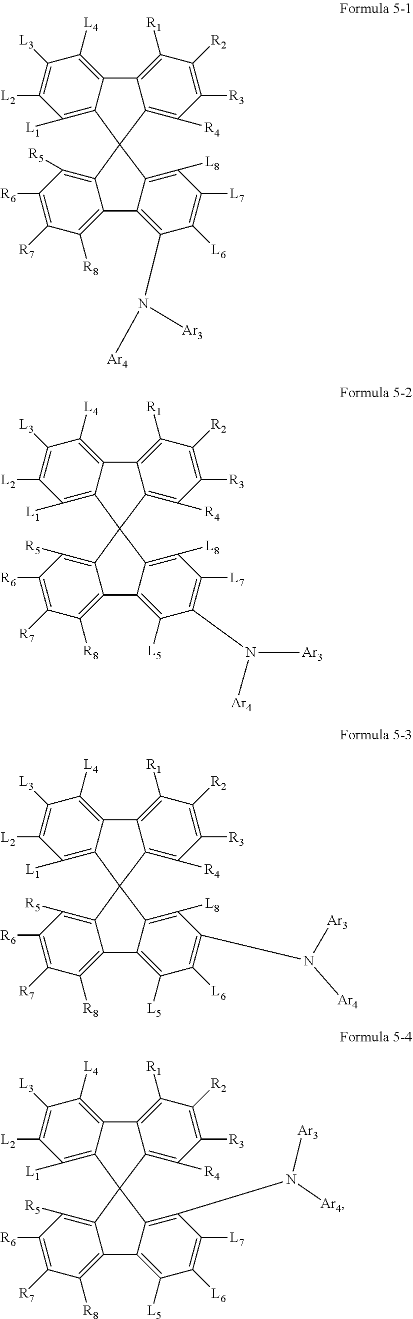
wherein, in Formula 5-1 to Formula 5-4,
R₁ to R₈, L₁ to L₈, Ar₃, and Ar₄ are the same as respectively defined in connection with Formula 1 and Formula 3.

17. The amine compound of claim 13, wherein Ar₁ to Ar₄ are each independently a substituted or unsubstituted phenyl group, a substituted or unsubstituted naphthyl group, or a substituted or unsubstituted biphenyl group.

18. The amine compound of claim 13, wherein the group represented by Formula 2 is represented by Formula 2-1: 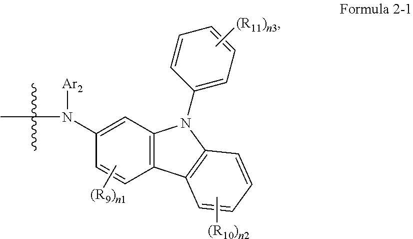
wherein, in Formula 2-1,
R₁₁ is a hydrogen atom, a deuterium atom, a halogen atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms,
n3 is an integer of 0 to 5, and
R₉, R₁₀, Ar₂, n1, and n2 are the same as respectively defined in connection with Formula 2.

19. The amine compound of claim 13, wherein Ar₁ to Ar₄ are each independently represented by any one among Formula 6-1 to Formula 6-6: 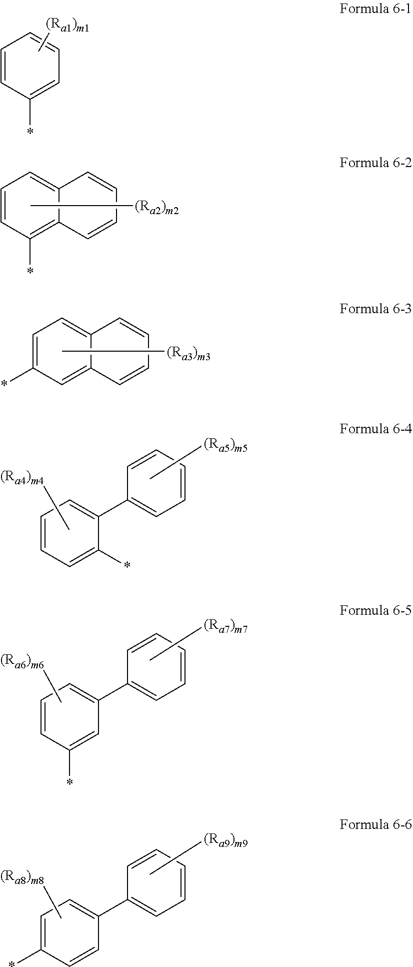
wherein, in Formula 6-1 to Formula 6-6,
Rₐ₁ to Rₐ₉ are each independently a hydrogen atom, a deuterium atom, a halogen atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms,
m1, m5, m7 and m9 are each independently an integer of 0 to 5,
m2 and m3 are each independently an integer of 0 to 7, and
m4, m6 and m8 are each independently an integer of 0 to 4.

20. The amine compound of claim 13, wherein the amine compound represented by Formula 1 is any one among compounds represented by Compound Group 1: 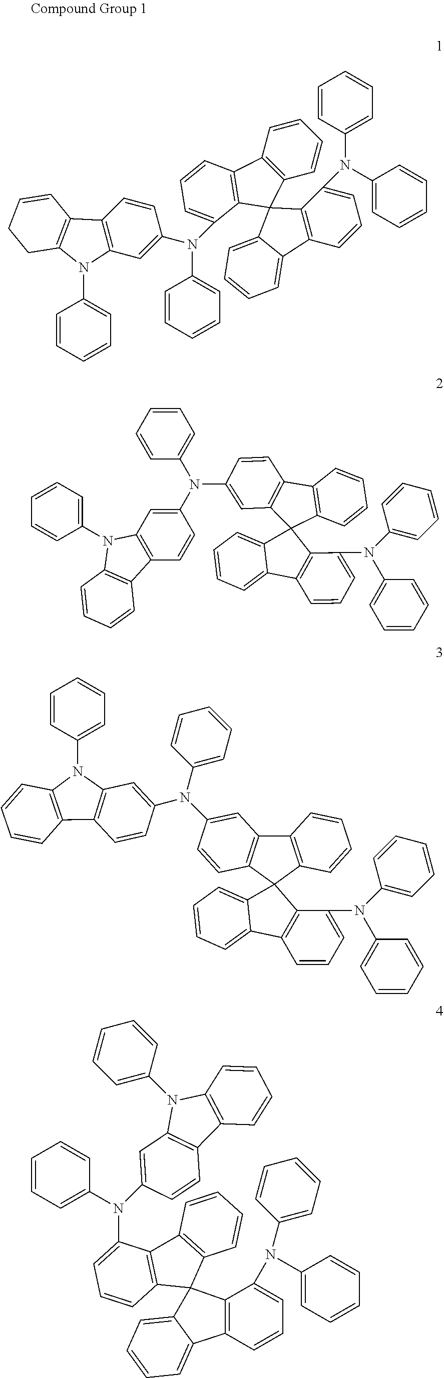 [SMILES: C1=Cc2c(n(-c3ccccc3)c3cc(N(c4ccccc4)c4cccc5c4C4(c6ccccc6-c6cccc(N(c7ccccc7)c7ccccc7)c64)c4ccccc4-5)ccc23)CC1 ; c1ccc(N(c2ccc3c(c2)C2(c4ccccc4-3)c3ccccc3-c3cccc(N(c4ccccc4)c4ccccc4)c32)c2ccc3c4ccccc4n(-c4ccccc4)c3c2)cc1 ; c1ccc(N(c2ccc3c(c2)-c2ccccc2C32c3ccccc3-c3cccc(N(c4ccccc4)c4ccccc4)c32)c2ccc3c4ccccc4n(-c4ccccc4)c3c2)cc1 ; c1ccc(N(c2ccc3c4ccccc4n(-c4ccccc4)c3c2)c2cccc3c2-c2ccccc2C32c3ccccc3-c3cccc(N(c4ccccc4)c4ccccc4)c32)cc1] 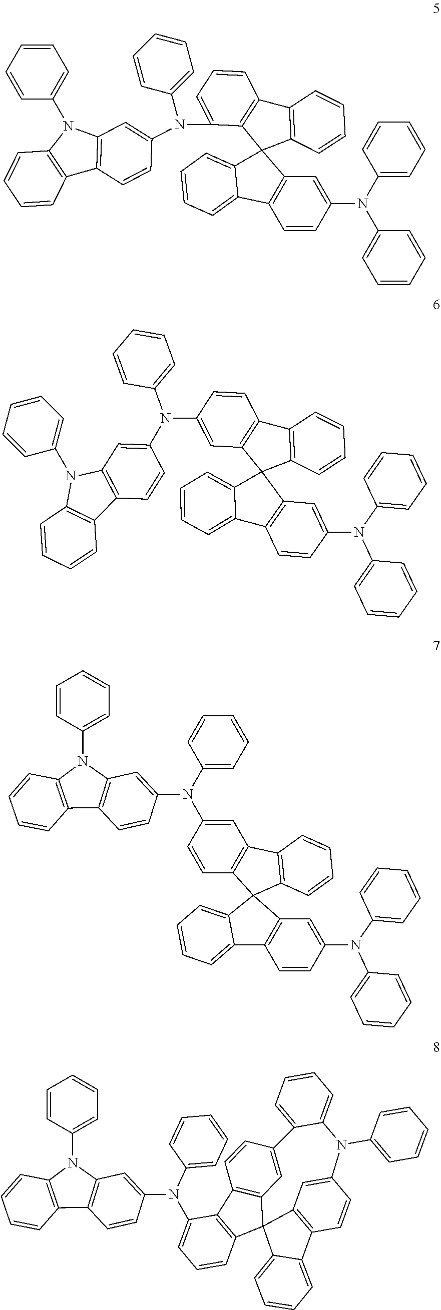 [SMILES: c1ccc(N(c2ccccc2)c2ccc3c(c2)C2(c4ccccc4-3)c3ccccc3-c3cccc(N(c4ccccc4)c4ccc5c6ccccc6n(-c6ccccc6)c5c4)c32)cc1 ; c1ccc(N(c2ccccc2)c2ccc3c(c2)C2(c4ccccc4-3)c3ccccc3-c3ccc(N(c4ccccc4)c4ccc5c6ccccc6n(-c6ccccc6)c5c4)cc32)cc1 ; c1ccc(N(c2ccccc2)c2ccc3c(c2)C2(c4ccccc4-c4cc(N(c5ccccc5)c5ccc6c7ccccc7n(-c7ccccc7)c6c5)ccc42)c2ccccc2-3)cc1 ; c1ccc(N2c3ccc4c(c3)C3(c5ccccc5-4)c4cc(ccc4-c4c(N(c5ccccc5)c5ccc6c7ccccc7n(-c7ccccc7)c6c5)cccc43)-c3ccccc32)cc1] 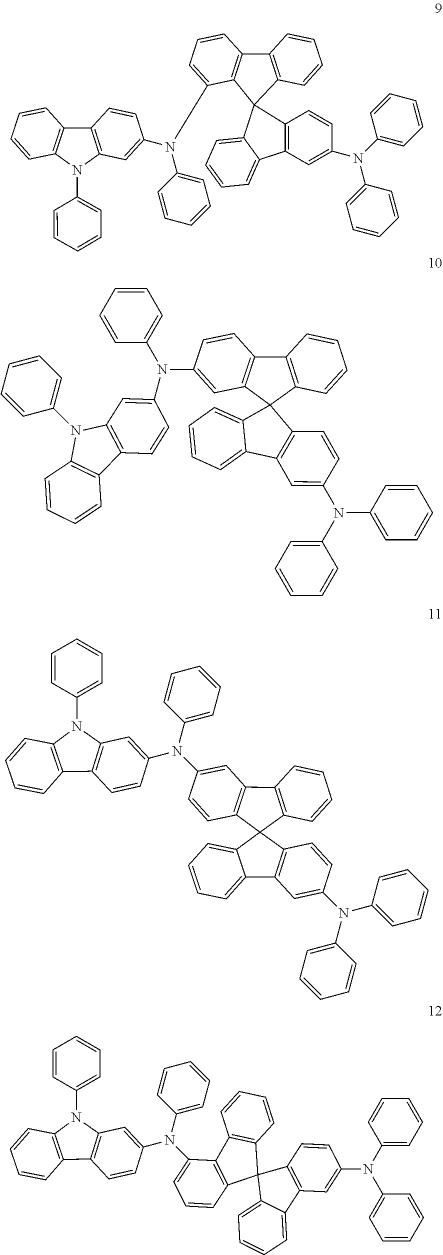 [SMILES: c1ccc(N(c2ccccc2)c2ccc3c(c2)-c2ccccc2C32c3ccccc3-c3cccc(N(c4ccccc4)c4ccc5c6ccccc6n(-c6ccccc6)c5c4)c32)cc1 ; c1ccc(N(c2ccccc2)c2ccc3c(c2)-c2ccccc2C32c3ccccc3-c3ccc(N(c4ccccc4)c4ccc5c6ccccc6n(-c6ccccc6)c5c4)cc32)cc1 ; c1ccc(N(c2ccccc2)c2ccc3c(c2)-c2ccccc2C32c3ccccc3-c3cc(N(c4ccccc4)c4ccc5c6ccccc6n(-c6ccccc6)c5c4)ccc32)cc1 ; c1ccc(N(c2ccccc2)c2ccc3c(c2)-c2ccccc2C32c3ccccc3-c3c(N(c4ccccc4)c4ccc5c6ccccc6n(-c6ccccc6)c5c4)cccc32)cc1] 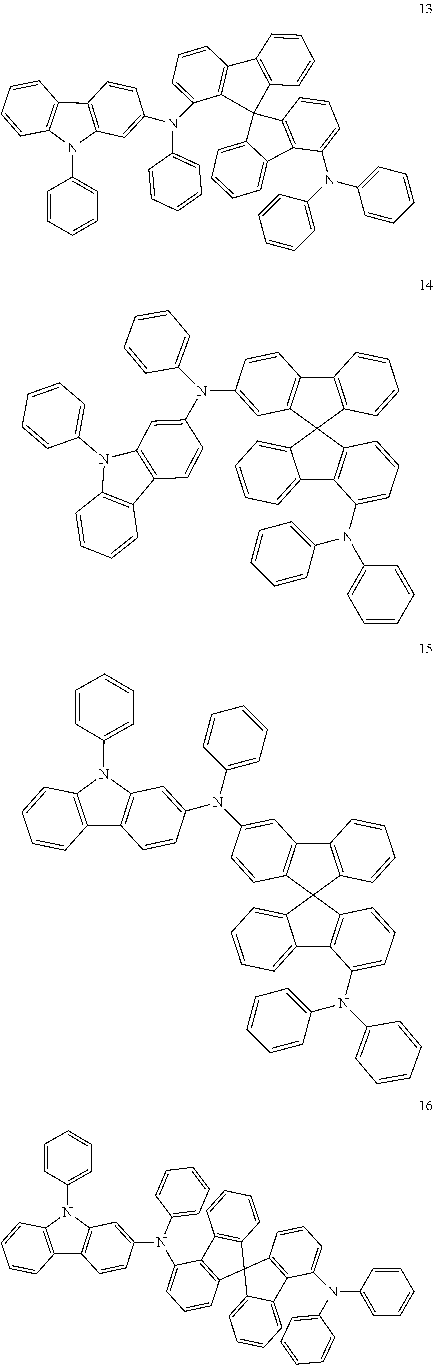 [SMILES: c1ccc(N(c2ccccc2)c2cccc3c2-c2ccccc2C32c3ccccc3-c3cccc(N(c4ccccc4)c4ccc5c6ccccc6n(-c6ccccc6)c5c4)c32)cc1 ; c1ccc(N(c2ccc3c(c2)C2(c4ccccc4-3)c3ccccc3-c3c(N(c4ccccc4)c4ccccc4)cccc32)c2ccc3c4ccccc4n(-c4ccccc4)c3c2)cc1 ; c1ccc(N(c2ccc3c(c2)-c2ccccc2C32c3ccccc3-c3c(N(c4ccccc4)c4ccccc4)cccc32)c2ccc3c4ccccc4n(-c4ccccc4)c3c2)cc1 ; c1ccc(N(c2ccccc2)c2cccc3c2-c2ccccc2C32c3ccccc3-c3c(N(c4ccccc4)c4ccc5c6ccccc6n(-c6ccccc6)c5c4)cccc32)cc1] 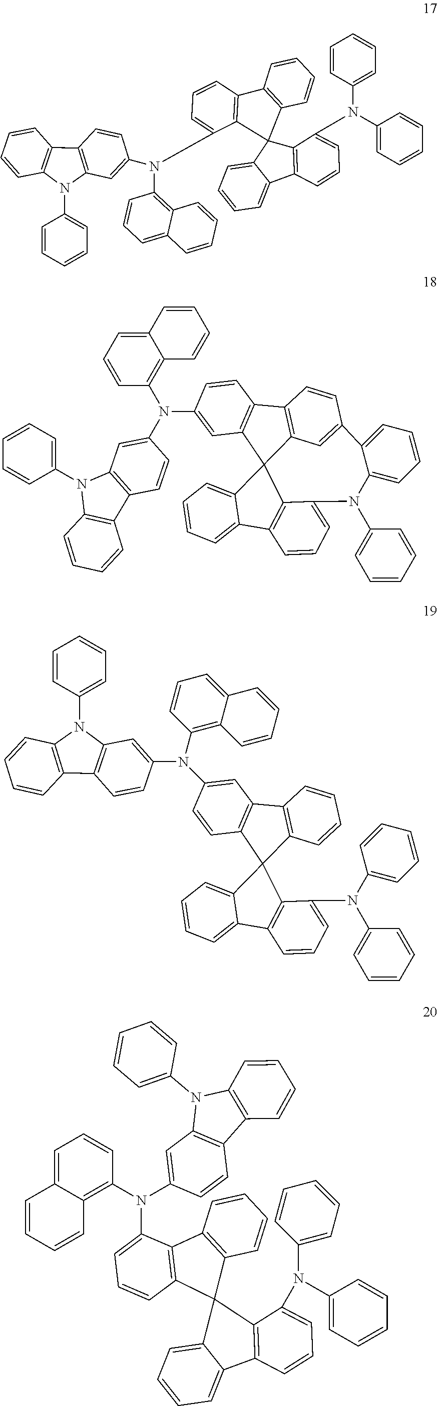 [SMILES: c1ccc(N(c2ccccc2)c2cccc3c2C2(c4ccccc4-3)c3ccccc3-c3cccc(N(c4ccc5c6ccccc6n(-c6ccccc6)c5c4)c4cccc5ccccc45)c32)cc1 ; c1ccc(N2c3ccccc3-c3ccc4c(c3)C3(c5cc(N(c6ccc7c8ccccc8n(-c8ccccc8)c7c6)c6cccc7ccccc67)ccc5-4)c4ccccc4-c4cccc2c43)cc1 ; c1ccc(N(c2ccccc2)c2cccc3c2C2(c4ccccc4-c4cc(N(c5ccc6c7ccccc7n(-c7ccccc7)c6c5)c5cccc6ccccc56)ccc42)c2ccccc2-3)cc1 ; c1ccc(N(c2ccccc2)c2cccc3c2C2(c4ccccc4-3)c3ccccc3-c3c(N(c4ccc5c6ccccc6n(-c6ccccc6)c5c4)c4cccc5ccccc45)cccc32)cc1] 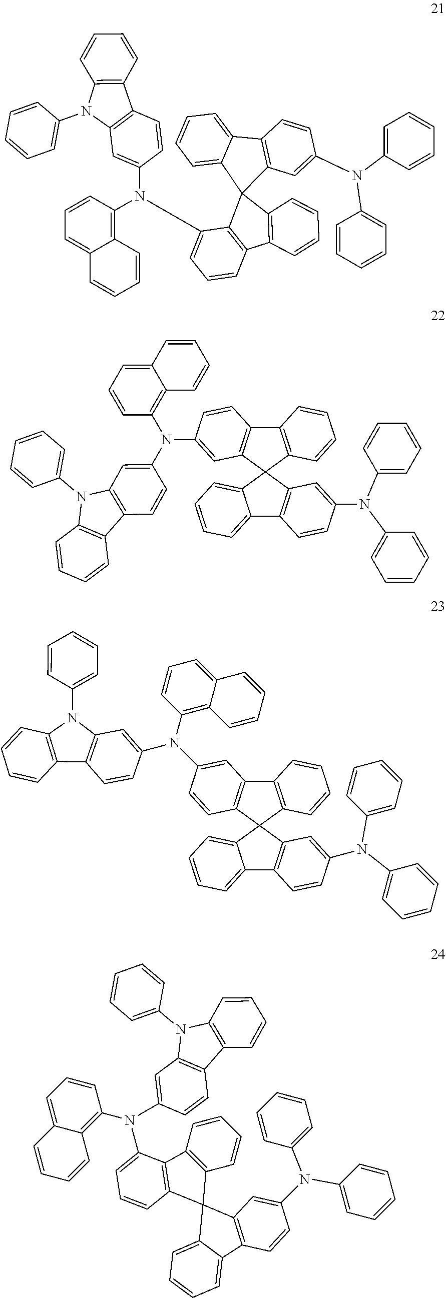 [SMILES: c1ccc(N(c2ccccc2)c2ccc3c(c2)C2(c4ccccc4-3)c3ccccc3-c3cccc(N(c4ccc5c6ccccc6n(-c6ccccc6)c5c4)c4cccc5ccccc45)c32)cc1 ; c1ccc(N(c2ccccc2)c2ccc3c(c2)C2(c4ccccc4-3)c3ccccc3-c3ccc(N(c4ccc5c6ccccc6n(-c6ccccc6)c5c4)c4cccc5ccccc45)cc32)cc1 ; c1ccc(N(c2ccccc2)c2ccc3c(c2)C2(c4ccccc4-c4cc(N(c5ccc6c7ccccc7n(-c7ccccc7)c6c5)c5cccc6ccccc56)ccc42)c2ccccc2-3)cc1 ; c1ccc(N(c2ccccc2)c2ccc3c(c2)C2(c4ccccc4-3)c3ccccc3-c3c(N(c4ccc5c6ccccc6n(-c6ccccc6)c5c4)c4cccc5ccccc45)cccc32)cc1] 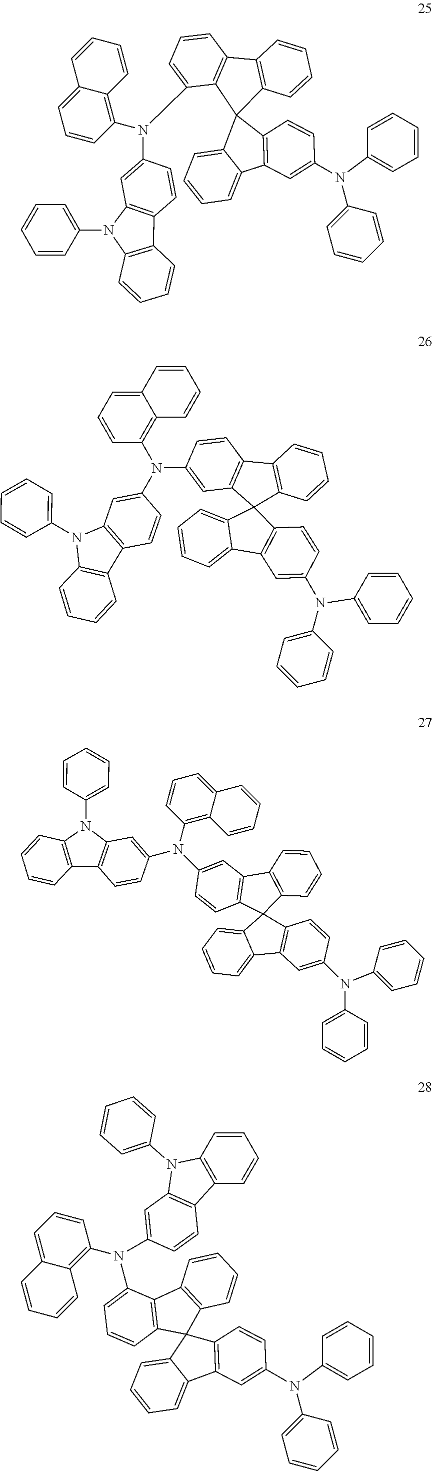 [SMILES: c1ccc(N(c2ccccc2)c2ccc3c(c2)-c2ccccc2C32c3ccccc3-c3cccc(N(c4ccc5c6ccccc6n(-c6ccccc6)c5c4)c4cccc5ccccc45)c32)cc1 ; c1ccc(N(c2ccccc2)c2ccc3c(c2)-c2ccccc2C32c3ccccc3-c3ccc(N(c4ccc5c6ccccc6n(-c6ccccc6)c5c4)c4cccc5ccccc45)cc32)cc1 ; c1ccc(N(c2ccccc2)c2ccc3c(c2)-c2ccccc2C32c3ccccc3-c3cc(N(c4ccc5c6ccccc6n(-c6ccccc6)c5c4)c4cccc5ccccc45)ccc32)cc1 ; c1ccc(N(c2ccccc2)c2ccc3c(c2)-c2ccccc2C32c3ccccc3-c3c(N(c4ccc5c6ccccc6n(-c6ccccc6)c5c4)c4cccc5ccccc45)cccc32)cc1] 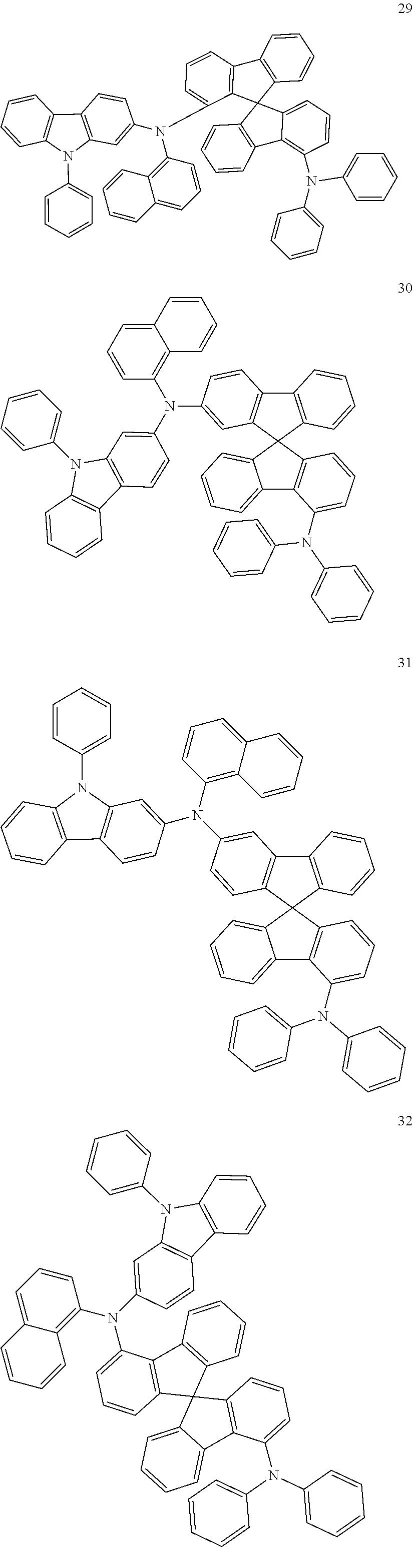 [SMILES: c1ccc(N(c2ccccc2)c2cccc3c2-c2ccccc2C32c3ccccc3-c3cccc(N(c4ccc5c6ccccc6n(-c6ccccc6)c5c4)c4cccc5ccccc45)c32)cc1 ; c1ccc(N(c2ccccc2)c2cccc3c2-c2ccccc2C32c3ccccc3-c3ccc(N(c4ccc5c6ccccc6n(-c6ccccc6)c5c4)c4cccc5ccccc45)cc32)cc1 ; c1ccc(N(c2ccccc2)c2cccc3c2-c2ccccc2C32c3ccccc3-c3cc(N(c4ccc5c6ccccc6n(-c6ccccc6)c5c4)c4cccc5ccccc45)ccc32)cc1 ; c1ccc(N(c2ccccc2)c2cccc3c2-c2ccccc2C32c3ccccc3-c3c(N(c4ccc5c6ccccc6n(-c6ccccc6)c5c4)c4cccc5ccccc45)cccc32)cc1] 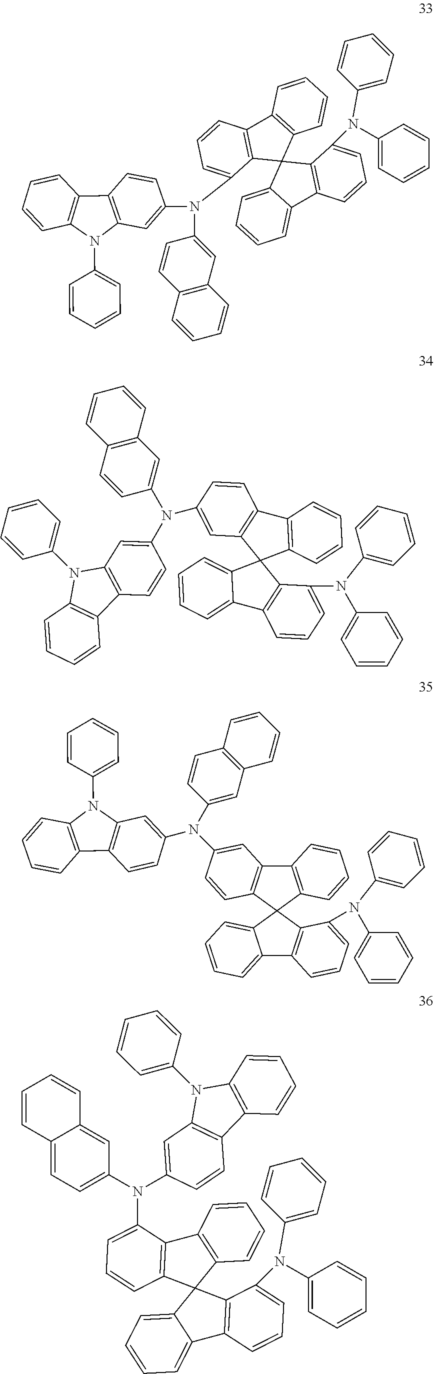 [SMILES: c1ccc(N(c2ccccc2)c2cccc3c2C2(c4ccccc4-3)c3ccccc3-c3cccc(N(c4ccc5ccccc5c4)c4ccc5c6ccccc6n(-c6ccccc6)c5c4)c32)cc1 ; c1ccc(N(c2ccccc2)c2cccc3c2C2(c4ccccc4-c4ccc(N(c5ccc6ccccc6c5)c5ccc6c7ccccc7n(-c7ccccc7)c6c5)cc42)c2ccccc2-3)cc1 ; c1ccc(N(c2ccccc2)c2cccc3c2C2(c4ccccc4-c4cc(N(c5ccc6ccccc6c5)c5ccc6c7ccccc7n(-c7ccccc7)c6c5)ccc42)c2ccccc2-3)cc1 ; c1ccc(N(c2ccccc2)c2cccc3c2C2(c4ccccc4-3)c3ccccc3-c3c(N(c4ccc5ccccc5c4)c4ccc5c6ccccc6n(-c6ccccc6)c5c4)cccc32)cc1] 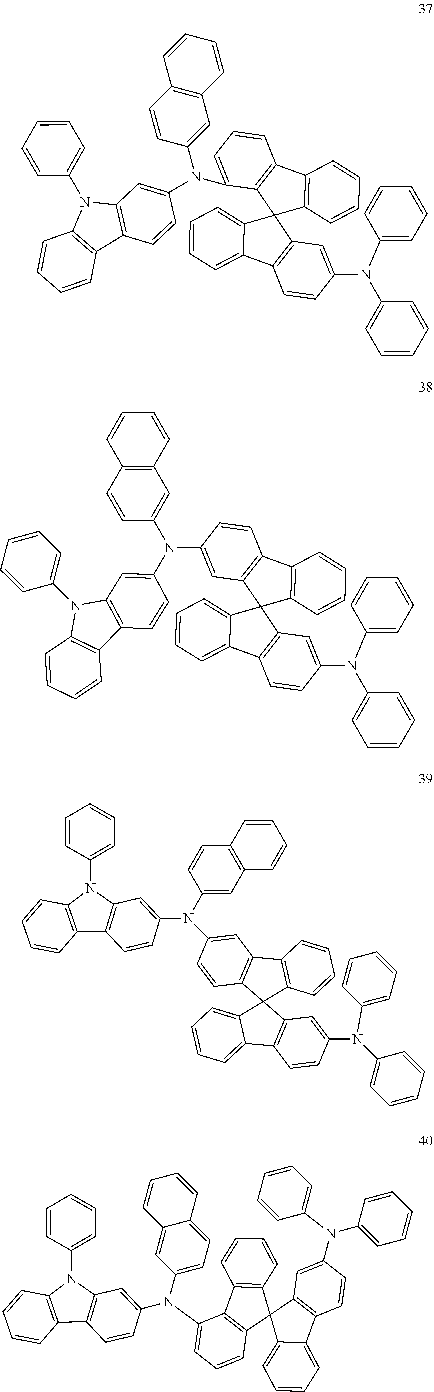 [SMILES: c1ccc(N(c2ccccc2)c2ccc3c(c2)C2(c4ccccc4-3)c3ccccc3-c3cccc(N(c4ccc5ccccc5c4)c4ccc5c6ccccc6n(-c6ccccc6)c5c4)c32)cc1 ; c1ccc(N(c2ccccc2)c2ccc3c(c2)C2(c4ccccc4-3)c3ccccc3-c3ccc(N(c4ccc5ccccc5c4)c4ccc5c6ccccc6n(-c6ccccc6)c5c4)cc32)cc1 ; c1ccc(N(c2ccccc2)c2ccc3c(c2)C2(c4ccccc4-c4cc(N(c5ccc6ccccc6c5)c5ccc6c7ccccc7n(-c7ccccc7)c6c5)ccc42)c2ccccc2-3)cc1 ; c1ccc(N(c2ccccc2)c2ccc3c(c2)C2(c4ccccc4-3)c3ccccc3-c3c(N(c4ccc5ccccc5c4)c4ccc5c6ccccc6n(-c6ccccc6)c5c4)cccc32)cc1] 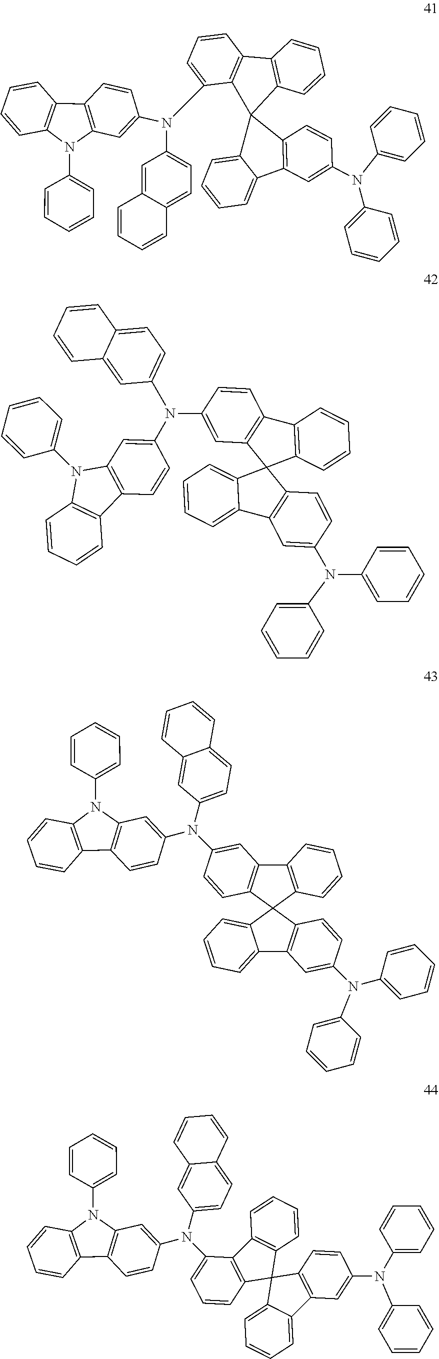 [SMILES: c1ccc(N(c2ccccc2)c2ccc3c(c2)-c2ccccc2C32c3ccccc3-c3cccc(N(c4ccc5ccccc5c4)c4ccc5c6ccccc6n(-c6ccccc6)c5c4)c32)cc1 ; c1ccc(N(c2ccccc2)c2ccc3c(c2)-c2ccccc2C32c3ccccc3-c3ccc(N(c4ccc5ccccc5c4)c4ccc5c6ccccc6n(-c6ccccc6)c5c4)cc32)cc1 ; c1ccc(N(c2ccccc2)c2ccc3c(c2)-c2ccccc2C32c3ccccc3-c3cc(N(c4ccc5ccccc5c4)c4ccc5c6ccccc6n(-c6ccccc6)c5c4)ccc32)cc1 ; c1ccc(N(c2ccccc2)c2ccc3c(c2)-c2ccccc2C32c3ccccc3-c3c(N(c4ccc5ccccc5c4)c4ccc5c6ccccc6n(-c6ccccc6)c5c4)cccc32)cc1] 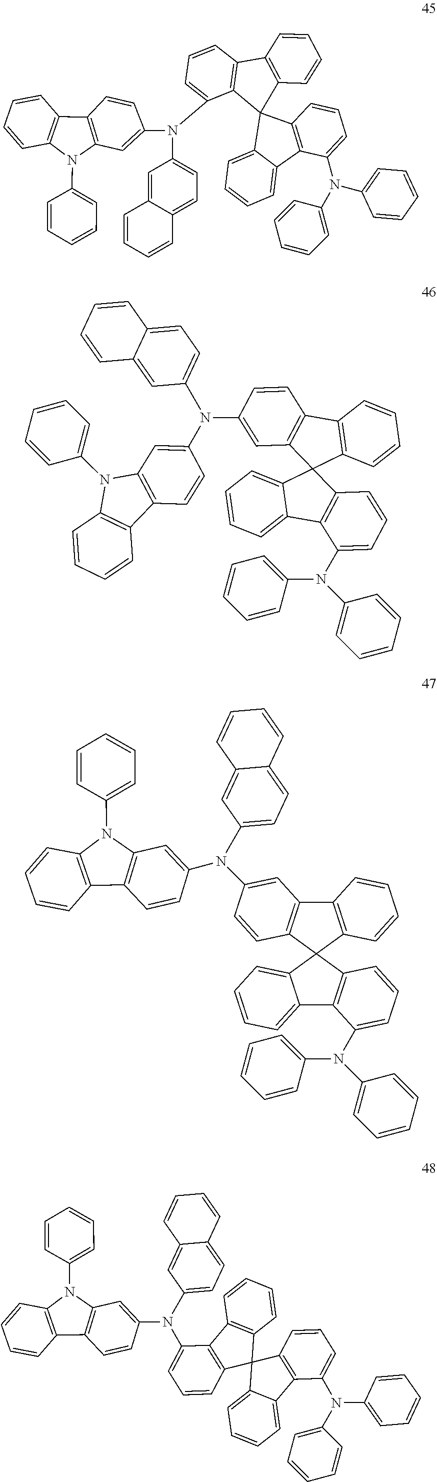 [SMILES: c1ccc(N(c2ccccc2)c2cccc3c2-c2ccccc2C32c3ccccc3-c3cccc(N(c4ccc5ccccc5c4)c4ccc5c6ccccc6n(-c6ccccc6)c5c4)c32)cc1 ; c1ccc(N(c2ccccc2)c2cccc3c2-c2ccccc2C32c3ccccc3-c3ccc(N(c4ccc5ccccc5c4)c4ccc5c6ccccc6n(-c6ccccc6)c5c4)cc32)cc1 ; c1ccc(N(c2ccccc2)c2cccc3c2-c2ccccc2C32c3ccccc3-c3cc(N(c4ccc5ccccc5c4)c4ccc5c6ccccc6n(-c6ccccc6)c5c4)ccc32)cc1 ; c1ccc(N(c2ccccc2)c2cccc3c2-c2ccccc2C32c3ccccc3-c3c(N(c4ccc5ccccc5c4)c4ccc5c6ccccc6n(-c6ccccc6)c5c4)cccc32)cc1] 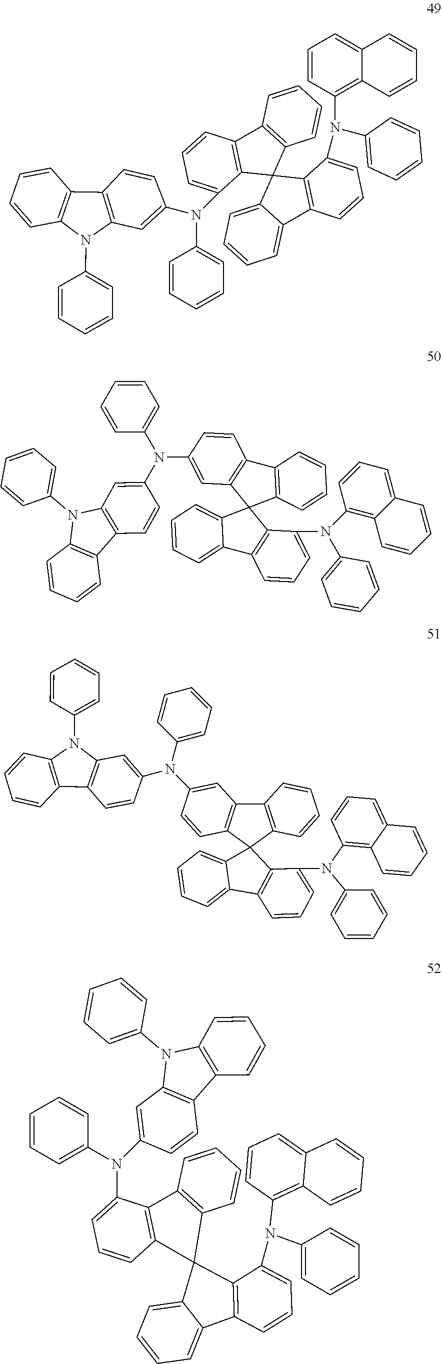 [SMILES: c1ccc(N(c2ccc3c4ccccc4n(-c4ccccc4)c3c2)c2cccc3c2C2(c4ccccc4-3)c3ccccc3-c3cccc(N(c4ccccc4)c4cccc5ccccc45)c32)cc1 ; c1ccc(N(c2ccc3c(c2)C2(c4ccccc4-3)c3ccccc3-c3cccc(N(c4ccccc4)c4cccc5ccccc45)c32)c2ccc3c4ccccc4n(-c4ccccc4)c3c2)cc1 ; c1ccc(N(c2ccc3c(c2)-c2ccccc2C32c3ccccc3-c3cccc(N(c4ccccc4)c4cccc5ccccc45)c32)c2ccc3c4ccccc4n(-c4ccccc4)c3c2)cc1 ; c1ccc(N(c2ccc3c4ccccc4n(-c4ccccc4)c3c2)c2cccc3c2-c2ccccc2C32c3ccccc3-c3cccc(N(c4ccccc4)c4cccc5ccccc45)c32)cc1] 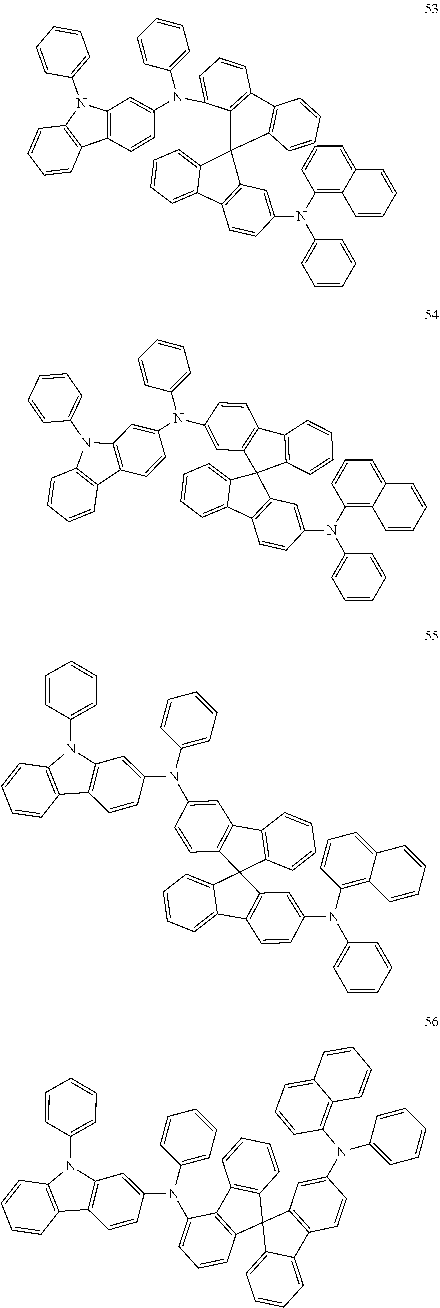 [SMILES: c1ccc(N(c2ccc3c4ccccc4n(-c4ccccc4)c3c2)c2cccc3c2C2(c4ccccc4-c4ccc(N(c5ccccc5)c5cccc6ccccc56)cc42)c2ccccc2-3)cc1 ; c1ccc(N(c2ccc3c(c2)C2(c4ccccc4-3)c3ccccc3-c3ccc(N(c4ccccc4)c4cccc5ccccc45)cc32)c2ccc3c4ccccc4n(-c4ccccc4)c3c2)cc1 ; c1ccc(N(c2ccc3c(c2)-c2ccccc2C32c3ccccc3-c3ccc(N(c4ccccc4)c4cccc5ccccc45)cc32)c2ccc3c4ccccc4n(-c4ccccc4)c3c2)cc1 ; c1ccc(N(c2ccc3c4ccccc4n(-c4ccccc4)c3c2)c2cccc3c2-c2ccccc2C32c3ccccc3-c3ccc(N(c4ccccc4)c4cccc5ccccc45)cc32)cc1] 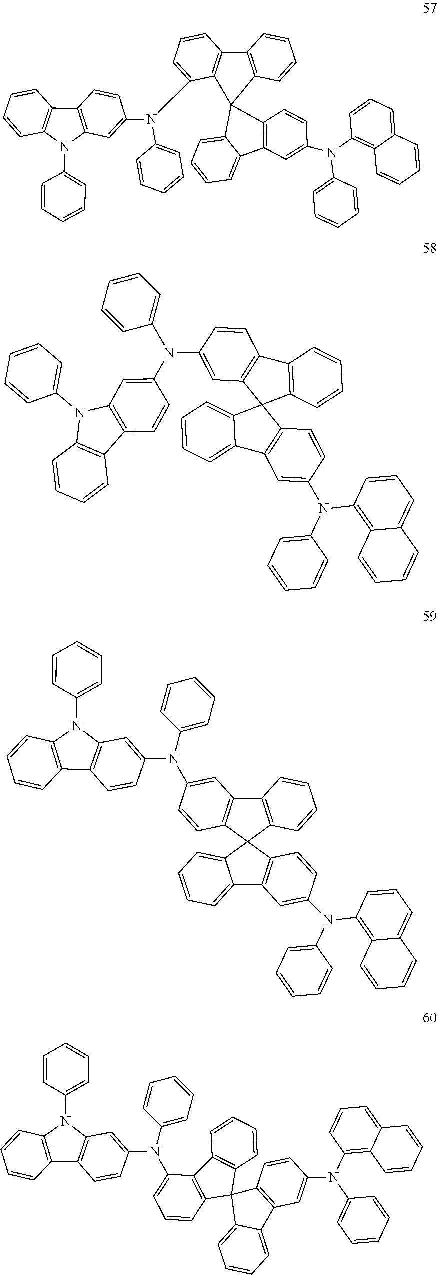 [SMILES: c1ccc(N(c2ccc3c4ccccc4n(-c4ccccc4)c3c2)c2cccc3c2C2(c4ccccc4-c4cc(N(c5ccccc5)c5cccc6ccccc56)ccc42)c2ccccc2-3)cc1 ; c1ccc(N(c2ccc3c(c2)C2(c4ccccc4-c4cc(N(c5ccccc5)c5cccc6ccccc56)ccc42)c2ccccc2-3)c2ccc3c4ccccc4n(-c4ccccc4)c3c2)cc1 ; c1ccc(N(c2ccc3c(c2)-c2ccccc2C32c3ccccc3-c3cc(N(c4ccccc4)c4cccc5ccccc45)ccc32)c2ccc3c4ccccc4n(-c4ccccc4)c3c2)cc1 ; c1ccc(N(c2ccc3c4ccccc4n(-c4ccccc4)c3c2)c2cccc3c2-c2ccccc2C32c3ccccc3-c3cc(N(c4ccccc4)c4cccc5ccccc45)ccc32)cc1] 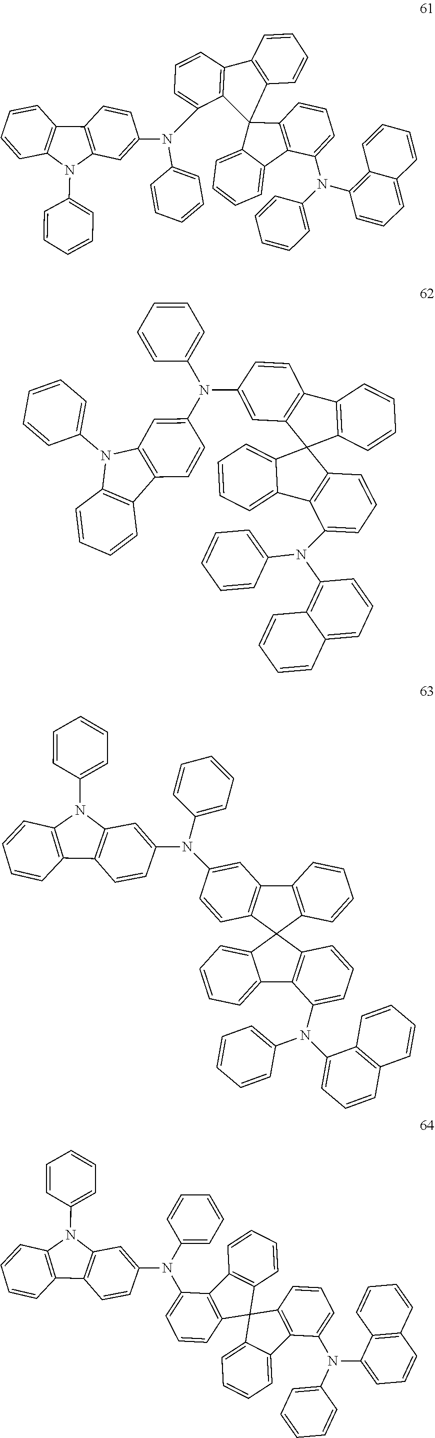 [SMILES: c1ccc(N(c2ccc3c4ccccc4n(-c4ccccc4)c3c2)c2cccc3c2C2(c4ccccc4-3)c3ccccc3-c3c(N(c4ccccc4)c4cccc5ccccc45)cccc32)cc1 ; c1ccc(N(c2ccc3c(c2)C2(c4ccccc4-3)c3ccccc3-c3c(N(c4ccccc4)c4cccc5ccccc45)cccc32)c2ccc3c4ccccc4n(-c4ccccc4)c3c2)cc1 ; c1ccc(N(c2ccc3c(c2)-c2ccccc2C32c3ccccc3-c3c(N(c4ccccc4)c4cccc5ccccc45)cccc32)c2ccc3c4ccccc4n(-c4ccccc4)c3c2)cc1 ; c1ccc(N(c2ccc3c4ccccc4n(-c4ccccc4)c3c2)c2cccc3c2-c2ccccc2C32c3ccccc3-c3c(N(c4ccccc4)c4cccc5ccccc45)cccc32)cc1] 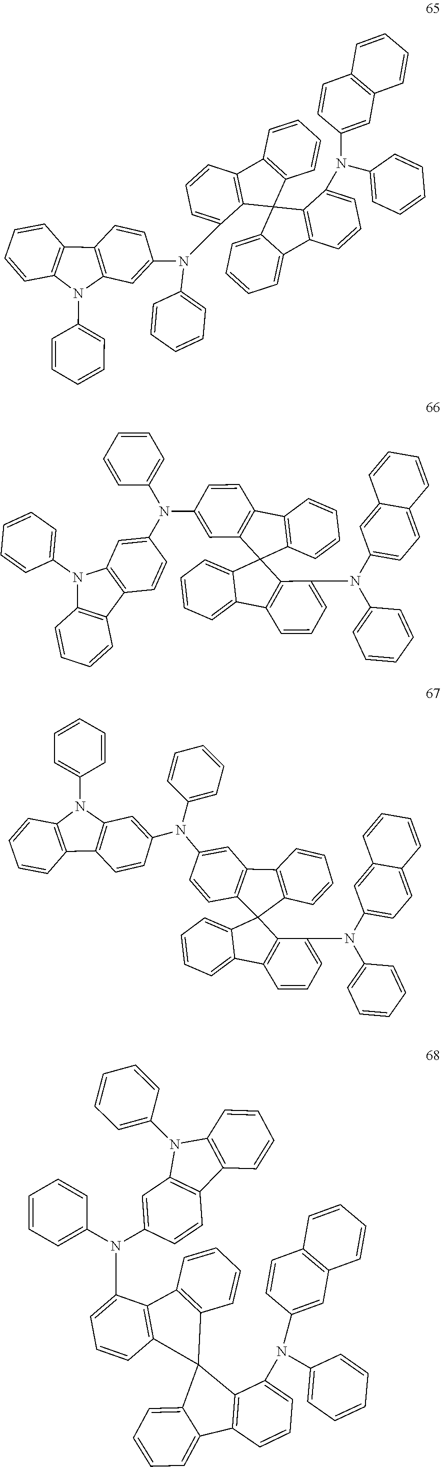 [SMILES: c1ccc(N(c2ccc3ccccc3c2)c2cccc3c2C2(c4ccccc4-3)c3ccccc3-c3cccc(N(c4ccccc4)c4ccc5c6ccccc6n(-c6ccccc6)c5c4)c32)cc1 ; c1ccc(N(c2ccc3c(c2)C2(c4ccccc4-3)c3ccccc3-c3cccc(N(c4ccccc4)c4ccc5ccccc5c4)c32)c2ccc3c4ccccc4n(-c4ccccc4)c3c2)cc1 ; c1ccc(N(c2ccc3c(c2)-c2ccccc2C32c3ccccc3-c3cccc(N(c4ccccc4)c4ccc5ccccc5c4)c32)c2ccc3c4ccccc4n(-c4ccccc4)c3c2)cc1 ; c1ccc(N(c2ccc3c4ccccc4n(-c4ccccc4)c3c2)c2cccc3c2-c2ccccc2C32c3ccccc3-c3cccc(N(c4ccccc4)c4ccc5ccccc5c4)c32)cc1] 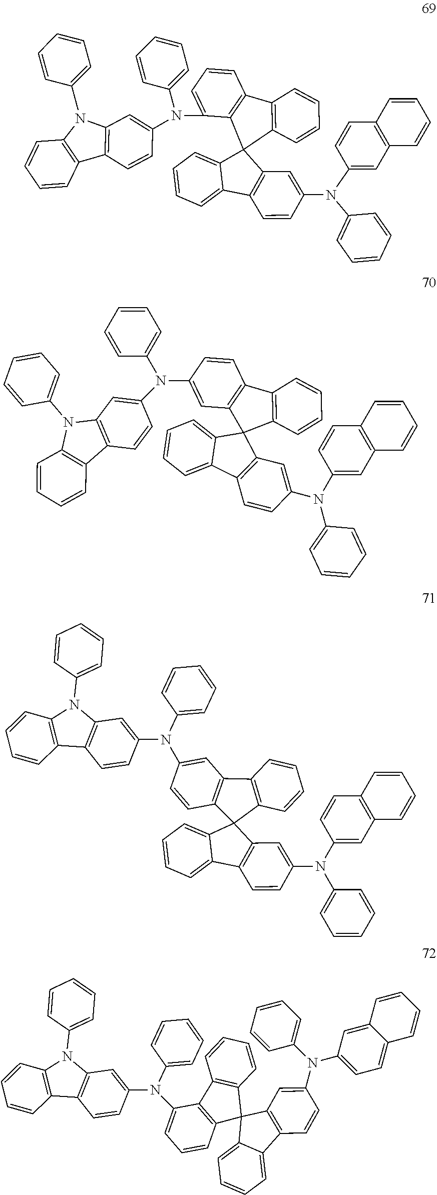 [SMILES: c1ccc(N(c2ccc3c(c2)C2(c4ccccc4-3)c3ccccc3-c3cccc(N(c4ccccc4)c4ccc5c6ccccc6n(-c6ccccc6)c5c4)c32)c2ccc3ccccc3c2)cc1 ; c1ccc(N(c2ccc3c(c2)C2(c4ccccc4-3)c3ccccc3-c3ccc(N(c4ccccc4)c4ccc5c6ccccc6n(-c6ccccc6)c5c4)cc32)c2ccc3ccccc3c2)cc1 ; c1ccc(N(c2ccc3c(c2)C2(c4ccccc4-c4cc(N(c5ccccc5)c5ccc6c7ccccc7n(-c7ccccc7)c6c5)ccc42)c2ccccc2-3)c2ccc3ccccc3c2)cc1 ; c1ccc(N(c2ccc3c(c2)C2(c4ccccc4-3)c3ccccc3-c3c(N(c4ccccc4)c4ccc5c6ccccc6n(-c6ccccc6)c5c4)cccc32)c2ccc3ccccc3c2)cc1] 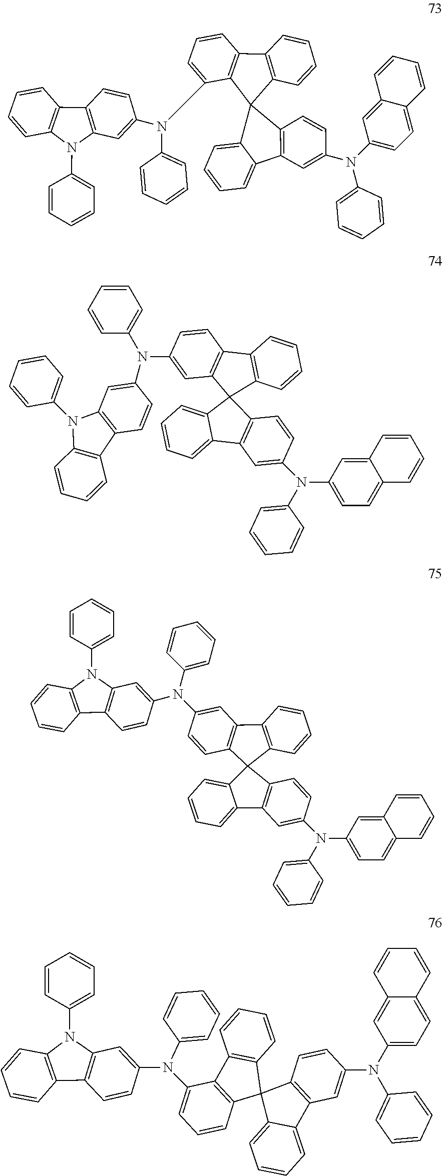 [SMILES: c1ccc(N(c2ccc3c(c2)-c2ccccc2C32c3ccccc3-c3cccc(N(c4ccccc4)c4ccc5c6ccccc6n(-c6ccccc6)c5c4)c32)c2ccc3ccccc3c2)cc1 ; c1ccc(N(c2ccc3c(c2)-c2ccccc2C32c3ccccc3-c3ccc(N(c4ccccc4)c4ccc5c6ccccc6n(-c6ccccc6)c5c4)cc32)c2ccc3ccccc3c2)cc1 ; c1ccc(N(c2ccc3c(c2)-c2ccccc2C32c3ccccc3-c3cc(N(c4ccccc4)c4ccc5c6ccccc6n(-c6ccccc6)c5c4)ccc32)c2ccc3ccccc3c2)cc1 ; c1ccc(N(c2ccc3c(c2)-c2ccccc2C32c3ccccc3-c3c(N(c4ccccc4)c4ccc5c6ccccc6n(-c6ccccc6)c5c4)cccc32)c2ccc3ccccc3c2)cc1] 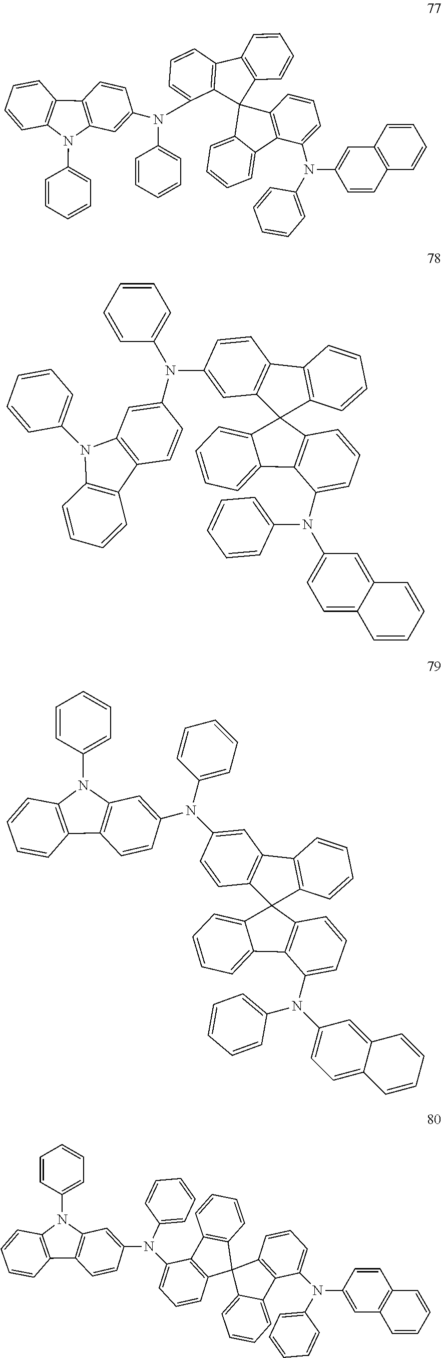 [SMILES: c1ccc(N(c2ccc3ccccc3c2)c2cccc3c2-c2ccccc2C32c3ccccc3-c3cccc(N(c4ccccc4)c4ccc5c6ccccc6n(-c6ccccc6)c5c4)c32)cc1 ; c1ccc(N(c2ccc3c(c2)C2(c4ccccc4-3)c3ccccc3-c3c(N(c4ccccc4)c4ccc5ccccc5c4)cccc32)c2ccc3c4ccccc4n(-c4ccccc4)c3c2)cc1 ; c1ccc(N(c2ccc3c(c2)-c2ccccc2C32c3ccccc3-c3c(N(c4ccccc4)c4ccc5ccccc5c4)cccc32)c2ccc3c4ccccc4n(-c4ccccc4)c3c2)cc1 ; c1ccc(N(c2ccc3ccccc3c2)c2cccc3c2-c2ccccc2C32c3ccccc3-c3c(N(c4ccccc4)c4ccc5c6ccccc6n(-c6ccccc6)c5c4)cccc32)cc1] 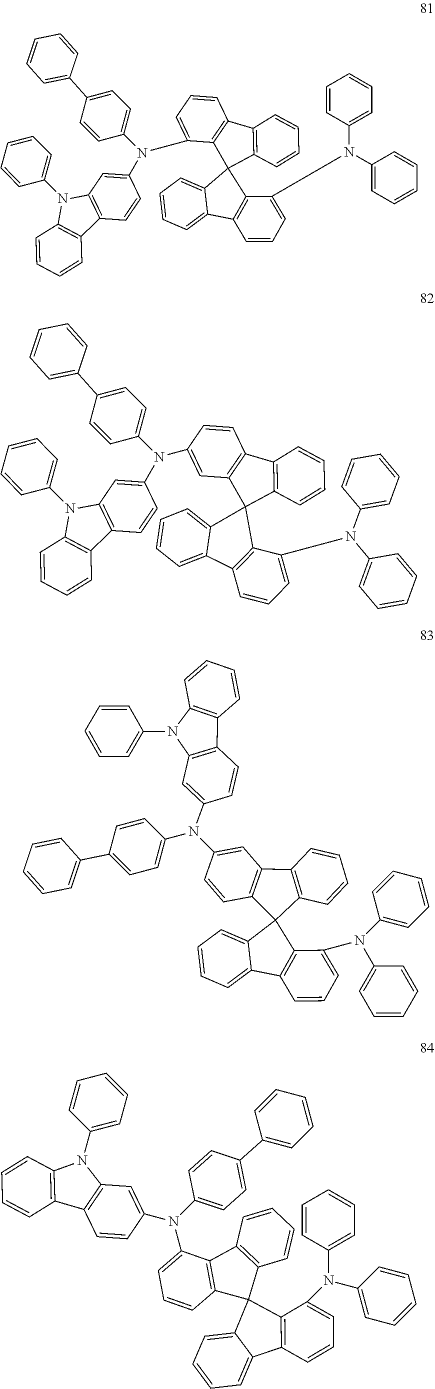 [SMILES: c1ccc(-c2ccc(N(c3ccc4c5ccccc5n(-c5ccccc5)c4c3)c3cccc4c3C3(c5ccccc5-c5cccc(N(c6ccccc6)c6ccccc6)c53)c3ccccc3-4)cc2)cc1 ; c1ccc(-c2ccc(N(c3ccc4c(c3)C3(c5ccccc5-4)c4ccccc4-c4cccc(N(c5ccccc5)c5ccccc5)c43)c3ccc4c5ccccc5n(-c5ccccc5)c4c3)cc2)cc1 ; c1ccc(-c2ccc(N(c3ccc4c(c3)-c3ccccc3C43c4ccccc4-c4cccc(N(c5ccccc5)c5ccccc5)c43)c3ccc4c5ccccc5n(-c5ccccc5)c4c3)cc2)cc1 ; c1ccc(-c2ccc(N(c3ccc4c5ccccc5n(-c5ccccc5)c4c3)c3cccc4c3-c3ccccc3C43c4ccccc4-c4cccc(N(c5ccccc5)c5ccccc5)c43)cc2)cc1] 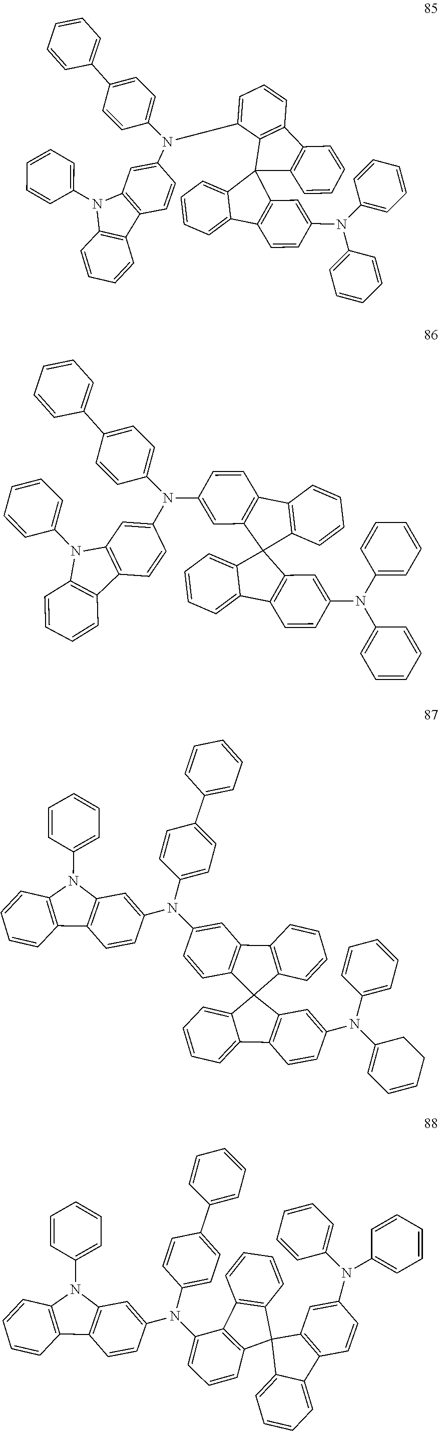 [SMILES: c1ccc(-c2ccc(N(c3ccc4c5ccccc5n(-c5ccccc5)c4c3)c3cccc4c3C3(c5ccccc5-c5ccc(N(c6ccccc6)c6ccccc6)cc53)c3ccccc3-4)cc2)cc1 ; c1ccc(-c2ccc(N(c3ccc4c(c3)C3(c5ccccc5-c5ccc(N(c6ccccc6)c6ccccc6)cc53)c3ccccc3-4)c3ccc4c5ccccc5n(-c5ccccc5)c4c3)cc2)cc1 ; C1=CCCC(N(c2ccccc2)c2ccc3c(c2)C2(c4ccccc4-c4cc(N(c5ccc(-c6ccccc6)cc5)c5ccc6c7ccccc7n(-c7ccccc7)c6c5)ccc42)c2ccccc2-3)=C1 ; c1ccc(-c2ccc(N(c3ccc4c5ccccc5n(-c5ccccc5)c4c3)c3cccc4c3-c3ccccc3C43c4ccccc4-c4ccc(N(c5ccccc5)c5ccccc5)cc43)cc2)cc1] 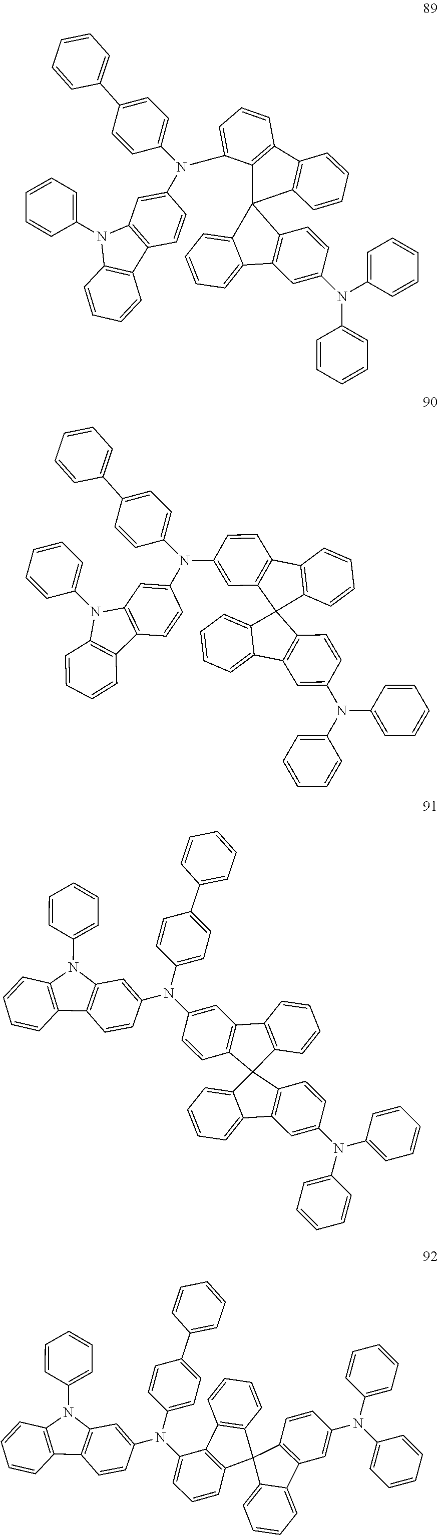 [SMILES: c1ccc(-c2ccc(N(c3ccc4c5ccccc5n(-c5ccccc5)c4c3)c3cccc4c3C3(c5ccccc5-c5cc(N(c6ccccc6)c6ccccc6)ccc53)c3ccccc3-4)cc2)cc1 ; c1ccc(-c2ccc(N(c3ccc4c(c3)C3(c5ccccc5-c5cc(N(c6ccccc6)c6ccccc6)ccc53)c3ccccc3-4)c3ccc4c5ccccc5n(-c5ccccc5)c4c3)cc2)cc1 ; c1ccc(-c2ccc(N(c3ccc4c(c3)-c3ccccc3C43c4ccccc4-c4cc(N(c5ccccc5)c5ccccc5)ccc43)c3ccc4c5ccccc5n(-c5ccccc5)c4c3)cc2)cc1 ; c1ccc(-c2ccc(N(c3ccc4c5ccccc5n(-c5ccccc5)c4c3)c3cccc4c3-c3ccccc3C43c4ccccc4-c4cc(N(c5ccccc5)c5ccccc5)ccc43)cc2)cc1] 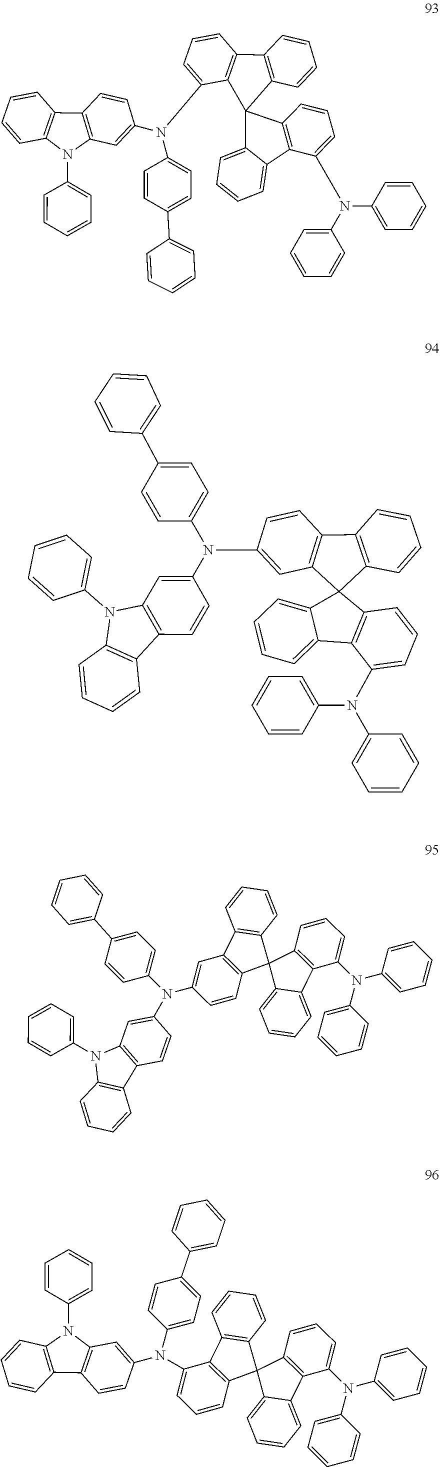 [SMILES: c1ccc(-c2ccc(N(c3ccc4c5ccccc5n(-c5ccccc5)c4c3)c3cccc4c3C3(c5ccccc5-4)c4ccccc4-c4c(N(c5ccccc5)c5ccccc5)cccc43)cc2)cc1 ; c1ccc(-c2ccc(N(c3ccc4c(c3)C3(c5ccccc5-4)c4ccccc4-c4c(N(c5ccccc5)c5ccccc5)cccc43)c3ccc4c5ccccc5n(-c5ccccc5)c4c3)cc2)cc1 ; c1ccc(-c2ccc(N(c3ccc4c(c3)-c3ccccc3C43c4ccccc4-c4c(N(c5ccccc5)c5ccccc5)cccc43)c3ccc4c5ccccc5n(-c5ccccc5)c4c3)cc2)cc1 ; c1ccc(-c2ccc(N(c3ccc4c5ccccc5n(-c5ccccc5)c4c3)c3cccc4c3-c3ccccc3C43c4ccccc4-c4c(N(c5ccccc5)c5ccccc5)cccc43)cc2)cc1] 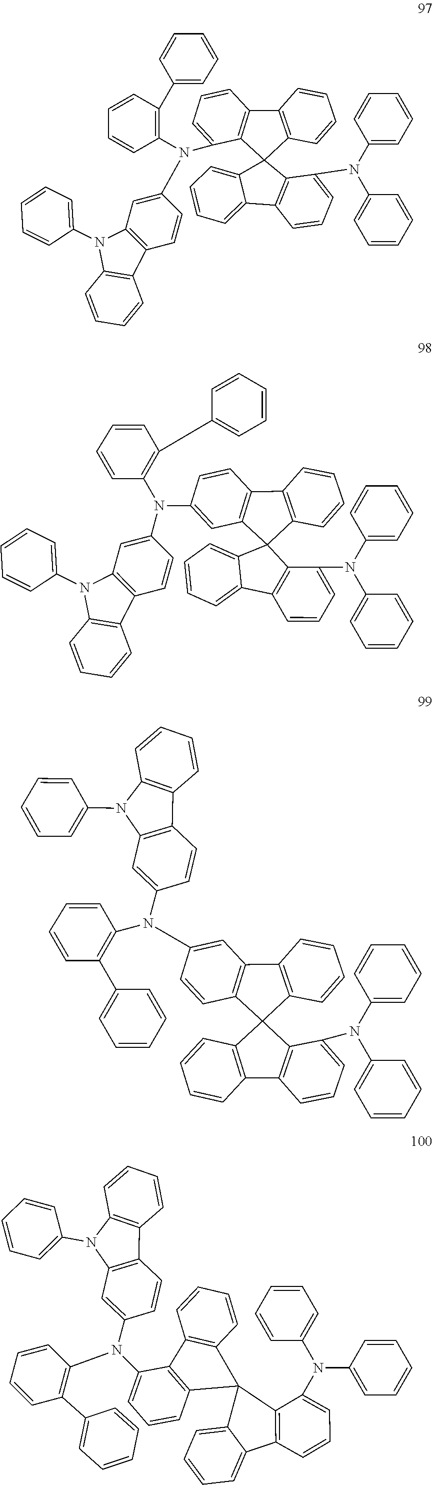 [SMILES: c1ccc(-c2ccccc2N(c2ccc3c4ccccc4n(-c4ccccc4)c3c2)c2cccc3c2C2(c4ccccc4-c4cccc(N(c5ccccc5)c5ccccc5)c42)c2ccccc2-3)cc1 ; c1ccc(-c2ccccc2N(c2ccc3c(c2)C2(c4ccccc4-3)c3ccccc3-c3cccc(N(c4ccccc4)c4ccccc4)c32)c2ccc3c4ccccc4n(-c4ccccc4)c3c2)cc1 ; c1ccc(-c2ccccc2N(c2ccc3c(c2)-c2ccccc2C32c3ccccc3-c3cccc(N(c4ccccc4)c4ccccc4)c32)c2ccc3c4ccccc4n(-c4ccccc4)c3c2)cc1 ; c1ccc(-c2ccccc2N(c2ccc3c4ccccc4n(-c4ccccc4)c3c2)c2cccc3c2-c2ccccc2C32c3ccccc3-c3cccc(N(c4ccccc4)c4ccccc4)c32)cc1] 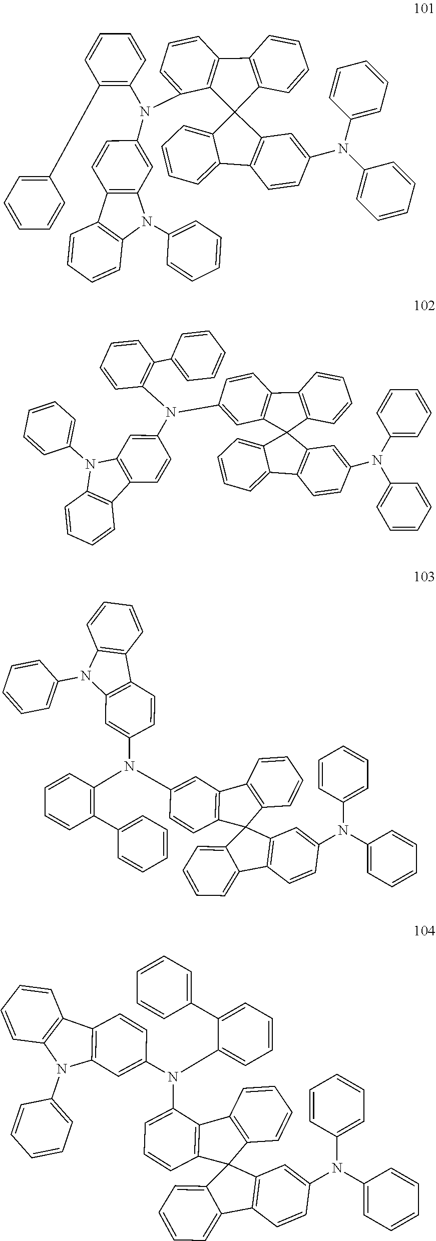 [SMILES: c1ccc(-c2ccccc2N(c2ccc3c4ccccc4n(-c4ccccc4)c3c2)c2cccc3c2C2(c4ccccc4-c4ccc(N(c5ccccc5)c5ccccc5)cc42)c2ccccc2-3)cc1 ; c1ccc(-c2ccccc2N(c2ccc3c(c2)C2(c4ccccc4-c4ccc(N(c5ccccc5)c5ccccc5)cc42)c2ccccc2-3)c2ccc3c4ccccc4n(-c4ccccc4)c3c2)cc1 ; c1ccc(-c2ccccc2N(c2ccc3c(c2)-c2ccccc2C32c3ccccc3-c3ccc(N(c4ccccc4)c4ccccc4)cc32)c2ccc3c4ccccc4n(-c4ccccc4)c3c2)cc1 ; c1ccc(-c2ccccc2N(c2ccc3c4ccccc4n(-c4ccccc4)c3c2)c2cccc3c2-c2ccccc2C32c3ccccc3-c3ccc(N(c4ccccc4)c4ccccc4)cc32)cc1] 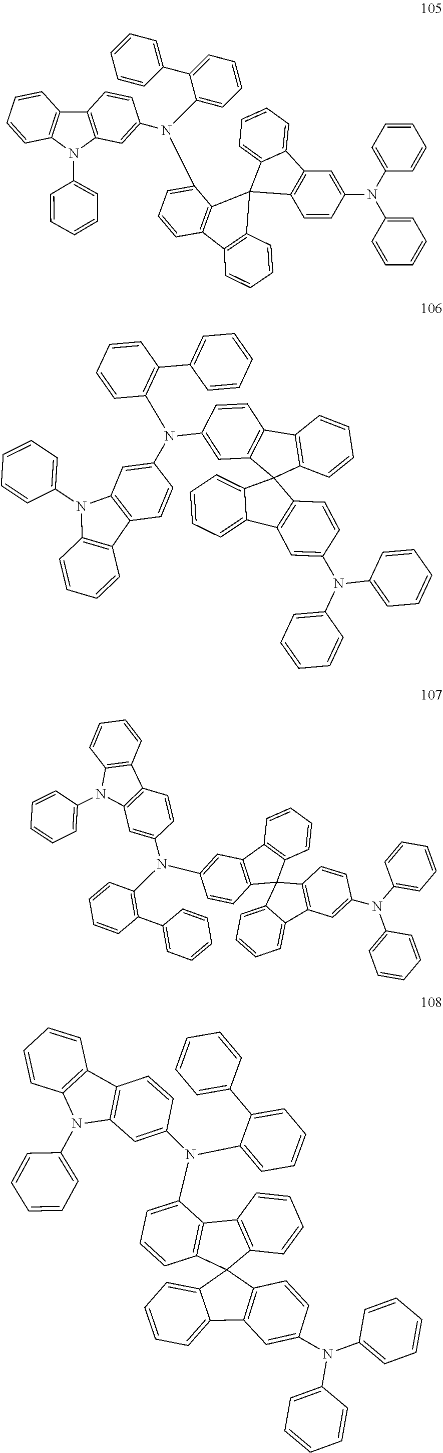 [SMILES: c1ccc(-c2ccccc2N(c2ccc3c4ccccc4n(-c4ccccc4)c3c2)c2cccc3c2C2(c4ccccc4-c4cc(N(c5ccccc5)c5ccccc5)ccc42)c2ccccc2-3)cc1 ; c1ccc(-c2ccccc2N(c2ccc3c(c2)C2(c4ccccc4-c4cc(N(c5ccccc5)c5ccccc5)ccc42)c2ccccc2-3)c2ccc3c4ccccc4n(-c4ccccc4)c3c2)cc1 ; c1ccc(-c2ccccc2N(c2ccc3c(c2)-c2ccccc2C32c3ccccc3-c3cc(N(c4ccccc4)c4ccccc4)ccc32)c2ccc3c4ccccc4n(-c4ccccc4)c3c2)cc1 ; c1ccc(-c2ccccc2N(c2ccc3c4ccccc4n(-c4ccccc4)c3c2)c2cccc3c2-c2ccccc2C32c3ccccc3-c3cc(N(c4ccccc4)c4ccccc4)ccc32)cc1] 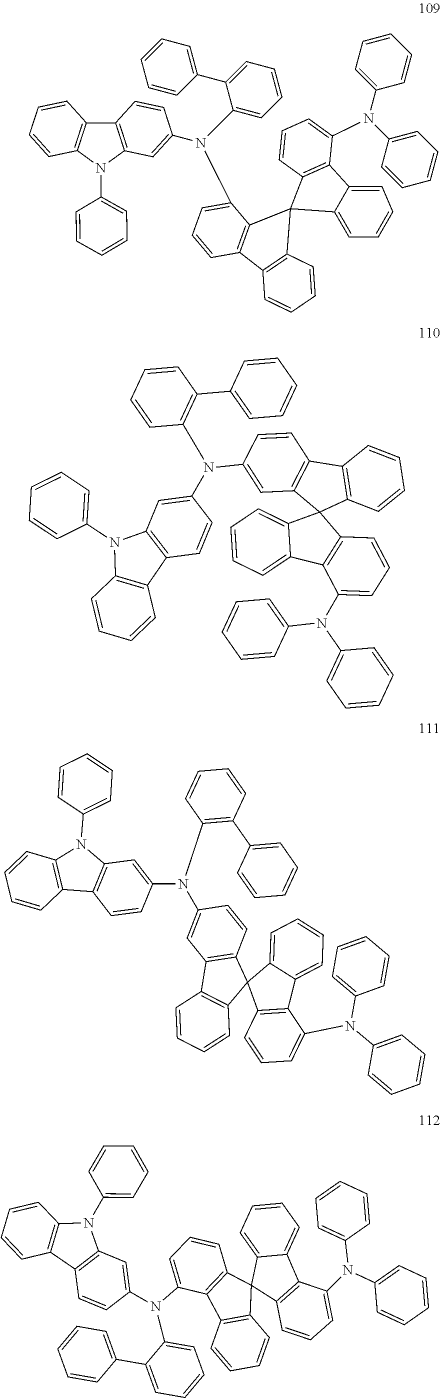 [SMILES: c1ccc(-c2ccccc2N(c2ccc3c4ccccc4n(-c4ccccc4)c3c2)c2cccc3c2C2(c4ccccc4-3)c3ccccc3-c3c(N(c4ccccc4)c4ccccc4)cccc32)cc1 ; c1ccc(-c2ccccc2N(c2ccc3c(c2)C2(c4ccccc4-3)c3ccccc3-c3c(N(c4ccccc4)c4ccccc4)cccc32)c2ccc3c4ccccc4n(-c4ccccc4)c3c2)cc1 ; c1ccc(-c2ccccc2N(c2ccc3c(c2)-c2ccccc2C32c3ccccc3-c3c(N(c4ccccc4)c4ccccc4)cccc32)c2ccc3c4ccccc4n(-c4ccccc4)c3c2)cc1 ; c1ccc(-c2ccccc2N(c2ccc3c4ccccc4n(-c4ccccc4)c3c2)c2cccc3c2-c2ccccc2C32c3ccccc3-c3c(N(c4ccccc4)c4ccccc4)cccc32)cc1] 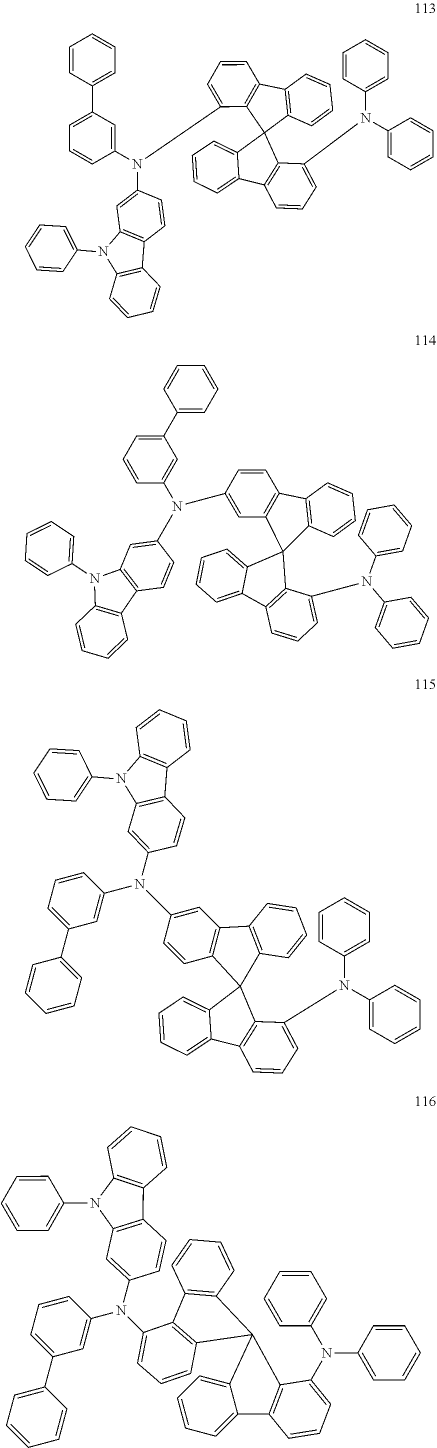 [SMILES: c1ccc(-c2cccc(N(c3ccc4c5ccccc5n(-c5ccccc5)c4c3)c3cccc4c3C3(c5ccccc5-c5cccc(N(c6ccccc6)c6ccccc6)c53)c3ccccc3-4)c2)cc1 ; c1ccc(-c2cccc(N(c3ccc4c(c3)C3(c5ccccc5-4)c4ccccc4-c4cccc(N(c5ccccc5)c5ccccc5)c43)c3ccc4c5ccccc5n(-c5ccccc5)c4c3)c2)cc1 ; c1ccc(-c2cccc(N(c3ccc4c(c3)-c3ccccc3C43c4ccccc4-c4cccc(N(c5ccccc5)c5ccccc5)c43)c3ccc4c5ccccc5n(-c5ccccc5)c4c3)c2)cc1 ; c1ccc(-c2cccc(N(c3ccc4c5ccccc5n(-c5ccccc5)c4c3)c3cccc4c3-c3ccccc3C43c4ccccc4-c4cccc(N(c5ccccc5)c5ccccc5)c43)c2)cc1] 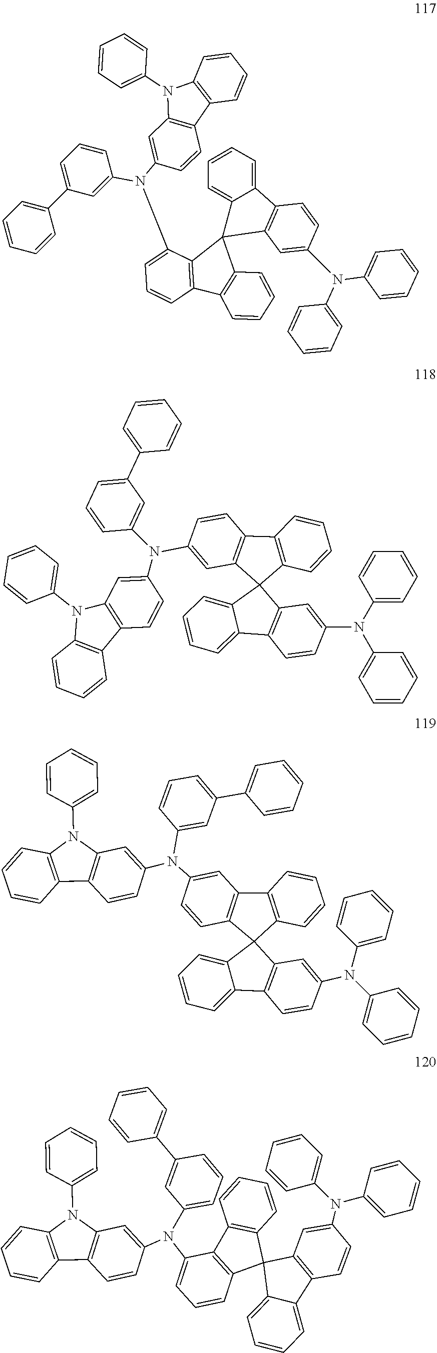 [SMILES: c1ccc(-c2cccc(N(c3ccc4c5ccccc5n(-c5ccccc5)c4c3)c3cccc4c3C3(c5ccccc5-c5ccc(N(c6ccccc6)c6ccccc6)cc53)c3ccccc3-4)c2)cc1 ; c1ccc(-c2cccc(N(c3ccc4c(c3)C3(c5ccccc5-c5ccc(N(c6ccccc6)c6ccccc6)cc53)c3ccccc3-4)c3ccc4c5ccccc5n(-c5ccccc5)c4c3)c2)cc1 ; c1ccc(-c2cccc(N(c3ccc4c(c3)-c3ccccc3C43c4ccccc4-c4ccc(N(c5ccccc5)c5ccccc5)cc43)c3ccc4c5ccccc5n(-c5ccccc5)c4c3)c2)cc1 ; c1ccc(-c2cccc(N(c3ccc4c5ccccc5n(-c5ccccc5)c4c3)c3cccc4c3-c3ccccc3C43c4ccccc4-c4ccc(N(c5ccccc5)c5ccccc5)cc43)c2)cc1] 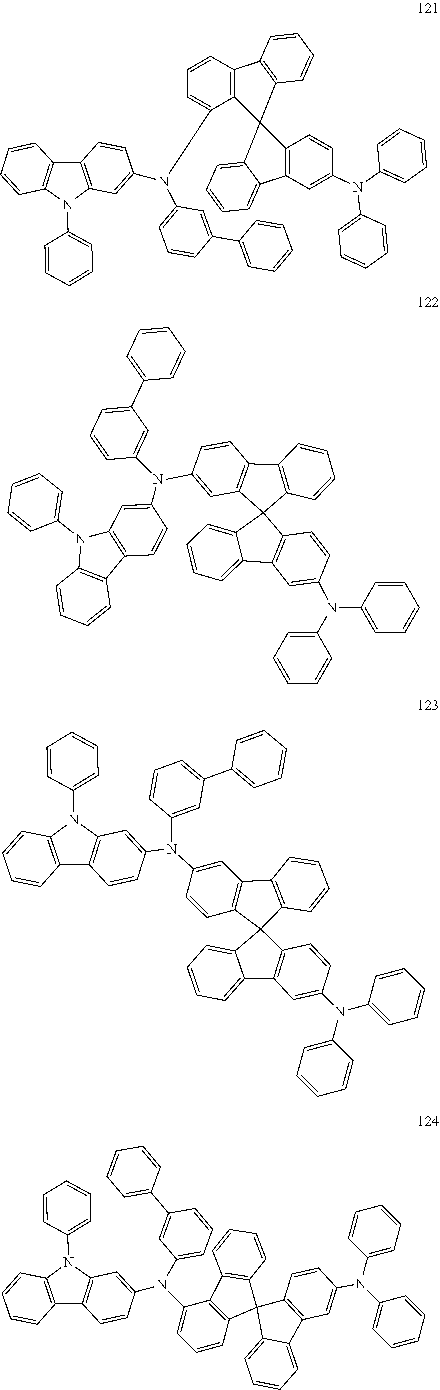 [SMILES: c1ccc(-c2cccc(N(c3ccc4c5ccccc5n(-c5ccccc5)c4c3)c3cccc4c3C3(c5ccccc5-c5cc(N(c6ccccc6)c6ccccc6)ccc53)c3ccccc3-4)c2)cc1 ; c1ccc(-c2cccc(N(c3ccc4c(c3)C3(c5ccccc5-c5cc(N(c6ccccc6)c6ccccc6)ccc53)c3ccccc3-4)c3ccc4c5ccccc5n(-c5ccccc5)c4c3)c2)cc1 ; c1ccc(-c2cccc(N(c3ccc4c(c3)-c3ccccc3C43c4ccccc4-c4cc(N(c5ccccc5)c5ccccc5)ccc43)c3ccc4c5ccccc5n(-c5ccccc5)c4c3)c2)cc1 ; c1ccc(-c2cccc(N(c3ccc4c5ccccc5n(-c5ccccc5)c4c3)c3cccc4c3-c3ccccc3C43c4ccccc4-c4cc(N(c5ccccc5)c5ccccc5)ccc43)c2)cc1] 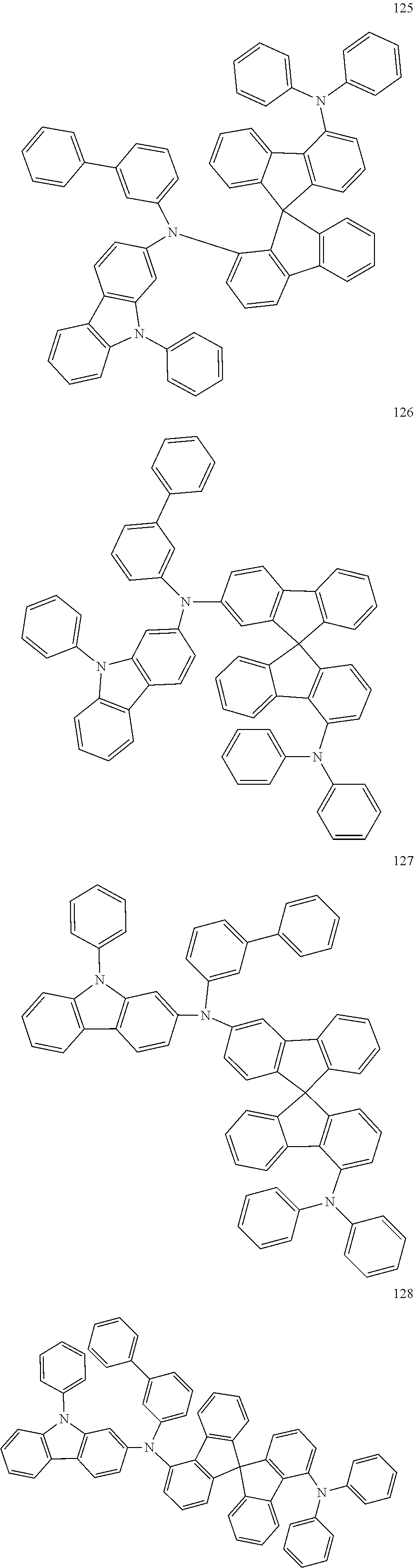 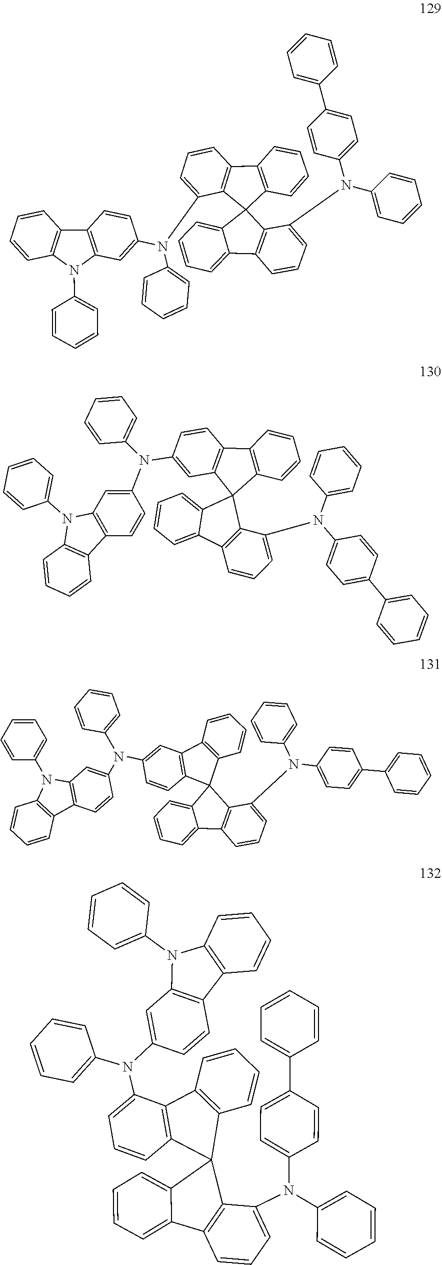 [SMILES: c1ccc(-c2ccc(N(c3ccccc3)c3cccc4c3C3(c5ccccc5-4)c4ccccc4-c4cccc(N(c5ccccc5)c5ccc6c7ccccc7n(-c7ccccc7)c6c5)c43)cc2)cc1 ; c1ccc(-c2ccc(N(c3ccccc3)c3cccc4c3C3(c5ccccc5-c5ccc(N(c6ccccc6)c6ccc7c8ccccc8n(-c8ccccc8)c7c6)cc53)c3ccccc3-4)cc2)cc1 ; c1ccc(-c2ccc(N(c3ccccc3)c3cccc4c3C3(c5ccccc5-c5cc(N(c6ccccc6)c6ccc7c8ccccc8n(-c8ccccc8)c7c6)ccc53)c3ccccc3-4)cc2)cc1 ; c1ccc(-c2ccc(N(c3ccccc3)c3cccc4c3C3(c5ccccc5-4)c4ccccc4-c4c(N(c5ccccc5)c5ccc6c7ccccc7n(-c7ccccc7)c6c5)cccc43)cc2)cc1] 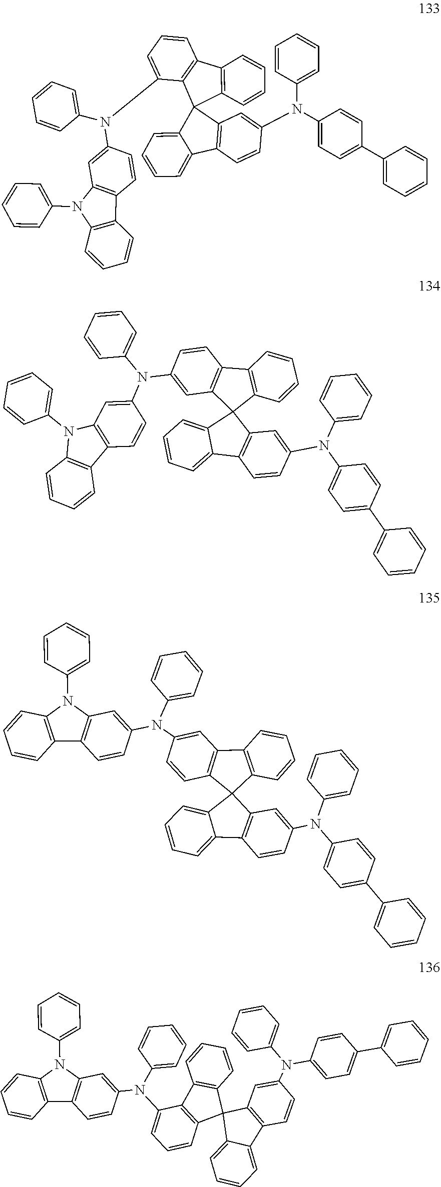 [SMILES: c1ccc(-c2ccc(N(c3ccccc3)c3ccc4c(c3)C3(c5ccccc5-4)c4ccccc4-c4cccc(N(c5ccccc5)c5ccc6c7ccccc7n(-c7ccccc7)c6c5)c43)cc2)cc1 ; c1ccc(-c2ccc(N(c3ccccc3)c3ccc4c(c3)C3(c5ccccc5-4)c4ccccc4-c4ccc(N(c5ccccc5)c5ccc6c7ccccc7n(-c7ccccc7)c6c5)cc43)cc2)cc1 ; c1ccc(-c2ccc(N(c3ccccc3)c3ccc4c(c3)C3(c5ccccc5-c5cc(N(c6ccccc6)c6ccc7c8ccccc8n(-c8ccccc8)c7c6)ccc53)c3ccccc3-4)cc2)cc1 ; c1ccc(-c2ccc(N(c3ccccc3)c3ccc4c(c3)C3(c5ccccc5-4)c4ccccc4-c4c(N(c5ccccc5)c5ccc6c7ccccc7n(-c7ccccc7)c6c5)cccc43)cc2)cc1] 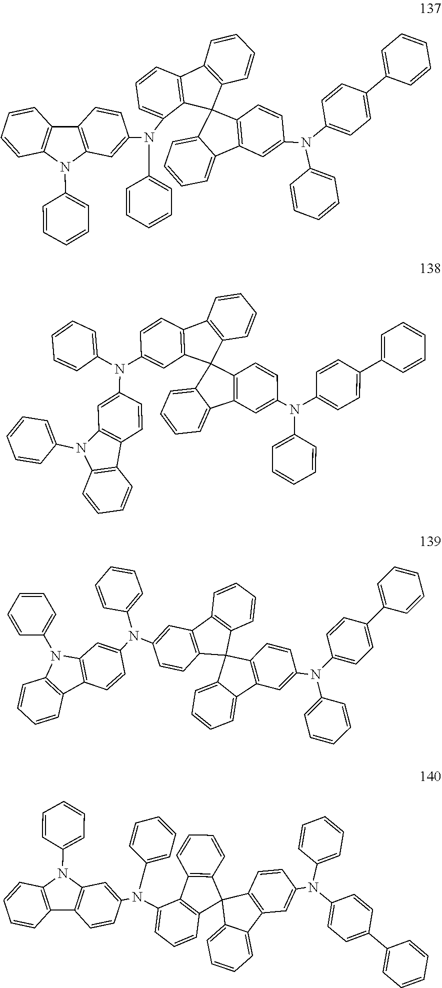 [SMILES: c1ccc(-c2ccc(N(c3ccccc3)c3ccc4c(c3)-c3ccccc3C43c4ccccc4-c4cccc(N(c5ccccc5)c5ccc6c7ccccc7n(-c7ccccc7)c6c5)c43)cc2)cc1 ; c1ccc(-c2ccc(N(c3ccccc3)c3ccc4c(c3)-c3ccccc3C43c4ccccc4-c4ccc(N(c5ccccc5)c5ccc6c7ccccc7n(-c7ccccc7)c6c5)cc43)cc2)cc1 ; c1ccc(-c2ccc(N(c3ccccc3)c3ccc4c(c3)-c3ccccc3C43c4ccccc4-c4cc(N(c5ccccc5)c5ccc6c7ccccc7n(-c7ccccc7)c6c5)ccc43)cc2)cc1 ; c1ccc(-c2ccc(N(c3ccccc3)c3ccc4c(c3)-c3ccccc3C43c4ccccc4-c4c(N(c5ccccc5)c5ccc6c7ccccc7n(-c7ccccc7)c6c5)cccc43)cc2)cc1] 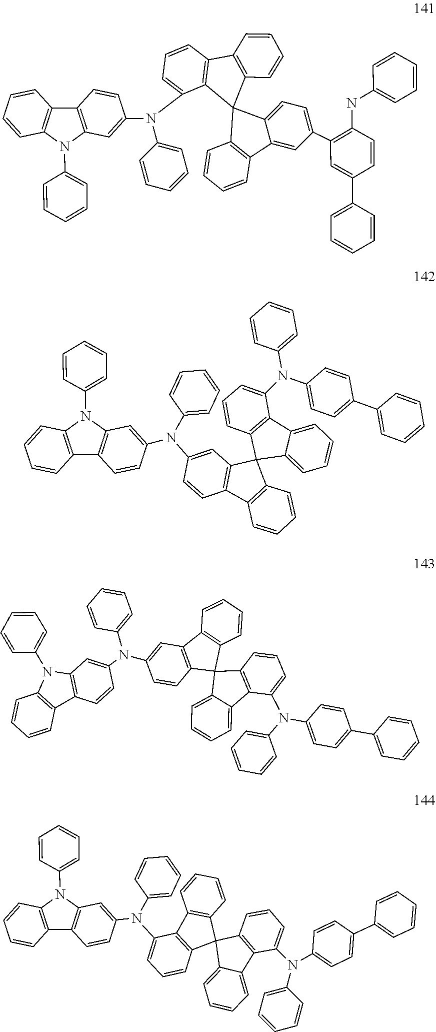 [SMILES: c1ccc(Nc2ccc(-c3ccccc3)cc2-c2ccc3c(c2)-c2ccccc2C32c3ccccc3-c3cccc(N(c4ccccc4)c4ccc5c6ccccc6n(-c6ccccc6)c5c4)c32)cc1 ; c1ccc(-c2ccc(N(c3ccccc3)c3cccc4c3-c3ccccc3C43c4ccccc4-c4ccc(N(c5ccccc5)c5ccc6c7ccccc7n(-c7ccccc7)c6c5)cc43)cc2)cc1 ; c1ccc(-c2ccc(N(c3ccccc3)c3cccc4c3-c3ccccc3C43c4ccccc4-c4cc(N(c5ccccc5)c5ccc6c7ccccc7n(-c7ccccc7)c6c5)ccc43)cc2)cc1 ; c1ccc(-c2ccc(N(c3ccccc3)c3cccc4c3-c3ccccc3C43c4ccccc4-c4c(N(c5ccccc5)c5ccc6c7ccccc7n(-c7ccccc7)c6c5)cccc43)cc2)cc1] 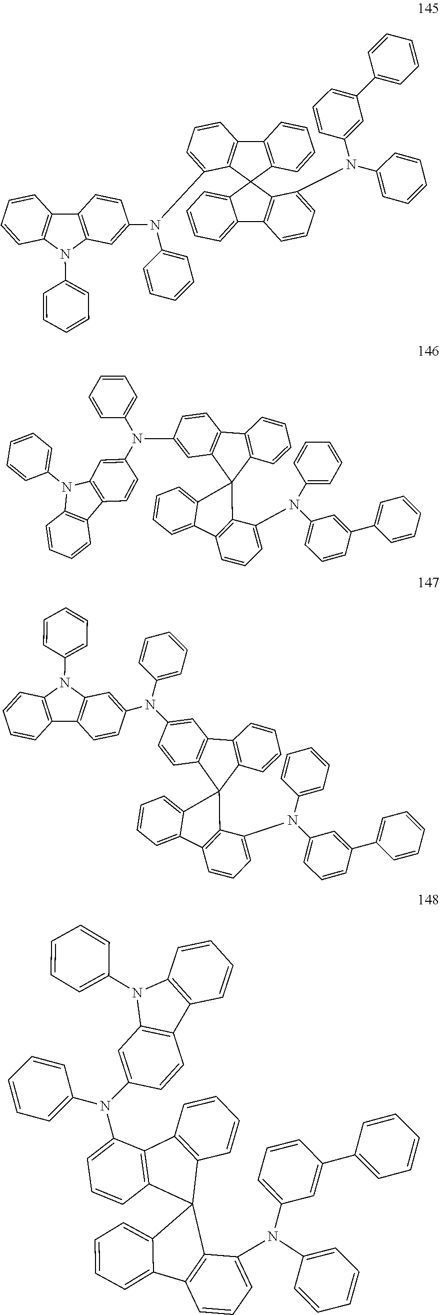 [SMILES: c1ccc(-c2cccc(N(c3ccccc3)c3cccc4c3C3(c5ccccc5-4)c4ccccc4-c4cccc(N(c5ccccc5)c5ccc6c7ccccc7n(-c7ccccc7)c6c5)c43)c2)cc1 ; c1ccc(-c2cccc(N(c3ccccc3)c3cccc4c3C3(c5ccccc5-c5ccc(N(c6ccccc6)c6ccc7c8ccccc8n(-c8ccccc8)c7c6)cc53)c3ccccc3-4)c2)cc1 ; c1ccc(-c2cccc(N(c3ccccc3)c3cccc4c3C3(c5ccccc5-c5cc(N(c6ccccc6)c6ccc7c8ccccc8n(-c8ccccc8)c7c6)ccc53)c3ccccc3-4)c2)cc1 ; c1ccc(-c2cccc(N(c3ccccc3)c3cccc4c3C3(c5ccccc5-4)c4ccccc4-c4c(N(c5ccccc5)c5ccc6c7ccccc7n(-c7ccccc7)c6c5)cccc43)c2)cc1] 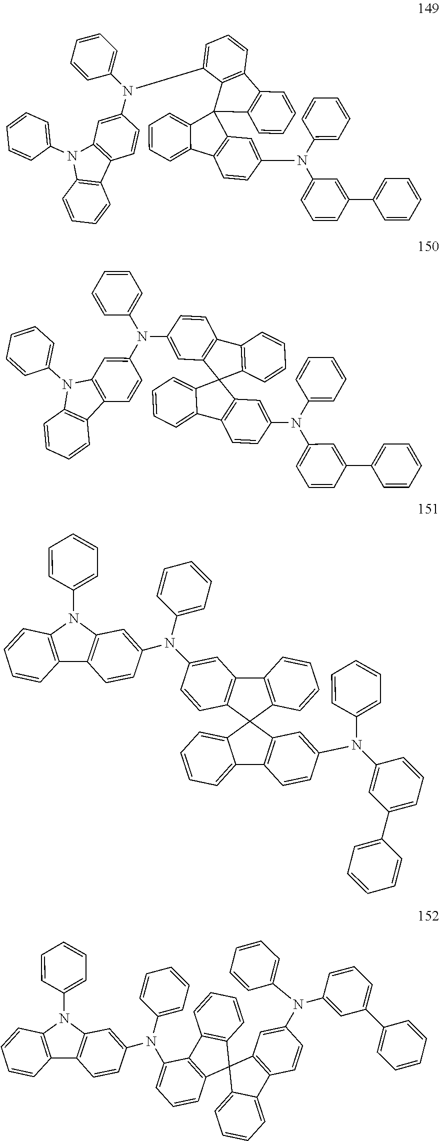 [SMILES: c1ccc(-c2cccc(N(c3ccccc3)c3ccc4c(c3)C3(c5ccccc5-4)c4ccccc4-c4cccc(N(c5ccccc5)c5ccc6c7ccccc7n(-c7ccccc7)c6c5)c43)c2)cc1 ; c1ccc(-c2cccc(N(c3ccccc3)c3ccc4c(c3)C3(c5ccccc5-4)c4ccccc4-c4ccc(N(c5ccccc5)c5ccc6c7ccccc7n(-c7ccccc7)c6c5)cc43)c2)cc1 ; c1ccc(-c2cccc(N(c3ccccc3)c3ccc4c(c3)C3(c5ccccc5-c5cc(N(c6ccccc6)c6ccc7c8ccccc8n(-c8ccccc8)c7c6)ccc53)c3ccccc3-4)c2)cc1 ; c1ccc(-c2cccc(N(c3ccccc3)c3ccc4c(c3)C3(c5ccccc5-4)c4ccccc4-c4c(N(c5ccccc5)c5ccc6c7ccccc7n(-c7ccccc7)c6c5)cccc43)c2)cc1] 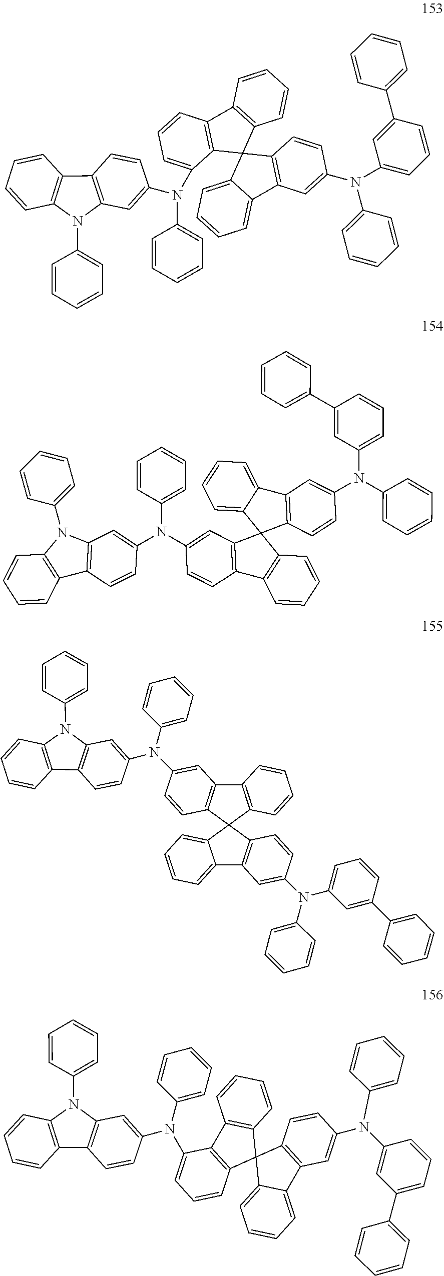 [SMILES: c1ccc(-c2cccc(N(c3ccccc3)c3ccc4c(c3)-c3ccccc3C43c4ccccc4-c4cccc(N(c5ccccc5)c5ccc6c7ccccc7n(-c7ccccc7)c6c5)c43)c2)cc1 ; c1ccc(-c2cccc(N(c3ccccc3)c3ccc4c(c3)-c3ccccc3C43c4ccccc4-c4ccc(N(c5ccccc5)c5ccc6c7ccccc7n(-c7ccccc7)c6c5)cc43)c2)cc1 ; c1ccc(-c2cccc(N(c3ccccc3)c3ccc4c(c3)-c3ccccc3C43c4ccccc4-c4cc(N(c5ccccc5)c5ccc6c7ccccc7n(-c7ccccc7)c6c5)ccc43)c2)cc1 ; c1ccc(-c2cccc(N(c3ccccc3)c3ccc4c(c3)-c3ccccc3C43c4ccccc4-c4c(N(c5ccccc5)c5ccc6c7ccccc7n(-c7ccccc7)c6c5)cccc43)c2)cc1] 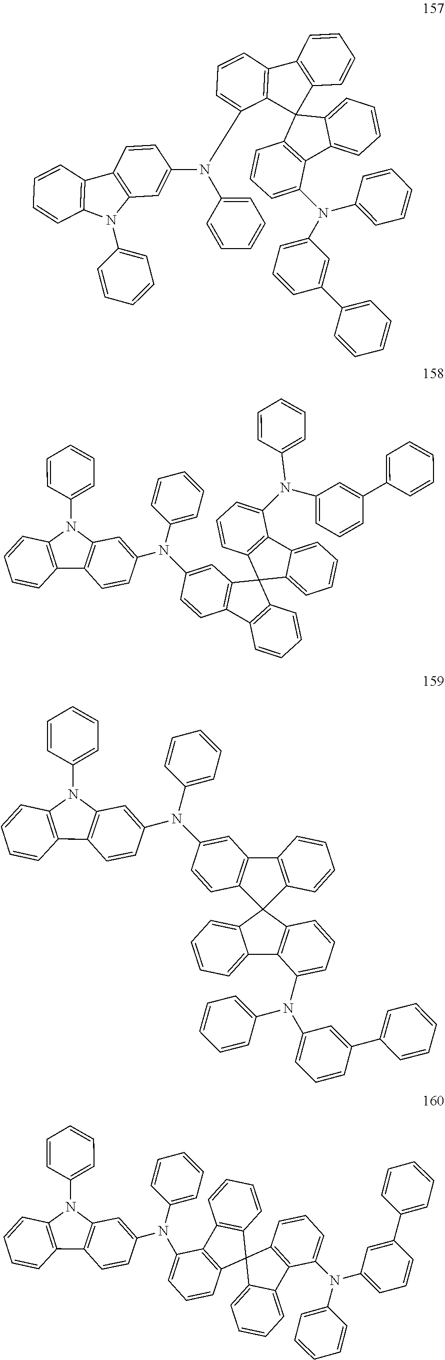 [SMILES: c1ccc(-c2cccc(N(c3ccccc3)c3cccc4c3-c3ccccc3C43c4ccccc4-c4cccc(N(c5ccccc5)c5ccc6c7ccccc7n(-c7ccccc7)c6c5)c43)c2)cc1 ; c1ccc(-c2cccc(N(c3ccccc3)c3cccc4c3-c3ccccc3C43c4ccccc4-c4ccc(N(c5ccccc5)c5ccc6c7ccccc7n(-c7ccccc7)c6c5)cc43)c2)cc1 ; c1ccc(-c2cccc(N(c3ccccc3)c3cccc4c3-c3ccccc3C43c4ccccc4-c4cc(N(c5ccccc5)c5ccc6c7ccccc7n(-c7ccccc7)c6c5)ccc43)c2)cc1 ; c1ccc(-c2cccc(N(c3ccccc3)c3cccc4c3-c3ccccc3C43c4ccccc4-c4c(N(c5ccccc5)c5ccc6c7ccccc7n(-c7ccccc7)c6c5)cccc43)c2)cc1] 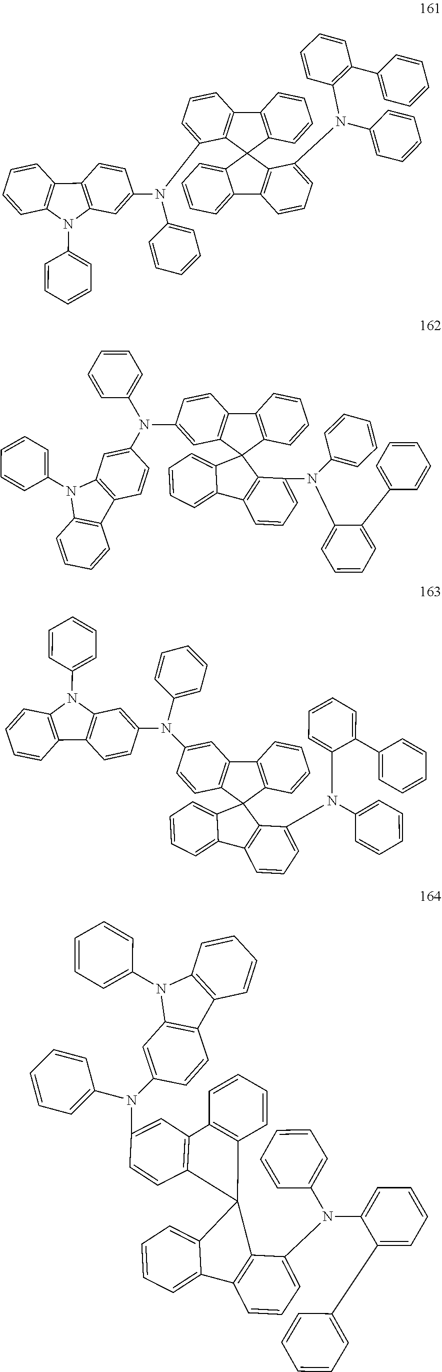 [SMILES: c1ccc(-c2ccccc2N(c2ccccc2)c2cccc3c2C2(c4ccccc4-c4cccc(N(c5ccccc5)c5ccc6c7ccccc7n(-c7ccccc7)c6c5)c42)c2ccccc2-3)cc1 ; c1ccc(-c2ccccc2N(c2ccccc2)c2cccc3c2C2(c4ccccc4-c4ccc(N(c5ccccc5)c5ccc6c7ccccc7n(-c7ccccc7)c6c5)cc42)c2ccccc2-3)cc1 ; c1ccc(-c2ccccc2N(c2ccccc2)c2cccc3c2C2(c4ccccc4-c4cc(N(c5ccccc5)c5ccc6c7ccccc7n(-c7ccccc7)c6c5)ccc42)c2ccccc2-3)cc1 ; c1ccc(-c2ccccc2N(c2ccccc2)c2cccc3c2C2(c4ccccc4-c4cc(N(c5ccccc5)c5ccc6c7ccccc7n(-c7ccccc7)c6c5)ccc42)c2ccccc2-3)cc1] 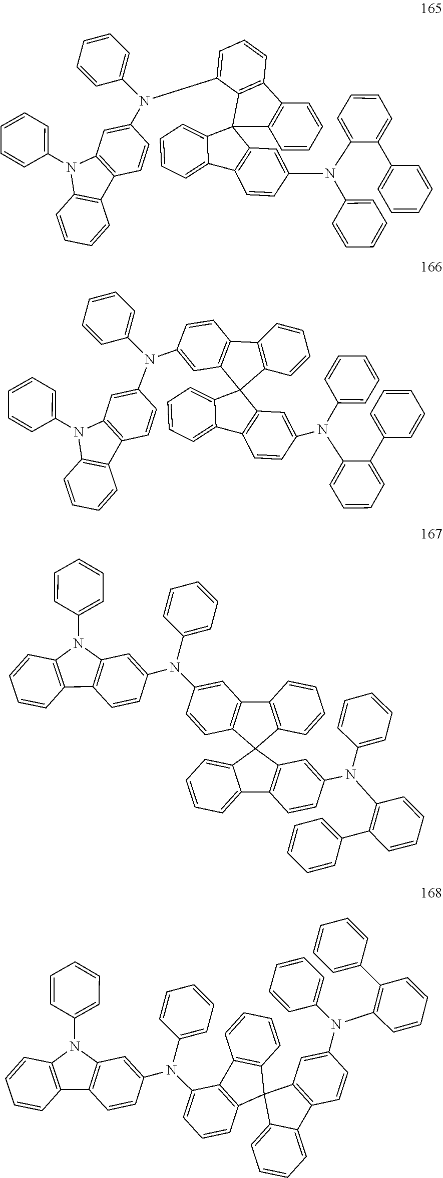 [SMILES: c1ccc(-c2ccccc2N(c2ccccc2)c2ccc3c(c2)C2(c4ccccc4-3)c3ccccc3-c3cccc(N(c4ccccc4)c4ccc5c6ccccc6n(-c6ccccc6)c5c4)c32)cc1 ; c1ccc(-c2ccccc2N(c2ccccc2)c2ccc3c(c2)C2(c4ccccc4-c4ccc(N(c5ccccc5)c5ccc6c7ccccc7n(-c7ccccc7)c6c5)cc42)c2ccccc2-3)cc1 ; c1ccc(-c2ccccc2N(c2ccccc2)c2ccc3c(c2)C2(c4ccccc4-c4cc(N(c5ccccc5)c5ccc6c7ccccc7n(-c7ccccc7)c6c5)ccc42)c2ccccc2-3)cc1 ; c1ccc(-c2ccccc2N(c2ccccc2)c2ccc3c(c2)C2(c4ccccc4-3)c3ccccc3-c3c(N(c4ccccc4)c4ccc5c6ccccc6n(-c6ccccc6)c5c4)cccc32)cc1] 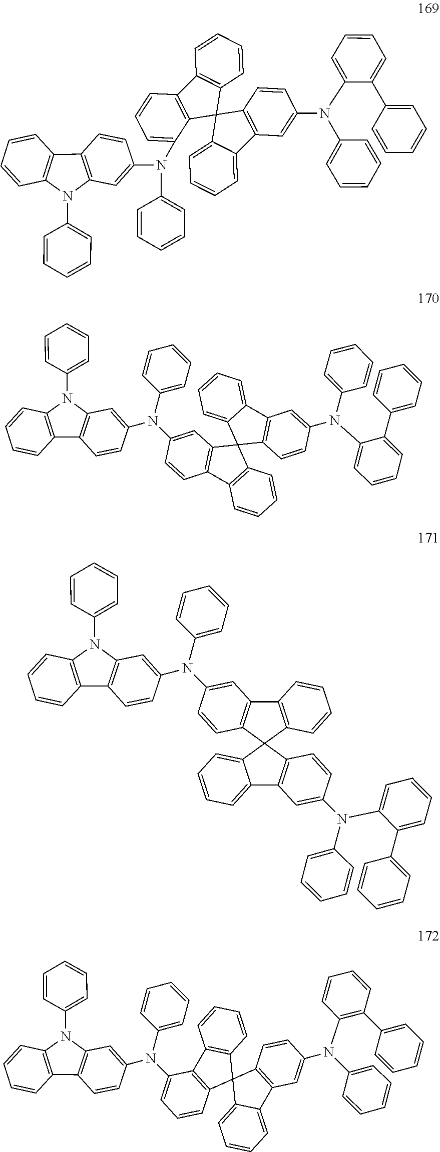 [SMILES: c1ccc(-c2ccccc2N(c2ccccc2)c2ccc3c(c2)-c2ccccc2C32c3ccccc3-c3cccc(N(c4ccccc4)c4ccc5c6ccccc6n(-c6ccccc6)c5c4)c32)cc1 ; c1ccc(-c2ccccc2N(c2ccccc2)c2ccc3c(c2)-c2ccccc2C32c3ccccc3-c3ccc(N(c4ccccc4)c4ccc5c6ccccc6n(-c6ccccc6)c5c4)cc32)cc1 ; c1ccc(-c2ccccc2N(c2ccccc2)c2ccc3c(c2)-c2ccccc2C32c3ccccc3-c3cc(N(c4ccccc4)c4ccc5c6ccccc6n(-c6ccccc6)c5c4)ccc32)cc1 ; c1ccc(-c2ccccc2N(c2ccccc2)c2ccc3c(c2)-c2ccccc2C32c3ccccc3-c3c(N(c4ccccc4)c4ccc5c6ccccc6n(-c6ccccc6)c5c4)cccc32)cc1] 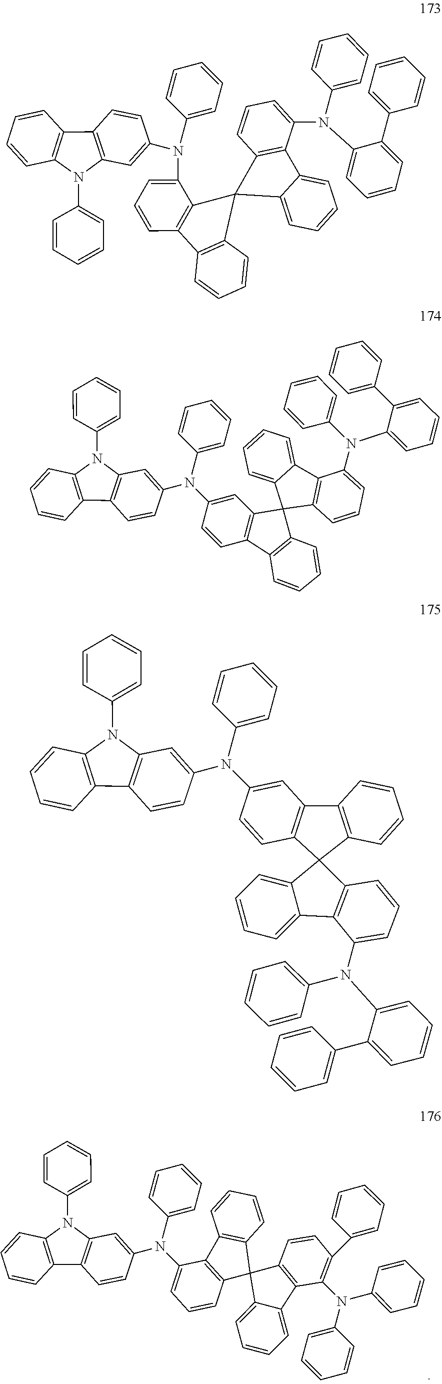 [SMILES: c1ccc(-c2ccccc2N(c2ccccc2)c2cccc3c2-c2ccccc2C32c3ccccc3-c3cccc(N(c4ccccc4)c4ccc5c6ccccc6n(-c6ccccc6)c5c4)c32)cc1 ; c1ccc(-c2ccccc2N(c2ccccc2)c2cccc3c2-c2ccccc2C32c3ccccc3-c3ccc(N(c4ccccc4)c4ccc5c6ccccc6n(-c6ccccc6)c5c4)cc32)cc1 ; c1ccc(-c2ccccc2N(c2ccccc2)c2cccc3c2-c2ccccc2C32c3ccccc3-c3cc(N(c4ccccc4)c4ccc5c6ccccc6n(-c6ccccc6)c5c4)ccc32)cc1 ; c1ccc(-c2ccc3c(c2N(c2ccccc2)c2ccccc2)-c2ccccc2C32c3ccccc3-c3c(N(c4ccccc4)c4ccc5c6ccccc6n(-c6ccccc6)c5c4)cccc32)cc1]
